(19) United States
(12) Patent Application Publication
(10) Pub. No.: US 2025/0255908 A1
(43) Pub. Date: Aug. 14, 2025

(54) COMPOSITIONS AND METHODS FOR NEURALGENESIS

(71) Applicant: The Johns Hopkins University, Baltimore, MD (US)

(72) Inventors: Thomas Hartung, Baltimore, MD (US); David Pamies, Baltimore, MD (US); Helena T. Hogberg, Baltimore, MD (US)

(21) Appl. No.: 18/922,031

(22) Filed: Oct. 21, 2024

Related U.S. Application Data
(63) Continuation of application No. 16/077,411, filed on Aug. 10, 2018, now Pat. No. 12,156,890.

(51) Int. Cl. A61K 35/30; A61K 38/41; C12N 5/00; C12N 5/074; G01N 33/50; G01N 33/58; G01N 33/68

(52) U.S. Cl. CPC A61K 35/30; A61K 38/41; C12N 5/0062; C12N 5/0696; G01N 33/50; G01N 33/58; G01N 33/6893; C12N 2501/11; C12N 2501/115; C12N 2501/13; C12N 2501/22

(57) ABSTRACT

The present invention relates to novel compositions and methods to produce 3D organ equivalents of the brain (i.e. “mini-brains”). The invention also relates to methods of using human induced pluripotent stem cells, a combination of growth and other soluble factors and gyratory shaking. Cells from healthy or diseased donors or animals can be used to allow testing different genetic backgrounds. The model can be further enhanced by using genetically modified cells, adding micro-glia or their precursors or indicator cells (e.g. with reporter genes or tracers) as well as adding endothelial cells to form a blood-brain-barrier.

### RELATED APPLICATIONS

This application claims the benefit of priority under 35 U.S.C. § 119(e) to U.S. Provisional Application No. 62/294,112, filed Feb. 11, 2016, which is incorporated herein by reference in its entirety.

### STATEMENT AS TO FEDERALLY SPONSORED RESEARCH

The invention was made with government support under the following grant awarded by the National Institute of Health (NIH): U18TR000547. The government has certain rights in the invention.

### SEQUENCE LISTING

The instant application contains a Sequence Listing which has been submitted electronically in ASCII format and is hereby incorporated by reference in its entirety. Said ASCII copy, created on Mar. 21, 2017, is named 48317-518001WO_SL.txt and is 329,024 bytes in size.

### BACKGROUND OF THE INVENTION

Simple neural in vitro systems do not reflect the physiology, cellular interactions, or genetics of mammalian brain tissue. Accordingly, there is an unmet need to develop human models of brain disorders and/or diseases.

### SUMMARY OF THE INVENTION

The present invention provides brain microphysiological systems (BMPS) that can be produced from induced pluripotent stem cells (iPSCs). Furthermore, the invention provides for reproducible BMPS that differentiate into mature neurons and glial cells (astrocytes and oligodendrocytes) in the central nervous system. This model is electrophysiologically active in a spontaneous manner and may be reproduced with patient cells. The derivation of 3D BMPS from iPSCs has applications in the study and treatment of neurological diseases.

In an aspect, the disclosure provides an in vitro brain microphysiological system (BMPS), comprising two or more neural cell types aggregated into a spheroid mass, wherein the spheroid mass has a diameter that is less than about 500 μm and the in vitro BMPS is electrophysiologically active in a spontaneous manner.

In an embodiment, the two or more neural cell types comprise at least a mature neuron and glial cell.

In an embodiment, the two or more neural cell types further comprise cells selected from the group consisting of astrocytes, polydendrocytes, oligodendrocytes, and combinations thereof.

In an embodiment, the in vitro BMPS has neural characteristics selected from the group consisting of synaptogenesis, neuron-neuron interactions, neuronal-glial interactions, axon myelination, and combinations thereof.

In an embodiment, two or more neural cell types of the in vitro BMPS express one or more biomarker selected from the group consisting of GRIN1, GAD1, GABA, TH, LMX1A, FOXO1, FOXA2, FOXO4, CNP, MBP, TH, TUBIII, NEUN, SLC1A6, and any combination thereof.

In an aspect, the disclosure provides a synthetic neurological organ comprising two or more neural cell types aggregated into a spheroid mass, wherein the spheroid mass has a diameter that is less than 500 μm and the in vitro BMPS is electrophysiologically active in a spontaneous manner.

In an embodiment, the two or more neural cell types comprise at least a mature neuron and glial cells.

In an embodiment, the mature neuron and glial cells further comprise cells selected from the group consisting of astrocytes, polydendrocytes, oligodendrocytes, and combinations thereof.

In an embodiment, the synthetic neurological organ further comprises neural characteristics selected from the group consisting of synaptogenesis, neuron-neuron interactions, neuronal-glial interactions, axon myelination, and combinations thereof.

In an embodiment, the synthetic neurological organ mimics the microenvironment of the central nervous system (CNS).

In an aspect, the disclosure provides a method of reproducibly producing an in vitro brain microphysiological system (BMPS), comprising: inducing one or more pluripotent stem cell (PSC) types; differentiating the one or more PSC types to form one or more neural progenitor cell (NPC) types; exposing the one or more NPC types to gyratory shaking or stirring; and differentiating the one or more NPC types into one or more neural cell types aggregated into a spheroid mass, wherein the spheroid mass has a diameter that is less than 500 μm.

In an embodiment, the one or more pluripotent stem cells are selected from the group consisting of human or animal embryonic stem cells, iPSC, adult stem cells, fibroblasts, embryonic fibroblasts, peripheral blood mononuclear cells, neuronal precursor cells, mesenchymal stem cells, and combinations thereof.

In an embodiment, inducing further comprises: adding micro-glia or micro-glia precursor cells.

In an embodiment, the micro-glia or micro-glia precursor cells are selected from the group consisting of monocytes, human monocytes, pro-monocyte cell lines, iPSC-derived monocytes, hematopoetic stem cells, isolated microglia, immortalized microglia, and combinations thereof.

In an embodiment, gyratory shaking comprises constant or regular gyratory shaking or stirring for 2 or more, 3 or more, 4 or more, 5 or more, 6 or more, 7 or more, or 8 or more weeks.

In an embodiment, the one or more growth factors are selected from the group consisting of GDNF, BDNF, GM-CSF, B27, basic FGF, basic EGF, NGF, CNTF, and any combination thereof.

In an aspect, the disclosure provides a method of cryopreserving an in vitro brain microphysiological system (BMPS), comprising: differentiating BMPS aggregates into one or more mature neurons; incubating the aggregates in a cryopreserving medium; and exposing the aggregates to freezing temperatures of −60° C. or colder.

In an embodiment, differentiating further comprises: inducing differentiation of one or more pluripotent stem cell types by incubation with one or more growth factors.

In an embodiment, the one or more pluripotent stem cells are selected from a group consisting of human or animal embryonic stem cells, iPSC, adult stem cells, fibroblasts, embryonic fibroblasts, peripheral blood mononuclear cells, neuronal precursor cells, mesenchymal stem cells, and combinations thereof.

In an embodiment, inducing further comprises: adding micro-glia precursor cells.

In an embodiment, micro-glia precursor cells are selected from the group consisting of monocytes, human monocytes, iPSC-derived monocytes, hematopoetic stem cells, pro-monocyte cell lines, isolated microglia, immortalized microglia, and combinations thereof.

In an embodiment, the one or more growth factors are selected from the group consisting of GDNF, BDNF, GM-CSF, B27, basic FGF, basic EGF, NGF, CNTF, and any combination thereof.

In an embodiment, the cryopreserving medium is a medium selected from the group consisting of regular cryopreservation medium (95% FBS and 5% DMSO), STEMdiff Neural Progenitor Freezing Medium (Stem Cells Technologies), solutions with cryoprotectants, and combinations thereof.

In an embodiment, exposing the aggregates to freezing temperatures further comprises freezing aggregates over a temperature gradient of about 1° C. per hour to below −60° C. over up to 48 hours.

In an embodiment, cryopreserving further comprises additives selected from the group consisting of DMSO, HES, glycerol, serum, and any combination or derivative thereof.

In an aspect, the disclosure provides a method of transporting a brain microphysiological system (BMPS) or mini-brain, comprising: producing the BMPS or mini-brain of claim 1, incubating the BMPS or mini-brain at 37° C., and maintaining the temperature at 37° C. with constant application of heat while moving the BMPS or mini-brain.

In an embodiment, maintaining the temperature comprises use of heating pads, heaters, insulation, insulated boxes, heat packs, electric blankets, chemical pads, and combinations thereof.

In an aspect, the disclosure provides a method of studying a neurological disease or disorder comprising: producing an in vitro brain microphysiological system (BMPS); exposing the in vitro BMPS to conditions that replicate or induce the neurological disease or disorder; adding an agent to treat the neurological disease or disorder; and assessing the effect of the agent on the neurological disease or disorder.

In an embodiment, the neurological disease or disorder is selected from the group consisting of neurodegenerative disorder, muscular dystrophy, Parkinson's Disease, Huntington's Disease, Autism Spectrum Disorder and other neurodevelopmental disorders, Down's Syndrome, Multiple Sclerosis, Amyotrophic lateral sclerosis, brain cancer, encephalitis, infection, trauma, stroke, and paralysis.

In an aspect, the disclosure provides a method of treating a patient having a neurological disease or disorder, comprising: extracting a stem cell from the patient with a genetic background pre-disposed for the neurological disease or disorder; producing a brain microphysiological system (BMPS) or mini-brain with the genetic background; treating the BMPS or mini-brain with an agent targeting the neurological disease or disorder; and assessing the effect of the agent on the BMPS or mini-brain.

In an embodiment, the neurological disease or disorder is selected from the group consisting of neurodegenerative disorder, muscular dystrophy, Parkinson's Disease, Huntington's Disease, Autism Spectrum Disorder and other neurodevelopmental disorders, Down's Syndrome, Multiple Sclerosis, Amyotrophic lateral sclerosis, brain cancer, encephalitis, infection, trauma, stroke, and paralysis.

In an embodiment, the BMPS includes two or more neuronal cell types that include one or more genetically modified cells. The BMPS wherein the one or more genetically modified cells include one or more reporter genes. The BMPS further comprises one or more endothelial cells capable of forming a blood-brain-barrier.

In an embodiment, the synthetic neurological organ may include two or more neural cell types that include one or more genetically modified cells. The synthetic neurological organ including one or more genetically modified cells that include one or more reporter genes. The synthetic neurological organ further comprising one or more endothelial cells capable of forming a blood-brain-barrier.

In an aspect, the disclosure provides a method of reproducibly producing an in vitro brain microphysiological system (BMPS), comprising: exposing one or more NPC types to gyratory shaking or stirring; and differentiating the one or more NPC types into one or more neural cell types aggregated into a spheroid mass, wherein the spheroid mass has a diameter that is less than 500 μm.

In an embodiment, the spheroid mass has a diameter that is less than about 450 μm, 400 μm, 350 μm, or 300 μm, or a diameter that is between about 350 μm and about 300 μm, or a diameter that is between about 330 μm and about 300 μm, or a diameter that is about 310 μm.

In an embodiment, the two or more neural cell types of the in vitro BMPS express one or more biomarker selected from the group consisting of GRIN1, GAD1, GABA, TH, LMX1A, FOXO1, FOXA2, FOXO4, CNP, MBP, TH, TUBIII, NEUN, SLC1A6, and any combination thereof.

In an embodiment, the two or more neural cell types of the in vitro BMPS express one or more biomarker selected from the group consisting of GRIN1, GAD1, GABA, TH, LMX1A, FOXO1, FOXA2, FOXO4, CNP, MBP, TH, TUBIII, NEUN, SLC1A6, and any combination thereof.

In an embodiment, the two or more neural cell types of the in vitro BMPS express one or more biomarker selected from the group consisting of GRIN1, GAD1, GABA, TH, LMX1A, FOXO1, FOXA2, FOXO4, CNP, MBP, TH, TUBIII, NEUN, SLC1A6, and any combination thereof.

In an embodiment, inducing comprises a single PSC.

In an embodiment, the an in vitro brain microphysiological system (BMPS) may be produced according to the above described method.

It is also contemplated within the scope of the invention that the addition of other cells inside (see e.g., FIG. 6) and outside (see e.g., FIG. 7) the BMPS may be used to modify the structure/composition of the BMPS, such as, e.g., by forming a blood-brain-barrier. It is also contemplated that the BMPS described herein may include genetically modified pluripotent stem cells, or be combined with other organoids (see e.g., Example 11).

### Definitions

By “agent” is meant any small compound, antibody, nucleic acid molecule, or polypeptide, or fragments thereof.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.

By “ameliorate” is meant decrease, suppress, attenuate, diminish, arrest, or stabilize the development or progression of a disease.

In this disclosure, “comprises,” “comprising,” “containing,” and “having” and the like may have the meaning ascribed to them in U.S. Patent law and may mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of the analyte to be detected.

By “effective amount” is meant the amount of an agent needed to ameliorate the symptoms of a neurological disease relative to an untreated patient. The effective amount of active agent(s) used to practice the present invention for therapeutic treatment of a neurological disease varies depending upon the manner of administration, the age, body weight, and general health of the subject. Ultimately, the attending physician or veterinarian will decide the appropriate amount and dosage regimen. Such amount is referred to as an “effective” amount.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, 100, 200, 300, 400, 500, 600, 700, 800, 900, or 1000 nucleotides or amino acids, or more.

By “gene” is meant a locus (or region) of DNA that encodes a functional RNA or protein product, and is the molecular unit of heredity.

By “marker” is meant any protein or polynucleotide having an alteration in expression level or activity that is associated with a disease or disorder.

By “modulate” is meant alter (increase or decrease). Such alterations are detected by standard art known methods such as those described herein.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9.

With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

By “reduces” is meant a negative alteration of at least 10%, 25%, 50%, 75%, or 100%.

By “reference” is meant a standard or control condition.

By “pluripotency” is meant stem cells with the potential to differentiate into any of the three germ layers: endoderm (e.g., interior stomach lining, gastrointestinal tract, the lungs), mesoderm (e.g., muscle, bone, blood, urogenital), or ectoderm (e.g., epidermal tissues and nervous system). However, one of skill in the art will understand that cell pluripotency is a continuum, ranging from the completely pluripotent cell that can form every cell of the embryo proper, e.g., embryonic stem cells and iPSCs (see below), to the incompletely or partially pluripotent cell that can form cells of all three germ layers but that may not exhibit all the characteristics of completely pluripotent cells. Induced pluripotent stem cells, commonly abbreviated as iPS cells or iPSCs are a type of pluripotent stem cell artificially derived from a non-pluripotent cell, typically an adult somatic cell, by inducing a “forced” expression of certain genes and transcription factors. These transcription factors play a key role in determining the state of these cells and also highlight the fact that these somatic cells do preserve the same genetic information as early embryonic cells. The ability to induce cells into a pluripotent state was initially pioneered using mouse fibroblasts and four transcription factors, Oct4, Sox2, Klf4 and c-Myc; —a process called reprogramming. The successful induction of human iPSCs derived from human dermal fibroblasts has been performed using methods similar to those used for the induction of mouse cells. These induced cells exhibit similar traits to those of embryonic stem cells (ESCs) but do not require the use of embryos. Some of the similarities between ESCs and iPSCs include pluripotency, morphology, self-renewal ability, a trait that implies that they can divide and replicate indefinitely, and gene expression.

By “stem cells” is meant undifferentiated biological cells that can differentiate into specialized cells and can divide (through mitosis) to produce more stem cells. They are found in multicellular organisms. In mammals, there are two broad types of stem cells: embryonic stem cells, which are isolated from the inner cell mass of blastocysts, and adult stem cells, which are found in various tissues. In adult organisms, stem cells and progenitor cells act as a repair system for the body, replenishing adult tissues. In a developing embryo, stem cells can differentiate into all the specialized cells—ectoderm, endoderm and mesoderm (see induced pluripotent stem cells)—but also maintain the normal turnover of regenerative organs, such as blood, skin, or intestinal tissues. There are three known accessible sources of autologous adult stem cells in humans: 1. Bone marrow, which requires extraction by harvesting, that is, drilling into bone (typically the femur or iliac crest). 2. Adipose tissue (lipid cells), which requires extraction by liposuction. 3. Blood, which requires extraction through apheresis, wherein blood is drawn from the donor (similar to a blood donation), and passed through a machine that extracts the stem cells and returns other portions of the blood to the donor. Stem cells can also be taken from umbilical cord blood just after birth. Of all stem cell types, autologous harvesting involves the least risk. By definition, autologous cells are obtained from one's own body.

By “subject” is meant a mammal, including, but not limited to, a human or non-human mammal, such as a bovine, equine, canine, ovine, or feline.

As used herein, the terms “treat,” treating,” “treatment,” and the like refer to reducing or ameliorating a neurological disorder and/or symptoms associated therewith. It will be appreciated that, although not precluded, treating a disorder or condition does not require that the disorder, condition or symptoms associated therewith be completely eliminated.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

Unless specifically stated or obvious from context, as used herein, the term “or” is understood to be inclusive. Unless specifically stated or obvious from context, as used herein, the terms “a,” “an,” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

A “therapeutically effective amount” is an amount sufficient to effect beneficial or desired results, including clinical results. An effective amount can be administered in one or more administrations.

By “GRIN1 polypeptide” (or glutamate ionotropic receptor NMDA type subunit 1) is meant a polypeptide or fragment thereof having at least about 85% amino acid identity to NCBI Accession No. Q05586.

| (SEQ ID NO: 1) | |
|---|---|
| 1 | mstmrlltla llfscsvara acdpkivnig avlstrkheq mfreavnqan krhgswkiql |
| 61 | natsvthkpn aiqmalsvce dlissqvyai lvshpptpnd hftptpvsyt agfyripvlg |
| 121 | lttrmsiysd ksihlsflrt vppyshqssv wfemmrvysw nhiillvsdd hegraaqkrl |
| 181 | etlleeresk aekvlqfdpg tknvtallme akelearvii lsaseddaat vyraaamlnm |
| 241 | tgsgyvwlvg ereisgnalr yapdgilglq lingknesah isdavgvvaq avhelleken |
| 301 | itdpprgcvg ntniwktgpl fkrvlmssky adgvtgrvef nedgdrkfan ysimnlqnrk |
| 361 | lvqvgiyngt hvipndrkii wpggetekpr gyqmstrlki vtihqepfvy vkptlsdgtc |
| 421 | keeftvngdp vkkvictgpn dtspgsprht vpqccygfci dlliklartm nftyevhlva |
| 481 | dgkfgtqerv nnsnkkewng mmgellsgqa dmivapltin neraqyiefs kpfkyqglti |
| 541 | lvkkeiprst ldsfmqpfqs tlwllvglsv hvvavmlyll drfspfgrfk vnseeeeeda |
| 601 | ltlssamwfs wgvllnsgig egaprsfsar ilgmvwagfa miivasytan laaflvldrp |
| 661 | eeritgindp rlrnpsdkfi yatvkqssvd iyfrrqvels tmyrhmekhn yesaaeaiqa |
| 721 | vrdnklhafi wdsavlefea sqkcdlvttg elffrsgfgi gmrkdspwkq nvslsilksh |
| 781 | engfmedldk twvryqecds rsnapatltf enmagvfmlv aggivagifl ifieiaykrh |
| 841 | kdarrkqmql afaavnvwrk nlqdrksgra epdpkkkatf raitstlass fkrrrsskdt |
| 901 | stgggrgalq nqkdtvlprr aiereegqlq lcsrhres |

By “GRIN1 nucleic acid molecule” (or glutamate ionotropic receptor NMDA type subunit 1) is meant a polynucleotide encoding an GRIN1 polypeptide. An exemplary GRIN1 nucleic acid molecule (e.g., mRNA) is provided at NCBI Accession No. NM_007327.

| (SEQ ID NO: 2) | |
|---|---|
| 1 | gtcgccgcag cgtccggacc ggaaccagcg ccgtccgcgg agccgccgcc gccgccgccg |
| 61 | ggccctttcc aagccgggcg ctcggagctg tgcccggccc cgcttcagca ccgcggacag |
| 121 | cgccggccgc gtggggctga gccccgagcc cccgcgcacg cttcagcgcc ccttccctcg |
| 181 | gccgacgtcc cgggaccgcc gctccggggg agacgtggcg tccgcagccc gcggggccgg |
| 241 | gcgagcgcag gacggcccgg aagccccgcg ggggatgcgc cgagggcccc gcgttcgcgc |
| 301 | cgcgcagagc caggcccgcg gcccgagccc atgagcacca tgcgcctgct gacgctcgcc |
| 361 | ctgctgttct cctgctccgt cgcccgtgcc gcgtgcgacc ccaagatcgt caacattggc |
| 421 | gcggtgctga gcacgcggaa gcacgagcag atgttccgcg aggccgtgaa ccaggccaac |
| 481 | aagcggcacg gctcctggaa gattcagctc aatgccacct ccgtcacgca caagcccaac |
| 541 | gccatccaga tggctctgtc ggtgtgcgag gacctcatct ccagccaggt ctacgccatc |
| 601 | ctagttagcc atccacctac ccccaacgac cacttcactc ccacccctgt ctcctacaca |
| 661 | gccggcttct accgcatacc cgtgctgggg ctgaccaccc gcatgtccat ctactcggac |
| 721 | aagagcatcc acctgagctt cctgcgcacc gtgccgccct actcccacca gtccagcgtg |
| 781 | tggtttgaga tgatgcgtgt ctacagctgg aaccacatca tcctgctggt cagcgacgac |
| 841 | cacgagggcc gggcggctca gaaacgcctg gagacgctgc tggaggagcg tgagtccaag |
| 901 | gcagagaagg tgctgcagtt tgacccaggg accaagaacg tgacggccct gctgatggag |
| 961 | gcgaaagagc tggaggcccg ggtcatcatc ctttctgcca gcgaggacga tgctgccact |
| 1021 | gtataccgcg cagccgcgat gctgaacatg acgggctccg ggtacgtgtg gctggtcggc |
| 1081 | gagcgcgaga tctcggggaa cgccctgcgc tacgccccag acggcatcct cgggctgcag |
| 1141 | ctcatcaacg gcaagaacga gtcggcccac atcagcgacg ccgtgggcgt ggtggcccag |
| 1201 | gccgtgcacg agctcctcga gaaggagaac atcaccgacc cgccgcgggg ctgcgtgggc |
| 1261 | aacaccaaca tctggaagac cgggccgctc ttcaagagag tgctgatgtc ttccaagtat |
| 1321 | gcggatgggg tgactggtcg cgtggagttc aatgaggatg gggaccggaa gttcgccaac |
| 1381 | tacagcatca tgaacctgca gaaccgcaag ctggtgcaag tgggcatcta caatggcacc |
| 1441 | cacgtcatcc ctaatgacag gaagatcatc tggccaggcg gagagacaga gaagcctcga |
| 1501 | gggtaccaga tgtccaccag actgaagatt gtgacgatcc accaggagcc cttcgtgtac |
| 1561 | gtcaagccca cgctgagtga tgggacatgc aaggaggagt tcacagtcaa cggcgaccca |
| 1621 | gtcaagaagg tgatctgcac cgggcccaac gacacgtcgc cgggcagccc ccgccacacg |
| 1681 | gtgcctcagt gttgctacgg cttttgcatc gacctgctca tcaagctggc acggaccatg |
| 1741 | aacttcacct acgaggtgca cctggtggca gatggcaagt tcggcacaca ggagcgggtg |
| 1801 | aacaacagca acaagaagga gtggaatggg atgatgggcg agctgctcag cgggcaggca |
| 1861 | gacatgatcg tggcgccgct aaccataaac aacgagcgcg cgcagtacat cgagttttcc |
| 1921 | aagcccttca agtaccaggg cctgactatt ctggtcaaga aggagattcc ccggagcacg |
| 1981 | ctggactcgt tcatgcagcc gttccagagc acactgtggc tgctggtggg gctgtcggtg |
| 2041 | cacgtggtgg ccgtgatgct gtacctgctg gaccgcttca gccccttcgg ccggttcaag |
| 2101 | gtgaacagcg aggaggagga ggaggacgca ctgaccctgt cctcggccat gtggttctcc |
| 2161 | tggggcgtcc tgctcaactc cggcatcggg gaaggcgccc ccagaagctt ctcagcgcgc |
| 2221 | atcctgggca tggtgtgggc cggctttgcc atgatcatcg tggcctccta caccgccaac |
| 2281 | ctggcggcct tcctggtgct ggaccggccg gaggagcgca tcacgggcat caacgaccct |
| 2341 | cggctgagga acccctcgga caagtttatc tacgccacgg tgaagcagag ctccgtggat |
| 2401 | atctacttcc ggcgccaggt ggagctgagc accatgtacc ggcatatgga gaagcacaac |
| 2461 | tacgagagtg cggcggaggc catccaggcc gtgagagaca acaagctgca tgccttcatc |
| 2521 | tgggactcgg cggtgctgga gttcgaggcc tcgcagaagt gcgacctggt gacgactgga |
| 2581 | gagctgtttt tccgctcggg cttcggcata ggcatgcgca aagacagccc ctggaagcag |
| 2641 | aacgtctccc tgtccatcct caagtcccac gagaatggct tcatggaaga cctggacaag |
| 2701 | acgtgggttc ggtatcagga atgtgactcg cgcagcaacg cccctgcgac ccttactttt |
| 2761 | gagaacatgg ccggggtctt catgctggta gctgggggca tcgtggccgg gatcttcctg |
| 2821 | attttcatcg agattgccta caagcggcac aaggatgctc gccggaagca gatgcagctg |
| 2881 | gcctttgccg ccgttaacgt gtggcggaag aacctgcagg atagaaagag tggtagagca |
| 2941 | gagcctgacc ctaaaaagaa agccacattt agggctatca cctccaccct ggcttccagc |
| 3001 | ttcaagaggc gtaggtcctc caaagacacg agcaccgggg gtggacgcgg cgctttgcaa |
| 3061 | aaccaaaaag acacagtgct gccgcgacgc gctattgaga gggaggaggg ccagctgcag |
| 3121 | ctgtgttccc gtcataggga gagctgagac tccccgcccg ccctcctctg ccccctcccc |
| 3181 | cgcagacaga cagacagacg gacgggacag cggcccggcc cacgcagagc cccggagcac |
| 3241 | cacggggtcg ggggaggagc acccccagcc tcccccaggc tgcgcctgcc cgcccgccgg |
| 3301 | ttggccggct ggccggtcca ccccgtcccg gccccgcgcg tgcccccagc gtggggctaa |
| 3361 | cgggcgcctt gtctgtgtat ttctattttg cagcagtacc atcccactga tatcacgggc |
| 3421 | ccgctcaacc tctcagatcc ctcggtcagc accgtggtgt gaggcccccg gaggcgccca |
| 3481 | cctgcccagt tagcccggcc aaggacactg atgggtcctg ctgctcggga aggcctgagg |
| 3541 | gaagcccacc cgccccagag actgcccacc ctgggcctcc cgtccgtccg cccgcccacc |
| 3601 | ccgctgcctg gcgggcagcc cctgctggac caaggtgcgg accggagcgg ctgaggacgg |
| 3661 | ggcagagctg agtcggctgg gcagggccgc agggcgctcc ggcagaggca gggccctggg |
| 3721 | gtctctgagc agtggggagc gggggctaac tggccccagg cggaggggct tggagcagag |
| 3781 | acggcagccc catccttccc gcagcaccag cctgagccac agtggggccc atggccccag |
| 3841 | ctggctgggt cgcccctcct cgggcgcctg cgctcctctg cagcctgagc tccaccctcc |
| 3901 | cctcttcttg cggcaccgcc cacccacacc ccgtctgccc cttgacccca cacgccgggg |
| 3961 | ctggccctgc cctcccccac ggccgtccct gacttcccag ctggcagcgc ctcccgccgc |
| 4021 | ctcgggccgc ctcctccaga ctcgagaggg ctgagcccct cctctcctcg tccggcctgc |
| 4081 | agcccagaac gggcctcccc gggggtcccc ggacgctggc tcgggactgt cttcaaccct |
| 4141 | gccctgcacc ttgggcacgg gagagcgcca cccgcccgcc cccgccctcg ctccgggtgc |
| 4201 | gtgaccggcc cgccaccttg tacagaacca gcactcccag ggcccgagcg cgtgccttcc |
| 4261 | ccgtgcggcc cgtgcgcagc cgcgctctgc ccctccgtcc ccagggtgca ggcgcgcacc |
| 4321 | gcccaacccc cacctcccgg tgtatgcagt ggtgatgcct aaaggaatgt cacgcagttt |
| 4381 | tcaaaaaaaa aaaaaaaaaa |

By “GAD1 polypeptide” (or glutamate decarboxylase 1) is meant a polypeptide or fragment thereof having at least about 85% amino acid identity to NCBI Accession No. Q99259.

| (SEQ ID NO: 3) | |
|---|---|
| 1 | masstpsssa tssnagadpn ttnlrpttyd twcgvahgct rklglkicgf lqrtnsleek |
| 61 | srlvsafker qssknllsce nsdrdarfrr tetdfsnlfa rdllpaknge eqtvqfllev |
| 121 | vdillnyvrk tfdrstkvld fhhphqlleg megfnlelsd hpesleqilv dcrdtlkygv |
| 181 | rtghprffnq lstgldiigl agewltstan tnmftyeiap vfvlmeqitl kkmreivgws |
| 241 | skdgdgifsp ggaisnmysi maarykyfpe vktkgmaavp klvlftseqs hysikkagaa |
| 301 | lgfgtdnvil ikcnergkii padfeakile akqkgyvpfy vnatagttvy gafdpiqeia |
| 361 | dicekynlwl hvdaawgggl lmsrkhrhkl ngieransvt wnphkmmgvl lqcsailvke |
| 421 | kgilqgcnqm cagylfqpdk qydvsydtgd kaiqcgrhvd ifkfwlmwka kgtvgfenqi |
| 481 | nkclelaeyl yakiknreef emvfngepeh tnvcfwyipq slrgvpdspq rreklhkvap |
| 541 | kikalmmesg ttmvgyqpqg dkanffrmvi snpaatqsdi dflieeierl gqdl |

By “GAD1 nucleic acid molecule” (or glutamate decarboxylase 1) is meant a polynucleotide encoding an GAD1 polypeptide. An exemplary GAD1 nucleic acid molecule (e.g., mRNA) is provided at NCBI Accession No. BC036552.

| (SEQ ID NO: 4) | |
|---|---|
| 1 | agcgtgtggt agaggagaaa cgctgaaacc ggaccgaaac ctcgccctag gcttagcgat |
| 61 | ggctaaaaac cggctgggac aagagggagg caagcaacat tccgactcgc tgctttctgg |
| 121 | ctgtctggag tgcaaggtga ctgtggttct tctctggcca agtccgaggg agaacgtaaa |
| 181 | gatatgggcc tttttccccc tctcaccttg tctcaccaaa gtccctagtc cccggagcag |
| 241 | ttagcctctt tctttccagg gaattagcca gacacaacaa cgggaaccag acaccgaacc |
| 301 | agacatgccc gccccgtgcg ccctcccccc gctggcccac acgccggctg ctgagtgccc |
| 361 | aatggggctt gtagcggctc ggctggaaaa tcgctcactg agcgctcccc tgtgctccta |
| 421 | gcccagtccc ccacaccctt gcgtcttgta ctggccttgg acccccaccc cgaccccgac |
| 481 | cccgcctcgt ctcggcgctt cactccaggt cgcgccgatg caccgccaga ctcgagagcg |
| 541 | gcccagggct acgctccctg cgccccagta ccggagctag cgcgcacgtc tcctccgctg |
| 601 | cccccacccc tgcgcacccc taccaggcag gctcgctgcc tttcctccct cttgtctctc |
| 661 | cagagccgga tcttcaaggg gagcctccgt gcccccggct gctcagtccc tccggtgtgc |
| 721 | aggaccccgg aagtcctccc cgcacagctc tcgcttctct ttgcagcctg tttctgcgcc |
| 781 | ggaccagtcg aggactctgg acagtagagg ccccgggacg accgagctga tggcgtcttc |
| 841 | gaccccatct tcgtccgcaa cctcctcgaa cgcgggagcg gaccccaata ccactaacct |
| 901 | gcgccccaca acgtacgata cctggtgcgg cgtggcccat ggatgcacca gaaaactggg |
| 961 | gctcaagatc tgcggcttct tgcaaaggac caacagcctg gaagagaaga gtcgccttgt |
| 1021 | gagtgccttc aaggagaggc aatcctccaa gaacctgctt tcctgtgaaa acagcgaccg |
| 1081 | ggatgcccgc ttccggcgca cagagactga cttctctaat ctgtttgcta gagatctgct |
| 1141 | tccggctaag aacggtgagg agcaaaccgt gcaattcctc ctggaagtgg tggacatact |
| 1201 | cctcaactat gtccgcaaga catttgatcg ctccaccaag gtgctggact ttcatcaccc |
| 1261 | acaccagttg ctggaaggca tggagggctt caacttggag ctctctgacc accccgagtc |
| 1321 | cctggagcag atcctggttg actgcagaga caccttgaag tatggggttc gcacaggtca |
| 1381 | tcctcgattt ttcaaccagc tctccactgg attggatatt attggcctag ctggagaatg |
| 1441 | gctgacatca acggccaata ccaacatgtt tacatatgaa attgcaccag tgtttgtcct |
| 1501 | catggaacaa ataacactta agaagatgag agagatagtt ggatggtcaa gtaaagatgg |
| 1561 | tgatgggata ttttctcctg ggggcgccat atccaacatg tacagcatca tggctgctcg |
| 1621 | ctacaagtac ttcccggaag ttaagacaaa gggcatggcg gctgtgccta aactggtcct |
| 1681 | cttcacctca gaacagagtc actattccat aaagaaagct ggggctgcac ttggctttgg |
| 1741 | aactgacaat gtgattttga taaagtgcaa tgaaaggggg aaaataattc cagctgattt |
| 1801 | tgaggcaaaa attcttgaag ccaaacagaa gggatatgtt cccttttatg tcaatgcaac |
| 1861 | tgctggcacg actgtttatg gagcttttga tccgatacaa gagattgcag atatatgtga |
| 1921 | gaaatataac ctttggttgc atgtcgatgg atttaacttc tcacaattgg ccaataggat |
| 1981 | catctgcctt gctactgaac taatgactaa caaaggctgt gtcacgtggc atcccaacta |
| 2041 | ttcagtaaac atgcatcatg gctgcctggg gaggtgggct gctcatgtcc aggaagcacc |
| 2101 | accataaact caacggcata gaaagggcca actcagtcac ctggaaccct cacaagatga |
| 2161 | tgggcgtgct gttgcagtgc tctgccattc tcgtcaagga aaagggtata ctccaaggat |
| 2221 | gcaaccagat gtgtgcagga tacctcttcc agccagacaa gcagtatgat gtctcctacg |
| 2281 | acaccgggga caaggcaatt cagtgtggcc gccacgtgga tatcttcaag ttctggctga |
| 2341 | tgtggaaagc aaagggcaca gtgggatttg aaaaccagat caacaaatgc ctggaactgg |
| 2401 | ctgaatacct ctatgccaag attaaaaaca gagaagaatt tgagatggtt ttcaatggcg |
| 2461 | agcctgagca cacaaacgtc tgtttttggt atattccaca aagcctcagg ggtgtgccag |
| 2521 | acagccctca acgacgggaa aagctacaca aggtggctcc aaaaatcaaa gccctgatga |
| 2581 | tggagtcagg tacgaccatg gttggctacc agccccaagg ggacaaggcc aacttcttcc |
| 2641 | ggatggtcat ctccaaccca gccgctaccc agtctgacat tgacttcctc attgaggaga |
| 2701 | tagaaagact gggccaggat ctgtaatcat ccttcgcaga acatgagttt atgggaatgc |
| 2761 | cttttccctc tggcactcca gaacaaacct ctatatgttg ctgaaacaca caggccattt |
| 2821 | cattgaggga aaacataata tcttgaagaa tattgttaaa accttactta aagcttgttt |
| 2881 | gttctagtta gcaggaaata gtgttctttt taaaaagttg cacattagga acagagtata |
| 2941 | tatgtacagt tatacatacc tctctctata tatacatgta tagtgagtgt ggcttagtaa |
| 3001 | tagatcacgg catgtttccc gctccaagag aattcacttt accttcagca gttaccgagg |
| 3061 | agctaaacat gctgccaacc agcttgtcca acaactccag gaaaactgtt tttcaaaacg |
| 3121 | ccatgtccta ggggccaagg gaaatgctgt tggtgagaat cgacctcact gtcagcgttt |
| 3181 | ctccacctga agtgatgatg gatgagaaaa aacaccacca aatgacaagt cacaccctcc |
| 3241 | ccattagtat cctgttaggg gaaaatagta gcagagtcat tgttacaggt gtactatggc |
| 3301 | tgtattttta gagattaatt tgtgtagatt gtgtaaattc ctgttgtctg accttggtgg |
| 3361 | tgggaggggg agactatgtg tcatgatttc aatgattgtt taattgtagg tcaatgaaat |
| 3421 | atttgcttat ttatattcag agatgtacca tgttaaagag gcgtcttgta ttttcttccc |
| 3481 | atttgtaatg tatcttattt atatatgaag taagttctga aaactgttta tggtattttc |
| 3541 | gtgcatttgt gagccaaaga gaaaagatta aaattagtga gatttgtatt tatattagag |
| 3601 | tgcccttaaa ataatgattt aagcatttta ctgtctgtaa gagaattcta agattgtaca |
| 3661 | taaagtcata tatatggaaa tcctgttact taaatagcat ctgctcttct cttacgctct |
| 3721 | ctgtctggct gtacgtctgg tgttctcaat gcttttctag caactgttgg ataataacta |
| 3781 | gatctcctgt aattttgtag tagttgatga ccaatctctg ttactcgctt agctgaaacc |
| 3841 | taaggcaaca tttccgaaga ccttctgaag atctcagata aagtgaccag gctcacaact |
| 3901 | gtttttgaag aagggaaatt cacactgtgc gttttagagt atgcaagaag aatataaata |
| 3961 | aataaaaata ttctccatgg agaatttgaa caaaaaaaaa aaaaaaa |

By “GABA polypeptide” (or gamma-Aminobutyric acid) is meant a polypeptide or fragment thereof having at least about 85% amino acid identity to NCBI Accession No. P30531.

| (SEQ ID NO: 5) | |
|---|---|
| 1 | matngskvad gqistevsea pvandkpktl vvkvqkkaad lpdrdtwkgr fdflmscvgy |
| 61 | aiglgnvwrf pylcgknggg aflipyfltl ifagvplfll ecslgqytsi gglgvwklap |
| 121 | mfkgvglaaa vlsfwlniyy iviiswaiyy lynsftttlp wkqcdnpwnt drcfsnysmv |
| 181 | nttnmtsavv efwernmhqm tdgldkpgqi rwplaitlai awilvyfciw kgvgwtgkvv |
| 241 | yfsatypyim liilffrgvt lpgakegilf yitpnfrkls dsevwldaat qiffsyglgl |
| 301 | gslialgsyn sfhnnvyrds iivccinsct smfagfvifs ivgfmahvtk rsiadvaasg |
| 361 | pglaflaype avtqlpispl wailffsmll mlgidsqfct vegfitalvd eyprllrnrr |
| 421 | elfiaavcii syliglsnit qggiyvfklf dyysasgmsl lflvffecvs iswfygvnrf |
| 481 | ydniqemvgs rpciwwklcw sfftpiivag vfifsavqmt pltmgnyvfp kwgqgvgwlm |
| 541 | alssmvlipg ymaymfltlk gslkqriqvm vqpsedivrp engpeqpqag sstskeayi |

By “GABA nucleic acid molecule” (or gamma-Aminobutyric acid) is meant a polynucleotide encoding an GABA polypeptide. An exemplary GABA nucleic acid molecule (e.g., mRNA) is provided at NCBI Accession No. U76343.

| (SEQ ID NO: 6) | |
|---|---|
| 1 | gtagcttcac taaggtggga tggatagcag ggtctcaggc acaaccagta atggagagac |
| 61 | aaaaccantg tatcacaaga tggagtttgt gctgtcagtg gctggggaga tcattggctt |
| 121 | aggcaacgtc tggaggtttc cctatctctg ctacaaaaat gggggaggtg ccttcttcat |
| 181 | cccctacctc gtcttcctct ttacctgtgg cattcctgtc ttccttctgg agacagcact |
| 241 | aggccagtac actagccagg gaggcgtcac agcctggagg aagatctgcc ccatctttga |
| 301 | gggcattggc tatgcctccc agatgatcgt catcctcctc aacgtctact acatcattgt |
| 361 | gttggcctgg gccctgttct acctcttcag cagcttcacc atcgacctgc cctggggcgg |
| 421 | ctgctaccat gagtggaaca cagaacactg tatggagttc cagaagacca acggctccct |
| 481 | gaatggtacc tctgagaatg ccacctctcc tgtcatcgag ttctgggagc ggcgggtctt |
| 541 | gaagatctct gatgggatcc agcacctggg ggccctgcgc tgggagctgg ctctgtgcct |
| 601 | cctgctggcc tgggtcatct gctacttctg catctggaag ggggtgaagt ccacaggcaa |
| 661 | ggtggtgtac ttcacggcca catttcctta cctcatgctg gtggtcctgt taattcgagg |
| 721 | ggtgacgttg cctggggcag cccaaggaat tcagttttac ctgtacccaa acctcacgcg |
| 781 | tctgtgggat ccccaggtgt ggatggatgc aggcacccag atattcttct ccttcgccat |
| 841 | ctgtcttggg tgcctgacag ccctgggcag ctacaacaag taccacaaca actgctacag |
| 901 | cggcaccagc tttgtggccg gctttgccat cttctccatc ctgggcttca tgtctcagga |
| 961 | gcagggggtg cccatttctg aggtggccga gtcaggccct ggcctggctt tcatcgctta |
| 1021 | cccgcgggct gtggtgatgc tgcccttctc tcctctctgg gcctgctgtt tcttcttcat |
| 1081 | ggtcgttctc ctgggactgg atagccagtt tgtgtgtgta gaaagcctgg tgacagcgct |
| 1141 | ggtggacatg taccctcacg tgttccgcaa gaagaaccgg agggaagtcc tcatccttgg |
| 1201 | agtatctgtc gtctccttcc ctgtggggct gatcatgctc acagagggcg gaatgtacgt |
| 1261 | gttccagctc tttgactact atgcggccag tggcatgtgc ctcctgttcg tggccatctt |
| 1321 | cgagtccctc tgtgtggctt gggtttacgg agccaagcgc ttctacgaca acatcgaaga |
| 1381 | catgattggg tacaggccat ggcctcttat caaatactgt tggctcttcc tcacaccagc |
| 1441 | tgtgtgcaca gccacctttc tcttctccct gataaagtac actccgctga cctacaacaa |
| 1501 | gaagtacacg tacccgtggt ggggcgatgc cctgggctgg ctcctggctc tgtcctcctg |
| 1561 | gtctgcattc ctgcctggag cctctacaga ctcggaaccc tcaagggccc cttcagagag |
| 1621 | agaatccgtc agctcatgtg cccagccgag gacctgcccc agcggaaccc agcaggaccc |
| 1681 | tcggctcccg ccacccccag gacctcactg ctcagactca cagagctaga gtctcactgc |
| 1741 | tagggggcag gcccttggat ggtgcctgtg tgcctggcct tggggatggc tgtggaggga |
| 1801 | acgtggcaga agcagcccca tgtgttccct gcccccgacc tggagtggat aagacaagag |
| 1861 | gggtattttg gagtccacct gctgagctgg aggcctccca ctgcaacttt tcagctcagg |
| 1921 | ggttgttgaa cagatgtgaa aaggccagtg ccaagagtgt ccctcggaga cccttgaagg |
| 1981 | c |

By “TH polypeptide” (or Tyrosine Hydroxylase) is meant a polypeptide or fragment thereof having at least about 85% amino acid identity to NCBI Accession No. NP_002692.

| (SEQ ID NO: 7) | |
|---|---|
| 1 | mptpdattpq akgfrravse ldakqaeaim vrgqgapgps ltgspwpgta apaasytptp |
| 61 | rsprfigrrq sliedarker eaavaaaaaa vpsepgdple avafeekegk avlnllfspr |
| 121 | atkpsalsra vkvfetfeak ihhletrpaq rpraggphle yfvrlevrrg dlaallsgvr |
| 181 | qvsedvrspa gpkvpwfprk vseldkchhl vtkfdpdldl dhpgfsdqvy rqrrkliaei |
| 241 | afqyrhgdpi prveytaeei atwkevyttl kglyathacg ehleafalle rfsgyredni |
| 301 | pqledvsrfl kertgfqlrp vagllsardf laslafrvfq ctqyirhass pmhspepdcc |
| 361 | hellghvpml adrtfaqfsq diglaslgas deeieklstl ywftvefglc kqngevkayg |
| 421 | agllssygel lhclseepei rafdpeaaav qpyqdqtyqs vyfvsesfsd akdklrsyas |
| 481 | riqrpfsvkf dpytlaidvl dspqavrrsl egvqdeldtl ahalsaig |

By “TH nucleic acid molecule” (or Tyrosine Hydroxylase) is meant a polynucleotide encoding an TH polypeptide. An exemplary TH nucleic acid molecule is provided at NCBI Accession No. NG_008128.

| (SEQ ID NO: 8) | |
|---|---|
| 1 | gcgggggggc agtgtgtgct ccagcatgtg tgtgtgtgtg tgcatgtaca cgtgtgcacc |
| 61 | tgtatcgcct gtgtgtgtgc atgtgatgtg tacacgtgtc atgcatgcac gcacatgtgt |
| 121 | agtgtgtgct cgtgtgtggt gtgtgcctgt gtcatgtatg agcacacttg tatatgttgt |
| 181 | gtgtactgtg tcatatatga gtgtgtttgc ctgtgtagtg catgcacatc cgtgtgtgca |
| 241 | tctggtgtgt ccgtgggtca ttacgagtgc atcgtatgtg tatcgtgtac atgagtacac |
| 301 | ttgtatgtgt ggtgtgtaca ggtgccatgt aagtgtgctt gtacatatat gcatgcatgt |
| 361 | gtcatatgca tctgtgtgtg catgtgtgtg gtgcacacat gtgttatgtc tgagtgtgcc |
| 421 | tgtatgtgtg ctatgtacac gtcatgtgtg agtgtgcttg catgtgcagt gtgtggatgc |
| 481 | tgcttgtacc tgtggtgtgt acctgtgtca tgggtgctca cacgtgcatg gagtgttgtg |
| 541 | tgtgtgcttg tgtgccccat gtgtgcatgt gtgtgtgcct cacacagatg cctgcatttg |
| 601 | cctaggcact tgcaagagga caccatgctg gctctcaaag atcacagggc cacctgagcc |
| 661 | ctgtgcacac cacagccagg ccatggctag accctgcaga gccacagggc gatgcctgtc |
| 721 | agccagggga cccagaacac ctcctgggct cctccccagc acatggctgg gctcctccag |
| 781 | caggcctgga tttgggaagg gcccgtggtg ggcaaggctg gtgctgggga gcaggcctgg |
| 841 | tggcctcaga gactcgccct gtgggcggag cagcctcaca gccaggtcga agtcagcact |
| 901 | ctgaccctgc cccacgcggg gagtgggcac cagtcccagg gcacagacgt gctgggtgat |
| 961 | taatctgggt gattaagcct cgggctgaga ggctgttgag agagaacacg ctccattgtg |
| 1021 | gagctggctc agcattcctt acggccatgg tggcaggggc tgtaaccaca gggacggcgg |
| 1081 | aagtggtgga gggtggtggg gtatggaggg aagcccagag ggctccgtgc aggaaggtgg |
| 1141 | agcctggtgc aatggagggg acagcaaggg ctcctcagac ctctgcgggg cccccactcc |
| 1201 | cctggtcacc tgttttgtct ctgatctggc ctgggtcggc cctcactcct ggccccacct |
| 1261 | catagccccc cctggtgggg ctccgctcca gcccttctcc ttcccagggg ccagtatgct |
| 1321 | ggccccaggg gtctcttggg gcgtgacctc ggcctccaga gaaccctgtc ccagctctgc |
| 1381 | ccttccctct ggggtctctg tagatgggac gctggtcaca gcagcctgtc tgatttgttc |
| 1441 | cctgtggcct aggttcctga gccccacagt gccaggggat ggatgccacc ggatctttga |
| 1501 | aagaccagtg tcaggccggg cgcagtggct cacgcctgta atcccagcac tttgggaggc |
| 1561 | cgaggtgggc ggatcacgaa gtcaggagat cgagaccatc ctggctaaca cagtgaaacc |
| 1621 | ccgtctccac taaaaataca aaaagttagc tgggcgtggt ggtgggcgcc tgtagtccca |
| 1681 | gctacttggg aggctgaggc aggagaatgg cgtgaaccgg ggaggcggag cttgcagtga |
| 1741 | gccgagatcg cgccattgca ctccagcctg ggtgacagag cgagactcgg tctcaaaaaa |
| 1801 | aaagaaaaaa aggaaagacc agtgtcttgg gagttgggaa acctgggctg gagactcact |
| 1861 | gcatgacccc tgagaagttg cacctcagaa cctcagtcct cgcatctgca gaatgggtct |
| 1921 | gtgaacacct cagctgcccg aacgtggatg ccgcaggctg acccagcact gagctctacc |
| 1981 | aagaccaggg gccagccgtg tgctccctcc aggcctgtgc ccagcgtgga gaggcctcgt |
| 2041 | cccgtgggcg ctggagtgga gccttcctgg tgtttgtgga catctctgga gagggccaga |
| 2101 | ggcaggtggg tgacacgggg catggctcaa tcatgggtgg tccagactgg agaggtaccc |
| 2161 | tcgggctggg agcggggagg ctggccaggg tagacttttg gggcctccat ggataccctc |
| 2221 | accatctgga atcggagagg ggcacggcac aaaggagggc ggggccaggg ccaggactgg |
| 2281 | agtcgggggc acctctgtgc caacaggggc cttggatctg gggtacagca tggttccccg |
| 2341 | gccctgaagg ggctggcgtg tgggacaggc ttcccaggaa tggataggca gggatggatg |
| 2401 | ctgcctgatt ggggcgggag gctggaggca gggcaggtgc aggcacctga gggcagcact |
| 2461 | cacctccaca ggggtccagg ggcctcccca gcctcagcac ctggcctggg ctcctgcctc |
| 2521 | cagagagcct ggccccaagg aagagtctag taagcttagt tcccatcggg cttccatgaa |
| 2581 | agcacaactg gcccggcagg aaaccgaatt aaaaagcaat atttgtatca gtggaagaca |
| 2641 | tttgctgaaa ggttaaatcc acatccggca gtgtgggcca tgagcctccg gcgtggtgtt |
| 2701 | catcaggcat gtctctcctc ctggcctggg cacctgagca ctggggccgc cctgggcaga |
| 2761 | gctggggcgg ggtgctgggg ggcctggagc tgcctcaccg agggatcctc agcagccgac |
| 2821 | cctgggggag gcaaatgaga ctctttctgg ggaccttgag gggagctcgg gggagccatg |
| 2881 | cagagcttca ccaggcctgg acactgggca tggaggctgg gccacccaag ggccatcacc |
| 2941 | agggactcag gtgggtgggc ctcagccctg ggtgacagaa gctcacgggc cgcagggcga |
| 3001 | ggccagaggc tgagccttca ggctgaggtc ttggaggcaa atccctccaa cgcccttctg |
| 3061 | agcaggcacc cagacctact gtgggcagga cccacaggag gtggaggcct ttggggaaca |
| 3121 | ctgtggaggg gcatagcatc tccgagagag gacagggtct gcactgggtg ctgagagaca |
| 3181 | gcaggggccg agcggtaggc ttccctgccc ccagggatgt tccagaggag cgcaagggag |
| 3241 | gggcattaat atcgtggcaa gaaagggcag gcattgcaga gtgagcagcg acggaactgg |
| 3301 | gttttgtggg atgcatagga gttcacccgg ataagaggtg ggtgaggaat gacactgcaa |
| 3361 | accggggatc acggagcccc aaatccttct gggccaggaa gtgggaaggg ttggggggtc |
| 3421 | ttccctttgc tttgactgag cactcagcct gcctgcagag ggcagcgagg agccacggag |
| 3481 | gggtgtggga cagggatgcc atggctgaag cagttttagg aaaggtccca ggggctattg |
| 3541 | ttgaagagag aacggggagc ggggagtccc acagctgaca ggagcagagt gggccctgag |
| 3601 | agatgccagc tctggctgcc acagtgacca gccggggtag gccttcgaga agtcagggag |
| 3661 | cgtctagggc ttctggctcc tgctgggccc agggtgtcat cttgggctgc caacaccaga |
| 3721 | aagcccagca gatacaggaa gccccaagcc ctgtcggaaa cggttcttct ccaggaggga |
| 3781 | cagcggtggc agcgttcagc cgcaggccat gcactctggg gccacgtcct tccctctgta |
| 3841 | cagtccagca ttgtcaaggc aggctctggc catctctgct gaccccagag ggatggggag |
| 3901 | gcctcccctt ccaccagaag ggccagaagc caccctgggc aggggcatca ctctccctgg |
| 3961 | gtggggcagc ggctgggagc aggaggtgcc agtgggcgtg ggctggatgc gggtgcctgc |
| 4021 | ggggcggaca tggaacttgg gggaggctct aggctggggt tgtcctcaag ggagttctca |
| 4081 | ggtcacccca gggtcaccct caacccgggg cctggtgggg tagaggagaa actgcaaagg |
| 4141 | tctctccaag gggaaggcat cagggccctc agcactgagg gacgtgcgtg ctctttaaag |
| 4201 | aaggggccac aggaccccga gggaagccag gagctagcag tgggccatag aggggctgag |
| 4261 | tggggtgggt ggaagccgtc cctggccctg gtcgccctgg caaccctggt ggggactgtg |
| 4321 | atgcaggagg tggcagccat ttggaaacgc gtggcgtctc cttagagatg tcttcttcag |
| 4381 | cctcccaggg tcctccacac tggacaggtg ggccctcctg ggacattctg gaccccacgg |
| 4441 | ggcgagcttg ggaagccgct gcaagggcca cacctgcagg gcccgggggc tgtgggcaga |
| 4501 | tggcactcct aggaaccacg tctatgagac acacggcctg gaatcttctg gagaagcaaa |
| 4561 | caaattgcct cctgacatct gaggctggag gctggattcc ccgtcttggg gctttctggg |
| 4621 | tcggtctgcc acgaggttct ggtgttcatt aaaagtgtgc ccctgggctg ccagaaagcc |
| 4681 | cctccctgtg tgctctcttg agggctgtgg ggccaagggg accctggctg tctcagcccc |
| 4741 | ccgcagagca cgagcccctg gtccccgcaa gcccgcgggc tgaggatgat tcagacaggg |
| 4801 | ctggggagtg aaggcaatta gattccacgg acgagccctt tctcctgcgc ctccctcctt |
| 4861 | cctcacccac ccccgcctcc atcaggcaca gcaggcaggg gtgggggatg taaggagggg |
| 4921 | aaggtggggg acccagaggg ggctttgacg tcagctcagc ttataagagg ctgctgggcc |
| 4981 | agggctgtgg agacggagcc cggacctcca cactgagcca tgcccacccc cgacgccacc |
| 5041 | acgccacagg ccaagggctt ccgcagggcc gtgtctgagc tggacgccaa gcaggcagag |
| 5101 | gccatcatgg taagagggca ggtaggtgcc cggcggccgc agtggaccgg agcccagggc |
| 5161 | tggtgccagc tgcctctgct actccccagc ctggctggca gccccaggct cagggtccat |
| 5221 | gcaaacccct gggacgcggc gtggatgtgg aggcctgggc acagcggcat cccctgtgcc |
| 5281 | tggtgtttga gtccctgttg ggggagggtg aggtgatgcc tgtccctgtg tgtgcccctc |
| 5341 | ttaggccgac ctctctcggg ggtcgtgtgg gtctctgtgt cttgtttcat cttgaatctt |
| 5401 | aacgatcgga atgtggaaac aaatccatcc aaaaaatcca agatggccag aggtccccgg |
| 5461 | ctgctgcacc cagcccccac cctactccca cctgcccctg cctccctctg ccccagctgc |
| 5521 | cctagtcagc accccaacca gcctgcctgc ttggggaggc agccccaagg cccttcccag |
| 5581 | gctctagcag cagctcatgg tggggggtcc tgggcaaata gggggcaaaa ttcaaagggt |
| 5641 | atctgggctc tggggtgatt cccattggcc tgttcctccc ttatttccct cattcattca |
| 5701 | ttcattcatt cattcattca ccatggagtc tgtgttccct gtgacctgca ctcggaagcc |
| 5761 | ctgtgtacag gggactgtgt gggccaggct ggataatcgg gagcttttca gcccacagga |
| 5821 | ggggtcttcg gtgcctcctt gggcactcag aaccttgggc tccctggcac atttaaaatg |
| 5881 | ggtttttatt tatggacctt gattgaaatg tggtgtgagt tgtagcagtg tcatttccag |
| 5941 | gtaccttctc agggacacag ggcgccctcc cccgtcctcc cccgccctcc cctaccctcc |
| 6001 | cccaccaggc tccccatcag gcatcccctc cccagggcgc cccggggccc agcctcacag |
| 6061 | gctctccgtg gcctggaact gcagccccag ctgcatccta cacccccacc ccaagggtaa |
| 6121 | gtaagagggg actctgggag gggcttctgc tgctcccctt catgttccac aaccctggaa |
| 6181 | gctcaggatg aagctgattc ttctcttaca aggggcccag agccttcttg ggagttcagc |
| 6241 | tccaagggat gagccccagg tgtctgccaa gtccccctct gtccaggcct gggacggctc |
| 6301 | tgggatcgag gggtcagagg cgctgagccc agggagagac acctgcgccc agagctatga |
| 6361 | caaagggtgg agggatgaca aggcagccag gagcgggcgc ctgcggggtg gcacagaggg |
| 6421 | gcagggcccg aggacaggtg tcctgatggg agtgtgagaa agggtcccct gtgcggcagc |
| 6481 | caggagggta ggggggttgt tcactggggc cctgtggggg cagctccttc ctgagctgcc |
| 6541 | gttccctccc cggcagccga tgccactgtc catcaagaca tcgccctctt cccatcacta |
| 6601 | atccagttag cgcctggcct ggggatgagt gacacagcgt ctctgtctgt ctgctcgcca |
| 6661 | cagagtgggg agcaggcgag caccttccca gcccccactc ctcccccacc accactgctt |
| 6721 | ctgactgggc tgcccccatc gggaagggcg tgcaatgccc gcaggcacct cggctagcat |
| 6781 | ctgccccagc aggcacacag taggcgctca aaaacgtgct ctcatcccct gcctctgtgt |
| 6841 | gccatcagcg ctgcccgact gtgggaccag ctgtgggtgg aggtccccgg gtctcagcag |
| 6901 | gtggaggagg catgggtgcc ccttgtcccc acagtccccg cggttcattg ggcgcaggca |
| 6961 | gagcctcatc gaggacgccc gcaaggagcg ggaggcggcg gtggcagcag cggccgctgc |
| 7021 | agtcccctcg gagcccgggg accccctgga ggctgtggcc tttgaggaga aggaggggaa |
| 7081 | ggccgtgcta aacctgctct tctccccgag ggccaccaag ccctcggcgc tgtcccgagc |
| 7141 | tgtgaaggtg tttgaggtga gctggtggcc ttcgtgtccc tggggcaagt tcacctgtgg |
| 7201 | gtggggctgt gtgggctgag ttcctgaccc ctctatagca gaggtgcagc tgcccaggcc |
| 7261 | cccgaggccg gcacaggatg cagcagggga gtctcaggcc tcagctcagc ccccatggca |
| 7321 | tctagccaca cccccgtgtt tttgagggat cctgagccca cccctagggc tgaggctacc |
| 7381 | aagccccact gtgcctcttg ccttgcccat cccctggatc cccctcaccc accatttccc |
| 7441 | acgtgggggg ctcccagcag ggcagcacaa gaggcagggg cagggcagtg tgccctctcc |
| 7501 | cacccaccca gcacagtggc tcaggtgacc actgattgca ttagtcactc cggccccact |
| 7561 | gtgccccggg aggcaggtga cccagctccc ggaagaagct cccaaatgac attaaagcca |
| 7621 | gactccccgc cccccagctc ccagagccag ttttgtggcc cgagggccac tgcgacccac |
| 7681 | cgcccttgtt gctaggcaac aggaggtggg ggtggagcgg acccttctgg ccagtgtcct |
| 7741 | ggacgctcag gggccagtga gactcagggc ccatcctaca aacctggatg aggccaccag |
| 7801 | ggttgggggc accttctgac cagtggctga ggagccggac tgtgtggcat ggccttggga |
| 7861 | cacacacacc gagccgccca gaaccaggtt aagcctcaag cggtgacaac tcctggttag |
| 7921 | gcacgtaaca caaaatccaa cttgccagtg gcaaaccctg gcctggtggc cgacagctga |
| 7981 | cctgagcctg gaagaacggg atctgtgtgc tgctagcaca aaagtcaagg gcagggcctg |
| 8041 | gccagccagc cagatgtgcc tcctccccgc ccaccccacc ctctctctcc atctctgtct |
| 8101 | ctttctcctt ctctctctct tcctgctttt gctccctaag acgtttgaag ccaaaatcca |
| 8161 | ccatctagag acccggcccg cccagaggcc gcgagctggg ggcccccacc tggagtactt |
| 8221 | cgtgcgcctc gaggtgcgcc gaggggacct ggccgccctg ctcagtggtg tgcgccaggt |
| 8281 | gtcagaggac gtgcgcagcc ccgcggggcc caagggtgag gcggttttct gtccttgagg |
| 8341 | gccaccaaat gaccttgaga ggctggggtg caggggctcc tgcaggggga ccctacagtg |
| 8401 | accacgtggt ggtggcctgg ttccctctct gcgggctcca ctccgcaccc cgttttgcta |
| 8461 | cacatccgtg tccgggcctg gggccactcc aggatccccc cgcagctctc acagccccgg |
| 8521 | ctgcctctgc cccccggaag tcttgtaggg gaggctgctt caaggtgggt gacacagccc |
| 8581 | cacggctccg agctcaccaa gatctcttcc tccatcaccc ataaagtccc ctggttccca |
| 8641 | agaaaagtgt cagagctgga caagtgtcat cacctggtca ccaagttcga ccctgacctg |
| 8701 | gacttggacc acccggtgag tggtgcgccc ctcactcagg cctcctgccc ctgatcacat |
| 8761 | cccctaccct tagcccaacc ctggacagga gtctgtcggc tccaggagcc tccgtggcct |
| 8821 | gtgcccccac cccagcacag cctcctgacc cgtgcatccc ctctgccctc agggcttctc |
| 8881 | ggaccaggtg taccgccagc gcaggaagct gattgctgag atcgccttcc agtacaggca |
| 8941 | gtgaggggcc cctgcgctcg ggacccagac tccgtcctgc aggctgacgc tggacctggg |
| 9001 | gggtgggagg gaaggacaaa ggggaggacc catcttgtca ccagcatcag tgcctcctgc |
| 9061 | caggcagctc tgctccaggg ctttccatgt ccccaaatcc cagtggggaa actgaggccc |
| 9121 | aggggggcta gagcaacctg ccgaggccac atagccggct cacggcacag tcagctgggg |
| 9181 | tgcaccctcc tgtccatcct ccaacccaaa ggcctcgctg cactaggcgg gtgtggacct |
| 9241 | gtgcccagtg aagctccctc cctccctcct gcccttctca ctccccgagg ggacctgctg |
| 9301 | accactggcc ccctccccag cggcgacccg attccccgtg tggagtacac cgccgaggag |
| 9361 | attgccacct ggtgagacct ccgtgcagct aggggctggg gaggagcccg ggggatgcct |
| 9421 | cctggaatcc tggcgtgtga gggccgcctc cagggacctt ggcacaacag gagagactaa |
| 9481 | ggccgggaag aagagggact tgcagggctc agaatgttgg gttgggagga agaggctacc |
| 9541 | catcctgtcg ggccatcccc agtgtgctga gggaccgccc ctcatggccc cctatcccct |
| 9601 | gggattccct aaagccacca gcaaaagccc ctcccggggg cctgggtctt caggggtccc |
| 9661 | caagaggcct gcgttggtag gggctcaggc aggcagaggc acccacagtt caggaggggg |
| 9721 | gtttcgggca ctggggtggg gcattagagg gccctgagcc tggctgcccg caggaaggag |
| 9781 | gtctacacca cgctgaaggg cctctacgcc acgcacgcct gcggggagca cctggaggcc |
| 9841 | tttgctttgc tggagcgctt cagcggctac cgggaagaca atatccccca gctggaggac |
| 9901 | gtctcccgct tcctgaaggg tgtgcccaga cgggaggggc gcagagccgg ggggccgggg |
| 9961 | atggtcagcc aagcgcccca ccccagcgcg gctccagccc gtcccggctc ggcagtgacc |
| 10021 | cgcgtggccc cttgcagagc gcacgggctt ccagctgcgg cctgtggccg gcctgctgtc |
| 10081 | cgcccgggac ttcctggcca gcctggcctt ccgcgtgttc cagtgcaccc agtatatccg |
| 10141 | ccacgcgtcc tcgcccatgc actcccctga gccgtgagtg cgcgccctgg ccgccagccc |
| 10201 | gagggtgggg ggtgcgacgg gcggcccctc agcccccttc tccctcctac gcgcagggac |
| 10261 | tgctgccacg agctgctggg gcacgtgccc atgctggccg accgcacctt cgcgcagttc |
| 10321 | tcgcaggtac gccgcggcct cggagggagc cggggtcacc caggggctgg cttggcgccg |
| 10381 | ggggcgggcg gggatcgatg tgcgggtggg tgaagtgtgc tgcctgctcc cgggccccgc |
| 10441 | caaggaggct cggcgccccg agggtcgcgc ggcatagggc ggggctggag cggagcctcc |
| 10501 | cacggcctgt gctgccacct gccggctacc tgggaacggc gcccacgggc ttaggaatgt |
| 10561 | ggtcaaggag ggctgcctgg aggaggaggc ccggtggagg tgcggatcct gggcggccag |
| 10621 | ggaaggtctc tgccgccagg gaagtgtccc agagacccct ggaggggctg ctgacacccc |
| 10681 | cggtgccccc acctcgagca tgacccaggg ctgcctctcc ccatccttca tcctccctgc |
| 10741 | tccacaggac attggcctgg cgtccctggg ggcctcggat gaggaaattg agaagctgtc |
| 10801 | cacggtgggt tgacccctcc ctgcagggcc tggggtgtgg gtttgggggt ctgaatccag |
| 10861 | gcctcaccct cttgccgtcc aggctgaggc ctctccttcc acccacgaat tgtgaccctc |
| 10921 | accctggcct gcctgcatcc tggcctggcc tccctggggg tggtatcctg gtcacgggtg |
| 10981 | accaggggct gcccggtggg cggcagctgt ctctgggctg atgctgcccg gcttccccgc |
| 11041 | agctgtactg gttcacggtg gagttcgggc tgtgtaagca gaacggggag gtgaaggcct |
| 11101 | atggtgccgg gctgctgtcc tcctacgggg agctcctggt gagagtctct ccttgctgca |
| 11161 | gcccccagca gaggggcagg gctgggggac ggtgcaggga ggggacaggc tcccagtggg |
| 11221 | aggaaactga ggcctggacc tccaggactc aggctctgtt tgggagaagg cttgtctctg |
| 11281 | cccagtcctc accccacatt atcccaggcc tccgaaggcc cggcggggga gatgggggtg |
| 11341 | actctaccca aggaacccac ccagcgtcag gccacggtgc cccagttccc tcggggacct |
| 11401 | gggtgcagtg gagtcagtga tgccattggc ctcctgccag cactgcctgt ctgaggagcc |
| 11461 | tgagattcgg gccttcgacc ctgaggctgc ggccgtgcag ccctaccaag accagacgta |
| 11521 | ccagtcagtc tacttcgtgt ctgagagctt cagtgacgcc aaggacaagc tcaggtgggc |
| 11581 | taggctgcta gggcaagccc cccatggtgc ccccaaactg ggccagccag gccttccttc |
| 11641 | tggccttgag cagggctgga cctgtgagcc caggtcacag atgagaaaac cgacccctgg |
| 11701 | ttgcagcagc ccccacacag cagggacacc atccgtgaga aggaccccag cgtctgggga |
| 11761 | ggggcagacc tacaggactg ggggctgctg ggtggccggg tcaaggccag tcttggaggt |
| 11821 | gctgacagag cctgagcttt gtgaggacgt cctgtggaac ctgtcccggc cccctgccct |
| 11881 | gggatgggga gaagtcaggg ggatagacag agtcaaggtg ggggacaggg cgggagtggg |
| 11941 | gtccccaggg ctgggggcct ttggtgcagt gaccagagtg tcaggagagg ggagcaaagc |
| 12001 | cctctagcct catcctcata aaaggtctca tcattttccc tccagcctct tatgcactgg |
| 12061 | ggaaactgag gccaggggct atgtgtccag cggacagggg tgctgaattc cacccacagg |
| 12121 | cttagggata tggtcaagga aagcttcctg gaggaggccc agtggaggtt cagggaggga |
| 12181 | tggggtgccc ggcagtctct agtggaaaag gcgcctagcc tatctccccc atgaaccccc |
| 12241 | tcacccagcc ctggaagagg cctcagtgtc ccgcctgtga ccagttggct cagaaaagcc |
| 12301 | ctgggagctc tgagccactg tgaaggtgga aacgcggccc ctggcctccc ctctcctgga |
| 12361 | ggctgcagac tctgcccgcc agttgacgag ggctctgccg ctctcctccc caggagctat |
| 12421 | gcctcacgca tccagcgccc cttctccgtg aagttcgacc cgtacacgct ggccatcgac |
| 12481 | gtgctggaca gcccccaggc cgtgcggcgc tccctggagg gtgtccagga tgagctggac |
| 12541 | acccttgccc atgcgctgag tgccattggc taggtgcacg gcgtccctga gggcccttcc |
| 12601 | caacctcccc tggtcctgca ctgtcccgga gctcaggccc tggtgagggg ctgggtcccg |
| 12661 | ggtgcccccc atgccctccc tgctgccagg ctcccactgc ccctgcacct gcttctcagc |
| 12721 | gcaacagctg tgtgtgcccg tggtgaggtt gtgctgcctg tggtgaggtc ctgtcctggc |
| 12781 | tcccagggtc ctgggggctg ctgcactgcc ctccgccctt ccctgacact gtctgctgcc |
| 12841 | ccaatcaccg tcacaataaa agaaactgtg gtctctacac ctgcctggcc ccacatctgt |
| 12901 | gccacagaga cagaccctgg gatcctcaga ctcccacacc cccaccccag cctcactcag |
| 12961 | aggtttcgcc ctggcctcct tcctcctctg ggagatggct ggccgccctg gccaggcagc |
| 13021 | tggcccctcc gggcctggtt tccccgctca ccctgaggcc ccgcccagct ctgagcccca |
| 13081 | agcagctcca gaggctcggg caccctggcc gagctgcccc atctccgtgg ggtgccctcc |
| 13141 | caaggtgggg agccacgtga cagtgggagg gcctctctca ggcctggcag ggagcagggg |
| 13201 | tcacaaactg tgctggctgg gggtggtctc agaggtgggc ctgcaggcct aaccctccct |
| 13261 | gctgacaggg ctcccagccc ttgagagaaa cagggatgga ggaacagctg ccctgatgcc |
| 13321 | ctcacccacc cggagcaggc cctgcgaacc aaggggaacc tcagtgtggc ccccagcatg |
| 13381 | tgtgctgatg gggagggtct ggctgagctg gtgcccaggc agatggtctg ggcctgtctc |
| 13441 | cccagcgagg caggatgggg gctggatttc agactctgta agatgcccct ggcttactcg |
| 13501 | aggggcctgg acattgccct ccagagagag cacccaacac cctccaggct tgaccggcca |
| 13561 | gggtgtcccc ttcctacctt ggagagagca gccccagggc atcctgcagg gggtgctggg |
| 13621 | acaccagctg gccttcaagg tctctgcctc cctccagcca ccccactaca cgctgctggg |
| 13681 | atcctggatc tcagctcccc ggccgacaac actggcaaac tcctactcat ccacgaaggc |
| 13741 | cctcctgggc atggtggtcc ttcccagcct ggcagtctgt tcctcacaca ccttgttagt |
| 13801 | gcccagcccc tgaggttgca gctgggggtg tctctgaagg gctgtgagcc cccaggaagc |
| 13861 | cctggggaag tgcctgcctt gcctcccccc ggccctgcca gcgcctggct ctgccctcct |
| 13921 | acctgggctc cccccatcca gcctccctcc ctacacactc ctctcaagga ggcacccatg |
| 13981 | tcctctccag ctgccgggcc tcagagcact gtggcgtcct ggggcagcca ccgcatgtcc |
| 14041 | tgctgtggca tggctcaggg tggaaagggc ggaagggagg ggtcctgcag atagctggtg |
| 14101 | cccactacca aacccgctcg gggcaggaga gccaaaggct gggtgtgtgc agagcggccc |
| 14161 | cgagaggttc cgaggctgag gccagggtgg gacataggga tgcgaggggc cggggcacag |
| 14221 | gatactccaa cctgcctgcc cccatggtct catcctcctg cttctgggac ctcctgatcc |
| 14281 | tgcccctggt gctaagaggc aggtaggggc tgcaggcagc agggctcgga gcccatgccc |
| 14341 | cctcaccatg ggtcaggctg gacctccagg tgcctgttct ggggagctgg gagggccgga |
| 14401 | ggggtgtacc ccaggggctc agcccagatg acactatggg ggtgatggtg tcatgggacc |
| 14461 | tggccaggag aggggagatg ggctcccaga agaggagtgg gggctgagag ggtgcctggg |
| 14521 | gggccaggac ggagctgggc cagtgcacag cttcccacac ctgcccaccc ccagagtcct |
| 14581 | gccgccaccc ccagatcaca cggaagatga ggtccgagtg gcctgctgag gacttgctgc |
| 14641 | ttgtccccag gtccccaggt catgccctcc ttctgccacc ctggggagct gagggcctca |
| 14701 | gctggggctg ctgtcctaag gcagggtggg aactaggcag ccagcaggga ggggacccct |
| 14761 | ccctcactcc cactctccca cccccaccac cttggcccat ccatggcggc atcttgggcc |
| 14821 | atccgggact ggggacaggg gtcctgggga caggggtgtg gggacagggg tcctggg |

By “LMX1A polypeptide” (or LIM homeobox transcription factor 1-alpha) is meant a polypeptide or fragment thereof having at least about 85% amino acid identity to NCBI Accession No. Q8TE12.

| (SEQ ID NO: 9) | |
|---|---|
| 1 | mldglkmeen fqsaidtsas fssllgravs pksvcegcqr vildrfllrl ndsfwheqcv |
| 61 | qcasckeple ttcfyrdkkl yckydyeklf avkcggcfea iapnefvmra qksvyhlscf |
| 121 | cccvcerqlq kgdefvlkeg qllckgdyek erellslvsp aasdsgksdd eeslcksahg |
| 181 | agkgtaeegk dhkrpkrprt ilttqqrraf kasfevsskp crkvretlaa etglsvrvvq |
| 241 | vwfqnqrakm kklarrqqqq qqdqqntqrl ssaqtngggs agmegimnpy talptpqqll |
| 301 | aieqsvyssd pfrqgltppq mpgdhmhpyg aeplfhdlds ddtslsnlgd cflatseagp |
| 361 | lqsrvgnpid hlysmqnsyf ts |

By “LMX1A nucleic acid molecule” (or LIM homeobox transcription factor 1-alpha) is meant a polynucleotide encoding an LMX1A polypeptide. An exemplary LMX1A nucleic acid molecule is provided at NCBI Accession No. AH011517.

| (SEQ ID NO: 10) | |
|---|---|
| 1 | gtataggttg gggcggagtc ggattcggga tggaaaacct ggggcaaggg atgtaggtgg |
| 61 | gggtgagggg ggcaggagaa ggagaaacgc agttgggggg cggaggccta agtacataac |
| 121 | gtgttgactt caagtgaaat cagatcagcc agagcagttc gctgtgactg atctctcctc |
| 181 | ccaccctaca ttctcttggc tggaccctat cctcctggct gattctggtc gccctggaca |
| 241 | ctccctcagt tctttcccag gagtgcggtg gctgctggcg ccgagtccca gcgggcacgg |
| 301 | acgtcagacg catcgtttct tctcctctac aggtcctccc ggcccggccc gaacatgctg |
| 361 | gacggcctaa agatggagga gaacttccaa agcgcgatcg acacctcggc ctccttctcc |
| 421 | tcgctgctgg gtgagtgttc aggccgtgcg tcctgggcgc actctctttc cgcttggcgc |
| 481 | tgagctctgg agccccgctc tctgggacct ggtccgcgat agggaagcta gcgcccctct |
| 541 | tcatacacta aattgagccc catcactatc tgtccgtcag tgcttgtggg tcgtccctac |
| 601 | ccaaataaat ccaacaagcc gccccaggcc tcacgcactg ggcaccgaat tccccaaagc |
| 661 | cgcgaggggc gggcgagctt gttcgtaggc gtctgagtgg caagtgatta aaaataccca |
| 721 | gggctggatt tttaatctcg gagctgatcg acgtctcata aatgccgccc tcttctcgcg |
| 781 | gcctagaggc aatagcatcc gagacccgag gcctggagcg cccaagttcg aggaggcttc |
| 841 | tctcccccac caactccagc cccaatttca gccatgggca aggccgagag agacttttct |
| 901 | nnnnnnnnnn nnnnnnnnnn nnnnnnnnnn nnnnnnnnnn nnnnnnnnnn nnnnnnnnnn |
| 961 | nnnnnnnnnn nnnnnnnnnn nnnnnnnnnn nnnnnnnnnn gggtcccggc caggtttggc |
| 1021 | atggtctacc tgcccgggct gctcacccgc caacgtctgt tgtggctaca ggcagagcgg |
| 1081 | tgagccccaa gtctgtctgc gagggctgtc agcgggtcat cttggacagg tttctgctgc |
| 1141 | ggctcaacga cagcttctgg catgagcagt gcgtgcagtg cgcctcctgc aaagagcccc |
| 1201 | tggagaccac ctgcttctac cgggacaaga agctgtactg caagtatgac tacgagaagt |
| 1261 | aagtggccgc acccccgcag cgctccccgc gcactggcat nnnnnnnnnn nnnnnnnnnn |
| 1321 | nnnnnnnnnn nnnnnnnnnn nnnnnnnnnn nnnnnnnnnn nnnnnnnnnn nnnnnnnnnn |
| 1381 | nnnnnnnnnn nnnnnnnnnn atcccagttc ttgaagttcc ttttgctgtt gacttcaggg |
| 1441 | gagacccagg accaagccag attttactca tggtgcatgt acttcctttc tccctgctgc |
| 1501 | caggctgttt gctgttaaat gtgggggctg cttcgaggcc atcgctccca atgagtttgt |
| 1561 | tatgcgggcc cagaagagtg tataccacct gagctgcttc tgctgctgtg tctgcgagcg |
| 1621 | acagcttcag aagggtgatg agtttgtcct gaaggagggg cagctgctct gcaaagggga |
| 1681 | ctatgagaag gagcgggagc tgctcagcct ggtgagccca gcagcctcag actcaggtga |
| 1741 | gtgccaggtg gtgggcaggg ctgcggtggg gtgggtagag tggagttggg tggctgtctg |
| 1801 | cattgtttct tccctagatg nnnnnnnnnn nnnnnnnnnn nnnnnnnnnn nnnnnnnnnn |
| 1861 | nnnnnnnnnn nnnnnnnnnn nnnnnnnnnn nnnnnnnnnn nnnnnnnnnn nnnnnnnnnn |
| 1921 | catacagctc caggaactgg ctttcaggga ctcacaacat tgtcttttgc ttctttcagg |
| 1981 | taaaagtgat gatgaagaaa gtctctgcaa gtcagcccat ggggcaggga aaggaactgc |
| 2041 | tgaggaaggc aaggaccata agcgccccaa acgtccgaga accatcttga caactcaaca |
| 2101 | gaggcgagca ttcaaggcct catttgaagt atcctccaag ccctgcagga aggtatagga |
| 2161 | gggagcaggg aggaaaagga gctgggcccc acttctctgt gtgcactcag acccctctgg |
| 2221 | gatctcagtg ggcattgggg gtcacagtgg tgaggaaggc tgttcagaca gagcctgcac |
| 2281 | aggcggctca agcctgttgg agactccaga gatcactaag ctgtggccag ggtgtgatag |
| 2341 | actctcctga agctttcatg catgcacacc aactccaaat ggcccctgtc acacctttca |
| 2401 | tttcatagag cacaatggga acagtaataa tgataggtgt ccattgtggt gtagacccag |
| 2461 | atgctgtaaa gcaaagagta taaaaacaca gtggcttgca gtactctttt ttgagtctgg |
| 2521 | ctttttccac ttggtgtggt ggtttgggga ttcattcatt cctatttcag cattccactg |
| 2581 | tataggtgtg ccatgattgg tttgtccatg cacctgttga tgggtgtttg gggttgtttc |
| 2641 | tagtttggga ctgtttcaaa taggactgct atggacattc atgtaaaaaa aaatacagtg |
| 2701 | gtttaatgag acaggagttt attctcttct gtcacagtcc agaggtgagc aaggcaaggc |
| 2761 | tggtgggtgg ctctgttatc catctcctgt gtccaagcga ctgctccagt tgtcaccatg |
| 2821 | tttccagtca ccaggtagag aaagaggaaa tggagggcaa gcgccctgct ttttaaggat |
| 2881 | nnnnnnnnnn nnnnnnnnnn nnnnnnnnnn nnnnnnnnnn nnnnnnnnnn nnnnnnnnnn |
| 2941 | nnnnnnnnnn nnnnnnnnnn nnnnnnnnnn nnnnnnnnnn atgcatatgc atggcttata |
| 3001 | gctaaagcac aacaatagac taaagtctaa accacttgaa ggcctaattt ccagagcaag |
| 3061 | agaaatccag aaacacctct tgggaatgca catgtaaatt aataattatt attttgtttc |
| 3121 | tttacctggt gaaggacttt ctttctacct gaagggaagc aatgttctcg tgtttgtgtg |
| 3181 | tatgctcaac attaaaaact attcagctcc taaagcagat acagtctttt ggcctcctca |
| 3241 | agtattatat aggagatgtt ctacctccta ccctgagatg ccagtgtgtc tacatttctc |
| 3301 | gttcaatttt tccaaggtga gagagactct ggctgcagag acagggctga gtgtccgtgt |
| 3361 | cgtccaggtg tggttccaaa accagagagc gaaggtaacc tgcttcttac ttttatctgt |
| 3421 | ccccatgttg ctggtttcct gaaataatca cagtaggaca nnnnnnnnnn nnnnnnnnnn |
| 3481 | nnnnnnnnnn nnnnnnnnnn nnnnnnnnnn nnnnnnnnnn nnnnnnnnnn nnnnnnnnnn |
| 3541 | nnnnnnnnnn nnnnnnnnnn agccctctcc cggggaaggt gtcacttcca ggccccccct |
| 3601 | tactttgtga acatgctgca ggccacctga cttctaatcc tatggtcctc tccttatcag |
| 3661 | atgaagaagc tggccaggcg acagcagcag cagcagcaag atcagcagaa cacccagagg |
| 3721 | ctgagctctg gtaagctggt gcctcctccc aggcagttct ggctggaatc caggctgttc |
| 3781 | ctaccagagg cctcccacta cccagctctt tggatgacat atctggactc agtgaagcct |
| 3841 | agaccacacc cactggagaa ataaggcctt caagggaaga ctgagccacg aggaacttgt |
| 3901 | gagagggttg agggctcctg agctgcaggc ttagaactgc tgattgggga tggcactgac |
| 3961 | cttatccaca gcgtccaggc ctggatccca ccacagcgtc agggactgct tgcagagtca |
| 4021 | cagatacgtt cagtttctca tcttgcttag ttctccttcc aggctaattg atttaataga |
| 4081 | agacacctcg gtgacttggc tctttccaaa ataacataaa gtagtaaaaa taatgatagt |
| 4141 | aaaataacaa tgccttcctt tgttgaacac tcttatagat tggtgttctc atacatgctg |
| 4201 | acttgacttt tacaacaccc attcctggag gcgagtggag aagttgttat tatccctatg |
| 4261 | tcacagatga gcaaacaaag gctctgcaag attgaatgtg gccctagatc ggtaagggca |
| 4321 | gggggctggg actagaactc taactgtgtt ccacaggcca tgggccttct catctctacc |
| 4381 | cagatgtgct tttgaaaaag nnnnnnnnnn nnnnnnnnnn nnnnnnnnnn nnnnnnnnnn |
| 4441 | nnnnnnnnnn nnnnnnnnnn nnnnnnnnnn nnnnnnnnnn nnnnnnnnnn nnnnnnnnnn |
| 4501 | cacgttgaga atgacctggc ttcttctttg ttccacagct cagacaaacg gtggtgggag |
| 4561 | tgctgggatg gaaggaatca tgaaccccta cacggctctg cccaccccac agcagctcct |
| 4621 | ggccatcgag cagagtgtct acagctcaga tcccttccga cagggtctca ccccacccca |
| 4681 | gatgcctgga gaccacatgc acccttatgg taagagggac ttaagcccct cgggccctct |
| 4741 | cataacttgt gtgggtttct cattccctcc taaacacatc taggcagttc ccagatgctc |
| 4801 | nnnnnnnnnn nnnnnnnnnn nnnnnnnnnn nnnnnnnnnn nnnnnnnnnn nnnnnnnnnn |
| 4861 | nnnnnnnnnn nnnnnnnnnn nnnnnnnnnn nnnnnnnnnn aaatgagtca cttcttcaag |
| 4921 | accctcatgc cagtgtttca tctccatttc aggtgccgag ccccttttcc atgacctgga |
| 4981 | tagcgacgac acctccctca gtaacctggg tgattgtttc ctagcaacct cagaagctgg |
| 5041 | gcctctgcag tccagagtgg gaaaccccat tgaccatctg tactccatgc agaattctta |
| 5101 | cttcacatct tgagtcttcc cctagagttc tgtgactagg ctcccatatg gaacaaccat |
| 5161 | attctttgag gggtcactgg ctttaggaca gggaggccag ggaagaggtg ggttggggag |
| 5221 | ggagttttgt tggggatgct gttgtataat gatatggtgt agctcagcat ttccaaagac |
| 5281 | tgaatacatt atggattgca tagtttaatg |

By “FOXO1 polypeptide” (or Forkhead box protein O1) is meant a polypeptide or fragment thereof having at least about 85% amino acid identity to NCBI Accession No. Q12778.

| (SEQ ID NO: 11) | |
|---|---|
| 1 | maeapqvvei dpdfeplprp rsctwplprp efsqsnsats spapsgsaaa npdaaaglps |
| 61 | asaaavsadf msnlsllees edfpqapgsv aaavaaaaaa aatgglcgdf qgpeagclhp |
| 121 | appqppppgp lsqhppvppa aagplagqpr kssssrrnaw gnlsyadlit kaiessaekr |
| 181 | ltlsqiyewm vksvpyfkdk gdsnssagwk nsirhnlslh skfirvqneg tgksswwmln |
| 241 | peggksgksp rrraasmdnn skfaksrsra akkkaslqsg qegagdspgs qfskwpaspg |
| 301 | shsnddfdnw stfrprtssn astisgrlsp imteqddlge gdvhsmvypp saakmastlp |
| 361 | slseisnpen menlldnlnl lssptsltvs tqsspgtmmq qtpcysfapp ntslnspspn |
| 421 | yqkytygqss msplpqmpiq tlqdnkssyg gmsqyncapg llkelltsds pphndimtpv |
| 481 | dpgvaqpnsr vlgqnvmmgp nsvmstygsq ashnkmmnps shthpghaqq tsavngrplp |
| 541 | htvstmphts gmnrltqvkt pvqvplphpm qmsalggyss vsscngygrm gllhqeklps |
| 601 | dldgmfierl dcdmesiirn dlmdgdtldf nfdnvlpnqs fphsvkttth swvsg |

By “FOXO1 nucleic acid molecule” (or Forkhead box protein 01) is meant a polynucleotide (e.g., mRNA) encoding an FOXO1 polypeptide. An exemplary FOXO1 nucleic acid molecule is provided at NCBI Accession No. NM_002015.

| (SEQ ID NO: 12) | |
|---|---|
| 1 | gcagccgcca cattcaacag gcagcagcgc agcgggcgcg ccgctgggga gagcaagcgg |
| 61 | cccgcggcgt ccgtccgtcc ttccgtccgc ggccctgtca gctggagcgc ggcgcaggct |
| 121 | ctgccccggc ccggcggctc tggccggccg tccagtccgt gcggcggacc ccgaggagcc |
| 181 | tcgatgtgga tggccccgcg aagttaagtt ctgggctcgc gcttccactc cgccgcgcct |
| 241 | tcctcccagt ttccgtccgc tcgccgcacc ggcttcgttc ccccaaatct cggaccgtcc |
| 301 | cttcgcgccc cctccccgtc cgcccccagt gctgcgttct ccccctcttg gctctcctgc |
| 361 | ggctggggga ggggcggggg tcaccatggc cgaggcgcct caggtggtgg agatcgaccc |
| 421 | ggacttcgag ccgctgcccc ggccgcgctc gtgcacctgg ccgctgccca ggccggagtt |
| 481 | tagccagtcc aactcggcca cctccagccc ggcgccgtcg ggcagcgcgg ctgccaaccc |
| 541 | cgacgccgcg gcgggcctgc cctcggcctc ggctgccgct gtcagcgccg acttcatgag |
| 601 | caacctgagc ttgctggagg agagcgagga cttcccgcag gcgcccggct ccgtggcggc |
| 661 | ggcggtggcg gcggcggccg ccgcggccgc caccgggggg ctgtgcgggg acttccaggg |
| 721 | cccggaggcg ggctgcctgc acccagcgcc accgcagccc ccgccgcccg ggccgctgtc |
| 781 | gcagcacccg ccggtgcccc ccgccgccgc tgggccgctc gcggggcagc cgcgcaagag |
| 841 | cagctcgtcc cgccgcaacg cgtggggcaa cctgtcctac gccgacctca tcaccaaggc |
| 901 | catcgagagc tcggcggaga agcggctcac gctgtcgcag atctacgagt ggatggtcaa |
| 961 | gagcgtgccc tacttcaagg ataagggtga cagcaacagc tcggcgggct ggaagaattc |
| 1021 | aattcgtcat aatctgtccc tacacagcaa gttcattcgt gtgcagaatg aaggaactgg |
| 1081 | aaaaagttct tggtggatgc tcaatccaga gggtggcaag agcgggaaat ctcctaggag |
| 1141 | aagagctgca tccatggaca acaacagtaa atttgctaag agccgaagcc gagctgccaa |
| 1201 | gaagaaagca tctctccagt ctggccagga gggtgctggg gacagccctg gatcacagtt |
| 1261 | ttccaaatgg cctgcaagcc ctggctctca cagcaatgat gactttgata actggagtac |
| 1321 | atttcgccct cgaactagct caaatgctag tactattagt gggagactct cacccattat |
| 1381 | gaccgaacag gatgatcttg gagaagggga tgtgcattct atggtgtacc cgccatctgc |
| 1441 | cgcaaagatg gcctctactt tacccagtct gtctgagata agcaatcccg aaaacatgga |
| 1501 | aaatcttttg gataatctca accttctctc atcaccaaca tcattaactg tttcgaccca |
| 1561 | gtcctcacct ggcaccatga tgcagcagac gccgtgctac tcgtttgcgc caccaaacac |
| 1621 | cagtttgaat tcacccagcc caaactacca aaaatataca tatggccaat ccagcatgag |
| 1681 | ccctttgccc cagatgccta tacaaacact tcaggacaat aagtcgagtt atggaggtat |
| 1741 | gagtcagtat aactgtgcgc ctggactctt gaaggagttg ctgacttctg actctcctcc |
| 1801 | ccataatgac attatgacac cagttgatcc tggggtagcc cagcccaaca gccgggttct |
| 1861 | gggccagaac gtcatgatgg gccctaattc ggtcatgtca acctatggca gccaggcatc |
| 1921 | tcataacaaa atgatgaatc ccagctccca tacccaccct ggacatgctc agcagacatc |
| 1981 | tgcagttaac gggcgtcccc tgccccacac ggtaagcacc atgccccaca cctcgggtat |
| 2041 | gaaccgcctg acccaagtga agacacctgt acaagtgcct ctgccccacc ccatgcagat |
| 2101 | gagtgccctg gggggctact cctccgtgag cagctgcaat ggctatggca gaatgggcct |
| 2161 | tctccaccag gagaagctcc caagtgactt ggatggcatg ttcattgagc gcttagactg |
| 2221 | tgacatggaa tccatcattc ggaatgacct catggatgga gatacattgg attttaactt |
| 2281 | tgacaatgtg ttgcccaacc aaagcttccc acacagtgtc aagacaacga cacatagctg |
| 2341 | ggtgtcaggc tgagggttag tgagcaggtt acacttaaaa gtacttcaga ttgtctgaca |
| 2401 | gcaggaactg agagaagcag tccaaagatg tctttcacca actccctttt agttttcttg |
| 2461 | gttaaaaaaa aaaacaaaaa aaaaaaccct ccttttttcc tttcgtcaga cttggcagca |
| 2521 | aagacatttt tcctgtacag gatgtttgcc caatgtgtgc aggttatgtg ctgctgtaga |
| 2581 | taaggactgt gccattggaa atttcattac aatgaagtgc caaactcact acaccatata |
| 2641 | attgcagaaa agattttcag atcctggtgt gctttcaagt tttgtatata agcagtagat |
| 2701 | acagattgta tttgtgtgtg tttttggttt ttctaaatat ccaattggtc caaggaaagt |
| 2761 | ttatactctt tttgtaatac tgtgatgggc ctcatgtctt gataagttaa acttttgttt |
| 2821 | gtactacctg ttttctgcgg aactgacgga tcacaaagaa ctgaatctcc attctgcatc |
| 2881 | tccattgaac agccttggac ctgttcacgt tgccacagaa ttcacatgag aaccaagtag |
| 2941 | cctgttatca atctgctaaa ttaatggact tgttaaactt ttggaaaaaa aaagattaaa |
| 3001 | tgccagcttt gtacaggtct tttctatttt tttttgttta ttttgttatt tgcaaatttg |
| 3061 | tacaaacatt taaatggttc taatttccag ataaatgatt tttgatgtta ttgttgggac |
| 3121 | ttaagaacat ttttggaata gatattgaac tgtaataatg ttttcttaaa actagagtct |
| 3181 | actttgttac atagtcagct tgtaaatttt gtggaaccac aggtatttgg ggcagcattc |
| 3241 | ataattttca ttttgtattc taactggatt agtactaatt ttatacatgc ttaactggtt |
| 3301 | tgtacacttt gggatgctac ttagtgatgt ttctgactaa tcttaaatca ttgtaattag |
| 3361 | tacttgcata ttcaacgttt caggccctgg ttgggcagga aagtgatgta tagttatgga |
| 3421 | cactttgcgt ttcttattta ggataactta atatgttttt atgtatgtat tttaaagaaa |
| 3481 | tttcatctgc ttctactgaa ctatgcgtac tgcatagcat caagtcttct ctagagacct |
| 3541 | ctgtagtcct gggaggcctc ataatgtttg tagatcagaa aagggagatc tgcatctaaa |
| 3601 | gcaatggtcc tttgtcaaac gagggatttt gatccacttc accattttga gttgagcttt |
| 3661 | agcaaaagtt tcccctcata attctttgct cttgtttcag tccaggtgga ggttggtttt |
| 3721 | gtagttctgc cttgaggaat tatgtcaaca ctcatacttc atctcattct cccttctgcc |
| 3781 | ctgcagatta gattacttag cacactgtgg aagtttaagt ggaaggaggg aatttaaaaa |
| 3841 | tgggacttga gtggtttgta gaatttgtgt tcataagttc agatgggtag caaatggaat |
| 3901 | agaacttact taaaaattgg ggagatttat ttgaaaacca gctgtaagtt gtgcattgag |
| 3961 | attatgttaa aagccttggc ttaagaattt gaaaatttct ttagcctgta gcaacctaaa |
| 4021 | ctgtaattcc tatcattatg ttttattact ttccaattac ctgtaactga cagaccaaat |
| 4081 | taattggctt tgtgtcctat ttagtccatc agtattttca agtcatgtgg aaagcccaaa |
| 4141 | gtcatcacaa tgaagagaac aggtgcacag cactgttcct cttgtgttct tgagaaggat |
| 4201 | ctaatttttc tgtatatagc ccacatcaca cttgctttgt cttgtatgtt aattgcatct |
| 4261 | tcattggctt ggtatttcct aaatgtttaa caagaacaca agtgttcctg ataagatttc |
| 4321 | ctacagtaag ccagctctat tgtaagcttc ccactgtgat gatcattttt ttgaagattc |
| 4381 | attgaacagc caccactcta tcatcctcat tttggggcag tccaagacat agctggtttt |
| 4441 | agaaacccaa gttcctctaa gcacagcctc ccgggtatgt aactgaactt ggtgccaaag |
| 4501 | tacttgtgta ctaatttcta ttactacgta ctgtcacttt cctcccgtgc cattactgca |
| 4561 | tcataataca aggaacctca gagcccccat ttgttcatta aagaggcaac tacagccaaa |
| 4621 | atcactgtta aaatcttact acttcatgga gtagctctta ggaaaatata tcttcctcct |
| 4681 | gagtctgggt aattatacct ctcccaagcc cccattgtgt gttgaaatcc tgtcatgaat |
| 4741 | ccttggtagc tctctgagaa cagtgaagtc cagggaaagg catctggtct gtctggaaag |
| 4801 | caaacattat gtggcctctg gtagtttttt tcctgtaaga atactgactt tctggagtaa |
| 4861 | tgagtatata tcagttattg tacatgattg ctttgtgaaa tgtgcaaatg atatcaccta |
| 4921 | tgcagccttg tttgatttat tttctctggt ttgtactgtt attaaaagca tattgtatta |
| 4981 | tagagctatt cagatatttt aaatataaag atgtattgtt tccgtaatat agacgtatgg |
| 5041 | aatatattta ggtaatagat gtattacttg gaaagttctg ctttgacaaa ctgacaaagt |
| 5101 | ctaaatgagc acatgtatcc cagtgagcag taaatcaatg gaacatccca agaagaggat |
| 5161 | aaggatgctt aaaatggaaa tcattctcca acgatataca aattggactt gttcaactgc |
| 5221 | tggatatatg ctaccaataa ccccagcccc aacttaaaat tcttacattc aagctcctaa |
| 5281 | gagttcttaa tttataacta attttaaaag agaagtttct tttctggttt tagtttggga |
| 5341 | ataatcattc attaaaaaaa atgtattgtg gtttatgcga acagaccaac ctggcattac |
| 5401 | agttggcctc tccttgaggt gggcacagcc tggcagtgtg gccaggggtg gccatgtaag |
| 5461 | tcccatcagg acgtagtcat gcctcctgca tttcgctacc cgagtttagt aacagtgcag |
| 5521 | attccacgtt cttgttccga tactctgaga agtgcctgat gttgatgtac ttacagacac |
| 5581 | aagaacaatc tttgctataa ttgtataaag ccataaatgt acataaatta tgtttaaatg |
| 5641 | gcttggtgtc tttcttttct aattatgcag aataagctct ttattaggaa ttttttgtga |
| 5701 | agctattaaa tacttgagtt aagtcttgtc agccacaa |

By “FOXA2 polypeptide” (or Forkhead box protein A2) is meant a polypeptide or fragment thereof having at least about 85% amino acid identity to NCBI Accession No. Q9Y261.

| (SEQ ID NO: 13) | |
|---|---|
| 1 | mlgavkmegh epsdwssyya epegyssvsn mnaglgmngm ntymsmsaaa mgsgsgnmsa |
| 61 | gsmnmssyvg agmspslagm spgagamagm ggsagaagva gmgphlspsl splggqaaga |
| 121 | mgglapyanm nsmspmygqa glsrardpkt yrrsythakp pysyislitm aiqqspnkml |
| 181 | tlseiyqwim dlfpfyrqnq qrwqnsirhs lsfndcflkv prspdkpgkg sfwtlhpdsg |
| 241 | nmfengcylr rqkrfkcekq lalkeaagaa gsgkkaaaga qasqaqlgea agpasetpag |
| 301 | tesphssasp cqehkrgglg elkgtpaaal sppepapspg qqqqaaahll gpphhpglpp |
| 361 | eahlkpehhy afnhpfsinn lmsseqqhhh shhhhqphkm dlkayeqvmh ypgygspmpg |
| 421 | slamgpvtnk tgldasplaa dtsyyqgvys rpimnss |

By “FOXA2 nucleic acid molecule” (or Forkhead box protein A2) is meant a polynucleotide (e.g., mRNA) encoding an FOXA2 polypeptide. An exemplary FOXA2 nucleic acid molecule is provided at NCBI Accession No. NM_021784.

| (SEQ ID NO: 14) | |
|---|---|
| 1 | cccgcccact tccaactacc gcctccggcc tgcccaggga gagagaggga gtggagccca |
| 61 | gggagaggga gcgcgagaga gggagggagg aggggacggt gctttggctg actttttttt |
| 121 | aaaagagggt gggggtgggg ggtgattgct ggtcgtttgt tgtggctgtt aaattttaaa |
| 181 | ctgccatgca ctcggcttcc agtatgctgg gagcggtgaa gatggaaggg cacgagccgt |
| 241 | ccgactggag cagctactat gcagagcccg agggctactc ctccgtgagc aacatgaacg |
| 301 | ccggcctggg gatgaacggc atgaacacgt acatgagcat gtcggcggcc gccatgggca |
| 361 | gcggctcggg caacatgagc gcgggctcca tgaacatgtc gtcgtacgtg ggcgctggca |
| 421 | tgagcccgtc cctggcgggg atgtcccccg gcgcgggcgc catggcgggc atgggcggct |
| 481 | cggccggggc ggccggcgtg gcgggcatgg ggccgcactt gagtcccagc ctgagcccgc |
| 541 | tcggggggca ggcggccggg gccatgggcg gcctggcccc ctacgccaac atgaactcca |
| 601 | tgagccccat gtacgggcag gcgggcctga gccgcgcccg cgaccccaag acctacaggc |
| 661 | gcagctacac gcacgcaaag ccgccctact cgtacatctc gctcatcacc atggccatcc |
| 721 | agcagagccc caacaagatg ctgacgctga gcgagatcta ccagtggatc atggacctct |
| 781 | tccccttcta ccggcagaac cagcagcgct ggcagaactc catccgccac tcgctctcct |
| 841 | tcaacgactg tttcctgaag gtgccccgct cgcccgacaa gcccggcaag ggctccttct |
| 901 | ggaccctgca ccctgactcg ggcaacatgt tcgagaacgg ctgctacctg cgccgccaga |
| 961 | agcgcttcaa gtgcgagaag cagctggcgc tgaaggaggc cgcaggcgcc gccggcagcg |
| 1021 | gcaagaaggc ggccgccgga gcccaggcct cacaggctca actcggggag gccgccgggc |
| 1081 | cggcctccga gactccggcg ggcaccgagt cgcctcactc gagcgcctcc ccgtgccagg |
| 1141 | agcacaagcg agggggcctg ggagagctga aggggacgcc ggctgcggcg ctgagccccc |
| 1201 | cagagccggc gccctctccc gggcagcagc agcaggccgc ggcccacctg ctgggcccgc |
| 1261 | cccaccaccc gggcctgccg cctgaggccc acctgaagcc ggaacaccac tacgccttca |
| 1321 | accacccgtt ctccatcaac aacctcatgt cctcggagca gcagcaccac cacagccacc |
| 1381 | accaccacca accccacaaa atggacctca aggcctacga acaggtgatg cactaccccg |
| 1441 | gctacggttc ccccatgcct ggcagcttgg ccatgggccc ggtcacgaac aaaacgggcc |
| 1501 | tggacgcctc gcccctggcc gcagatacct cctactacca gggggtgtac tcccggccca |
| 1561 | ttatgaactc ctcttaagaa gacgacggct tcaggcccgg ctaactctgg caccccggat |
| 1621 | cgaggacaag tgagagagca agtgggggtc gagactttgg ggagacggtg ttgcagagac |
| 1681 | gcaagggaga agaaatccat aacaccccca ccccaacacc cccaagacag cagtcttctt |
| 1741 | cacccgctgc agccgttccg tcccaaacag agggccacac agatacccca cgttctatat |
| 1801 | aaggaggaaa acgggaaaga atataaagtt aaaaaaaagc ctccggtttc cactactgtg |
| 1861 | tagactcctg cttcttcaag cacctgcaga ttctgatttt tttgttgttg ttgttctcct |
| 1921 | ccattgctgt tgttgcaggg aagtcttact taaaaaaaaa aaaaaatttt gtgagtgact |
| 1981 | cggtgtaaaa ccatgtagtt ttaacagaac cagagggttg tactattgtt taaaaacagg |
| 2041 | aaaaaaaata atgtaagggt ctgttgtaaa tgaccaagaa aaagaaaaaa aaagcattcc |
| 2101 | caatcttgac acggtgaaat ccaggtctcg ggtccgatta atttatggtt tctgcgtgct |
| 2161 | ttatttatgg cttataaatg tgtattctgg ctgcaagggc cagagttcca caaatctata |
| 2221 | ttaaagtgtt atacccggtt ttatcccttg aatcttttct tccagatttt tcttttcttt |
| 2281 | acttggctta caaaatatac aggcttggaa attatttcaa gaaggaggga gggataccct |
| 2341 | gtctggttgc aggttgtatt ttattttggc ccagggagtg ttgctgtttt cccaacattt |
| 2401 | tattaataaa attttcagac ataaaaaa |

By “FOXO4 polypeptide” (or Forkhead box protein 04) is meant a polypeptide or fragment thereof having at least about 85% amino acid identity to NCBI Accession No. P98177.

| (SEQ ID NO: 15) | |
|---|---|
| 1 | mdpgnensat eaaaiidldp dfepqsrprs ctwplprpei anqpseppev epdlgekvht |
| 61 | egrsepillp srlpepaggp qpgilgavtg prkggsrrna wgnqsyaeli sqaiesapek |
| 121 | rltlaqiyew mvrtvpyfkd kgdsnssagw knsirhnlsl hskfikvhne atgksswwml |
| 181 | npeggksgka prrraasmds sskllrgrsk apkkkpsvlp appegatpts pvghfakwsg |
| 241 | spcsrnreea dmwttfrprs ssnassvstr lsplrpesev laeeipasvs syaggvpptl |
| 301 | neglelldgl nltsshslls rsglsgfslq hpgvtgplht yssslfspae gplsagegcf |
| 361 | sssqaleall tsdtppppad vlmtqvdpil sqaptllllg glpsssklat gvglcpkple |
| 421 | apgpsslvpt lsmiapppvm asapipkalg tpvltpptea asqdrmpqdl dldmymenle |
| 481 | cdmdniisdl mdegegldfn fepdp |

By “FOXO4 nucleic acid molecule” (or Forkhead box protein 04) is meant a polynucleotide (e.g., mRNA) encoding an FOXO4 polypeptide. An exemplary FOXO4 nucleic acid molecule is provided at NCBI Accession No. NM_005938.

| (SEQ ID NO: 16) | |
|---|---|
| 1 | aaaaggggga gggaactgcg gctaaggaga cgttcggtga tgggagcgca atatatgagg |
| 61 | ggatacagtg cctcaggttt aaaagagcag gaagctgagt gagaggttgc agaaaaagtg |
| 121 | tcttcgctcg gcagaggtta caggtggcat ctcagaaaga gctttgaggc tacaggctgt |
| 181 | agtcgggaag gggatcggag aactgtgtga agggacagct tagggactag cgtcctggga |
| 241 | ctagggggaa gttcgcgact ttctgaagac tggcaggaat gtgcctcctg gccctcgatg |
| 301 | cttcccccct gaggggaggc atcgtgaggg actgtggcag gcttcactga acgctgagcc |
| 361 | ggggaggtcc aactccacgt atggatccgg ggaatgagaa ttcagccaca gaggctgccg |
| 421 | cgatcataga cctagatccc gacttcgaac cccagagccg tccccgctcc tgcacctggc |
| 481 | cccttccccg accagagatc gctaaccagc cgtccgagcc gcccgaggtg gagccagatc |
| 541 | tgggggaaaa ggtacacacg gaggggcgct cagagccgat cctgttgccc tctcggctcc |
| 601 | cagagccggc cgggggcccc cagcccggaa tcctgggggc tgtaacaggt cctcggaagg |
| 661 | gaggctcccg ccggaatgcc tggggaaatc agtcatatgc agaactcatc agccaggcca |
| 721 | ttgaaagcgc cccggagaag cgactgacac ttgcccagat ctacgagtgg atggtccgta |
| 781 | ctgtacccta cttcaaggac aagggtgaca gcaacagctc agcaggatgg aagaactcga |
| 841 | tccgccacaa cctgtccctg cacagcaagt tcatcaaggt tcacaacgag gccaccggca |
| 901 | aaagctcttg gtggatgctg aaccctgagg gaggcaagag cggcaaagcc ccccgccgcc |
| 961 | gggccgcctc catggatagc agcagcaagc tgctccgggg ccgcagtaaa gcccccaaga |
| 1021 | agaaaccatc tgtgctgcca gctccacccg aaggtgccac tccaacgagc cctgtcggcc |
| 1081 | actttgccaa gtggtcaggc agcccttgct ctcgaaaccg tgaagaagcc gatatgtgga |
| 1141 | ccaccttccg tccacgaagc agttcaaatg ccagcagtgt cagcacccgg ctgtccccct |
| 1201 | tgaggccaga gtctgaggtg ctggcggagg aaataccagc ttcagtcagc agttatgcag |
| 1261 | ggggtgtccc tcccaccctc aatgaaggtc tagagctgtt agatgggctc aatctcacct |
| 1321 | cttcccattc cctgctatct cggagtggtc tctctggctt ctctttgcag catcctgggg |
| 1381 | ttaccggccc cttacacacc tacagcagct cccttttcag cccagcagag gggcccctgt |
| 1441 | cagcaggaga agggtgcttc tccagctccc aggctctgga ggccctgctc acctctgata |
| 1501 | cgccaccacc ccctgctgac gtcctcatga cccaggtaga tcccattctg tcccaggctc |
| 1561 | cgactcttct gttgctgggg gggcttcctt cctccagtaa gctggccacg ggcgtcggcc |
| 1621 | tgtgtcccaa gcccctagag gctccaggcc ccagcagtct ggttcccacc ctttctatga |
| 1681 | tagcaccacc tccagtcatg gcaagtgccc ccatccccaa ggctctgggg actcctgtgc |
| 1741 | tcacaccccc tactgaagct gcaagccaag acagaatgcc tcaggatcta gatcttgata |
| 1801 | tgtatatgga gaacctggag tgtgacatgg ataacatcat cagtgacctc atggatgagg |
| 1861 | gcgagggact ggacttcaac tttgagccag atccctgagt catgcctgga agctttgtcc |
| 1921 | cctgcttcag atgtggagcc aggcgtgttc atatctactc tttacccttg agccctcccc |
| 1981 | aggaatttgg gaccctgctt tagagctagg gtggggtctg gtcacacaca ggtgttgaag |
| 2041 | aaattataaa gataaagctg ccccatctgg ggacgatatg gggagggaga tgggagggga |
| 2101 | aaggggagag ggtttttctc actgtgccaa ttagggggta aggccccctc tcaggagcca |
| 2161 | tcatcggctt tccccattcc tacccactta ggctttgtag caagatgagc aatgctgttg |
| 2221 | gaaatgtgaa gtcaccagtg gccttacccc tgcctttggg agcaggattt ttttgtagag |
| 2281 | agtcttatct gagctgagcc aggctagctg gagcctggga tttctatgca gtggcccctt |
| 2341 | aggccagtga tgtgcggtgg gtgggctgtt taggggatct ggaagggcca aggtctgagc |
| 2401 | actggagtgg ctcgccaggc caaatcaccc ttagaaggct gcagataaca gaaaggcttt |
| 2461 | ttataaactt ttaaagaaat ataaacacaa atatagagat tttttaacca tggcagggtg |
| 2521 | ctagtggtgg gcagaatgct tttttttctt tctgaaggct ttgtgatagt gacatgatac |
| 2581 | aaacactaca gacaataaat attaggagac acagggaagt ggggagaggt ggggagtaat |
| 2641 | agtaaacaca gggaagagct cccctacgga ccaggtatag agaaaggtct atgcagaaat |
| 2701 | aggttagagt ttccctaaca aaaaagctaa cccaggtccc ctcattcctt caacttgtgc |
| 2761 | ctgggagtgt gtggtgttag ggtgcagcca cactcttcta tgacccagca tgggttagtg |
| 2821 | ctatggtggg agagtacatt gaaggcctgg aattagcttg gggccaggga agggactggg |
| 2881 | aggggagaga agagaaggag ggaaggattt aggatggtaa agttaggtac agagacctcc |
| 2941 | ctgttcaagg cccctgacag ctgtccctgc ccttcttccc cttccctgac tgcaggggtt |
| 3001 | atgtggaagt gtgtgtggca gcaggcagcg gggaggggag gaacagggaa gggggagctg |
| 3061 | gggagcttgg ctgagggtct gggaaatgag cagggatggg gggggatgtg gatcaggttt |
| 3121 | actagcacct gccagggagg ccatctgggg ctccttctcc accccagccc ccaaagcagc |
| 3181 | ccttccccca gtgccctttg catcgtcccc tcccccaccc ctgctgtggg ttcccatcat |
| 3241 | ttcctgtgtc agcgcctggc ctacccagat tgtatcatgt gctagattgg agtggggaag |
| 3301 | tgtgtcaaat caataaatga ataaattcaa taaatgccta taaccagcaa aaaaaaaaaa |
| 3361 | aaaaa |

By “CNP polypeptide” (or 2′,3′-cyclic-nucleotide 3′-phosphodiesterase) is meant a polypeptide or fragment thereof having at least about 85% amino acid identity to NCBI Accession No. P09543.

| (SEQ ID NO: 17) | |
|---|---|
| 1 | mnrgfsrksh tflpkiffrk msssgakdkp elqfpflqde dtvatlleck tlfilrglpg |
| 61 | sgkstlarvi vdkyrdgtkm vsadaykitp gargafseey krldedlaay crrrdirilv |
| 121 | lddtnherer leqlfemadq yqyqvvlvep ktawrldcaq lkeknqwqls addlkklkpg |
| 181 | lekdflplyf gwfltkksse tlrkagqvfl eelgnhkafk kelrqfvpgd eprekmdlvt |
| 241 | yfgkrppgvl hcttkfcdyg kapgaeeyaq qdvlkksysk aftltisalf vtpkttgarv |
| 301 | elseqqlqlw psdvdklspt dnlprgsrah itlgcaadve avqtgldlle ilrqekggsr |
| 361 | geevgelsrg klyslgngrw mltlaknmev raiftgyygk gkpvptqgsr kggalqscti |
| 421 | i |

By “CNP nucleic acid molecule” (or 2′,3′-cyclic-nucleotide 3′-phosphodiesterase) is meant a polynucleotide (e.g., mRNA) encoding an CNP polypeptide. An exemplary CNP nucleic acid molecule is provided at NCBI Accession No. BC011046.

| (SEQ ID NO: 18) | |
|---|---|
| 1 | ctccgcgcag gcgggcggcc ccggagcgct ggtgccggca gaggcggcga cggtggcgcc |
| 61 | cctcctcatc atgaggcttc tcccgaaaaa gccacacatt cctgcccaag atcttcttcc |
| 121 | gcaagatgtc atcctcaggg gccaaggaca agcctgagct gcagtttccc ttccttcagg |
| 181 | atgaggacac agtggccacg ctgctagagt gcaagacgct cttcatcttg cgcggcctgc |
| 241 | caggaagcgg caagtccacg ctggcacggg tcatcgtgga caagtaccgt gatggcacca |
| 301 | agatggtgtc ggctgacgct tacaagatca cccccggcgc tcgaggagcc ttctccgagg |
| 361 | agtacaagcg gctcgatgag gacctggctg cctactgccg ccgccgggac atcagaattc |
| 421 | ttgtgcttga tgacaccaac cacgaacggg aacggctgga gcagctcttt gaaatggccg |
| 481 | accagtacca gtaccaggtg gtgctggtgg agcccaagac ggcgtggcgg ctggactgtg |
| 541 | cccagctcaa ggagaagaac cagtggcagc tgtcggctga tgacctgaag aagctgaagc |
| 601 | ctgggctgga gaaggacttc ctgccgctct acttcggctg gttcctgacc aagaagagct |
| 661 | ctgagaccct ccgcaaagcc ggccaggtct tcctggaaga gctggggaac cacaaggcct |
| 721 | tcaagaagga gctgcgacaa ttcgtccctg gggatgagcc cagggagaag atggacttgg |
| 781 | tcacctactt tggaaagaga cccccaggcg tgctgcattg cacaaccaag ttttgtgact |
| 841 | acgggaaggc tcccggggca gaggagtacg ctcaacaaga tgtgttaaag aaatcttact |
| 901 | ccaaggcctt cacgctgacc atctctgccc tctttgtgac acccaagacg actggggccc |
| 961 | gggtggagtt aagcgagcag caactgcagt tgtggccgag tgatgtggac aagctgtcac |
| 1021 | ccactgacaa cctgccgcgg gggagccgcg cccacatcac cctcggctgt gcagctgacg |
| 1081 | tagaggccgt gcagacgggc cttgacctct tagagattct gcggcaggag aaggggggca |
| 1141 | gccgaggcga ggaggtgggc gagctaagcc ggggcaagct ctattccttg ggcaatgggc |
| 1201 | gctggatgct gaccctggcc aagaacatgg aggtcagggc catcttcacg gggtactacg |
| 1261 | ggaaaggcaa acctgtgccc acgcaaggta gccggaaggg gggcgccttg cagtcctgca |
| 1321 | ccatcatatg agtgttctca ccaccactta tgcccctaga agggaagggg agagggaaac |
| 1381 | gtgccctctg tttgatcctt gttttgtgac attttttttt tttttttttt tactcaaagt |
| 1441 | taacctacct gtaacttttt aaaaacttgt aaaataactg accctccctt cctgtccgcc |
| 1501 | ctcttcccct ctaatgctca cgctcccaac acaaggtggg cagggaggca ccattcagga |
| 1561 | acctggacca aagctgacga ggctgggcca agccagggat ggggccacag ccagaacccc |
| 1621 | gagccctact tccaggttct ggttagctca gccccagccc agcccagctg ctctgcccag |
| 1681 | agctgggtga gtggggagac acctcagagc cccgcaaaac ccactgaccg gaggcaaaag |
| 1741 | gcagtggggc tgggggtagt tttccatggt cacagagaac tagtggtggc tctgagaagg |
| 1801 | ggaggacctc tgggctttga ttccatctcc ttgtcttttt tctttgtttt tagagacagg |
| 1861 | gtcctgctat ttcccaagct ggagtgcagt ggtgcgatca tggctcactg cagcctcgaa |
| 1921 | ctcctgggct caagcaatcc tcctgagtga tcccatttct taatcagtgt agccccaaga |
| 1981 | aggctggggc tatttaccag ggtagaaaaa ggagcttacc tcccaccttt ggtcctaagt |
| 2041 | ccctgccccc tccccttcac accataacta ggtaacagtt tgataactag ggaagaaagc |
| 2101 | agaacagtta agcagccgcc acatccccgc tggctggggg cctcactcca ggaaggggct |
| 2161 | ggactggctg tcctttccag tggcctggct ccgctgtgtg gatggggaga tcggggccag |
| 2221 | aggcagaacc ctggtgagga agctccagtc ctgctctcta cccagcccat cttgcctcca |
| 2281 | tggtgcctct ggaggcctct gggcctcctc taacaggggc tggtgggcac caagagccaa |
| 2341 | tggagtagac ccctggctgg taagggccaa gtcccaccgg ttgcttctgg gaaggggttt |
| 2401 | ctaacactag tctgtgtgct gtggttcctg gggtgccctc cactgccctc tgttcagtaa |
| 2461 | cagggccttg ctaatcgggt tgtcactcaa caaaagtgct ttggatttaa gttactatcc |
| 2521 | tggctttgcc caacctcagc aacctgtaag actgataatg aaataaatca tgttaatcct |
| 2581 | agcaaaaaaa aaaaaaaa |

By “MBP polypeptide” (or myelin basic protein) is meant a polypeptide or fragment thereof having at least about 85% amino acid identity to NCBI Accession No. P02686.

| (SEQ ID NO: 19) | |
|---|---|
| 1 | mgnhagkrel naekastnse tnrgesekkr nlgelsrtts ednevfgead anqnngtssq |
| 61 | dtavtdskrt adpknawqda hpadpgsrph lirlfsrdap gredntfkdr psesdelqti |
| 121 | qedsaatses ldvmasqkrp sqrhgskyla tastmdharh gflprhrdtg ildsigrffg |
| 181 | gdrgapkrgs gkdshhpart ahygslpqks hgrtqdenpv vhffknivtp rtpppsqgkg |
| 241 | rglslsrfsw gaegqrpgfg yggrasdyks ahkgfkgvda qgtlskifkl ggrdsrsgsp |
| 301 | marr |

By “MBP nucleic acid molecule” (or myelin basic protein) is meant a polynucleotide (e.g., mRNA) encoding an MBP polypeptide. An exemplary MBP nucleic acid molecule is provided at NCBI Accession No. M13577.

| (SEQ ID NO: 20) | |
|---|---|
| 1 | gaaaacagtg cagccacctc cgagagcctg gatgtgatgg cgtcacagaa gagaccctcc |
| 61 | cagaggcacg gatccaagta cctggccaca gcaagtacca tggaccatgc caggcatggc |
| 121 | ttcctcccaa ggcacagaga cacgggcatc cttgactcca tcgggcgctt ctttggcggt |
| 181 | gacaggggtg cgccaaagcg gggctctggc aaggactcac accacccggc aagaactgct |
| 241 | cactatggct ccctgcccca gaagtcacac ggccggaccc aagatgaaaa ccccgtagtc |
| 301 | cacttcttca agaacattgt gacgcctcgc acaccacccc cgtcgcaggg aaaggggaga |
| 361 | ggactgtccc tgagcagatt tagctggggg gccgaaggcc agagaccagg atttggctac |
| 421 | ggaggcagag cgtccgacta taaatcggct cacaagggat tcaagggagt cgatgcccag |
| 481 | ggcacgcttt ccaaaatttt taagctggga ggaagagata gtcgctctgg atcacccatg |
| 541 | gctagacgct gaaaacccac ctggttccgg aatcctgtcc tcagcttctt aatataactg |
| 601 | ccttaaaact ttaatcccac ttgcccctgt tacctaatta gagcagatga cccctcccct |
| 661 | aatgcctgcg gagttgtgca cgtagtaggg tcaggccacg gcagcctacc ggcaatttcc |
| 721 | ggccaacagt taaatgagaa catgaaaaca gaaaacggtt aaaactgtcc ctttctgtgt |
| 781 | gaagatcacg ttccttcccc cgcaatgtgc ccccagacgc acgtgggtct tcagggggcc |
| 841 | aggtgcacag acgtccctcc acgttcaccc ctccaccctt ggactttctt ttcgccgtgg |
| 901 | ctcggcaccc ttgcgctttt gctggtcact gccatggagg cacacagctg cagagacaga |
| 961 | gaggacgtgg gcggcagaga ggactgttga catccaagct tcctttgttt ttttttcctg |
| 1021 | tccttctctc acctcctaaa gtagacttca tttttcctaa caggattaga cagtcaagga |
| 1081 | gtggcttact acatgtggga gctttttggt atgtgacatg cgggctgggc agctgttaga |
| 1141 | gtccaacgtg gggcagcaca gagagggggc cacctcccca ggccgtggct gcccacacac |
| 1201 | cccaattagc tgaattcgcg tgtggcagag ggaggaaaag gaggcaaacg tgggctgggc |
| 1261 | aatggcctca cataggaaac agggtcttcc tggagatttg gtgatggaga tgtcaagcag |
| 1321 | gtggcctctg gacgtcaccg ttgccctgca tggtggcccc agagcagcct ctatgaacaa |
| 1381 | cctcgtttcc aaaccacagc ccacagccgg agagtccagg aagacttgcg cactcagagc |
| 1441 | agaagggtag gagtcctcta gacagcctcg cagccgcgcc agtcgcccat agacactggc |
| 1501 | tgtgaccggg cgtgctggca gcggcagtgc acagtggcca gcactaaccc tccctgagaa |
| 1561 | gataaccggc tcattcactt cctcccagaa gacgcgtggt agcgagtagg cacaggcgtg |
| 1621 | cacctgctcc cgaattactc accgagacac acgggctgag cagacggccc ctgtgatgga |
| 1681 | gacaaagagc tcttctgacc atatccttct taacacccgc tggcatctcc tttcgcgcct |
| 1741 | ccctccctaa cctactgacc caccttttga ttttagcgca cctgtgattg ataggccttc |
| 1801 | caaagagtcc cacgctggca tcaccctccc cgaggacgga gatgaggagt agtcagcgtg |
| 1861 | atgccaaaac gcgtcttctt aatccaattc taattctgaa tgtttcgtgt gggcttaata |
| 1921 | ccatgtctat taatatatag cctcgatgat gagagagtta caaagaacaa aactccagac |
| 1981 | acaaacctcc aaatttttca gcagaagcac tctgcgtcgc tgagctgagg tcggctctgc |
| 2041 | gatccatacg tggccgcacc cacacagcac gtgctgtgac gatggctgaa cggaaagtgt |
| 2101 | acactgttcc tgaatattga aataaaacaa taaactttt |

By “TUBIII polypeptide” (or TUBB3, tubulin beta chain 3) is meant a polypeptide or fragment thereof having at least about 85% amino acid identity to NCBI Accession No. NP_001184110.

| (SEQ ID NO: 21) | |
|---|---|
| 1 | mdsvrsgafg hlfrpdnfif gqsgagnnwa kghytegael vdsvldvvrk ecencdclqg |
| 61 | fqlthslggg tgsgmgtlli skvreeypdr imntfsvvps pkvsdtvvep ynatlsihql |
| 121 | ventdetyci dnealydicf rtlklatpty gdlnhlvsat msgvttslrf pgqlnadlrk |
| 181 | lavnmvpfpr lhffmpgfap ltargsqqyr altvpeltqq mfdaknmmaa cdprhgrylt |
| 241 | vatvfrgrms mkevdeqmla iqsknssyfv ewipnnvkva vcdipprglk msstfignst |
| 301 | aiqelfkris eqftamfrrk aflhwytgeg mdemefteae snmndlvsey qqyqdataee |
| 361 | egemyeddee eseaqgpk |

By “TUBIII nucleic acid molecule” (or TUBB3, tubulin beta chain 3) is meant a polynucleotide (e.g., mRNA) encoding an TUBIII polypeptide. An exemplary TUBIII nucleic acid molecule is provided at NCBI Accession No. BC000748.

| (SEQ ID NO: 22) | |
|---|---|
| 1 | gcccggcccg cccgcgcccg tccgcagccg cccgccagac gcgcccagta tgagggagat |
| 61 | cgtgcacatc caggccggcc agtgcggcaa ccagatcggg gccaagttct gggaagtcat |
| 121 | cagtgatgag catggcatcg accccagcgg caactacgtg ggcgactcgg acttgcagct |
| 181 | ggagcggatc agcgtctact acaacgaggc ctcttctcac aagtacgtgc ctcgagccat |
| 241 | tctggtggac ctggaacccg gaaccatgga cagtgtccgc tcaggggcct ttggacatct |
| 301 | cttcaggcct gacaatttca tctttggtca gagtggggcc ggcaacaact gggccaaggg |
| 361 | tcactacacg gagggggcgg agctggtgga ttcggtcctg gatgtggtgc ggaaggagtg |
| 421 | tgaaaactgc gactgcctgc agggcttcca gctgacccac tcgctggggg gcggcacggg |
| 481 | ctccggcatg ggcacgttgc tcatcagcaa ggtgcgtgag gagtatcccg accgcatcat |
| 541 | gaacaccttc agcgtcgtgc cctcacccaa ggtgtcagac acggtggtgg agccctacaa |
| 601 | cgccacgctg tccatccacc agctggtgga gaacacggat gagacctact gcatcgacaa |
| 661 | cgaggcgctc tacgacatct gcttccgcac cctcaagctg gccacgccca cctacgggga |
| 721 | cctcaaccac ctggtatcgg ccaccatgag cggagtcacc acctccttgc gcttcccggg |
| 781 | ccagctcaac gctgacctgc gcaagctggc cgtcaacatg gtgcccttcc cgcgcctgca |
| 841 | cttcttcatg cccggcttcg cccccctcac agcccggggc agccagcagt accgggccct |
| 901 | gaccgtgccc gagctcaccc agcagatgtt cgatgccaag aacatgatgg ccgcctgcga |
| 961 | cccgcgccac ggccgctacc tgacggtggc caccgtgttc cggggccgca tgtccatgaa |
| 1021 | ggaggtggac gagcagatgc tggccatcca gagcaagaac agcagctact tcgtggagtg |
| 1081 | gatccccaac aacgtgaagg tggccgtgtg tgacatcccg ccccgcggcc tcaagatgtc |
| 1141 | ctccaccttc atcgggaaca gcacggccat ccaggagctg ttcaagcgca tctccgagca |
| 1201 | gttcacggcc atgttccggc gcaaggcctt cctgcactgg tacacgggcg agggcatgga |
| 1261 | cgagatggag ttcaccgagg ccgagagcaa catgaacgac ctggtgtccg agtaccagca |
| 1321 | gtaccaggac gccacggccg aggaagaggg cgagatgtac gaagacgacg aggaggagtc |
| 1381 | ggaggcccag ggccccaagt gaagctgctc gcagctggag tgagaggcag gtggcggccg |
| 1441 | gggccgaagc cagcagtgtc taaacccccg gagccatctt gctgccgaca ccctgctttc |
| 1501 | ccctcgccct agggctccct tgccgccctc ctgcagtatt tatggcctcg tcctccccac |
| 1561 | ctaggccacg tgtgagctgc tcctgtctct gtcttattgc agctccaggc ctgacgtttt |
| 1621 | acggttttgt tttttactgg tttgtgttta tattttcggg gatacttaat aaatctattg |
| 1681 | ctgtcagata cccttaaaaa aaaaaaaaaa aaaaaaaaaa |

By “NEUN polypeptide” (or Feminizing Locus on X-3, Fox-3, RNA-binding protein fox-1 homolog 3, or Hexaribonucleotide Binding Protein-3) is meant a polypeptide or fragment thereof having at least about 85% amino acid identity to NCBI Accession No. NP_001076044.

| (SEQ ID NO: 23) | |
|---|---|
| 1 | maqpyppaqy ppppqngipa eyapppphpt qdysgqtpvp tehgmtlytp aqthpeqpgs |
| 61 | eastqpiagt qtvpqtdeaa qtdsqplhps dptekqqpkr lhvsnipfrf rdpdlrqmfg |
| 121 | qfgkildvei ifnergskgf gfvtfetssd adrareklng tivegrkiev nnatarvmtn |
| 181 | kktgnpytng wklnpvvgav ygpefyavtg fpypttgtav ayrgahlrgr gravyntfra |
| 241 | apppppipty gavvyqdgfy gaeiyggyaa yryaqpaaaa aaysdsygrv yaaadpyhht |
| 301 | igpaatysig tm |

By “NEUN nucleic acid molecule” (or Feminizing Locus on X-3, Fox-3, RNA-binding protein fox-1 homolog 3, or Hexaribonucleotide Binding Protein-3) is meant a polynucleotide (e.g., mRNA) encoding an NEUN polypeptide. An exemplary NEUN nucleic acid molecule is provided at NCBI Accession No. NM_001082575.

| (SEQ ID NO: 24) | |
|---|---|
| 1 | gatacagcag cagctggtgc tcctggccag gctgtgcgtg ctctctctgc ctctctctct |
| 61 | cggactctct gctctctctc tctgactctc tcctctctct ctgttggcct ggtgaaatgt |
| 121 | tcttggctgt aggcacacag agccttggac tcaaggctgt tggagtcgag gacaccttga |
| 181 | cttcggtcct ggaggttgaa attctgcctc tgagaagcta acagtcttcc tgtggtcgcc |
| 241 | actcctcccc agcagccccc tccttgccaa ggacggtcca gaaggagccc cactggggcc |
| 301 | tccccgctca gcaaagcaga cctcacctcc cactaccagc ttgaagtcac agcagccaga |
| 361 | ggaaattctg ccaccatttt cccaggtctg cagcccctcc agctgggaac ctgctcctgg |
| 421 | agccatccct ctgcaaacag agagcccaga gtgcctcggg gaaaattggc tgaataaaag |
| 481 | agcgatcagg acgccacggc tccgcctgaa gcgatggccc agccctaccc ccccgcccag |
| 541 | tacccccctc cgccacagaa cggcatccct gccgagtacg ccccgccccc accgcacccc |
| 601 | acgcaggact actccggcca gaccccggtc cccacagagc atggcatgac cctgtacaca |
| 661 | ccagcacaga cccaccccga gcagccaggc tccgaggcca gcacacagcc catcgccggg |
| 721 | acccagacag tgccgcagac agacgaggcg gcacagacgg acagccagcc gctccacccc |
| 781 | tccgacccta cagagaagca gcagcccaag cggctacacg tctccaacat ccccttccgg |
| 841 | ttcagggacc ccgacttgcg gcaaatgttc gggcaattcg gaaaaatttt agacgtggag |
| 901 | atcattttta acgagcgggg ctccaagggt tttgggtttg taacttttga aactagctca |
| 961 | gatgctgacc gagcccggga gaagctgaat gggacgatcg tagagggacg gaaaattgag |
| 1021 | gtcaataatg ccacggcccg agtgatgacc aacaagaaga cggggaaccc ctacaccaac |
| 1081 | ggctggaagc taaatccagt ggtcggcgca gtctacgggc ctgaattcta tgcagtgacg |
| 1141 | gggttcccct accccaccac cggcacagcc gttgcctacc ggggcgcaca tcttcggggc |
| 1201 | cggggccggg ccgtgtataa tacatttcgg gctgcgccac ccccaccccc catcccgact |
| 1261 | tacggagcgg tcgtgtatca ggatggattt tatggtgctg agatttatgg aggctacgca |
| 1321 | gcctacagat acgctcagcc cgctgcagcg gcggcagcct acagcgacag ttacggcaga |
| 1381 | gtctacgcag ctgccgaccc gtaccatcac accatcgggc ccgcggcgac ctacagcatt |
| 1441 | ggaaccatgt gaaaccttcc accgtttcct tctcggacca tgaagggcaa aaacaaaaaa |
| 1501 | acaaaaaaaa tcacaaaaca aaaaaaacaa aaaaagatgt taagatccaa gcaacaaaaa |
| 1561 | aaaaaccaac caaaccaaga ggcatccaac caagtccaag tcccgcgtcc tggccacacg |
| 1621 | cccgcaccga gggagcacgc cggcaggggc gccgaggagc ggccccagga caggacggcc |
| 1681 | ccaccgcgtc ctggctggca gcacagtggg aacacgcccc tccgtctcag gcagtggggg |
| 1741 | agttggaggg gaaggggcct cccttgtggg acccgtgggg ggctctgttt tccatccagt |
| 1801 | cttcctttcc cagcccccaa ctcccaagac agacagtgtg gagcccagcg gcggcggagc |
| 1861 | aggcccgggc ctgagcaggc aggcgctgct agcaagactt gatctttgtg gccagctgtg |
| 1921 | ccagggggcc ggcggggctg aggggtgcgg gcagctttca tcccaggggc tccactgggc |
| 1981 | cccgtcaccc tcctgtcgcg tcccctgcgt cccacctccc tcctgcccgg cagtcccgcc |
| 2041 | cgtgccccca gcctggcgag gaagccgtcc aacagtagcc ccggggccag ctcccaacag |
| 2101 | aaagggctga cgtggctcca ggactcaggg gcgctccatg ggaggacgaa ggaagcccag |
| 2161 | ccagccagga gccactcctc acacctccaa gtgtggccaa gtgggccctg aggccaagga |
| 2221 | cttacttgct cttcctggcc atctctccct ttctggagga ggcccggggc ctgtgtacac |
| 2281 | caaggctgac ctcgtgctgc ctgctgggac ccagccctcc ctgccgctcc cctgtgagcc |
| 2341 | cagtccaccg tgggcgccca gggccaggga cgggccagcg cccggctgca tcgcgaggtt |
| 2401 | gggagtcaca gtggctgtgg gcctggacgg gcacagccag agcaggggcc catgggaagg |
| 2461 | gcaagggatg gggaagcctg ggccggcccc ttccctgctc ccaaggcagg tgtccaggtg |
| 2521 | gcgggagcag caccaaggac agccaggctt acccggtggg aggagcagga gcagagcagg |
| 2581 | tggcagggag gaacccctgg cgaggcaggg agcactgaag tagggaagca gcaaaaaata |
| 2641 | caggctccca acgtggctcc actgtctcat gaagtgtcaa aaatttaaaa atacacctca |
| 2701 | ctttctattc agcatcagct attgaaatgg aattctcctt ttctattccc gttgtacata |
| 2761 | gccccacgcc ctgcctccgg ctttgtcctc tgtacagagc cccctgtccc ctctgctgtt |
| 2821 | ccggaccctt ttcttgcagc agctcaaccc cccgactcac tcagatcccc aggactgcag |
| 2881 | ccgagccccg ggcttccttt cttaccattc tgtatgcttc caaggtgtga ccattcaaac |
| 2941 | taacagtatt attaagatta ttaataaaga tttctttctt caaaccagga aaaaaaaaaa |
| 3001 | aaaaaaa |

By “SLC1A6 polypeptide” (or Excitatory amino acid transporter 4; Sodium-dependent glutamate/aspartate transporter; Solute carrier family 1 member 6) is meant a polypeptide or fragment thereof having at least about 85% amino acid identity to NCBI Accession No. P48664.

| (SEQ ID NO: 25) | |
|---|---|
| 1 | msshgnslfl resgqrlgrv gwlqrlqesl qqralrtrlr lqtmtlehvl rflrrnafil |
| 61 | ltvsavvigv slafalrpyq ltyrqikyfs fpgellmrml qmlvlplivs slvtgmasld |
| 121 | nkatgrmgmr aavyymvtti iavfigilmv tiihpgkgsk eglhregrie tiptadafmd |
| 181 | lirnmfppnl veacfkqfkt qystrvvtrt mvrtengsep gasmpppfsv engtsflenv |
| 241 | tralgtlqem lsfeetvpvp gsanginalg lvvfsvafgl viggmkhkgr vlrdffdsln |
| 301 | eaimrlvgii iwyapvgilf liagkileme dmavlggqlg mytltvivgl flhagivlpl |
| 361 | iyflvthrnp fpfiggmlqa litamgtsss satlpitfrc leeglgvdrr itrfvlpvga |
| 421 | tvnmdgtaly ealaaifiaq vnnyelnlgq ittisitata asvgaagipq aglvtmvivl |
| 481 | tsvglptedi tliiavdwfl drlrtmtnvl gdsigaavie hlsqrelelq eaeltlpslg |
| 541 | kpykslmaqe kgasrgrggn esam |

By “SLC1A6 nucleic acid molecule” (or Excitatory amino acid transporter 4; Sodium-dependent glutamate/aspartate transporter; Solute carrier family 1 member 6) is meant a polynucleotide (e.g., mRNA) encoding an SLC1A6 polypeptide. An exemplary SLC1A6 nucleic acid molecule is provided at NCBI Accession No. BC040604.

| (SEQ ID NO: 26) | |
|---|---|
| 1 | ggcatagcgc gtcccggctc cgcgccggtg cctccacggt ccggtccccg cgccggtgct |
| 61 | gcacagtccc tggcgggtcc ccgcggcccc ggccgggcgc ttcgccgggc tccggctcct |
| 121 | gcatccgggc gcagcgcgca ggccgaggcg cgggcaggcc gcccccgccg ctccggacgc |
| 181 | cgggatgtaa gaggctccga aaagcagccc acgcatctca tcagatctaa gtgtctagag |
| 241 | gtcgggagaa ccaagtggga aagacccacc ctcacccctc accttgtaga aactgggaac |
| 301 | actagaaggg acattttctg agcaggaaac ccaagagaca gggttttacg ctgtcaccca |
| 361 | agttggagtg cagtggtacg atcatagctc attgcagcct caaactcctg ggttcaagcg |
| 421 | atcctcctgc tttagcctct tgagtagcta ggactacagg cacaggccac cgtgcctggc |
| 481 | taatttttaa tttttaaaaa agagacaggg tctggctatg ttgcccaggc tggccatgaa |
| 541 | ctcctgggct caagcggttc tccagccttc acctcccaaa gtgttgggat tgcaggcatg |
| 601 | agccactgcg tctggcccac agatgctaag tgctgtctgc tcttctccag gggtcagcaa |
| 661 | attttttcag caaatggccc aagagtaaat attttgagct ttgtggcccg tacaatctct |
| 721 | gtcccaacaa ctcaactcag gcattgtagc ttgaaagcag ctgtagacaa taggtaatcc |
| 781 | atgagtgtgg ctgtgtgcca ataaaacttt atttacaaaa acaagcagta ggctgaattt |
| 841 | gactagcaga ccatagtttg tcaataccgt attatgtctt gtaaggaaga gaaaggaacc |
| 901 | agacaaaact ctagcctcgg gagttttcct gactgttcag atcttagctg aatgatctcc |
| 961 | cttggtatct acaggcaact tcctgctgtg gcttagggac tggaaacata atatcccaga |
| 1021 | gggattccct gtgtagtctg tggttcactc tttgggattt tttttttttt tttcacagca |
| 1081 | aggagaagca gcattgtggt ttcaggagat gggtccattt ggagcaggat cctaagtggg |
| 1141 | gcttggcatt gggaatttgg attagctcta gaggacgcag gatctggaaa atcagggcag |
| 1201 | atttcccatc ccttggatat ggtggggagt tgaggagggc aaggaagatc ccagaaaagc |
| 1261 | cagtggcagc aaaacacaaa ggccagggac ctacgtactg gtaaaactga gacctccaag |
| 1321 | aaacctgcag ctcgacctgg ttgaattcag atagaccatg agcagccatg gcaacagcct |
| 1381 | gttccttcgg gagagcggcc agcggctggg ccgggtgggc tggctgcagc ggctgcagga |
| 1441 | aagcctgcag cagagagcac tgcgcacgcg cctgcgcctg cagaccatga ccctcgagca |
| 1501 | cgtgctgcgc ttcctgcgcc gaaacgcctt cattctgctg acggtcagcg ccgtggtcat |
| 1561 | tggggtcagc ctggcctttg ccctgcgccc atatcagctc acctaccgcc agatcaagta |
| 1621 | cttctctttt cctggagagc ttctgatgag gatgctgcag atgctggtgt tacctctcat |
| 1681 | tgtctccagc ctggtcacag gtatggcatc cctggacaac aaggccacgg ggcggatggg |
| 1741 | gatgcgggca gctgtgtact acatggtgac caccatcatc gcggtcttca tcggcatcct |
| 1801 | catggtcacc atcatccatc ccgggaaggg ctccaaggag gggctgcacc gggagggccg |
| 1861 | gatcgagacc atccccacag ctgatgcctt catggacctg atcagaaata tgtttccacc |
| 1921 | aaaccttgtg gaggcctgct tcaaacagtt caagacgcag tacagcacga gggtggtaac |
| 1981 | caggaccatg gtgaggacag agaacgggtc tgagccgggt gcctccatgc ctcctccatt |
| 2041 | ctcagtggag aacggaacca gcttcctgga aaatgtcact cgggccttgg gtaccctgca |
| 2101 | ggagatgctg agctttgagg agactgtacc cgtgcctggc tccgccaatg gcatcaacgc |
| 2161 | cctgggcctc gtggtcttct ctgtggcctt tgggctggtc attggtggca tgaaacacaa |
| 2221 | gggcagagtc ctcagggact tcttcgacag cctcaatgag gctattatga ggctggtggg |
| 2281 | catcattatc tggtgagtcc tggtctgtgc ccacgggaag gtggagccag agctgggaag |
| 2341 | tcaggctgtg gggaagctgc cgaagggctt gctggggacc tttggtcatt catttacgta |
| 2401 | ttgggtgatt cacttaccca ctcaccaact cattcattca tgtctttctg ggatgatttc |
| 2461 | atcactagtt cacttccttg ttcatctgtt cattcattca ttcttctatg cattggttag |
| 2521 | ttcatggaat atctcactct ttcattcatt catgtccttc tgcaatgatt cattcactgc |
| 2581 | tttgttcatc tgttcattca ctcattcttc tatgcattga tgaaatcact cattcagtga |
| 2641 | tttattcatc tatactcatg cttcaatgca ttgatttact catttcctca tgcatttatt |
| 2701 | cattcatcta tgcattggtt aaatcactgg ccaactcact aactcattca ttcattcaca |
| 2761 | cttttctgca atgatttgtt cacttgttca ctcccttgct tatctgttca ttcactcatt |
| 2821 | cttcaataca ttgaccaagc cattcactga catttattca gctacattta ttctttcatg |
| 2881 | cattggtctg gatttatttg gtcattcatt tatttatttt gcaaaattaa tgtattttta |
| 2941 | attgacaaat aaaaactgta tatattttca tgtgcaaaaa aaaaaaaaaa |

By “NOGOA polypeptide” (or neurite outgrowth inhibitor A; neurite outgrowth inhibitor isoform A; human reticulon-4; human reticulon-4 isoform A) is meant a polypeptide or fragment thereof having at least about 85% amino acid identity to NCBI Accession No. NP_065393.

| (SEQ ID NO: 27) | |
|---|---|
| 1 | medldqsplv sssdspprpq pafkyqfvre pedeeeeeee eeedededle elevlerkpa |
| 61 | aglsaapvpt apaagaplmd fgndfvppap rgplpaappv aperqpswdp spvsstvpap |
| 121 | splsaaavsp sklpeddepp arppppppas vspqaepvwt ppapapaapp stpaapkrrg |
| 181 | ssgsvdetlf alpaasepvi rssaenmdlk eqpgntisag qedfpsvlle taaslpslsp |
| 241 | lsaasfkehe ylgnlstvlp tegtlqenvs easkevseka ktllidrdlt efseleysem |
| 301 | gssfsvspka esavivanpr eeiivknkde eeklvsnnil hnqqelptal tklvkedevv |
| 361 | ssekakdsfn ekrvaveapm reeyadfkpf ervwevkdsk edsdmlaagg kiesnleskv |
| 421 | dkkcfadsle qtnhekdses snddtsfpst pegikdrsga yitcapfnpa atesiatnif |
| 481 | pllgdptsen ktdekkieek kaqivteknt stktsnpflv aaqdsetdyv ttdnltkvte |
| 541 | evvanmpegl tpdlvqeace selnevtgtk iayetkmdlv qtsevmqesl ypaaqlcpsf |
| 601 | eeseatpspv lpdivmeapl nsavpsagas viqpsssple assvnyesik hepenpppye |
| 661 | eamsvslkkv sgikeeikep eninaalqet eapyisiacd liketklsae papdfsdyse |
| 721 | makveqpvpd hselvedssp dsepvdlfsd dsipdvpqkq detvmlvkes ltetsfesmi |
| 781 | eyenkeklsa lppeggkpyl esfklsldnt kdtllpdevs tlskkekipl qmeelstavy |
| 841 | snddlfiske aqiretetfs dsspieiide fptlissktd sfsklareyt dlevshksei |
| 901 | anapdgagsl pctelphdls lkniqpkvee kisfsddfsk ngsatskvll lppdvsalat |
| 961 | qaeiesivkp kvlvkeaekk lpsdtekedr spsaifsael sktsvvdlly wrdikktgvv |
| 1021 | fgaslfllls ltvfsivsvt ayialallsv tisfriykgv iqaiqksdeg hpfraylese |
| 1081 | vaiseelvqk ysnsalghvn ctikelrrlf lvddlvdslk favlmwvfty vgalfngltl |
| 1141 | lilalislfs vpviyerhqa qidhylglan knvkdamaki qakipglkrk ae |

By “NOGOA nucleic acid molecule” (or neurite outgrowth inhibitor A; neurite outgrowth inhibitor isoform A; human reticulon-4; human reticulon-4 isoform A) is meant a polynucleotide encoding an NOGOA polypeptide. An exemplary NOGOA nucleic acid molecule (e.g., mRNA) is provided at NCBI Accession No. NM_020532.

| (SEQ ID NO: 28) | |
|---|---|
| 1 | agtccctgcc ctcccctggg gagggtgagt cacgccaaac tgggcggaga gtccgctggc |
| 61 | ctcactccta gctcatctgg gcggcggcgg caagtgggga cagggcgggt ggcgcatcac |
| 121 | cggcgcggag gcaggaggag cagtctcatt gttccgggag ccgtcaccac agtaggtccc |
| 181 | tcggctcagt cggcccagcc cctctcagtc ctccccaacc cccacaaccg cccgcggctc |
| 241 | tgagacgcgg ccccggcggc ggcggcagca gctgcagcat catctccacc ctccagccat |
| 301 | ggaagacctg gaccagtctc ctctggtctc gtcctcggac agcccacccc ggccgcagcc |
| 361 | cgcgttcaag taccagttcg tgagggagcc cgaggacgag gaggaagaag aggaggagga |
| 421 | agaggaggac gaggacgaag acctggagga gctggaggtg ctggagagga agcccgccgc |
| 481 | cgggctgtcc gcggccccag tgcccaccgc ccctgccgcc ggcgcgcccc tgatggactt |
| 541 | cggaaatgac ttcgtgccgc cggcgccccg gggacccctg ccggccgctc cccccgtcgc |
| 601 | cccggagcgg cagccgtctt gggacccgag cccggtgtcg tcgaccgtgc ccgcgccatc |
| 661 | cccgctgtct gctgccgcag tctcgccctc caagctccct gaggacgacg agcctccggc |
| 721 | ccggcctccc cctcctcccc cggccagcgt gagcccccag gcagagcccg tgtggacccc |
| 781 | gccagccccg gctcccgccg cgcccccctc caccccggcc gcgcccaagc gcaggggctc |
| 841 | ctcgggctca gtggatgaga ccctttttgc tcttcctgct gcatctgagc ctgtgatacg |
| 901 | ctcctctgca gaaaatatgg acttgaagga gcagccaggt aacactattt cggctggtca |
| 961 | agaggatttc ccatctgtcc tgcttgaaac tgctgcttct cttccttctc tgtctcctct |
| 1021 | ctcagccgct tctttcaaag aacatgaata ccttggtaat ttgtcaacag tattacccac |
| 1081 | tgaaggaaca cttcaagaaa atgtcagtga agcttctaaa gaggtctcag agaaggcaaa |
| 1141 | aactctactc atagatagag atttaacaga gttttcagaa ttagaatact cagaaatggg |
| 1201 | atcatcgttc agtgtctctc caaaagcaga atctgccgta atagtagcaa atcctaggga |
| 1261 | agaaataatc gtgaaaaata aagatgaaga agagaagtta gttagtaata acatccttca |
| 1321 | taatcaacaa gagttaccta cagctcttac taaattggtt aaagaggatg aagttgtgtc |
| 1381 | ttcagaaaaa gcaaaagaca gttttaatga aaagagagtt gcagtggaag ctcctatgag |
| 1441 | ggaggaatat gcagacttca aaccatttga gcgagtatgg gaagtgaaag atagtaagga |
| 1501 | agatagtgat atgttggctg ctggaggtaa aatcgagagc aacttggaaa gtaaagtgga |
| 1561 | taaaaaatgt tttgcagata gccttgagca aactaatcac gaaaaagata gtgagagtag |
| 1621 | taatgatgat acttctttcc ccagtacgcc agaaggtata aaggatcgtt caggagcata |
| 1681 | tatcacatgt gctcccttta acccagcagc aactgagagc attgcaacaa acatttttcc |
| 1741 | tttgttagga gatcctactt cagaaaataa gaccgatgaa aaaaaaatag aagaaaagaa |
| 1801 | ggcccaaata gtaacagaga agaatactag caccaaaaca tcaaaccctt ttcttgtagc |
| 1861 | agcacaggat tctgagacag attatgtcac aacagataat ttaacaaagg tgactgagga |
| 1921 | agtcgtggca aacatgcctg aaggcctgac tccagattta gtacaggaag catgtgaaag |
| 1981 | tgaattgaat gaagttactg gtacaaagat tgcttatgaa acaaaaatgg acttggttca |
| 2041 | aacatcagaa gttatgcaag agtcactcta tcctgcagca cagctttgcc catcatttga |
| 2101 | agagtcagaa gctactcctt caccagtttt gcctgacatt gttatggaag caccattgaa |
| 2161 | ttctgcagtt cctagtgctg gtgcttccgt gatacagccc agctcatcac cattagaagc |
| 2221 | ttcttcagtt aattatgaaa gcataaaaca tgagcctgaa aaccccccac catatgaaga |
| 2281 | ggccatgagt gtatcactaa aaaaagtatc aggaataaag gaagaaatta aagagcctga |
| 2341 | aaatattaat gcagctcttc aagaaacaga agctccttat atatctattg catgtgattt |
| 2401 | aattaaagaa acaaagcttt ctgctgaacc agctccggat ttctctgatt attcagaaat |
| 2461 | ggcaaaagtt gaacagccag tgcctgatca ttctgagcta gttgaagatt cctcacctga |
| 2521 | ttctgaacca gttgacttat ttagtgatga ttcaatacct gacgttccac aaaaacaaga |
| 2581 | tgaaactgtg atgcttgtga aagaaagtct cactgagact tcatttgagt caatgataga |
| 2641 | atatgaaaat aaggaaaaac tcagtgcttt gccacctgag ggaggaaagc catatttgga |
| 2701 | atcttttaag ctcagtttag ataacacaaa agataccctg ttacctgatg aagtttcaac |
| 2761 | attgagcaaa aaggagaaaa ttcctttgca gatggaggag ctcagtactg cagtttattc |
| 2821 | aaatgatgac ttatttattt ctaaggaagc acagataaga gaaactgaaa cgttttcaga |
| 2881 | ttcatctcca attgaaatta tagatgagtt ccctacattg atcagttcta aaactgattc |
| 2941 | attttctaaa ttagccaggg aatatactga cctagaagta tcccacaaaa gtgaaattgc |
| 3001 | taatgccccg gatggagctg ggtcattgcc ttgcacagaa ttgccccatg acctttcttt |
| 3061 | gaagaacata caacccaaag ttgaagagaa aatcagtttc tcagatgact tttctaaaaa |
| 3121 | tgggtctgct acatcaaagg tgctcttatt gcctccagat gtttctgctt tggccactca |
| 3181 | agcagagata gagagcatag ttaaacccaa agttcttgtg aaagaagctg agaaaaaact |
| 3241 | tccttccgat acagaaaaag aggacagatc accatctgct atattttcag cagagctgag |
| 3301 | taaaacttca gttgttgacc tcctgtactg gagagacatt aagaagactg gagtggtgtt |
| 3361 | tggtgccagc ctattcctgc tgctttcatt gacagtattc agcattgtga gcgtaacagc |
| 3421 | ctacattgcc ttggccctgc tctctgtgac catcagcttt aggatataca agggtgtgat |
| 3481 | ccaagctatc cagaaatcag atgaaggcca cccattcagg gcatatctgg aatctgaagt |
| 3541 | tgctatatct gaggagttgg ttcagaagta cagtaattct gctcttggtc atgtgaactg |
| 3601 | cacgataaag gaactcaggc gcctcttctt agttgatgat ttagttgatt ctctgaagtt |
| 3661 | tgcagtgttg atgtgggtat ttacctatgt tggtgccttg tttaatggtc tgacactact |
| 3721 | gattttggct ctcatttcac tcttcagtgt tcctgttatt tatgaacggc atcaggcaca |
| 3781 | gatagatcat tatctaggac ttgcaaataa gaatgttaaa gatgctatgg ctaaaatcca |
| 3841 | agcaaaaatc cctggattga agcgcaaagc tgaatgaaaa cgcccaaaat aattagtagg |
| 3901 | agttcatctt taaaggggat attcatttga ttatacgggg gagggtcagg gaagaacgaa |
| 3961 | ccttgacgtt gcagtgcagt ttcacagatc gttgttagat ctttattttt agccatgcac |
| 4021 | tgttgtgagg aaaaattacc tgtcttgact gccatgtgtt catcatctta agtattgtaa |
| 4081 | gctgctatgt atggatttaa accgtaatca tatctttttc ctatctatct gaggcactgg |
| 4141 | tggaataaaa aacctgtata ttttactttg ttgcagatag tcttgccgca tcttggcaag |
| 4201 | ttgcagagat ggtggagcta gaaaaaaaaa aaaaaaagcc cttttcagtt tgtgcactgt |
| 4261 | gtatggtccg tgtagattga tgcagatttt ctgaaatgaa atgtttgttt agacgagatc |
| 4321 | ataccggtaa agcaggaatg acaaagcttg cttttctggt atgttctagg tgtattgtga |
| 4381 | cttttactgt tatattaatt gccaatataa gtaaatatag attatatatg tatagtgttt |
| 4441 | cacaaagctt agacctttac cttccagcca ccccacagtg cttgatattt cagagtcagt |
| 4501 | cattggttat acatgtgtag ttccaaagca cataagctag aagaagaaat atttctagga |
| 4561 | gcactaccat ctgttttcaa catgaaatgc cacacacata gaactccaac atcaatttca |
| 4621 | ttgcacagac tgactgtagt taattttgtc acagaatcta tggactgaat ctaatgcttc |
| 4681 | caaaaatgtt gtttgtttgc aaatatcaaa cattgttatg caagaaatta ttaattacaa |
| 4741 | aatgaagatt tataccattg tggtttaagc tgtactgaac taaatctgtg gaatgcattg |
| 4801 | tgaactgtaa aagcaaagta tcaataaagc ttatagactt aaaaaaaaaa aaaaaaaaaa |
| 4861 | aaaaaaaaaa a |

By “oligodendrocyte O1 polypeptide” (or oligodendrocyte marker O1; oligodendrocyte transcription factor 1: olig1) is meant a polypeptide or fragment thereof having at least about 85% amino acid identity to NCBI Accession No. Q8TAK6.

| (SEQ ID NO: 29) | |
|---|---|
| 1 | myyavsqarv navpgtmlrp qrpgdlqlga slyelvgyrq ppssssssts stsstsssst |
| 61 | tapllpkaar ekpeapaepp gpgpgsgahp ggsarpdake eqqqqlrrki nsrerkrmqd |
| 121 | lnlamdalre vilpysaahc qgapgrklsk iatlllarny illlgsslqe lrralgegag |
| 181 | paaprlllag lpllaaapgs vllapgavgp pdalrpakyl slaldeppcg qfalpgggag |
| 241 | gpglctcavc kfphlvpasl glaavqaqfs k |

By “oligodendrocyte O1 nucleic acid molecule” (or oligodendrocyte marker O1; oligodendrocyte transcription factor 1; olig1) is meant a polynucleotide encoding an oligodendrocyte O1 polypeptide. An exemplary oligodendrocyte O1 nucleic acid molecule (e.g., mRNA) is provided at NCBI Accession No. NM_138983.

| (SEQ ID NO: 30) | |
|---|---|
| 1 | gttctagatc gtttccccgc gcgcaggtcc gcggggaggg gcggcctgcc gaccggccca |
| 61 | ccccagggcg ttcctgaagg gcgtcctcgg ccgcccccac cgcctcccag atgtactatg |
| 121 | cggtttccca ggcgcgcgtg aacgcggtcc ccgggaccat gctgcggcca cagcggcccg |
| 181 | gagacttgca gctcggggcc tccctctacg agctggtggg ctacaggcag ccgccctcct |
| 241 | cctcctcctc ctccacctcc tccacctcct ccacttcctc ctcctccacg acggcccccc |
| 301 | tcctccccaa ggctgcgcgc gagaagccgg aggcgccggc cgagcctcca ggccccgggc |
| 361 | ccgggtcagg cgcgcacccg ggcggcagcg cccggccgga cgccaaggag gagcagcagc |
| 421 | agcagctgcg gcgcaagatc aacagccgcg agcggaagcg catgcaggac ctgaacctgg |
| 481 | ccatggacgc cctgcgcgag gtcatcctgc cctactcagc ggcgcactgc cagggcgcgc |
| 541 | ccggccgcaa gctctccaag atagccacgc tgctgctcgc ccgcaactac atcctactgc |
| 601 | tgggcagctc gctgcaggag ctgcgccgcg cgctgggcga gggcgccggg cccgccgcgc |
| 661 | cgcgcctgct gctggccggg ctgcccctgc tcgccgccgc gcccggctcc gtgctgctgg |
| 721 | cgcccggcgc cgtaggaccc cccgacgcgc tgcgccccgc caagtacctg tcgctggcgc |
| 781 | tggacgagcc gccgtgcggc cagttcgctc tccccggcgg cggcgcaggc ggccccggcc |
| 841 | tctgcacctg cgccgtgtgc aagttcccgc acctggtccc ggccagcctg ggcctggccg |
| 901 | ccgtgcaggc gcaattctcc aagtgagggc gggtctgggc ctggggcgcg acctcggccc |
| 961 | ggcctccctt cgctcagctt ctccgcgccc ctgctccctg cgtctgggag agcgaggccg |
| 1021 | agcaaggaaa gcatttcgaa ccttccagtc cagaggaagg gactgtcggg cacccccttc |
| 1081 | cccgccccca cccctgggac gttaaagtga ccagagcgga tgttcgatgg cgcctcgggg |
| 1141 | cagtttgggg ttctgggtcg gttccagcgg ctttaggcag aaagtgctcg ctctcaccca |
| 1201 | gcacatctct ctccttgtcc ctggagttgc gcgcttcgcg gggccgatgt agaacttagg |
| 1261 | gcgccttgcc gtggttggcg cgccccgggt gcagcgagag gccatccccg agcgctacct |
| 1321 | ccccggagcg gagcacgcgg gctcccagta ctaggggctg cgctcgagca gtggcggggg |
| 1381 | cggaggggtg gttcttttcc ttctcctccg ccagaggcca cgggcgccct tgttcccgcc |
| 1441 | ggccaggtcc tatcaaagga ggctgccgga actcaagagg cagaaaaaga ccagttaggc |
| 1501 | ggtgcagacg gtctgggacg tggcagacgg acggaccctc ggcggacagg tggtcggcgt |
| 1561 | cggggtgcgg tgggtagggg cgaggacaac gcagggtgcg ctgggttggg acgtgggtcc |
| 1621 | acttttgtag accagctgtt tggagagctg tatttaagac tcgcgtatcc agtgttttgt |
| 1681 | cgcagagagt tttcactctt aaatcctggg ggtttcttag aaagcaactt agaactcgag |
| 1741 | attcaccttt cgtttccctt tccccaaaag tagcgtaacc aacatttaag cttgcttaaa |
| 1801 | aacgaaaacc aaccgccttg catccagtgt tcccgattta ctaaaatagg taaccaggcg |
| 1861 | tctcacagtc gccgtcctgt caagagcgct aatgaacgtt ctcattaaca cgcaggagta |
| 1921 | ccgggagccc tgaaccgccc gctgctcggc ggatcccagc tgcggtggcg acggcgggaa |
| 1981 | ggcgctttcc gctgttcctc agcgggccgg gcccttgacc agcgcggccc gcaggtcttc |
| 2041 | cttctcgccg tcttgcagtt gaagagctac atacgtagtc agtttcgatt tgttacagac |
| 2101 | gttaacaaat tcctttaccc aaggttatgc tatgaccttt ccgcagttta ctttgatttt |
| 2161 | ctatgtttaa ggttttggtt gttggtagta gccgaattta actggcactt tattttactt |
| 2221 | ctaaccttgt ttcctgacgg tgtacagaat caacaaaata aaacatttaa agtctgattt |
| 2281 | tttaaaaaaa aaaaaaaa |

By “oligodendrocyte O2 polypeptide” (or oligodendrocyte marker O2; oligodendrocyte transcription factor 2; olig2) is meant a polypeptide or fragment thereof having at least about 85% amino acid identity to NCBI Accession No. Q13516.

| (SEQ ID NO: 31) | |
|---|---|
| 1 | mdsdaslvss rpsspepddl flparskgss gsaftggtvs sstpsdcppe lsaelrgamg |
| 61 | sagahpgdkl ggsgfkssss stssstssaa asstkkdkkq mtepelqqlr lkinsrerkr |
| 121 | mhdlniamdg lrevmpyahg psvrklskia tlllarnyil mltnsleemk rlvseiyggh |
| 181 | hagfhpsacg glahsaplpa atahpaaaah aahhpavhhp ilppaaaaaa aaaaaaavss |
| 241 | aslpgsglps vgsirpphgl lkspsaaaaa plggggggsg asggfqhwgg mpcpcsmcqv |
| 301 | ppphhhvsam gagslprlts dak |

By “oligodendrocyte O2 nucleic acid molecule” (or oligodendrocyte marker O2; oligodendrocyte transcription factor 2; olig2) is meant a polynucleotide encoding an oligodendrocyte O2 polypeptide. An exemplary oligodendrocyte O2 nucleic acid molecule (e.g., mRNA) is provided at NCBI Accession No. NM_005806.

| (SEQ ID NO: 32) | |
|---|---|
| 1 | gggtgcttat tatagatcga cgcgacacca gcgcccggtg ccaggttctc ccctgaggct |
| 61 | tttcggagcg agctcctcaa atcgcatcca gagtaagtgt ccccgcccca cagcagccgc |
| 121 | agcctagatc ccagggacag actctcctca actcggctgt gacccagaat gctccgatac |
| 181 | agggggtctg gatccctact ctgcgggcca tttctccaga gcgactttgc tcttctgtcc |
| 241 | tccccacact caccgctgca tctccctcac caaaagcgag aagtcggagc gacaacagct |
| 301 | ctttctgccc aagccccagt cagctggtga gctccccgtg gtctccagat gcagcacatg |
| 361 | gactctgggc cccgcgccgg ctctgggtgc atgtgcgtgt gcgtgtgttt gctgcgtggt |
| 421 | gtcgatggag ataaggtgga tccgtttgag gaaccaaatc attagttctc tatttagatc |
| 481 | tccattctcc ccaaagaaag gccctcactt cccactcgtt tattccagcc cgggggctca |
| 541 | gttttcccac acctaactga aagcccgaag cctctagaat gccacccgca ccccgagggt |
| 601 | caccaacgct ccctgaaata acctgttgca tgagagcaga ggggagatag agagagctta |
| 661 | attataggta cccgcgtgca gctaaaagga gggccagaga tagtagcgag ggggacgagg |
| 721 | agccacgggc cacctgtgcc gggaccccgc gctgtggtac tgcggtgcag gcgggagcag |
| 781 | cttttctgtc tctcactgac tcactctctc tctctctccc tctctctctc tctcattctc |
| 841 | tctcttttct cctcctctcc tggaagtttt cgggtccgag ggaaggagga ccctgcgaaa |
| 901 | gctgcgacga ctatcttccc ctggggccat ggactcggac gccagcctgg tgtccagccg |
| 961 | cccgtcgtcg ccagagcccg atgacctttt tctgccggcc cggagtaagg gcagcagcgg |
| 1021 | cagcgccttc actgggggca ccgtgtcctc gtccaccccg agtgactgcc cgccggagct |
| 1081 | gagcgccgag ctgcgcggcg ctatgggctc tgcgggcgcg catcctgtgg acaagctagg |
| 1141 | aggcagtggc ttcaagtcat cctcgtccag cacctcgtcg tctacgtcgt cggcggctgc |
| 1201 | gtcgtccacc aagaaggaca agaagcaaat gacagagccg gagctgcagc agctgcgtct |
| 1261 | caagatcaac agccgcgagc gcaagcgcat gcacgacctc aacatcgcca tggatggcct |
| 1321 | ccgcgaggtc atgccgtacg cacacggccc ttcggtgcgc aagctttcca agatcgccac |
| 1381 | gctgctgctg gcgcgcaact acatcctcat gctcaccaac tcgctggagg agatgaagcg |
| 1441 | actggtgagc gagatctacg ggggccacca cgctggcttc cacccgtcgg cctgcggcgg |
| 1501 | cctggcgcac tccgcgcccc tgcccgccgc caccgcgcac ccggcagcag cagcgcacgc |
| 1561 | cgcacatcac cccgcggtgc accaccccat cctgccgccc gccgccgcag cggctgctgc |
| 1621 | cgccgctgca gccgcggctg tgtccagcgc ctctctgccc ggatccgggc tgccgtcggt |
| 1681 | cggctccatc cgtccaccgc acggcctact caagtctccg tctgctgccg cggccgcccc |
| 1741 | gctggggggc gggggcggcg gcagtggggc gagcgggggc ttccagcact ggggcggcat |
| 1801 | gccctgcccc tgcagcatgt gccaggtgcc gccgccgcac caccacgtgt cggctatggg |
| 1861 | cgccggcagc ctgccgcgcc tcacctccga cgccaagtga gcctactggc gccggcgcgt |
| 1921 | tctggcgaca ggggagccag gggccgcggg gaagcgagga ctggcctgcg ctgggctcgg |
| 1981 | gagctctgtc gcgaggaggg gcgcaggacc atggactggg ggtggggcat ggtggggatt |
| 2041 | tcagcatctg cgaacccaag caatgggggc gcccacagag cagtggggag tgaggggatg |
| 2101 | ttctctccgg gacctgatcg agcgctgtct ggctttaacc tgagctggtc cagtagacat |
| 2161 | cgttttatga aaaggtaccg ctgtgtgcat tcctcactag aactcatccg acccccgacc |
| 2221 | cccacctccg ggaaaagatt ctaaaaactt ctttccctga gagcgtggcc tgacttgcag |
| 2281 | actcggcttg ggcagcactt cgggggggga gggggtgtta tgggaggggg acacattggg |
| 2341 | gccttgctcg tcttcctcct ttcttggcgg gtgggagact ccgggtagcc gcactgcaga |
| 2401 | agcaacagcc cgaccgcgcc ctccagggtc gtccctggcc caaggccagg ggccacaagt |
| 2461 | tagttggaag ccggcgttcg gtatcagaag cgctgatggt catatccaat ctcaatatct |
| 2521 | gggtcaatcc acaccctctt agaactgtgg ccgttcctcc ctgtctctcg ttgatttggg |
| 2581 | agaatatggt tttctaataa atctgtggat gttccttctt caacagtatg agcaagttta |
| 2641 | tagacattca gagtagaacc acttgtggat tggaataacc caaaactgcc gatttcaggg |
| 2701 | gcgggtgcat tgtagttatt attttaaaat agaaactacc ccaccgactc atctttcctt |
| 2761 | ctctaagcac aaagtgattt ggttattttg gtacctgaga acgtaacaga attaaaaggc |
| 2821 | agttgctgtg gaaacagttt gggttatttg ggggttctgt tggcttttta aaattttctt |
| 2881 | ttttggatgt gtaaatttat caatgatgag gtaagtgcgc aatgctaagc tgtttgctca |
| 2941 | cgtgactgcc agccccatcg gagtctaagc cggctttcct ctattttggt ttatttttgc |
| 3001 | cacgtttaac acaaatggta aactcctcca cgtgcttcct gcgttccgtg caagccgcct |
| 3061 | cggcgctgcc tgcgttgcaa actgggcttt gtagcgtctg ccgtgtaaca cccttcctct |
| 3121 | gatcgcaccg cccctcgcag agagtgtatc atctgtttta tttttgtaaa aacaaagtgc |
| 3181 | taaataatat ttattacttg tttggttgca aaaacggaat aaatgactga gtgttgagat |
| 3241 | tttaaataaa atttaaagca aaaaaaaaaa aaaaa |

By “oligodendrocyte O4 polypeptide” (or oligodendrocyte marker O4; oligodendrocyte transcription factor 4; olig4) is meant a polypeptide or fragment thereof having at least about 85% amino acid identity to NCBI Accession No. Q05586.

By “oligodendrocyte O4 nucleic acid molecule” (or oligodendrocyte marker O4; oligodendrocyte transcription factor 4; olig4) is meant a polynucleotide encoding an oligodendrocyte O4 polypeptide. An exemplary oligodendrocyte O4 nucleic acid molecule (e.g., mRNA) is provided at NCBI Accession No. NM_007327.

By “GFAP” (or Glial fibrillary acidic protein) is meant a polypeptide or fragment thereof having at least about 85% amino acid identity to NCBI Accession No. P14136.

| (SEQ ID NO: 33) | |
|---|---|
| 1 | merrritsaa rrsyvssgem mvgglapgrr lgpgtrlsla rmppplptrv dfslagalna |
| 61 | gfketraser aemmelndrf asyiekvrfl eqqnkalaae lnqlrakept kladvyqael |
| 121 | relrlrldql tansarleve rdnlaqdlat vrqklqdetn lrleaennla ayrqeadeat |
| 181 | larldlerki esleeeirfl rkiheeevre lqeqlarqqv hveldvakpd ltaalkeirt |
| 241 | qyeamassnm heaeewyrsk fadltdaaar naellrqakh eandyrrqlq sltcdleslr |
| 301 | gtneslerqm reqeerhvre aasyqealar leeegqslkd emarhlqeyq dllnvklald |
| 361 | ieiatyrkll egeenritip vqtfsnlqir etsldtksvs eghlkrnivv ktvemrdgev |
| 421 | ikeskqehkd vm |

By “GFAP nucleic acid molecule” (or Glial fibrillary acidic protein) is meant a polynucleotide encoding an GFAP polypeptide. An exemplary GFAP nucleic acid molecule (e.g., mRNA) is provided at NCBI Accession No. NM_002055.

| (SEQ ID NO: 34) | |
|---|---|
| 1 | gcaggatgga gaggagacgc atcacctccg ctgctcgccg ctcctacgtc tcctcagggg |
| 61 | agatgatggt ggggggcctg gctcctggcc gccgtctggg tcctggcacc cgcctctccc |
| 121 | tggctcgaat gccccctcca ctcccgaccc gagtggattt ctccctggct ggggcactca |
| 181 | atgctggctt caaggagacc cgggccagtg agcgggcaga gatgatggag ctcaatgacc |
| 241 | gctttgccag ctacatcgag aaggttcgct tcctggaaca gcaaaacaag gcgctggctg |
| 301 | ctgagctgaa ccagctgcgg gccaaggagc ccaccaagct ggcagacgtc taccaggctg |
| 361 | agctgcgaga gctgcggctg cggctcgatc aactcaccgc caacagcgcc cggctggagg |
| 421 | ttgagaggga caatctggca caggacctgg ccactgtgag gcagaagctc caggatggaa |
| 481 | ccaacctgag gctggaagcc gagaacaacc tggctgccta tagacaggaa gcagatgaag |
| 541 | ccaccctggc ccgtctggat ctggagagga agattgagtc gctggaggag gagatccggt |
| 601 | tcttgaggaa gatccacgag gaggaggttc gggaactcca ggagcagctg gcccgacagc |
| 661 | aggtccatgt ggagcttgac gtggccaagc cagacctcac cgcagccctg aaagagatcc |
| 721 | gcacgcagta tgaggcaatg gcgtccagca acatgcatga agccgaagag tggtaccgct |
| 781 | ccaagtttgc agacctgaca gacgctgctg cccgcaacgc ggagctgctc cgccaggcca |
| 841 | agcacgaagc caacgactac cggcgccagt tgcagtcctt gacctgcgac ctggagtctc |
| 901 | tgcgcggcac gaacgagtcc ctggagaggc agatgcgcga gcaggaggag cggcacgtgc |
| 961 | gggaggcggc cagttatcag gaggcgctgg cgcggctgga ggaagagggg cagagcctca |
| 1021 | aggacgagat ggcccgccac ttgcaggagt accaggacct gctcaatgtc aagctggccc |
| 1081 | tggacatcga gatcgccacc tacaggaagc tgctagaggg cgaggagaac cggatcacca |
| 1141 | ttcccgtgca gaccttctcc aacctgcaga ttcgagaaac cagcctggac accaagtctg |
| 1201 | tgtcagaagg ccacctcaag aggaacatcg tggtgaagac cgtggagatg cgggatggag |
| 1261 | aggtcattaa ggagtccaag caggagcaca aggatgtgat gtgaggcagg acccacctgg |
| 1321 | tggcctctgc cccgtctcat gaggggcccg agcagaagca ggatagttgc tccgcctctg |
| 1381 | ctggcacatt tccccagacc tgagctcccc accaccccag ctgctcccct ccctcctctg |
| 1441 | tccctaggtc agcttgctgc cctaggctcc gtcagtatca ggcctgcc |

By “s100b” (or S-100 protein beta chain; S-100 protein subunit beta; S100 calcium-binding protein B) is meant a polypeptide or fragment thereof having at least about 85% amino acid identity to NCBI Accession No. P04271.
1 mselekamva lidvfhqysg regdkhklkk selkelinne Ishfleeike qevvdkvmet
61 ldndgdgecd fqefmafvam vttacheffe he (SEQ ID NO: 35)

By “s100b nucleic acid molecule” (or S-100 protein beta chain; S-100 protein subunit beta; S100 calcium-binding protein B) is meant a polynucleotide encoding an s100b polypeptide. An exemplary s100b nucleic acid molecule (e.g., mRNA) is provided at NCBI Accession No. NM_006272.

| (SEQ ID NO: 36) | |
|---|---|
| 1 | gggcagaggg aataagaggc tgcctctgcc caccagtcct gccgcccagg acccgcagca |
| 61 | gagacgacgc ctgcagcaag gagaccagga aggggtgaga caaggaagag gatgtctgag |
| 121 | ctggagaagg ccatggtggc cctcatcgac gttttccacc aatattctgg aagggaggga |
| 181 | gacaagcaca agctgaagaa atccgaactg aaggagctca tcaacaatga gctttcccat |
| 241 | ttcttagagg aaatcaaaga gcaggaggtt gtggacaaag tcatggaaac actggacaat |
| 301 | gatggagacg gcgaatgtga cttccaggaa ttcatggcct ttgttgccat ggttactact |
| 361 | gcctgccacg agttctttga acatgagtga gattagaaag cagccaaacc tttcctgtaa |
| 421 | cagagacggt catgcaagaa agcagacagc aagggcttgc agcctagtag gagctgagct |
| 481 | ttccagccgt gttgtagcta attaggaagc ttgatttgct ttgtgattga aaaattgaaa |
| 541 | acctctttcc aaaggctgtt ttaacggcct gcatcattct ttctgctata ttaggcctgt |
| 601 | gtgtaagctg actggcccca gggactcttg ttaacagtaa cttaggagtc aggtctcagt |
| 661 | gataaagcgt gcaccgtgca gcccgccatg gccgtgtaga ccctaacccg gagggaaccc |
| 721 | tgactacaga aattaccccg gggcaccctt aaaacttcca ctacctttaa aaaacaaagc |
| 781 | cttatccagc attatttgaa aacactgctg ttctttaaat gcgttcctca tccatgcaga |
| 841 | taacagctgg ttggccggtg tggccctgca agggcgtggt ggcttcggcc tgcttcccgg |
| 901 | gatgcgcctg atcaccaggt gaacgctcag cgctggcagc gctcctggaa aaagcaactc |
| 961 | catcagaact cgcaatccga gccagctctg ggggctccag cgtggcctcc gtgacccatg |
| 1021 | cgattcaagt cgcggctgca ggatccttgc ctccaacgtg cctccagcac atgcggcttc |
| 1081 | cgagggcact accgggggct ctgagccacc gcgagggcct gcgttcaata aaaag |

By “SOX10 polypeptide” (or SRY-related HMG-box transcription factor) is meant a polypeptide or fragment thereof having at least about 85% amino acid identity to NCBI Accession No. NP_008872.1.

(SEQ ID NO: 37)
MAEEQDLSEVELSPVGSEEPRCLSPGSAPSLGPDGGGGGSGLRASPGPGE
LGKVKKEQQDGEADDDKFPVCIREAVSQVLSGYDWTLVPMPVRVNGASKS
KPHVKRPMNAFMVWAQAARRKLADQYPHLHNAELSKTLGKLWRLLNESDK
RPFIEEAERLRMQHKKDHPDYKYQPRRRKNGKAAQGEAECPGGEAEQGGT
AAIQAHYKSAHLDHRHPGEGSPMSDGNPEHPSGQSHGPPTPPTTPKTELQ
SGKADPKRDGRSMGEGGKPHIDFGNVDIGEISHEVMSNMETFDVAELDQY
LPPNGHPGHVSSYSAAGYGLGSALAVASGHSAWISKPPGVALPTVSPPGV
DAKAQVKTETAGPQGPPHYTDQPSTSQIAYTSLSLPHYGSAFPSISRPQF
DYSDHQPSGPYYGHSGQASGLYSAFSYMGPSQRPLYTAISDPSPSG
PQSHSPTHWEQPVYTTLSRP

By “SOX10 nucleic acid molecule” (or SRY-related HMG-box transcription factor) is meant a polynucleotide encoding an SOX10 polypeptide. An exemplary SOX10 nucleic acid molecule (e.g., mRNA) is provided at NCBI Accession No. NM_006941.3.

| (SEQ ID NO: 38) | |
|---|---|
| 1 | gtccggccag ggtggttggt ggtaaggatt caggctccgt cctaacgagg ccgtggcctg |
| 61 | aggctcaggg ccccccgccc ctccctccca gcccaccagc gtcacctccc agccccgagc |
| 121 | tggaccgcac accttgggac acggttttcc acttcctaag gacgagcccc agactggagg |
| 181 | agaggtccga ggaggtgggc gttggactct ttgcgaggac cccggcggct ggcccggggg |
| 241 | aggcggccga ggcggcggcg gcggcggccg ggggcgacat ggcggaggag caggacctat |
| 301 | cggaggtgga gctgagcccc gtgggctcgg aggagccccg ctgcctgtcc ccggggagcg |
| 361 | cgccctcgct agggcccgac ggcggcggcg gcggatcggg cctgcgagcc agcccggggc |
| 421 | caggcgagct gggcaaggtc aagaaggagc agcaggacgg cgaggcggac gatgacaagt |
| 481 | tccccgtgtg catccgcgag gccgtcagcc aggtgctcag cggctacgac tggacgctgg |
| 541 | tgcccatgcc cgtgcgcgtc aacggcgcca gcaaaagcaa gccgcacgtc aagcggccca |
| 601 | tgaacgcctt catggtgtgg gctcaggcag cgcgcaggaa gctcgcggac cagtacccgc |
| 661 | acctgcacaa cgctgagctc agcaagacgc tgggcaagct ctggaggctg ctgaacgaaa |
| 721 | gtgacaagcg ccccttcatc gaggaggctg agcggctccg tatgcagcac aagaaagacc |
| 781 | acccggacta caagtaccag cccaggcggc ggaagaacgg gaaggccgcc cagggcgagg |
| 841 | cggagtgccc cggtggggag gccgagcaag gtgggaccgc cgccatccag gcccactaca |
| 901 | agagcgccca cttggaccac cggcacccag gagagggctc ccccatgtca gatgggaacc |
| 961 | ccgagcaccc ctcaggccag agccatggcc cacccacccc tccaaccacc ccgaagacag |
| 1021 | agctgcagtc gggcaaggca gacccgaagc gggacgggcg ctccatgggg gagggcggga |
| 1081 | agcctcacat cgacttcggc aacgtggaca ttggtgagat cagccacgag gtaatgtcca |
| 1141 | acatggagac ctttgatgtg gctgagttgg accagtacct gccgcccaat gggcacccag |
| 1201 | gccatgtgag cagctactca gcagccggct atgggctggg cagtgccctg gccgtggcca |
| 1261 | gtggacactc cgcctggatc tccaagccac caggcgtggc tctgcccacg gtctcaccac |
| 1321 | ctggtgtgga tgccaaagcc caggtgaaga cagagaccgc ggggccccag gggcccccac |
| 1381 | actacaccga ccagccatcc acctcacaga tcgcctacac ctccctcagc ctgccccact |
| 1441 | atggctcagc cttcccctcc atctcccgcc cccagtttga ctactctgac catcagccct |
| 1501 | caggacccta ttatggccac tcgggccagg cctctggcct ctactcggcc ttctcctata |
| 1561 | tggggccctc gcagcggccc ctctacacgg ccatctctga ccccagcccc tcagggcccc |
| 1621 | agtcccacag ccccacacac tgggagcagc cagtatatac gacactgtcc cggccctaaa |
| 1681 | gggggccctg tcgccaccac cccccgccca gcccctgccc ccagcctgtg tgccctgttc |
| 1741 | cttgcccacc tcaggcctgg tggtggcagt ggaggaggct gaggaggctg aagaggctga |
| 1801 | caggtcgggg ggctttctgt ctggctcact gccctgatga cccacccgcc ccatccaggc |
| 1861 | tccagcagca aagccccagg agaacaggct ggacagagga gaaggaggtt gactgttgca |
| 1921 | cccacactga aagatgaggg gctgcacctt cccccaggaa tgaccctcta tcccaggacc |
| 1981 | tgagaagggc ctgctcaccc tcctcgggga ggggaagcac cagggttggt ggcatcggag |
| 2041 | gccttaccac tcctatgact cctgttttct ctctcacaga tagtgagggt ctgacatgcc |
| 2101 | catgccacct atgccacagt gcctaagggc taggccaccc agagactgtg cccggagctg |
| 2161 | gccgtgtctc ccactcaggg gctgagagta gctttgagga gcctcattgg ggagtggggg |
| 2221 | gttcgaggga cttagtggag ttctcatccc ttcaatgccc cctccctttc tgaaggcagg |
| 2281 | aaggagttgg cacagaggcc ccctgatcca attctgtgcc aataacctca ttctttgtct |
| 2341 | gagaaacagc ccccagtcct cctccactac aacctccatg accttgagac gcatcccagg |
| 2401 | aggtgacgag gcaggggctc caggaaagga atcagagaca attcacagag cctccctccc |
| 2461 | tgggctcctt gccagctccc tcttccctta ctaggctcta tggcccctgc tcagtcagcc |
| 2521 | ccactccctg ggcttcccag agagtgacag ctgctcaggc cctaaccctt ggctccagga |
| 2581 | gacacagggc ccagcaccca ggttgctgtc ggcaggctga agacactaga atcctgacct |
| 2641 | gtacattctg cccttgcctc ttaccccttg cctcccagtg gtatttgaat aaagtatgta |
| 2701 | gctatatctg cccctatttt cctgttctgc agccccccaa atccacatgt aactcattac |
| 2761 | tgtctcctgt tatttatctc agtagtcccc tctcctagcc actctagccc ctattaactc |
| 2821 | tgcattaagc attccacata ataaaattaa aggttccggt taaaaaaaaa aaaaaaaaaa |
| 2881 | aa |

By “SYN1 protein” (or Synaptin I protein) is meant a polypeptide or fragment thereof having at least about 85% amino acid identity to GenBank: AH006533.2.

(SEQ ID NO: 39)
MNYLRRRLSDSNFMANLPNGYMTDLQRPQPPPPPPGAHSPGATPGPGTAT
AERSSGVAPAASPAAPSPGSSGGGGFFSSLSNAVKQTTAAAAATFSEQVG
GGSGGAGRGGAASRVLLVIDEPHTDWAKYFKGKKIHGGIDIKVEQAEFSD
LNLVAHANGGFSVDMEVLRNGVKVVRSLKPDFVLIRQHAFSMARNGDYRS
LVIGLQYAGIPSVNSLHSVYNFCDKPWVFAQMVRLHKKLGTEEFPLIDQT
FYPNHKEMLSSTTYPVVVKMGHAHSGMGKVKVDNQHDFQDIASVVALTKT
YATAEPFIDAKYDVRVQKIGQNYKAYMRTSVSGNWKTNTGSAMLEQIAMS
DRYKLWVDTCSEIFGGLDICAVEALHGKDGRDHIIEVVGSSMPLIGDHQD
EDKQLIVELVVNKMAQALPRQRQRDASPGRGSHGQTPSPGALPLGRQTSQ
QPAGPPAQQRPPPQGGPPQPGPGPQRQGPPLQQRPPPQGQQHLSGLGPPA
GSPLPQRLPSPTSAPQQPASQAAPPTQGQGRQSRPVAGGPGAPPAARPPA
SPSPQRQAGPPQATRQTSVSGPAPPKASGAPPGGQQRQGPPQKPPGPAGP
TRQASQAGPVPRTGPPTTQQPRPSGPGPAGAPKPQLAQKPSQDVPPPATA
AAGGPPHPQLNKSQSLTNAFNLPEPAPPRPSLSQDEVKAETIRSLRKSFA
SLFSD

By “SYN1 nucleic acid molecule” (or synapsin I gene) is meant a polynucleotide encoding an SYN1polypeptide. An exemplary SYN1nucleic acid molecule (e.g., mRNA) is provided at GenBank: AH006533.2.

| (SEQ ID NO: 40) | |
|---|---|
| 1 | ctcgagagag aaggagagga cattcctggc agaagttaca acacatgcaa aggtacagag |
| 61 | gttgccccct tcctacccct ctccttagag gtgggttaga gatgtatcct ttttacagat |
| 121 | gaggaaacca aatctcagaa agattaagtc actttcccaa gtgtatggtg gaggccccac |
| 181 | ttgaacccag gcactgtgtc tccagacccc acactattac tgccttgttt aaaccagcca |
| 241 | actgatttaa tgaataaagg atgaacaaat gaataagtgg atgagtcacc tgaaaattct |
| 301 | gcaggcaaag agactccata tctacttact tcttgcctat cttctgccac ctctcctagt |
| 361 | ccaccatcac tgctcactat ggtcaaggtc ctacccaatc tggcccctgc taccacaacc |
| 421 | cccttcagct tgttccagcc acattggcac tggatgtttc ctcttcctgg cacattctta |
| 481 | aaaaaatgtg ttgatcataa agtgaacatg accctttggg aattaactgg agttcttgta |
| 541 | ttccctcatc tgtaaaatag acattatatt atccacccca ctggattgtt gtgagggtgg |
| 601 | gatgaaatga tgcatgtaaa cacgcttagc ttaagagttg ggtacaatca gtgaacaaat |
| 661 | gattatgaat tagtgctttt attgtagtca gaatcataaa gatttgacag gttcccatat |
| 721 | cccacctctg cttggactac ctcatttgct catatgcaaa gattatttgg tacctactgt |
| 781 | gtgtgcacca tgggatgggc ctgcctctgt ggaaagttct tgggtgcagg gggagacagc |
| 841 | catgggcact gatgacatca ggtagttatc gtgagttttg gcggtgtcca gagcaaaggg |
| 901 | atggtggcgt atataccaag tgtgttctgg tgtgggggtg gacacgcacc agggctaggg |
| 961 | ctgcagagaa tgtctgtgtt gcagatctag gtttctccat gatcatcggt gggaatgtgt |
| 1021 | tttgtctgca agtgtatgct catatgagtt tccctgggtc tctgtgtgtc agtgtgttac |
| 1081 | ctgtgtgtgt gggggtatgg gtgtatgcat gcatgtatgt aacatgccca tgtgtgttac |
| 1141 | tctggacttg tatgtctgta tgtataccta gattggcgtg tgttctgtct gtacatgccc |
| 1201 | tcgtatgttt cctcactttt gtgtgtgttt atatgtgtgt catttcttgt gtgccctcca |
| 1261 | ggcccccctt gccaccttgg gcaagggtgt gtacaccacc caagtgtcca cctccgcttg |
| 1321 | tctgatgctg tctgtgacgc ccccgctctc tgcctagctg agcctgtgtg gatgtgggag |
| 1381 | actaatctcc ccgcgggcac tgcgtgtgac ctcacccccc tctgtgaggg ggttatttct |
| 1441 | ctactttcgt gtctctgagt gtgcttccag tgcccccctc cccccaaaaa atgccttctg |
| 1501 | agttgaatat caacactaca aaccgagtat ctgcagactg cagagggccc tgcgtatgag |
| 1561 | tgcaagtggg ttttaggacc aggatgaggc ggggtggggg tgcctacctg acgaccgacc |
| 1621 | ccgacccact ggacaagcac ccaaccccca ttccccaaat tgcgcatccc ctatcagaga |
| 1681 | gggggagggg aaacaggatg cggcgaggcg cgtcgcgact gccagcttca gcaccgcgga |
| 1741 | cagtgccttc gcccccgcct ggcggcgcgc gccaccgccg cctcagcact gaaggcgcgc |
| 1801 | tgacgtcact cgccggtccc ccgcaaactc cccttcccgg ccaccttggt cgcgtccgcg |
| 1861 | ccgccgccgg cccagccgga ccgcaccacg cgaggcgcga gatagggggg cacgggcgcg |
| 1921 | accatctgcg ctgcggcgcc ggcgactcag cgctgcctca gtctgcggtg ggcagcggag |
| 1981 | gagtcgtgtc gtgcctgaga gcgcagctgt gctcctgggc accgcgcagt ccgcccccgc |
| 2041 | ggctcctggc cagaccaccc ctaggacccc ctgccccaag tcgcagccat gaactacctg |
| 2101 | cggcgccgcc tgtcggacag caactttatg gccaatctgc caaatgggta catgacagac |
| 2161 | ctgcagcgtc cgcagccgcc cccaccgccg cccggtgccc acagccccgg agccacgccc |
| 2221 | ggtcccggga ccgccactgc cgagaggtcc tccggggtcg ccccagcggc ctctccggcc |
| 2281 | gcccctagcc ccgggtcctc ggggggcggt ggcttcttct cgtcgctgtc caacgcggtc |
| 2341 | aagcagacca cggcggcggc agctgccacc ttcagcgagc aggtgggcgg cggctctggg |
| 2401 | ggcgcaggcc gcgggggagc cgcctccagg gtgctgctgg tcatcgacga gccgcacacc |
| 2461 | gactggtaag |

By “SYP protein” (or synaptophysin protein) is meant a polypeptide or fragment thereof having at least about 85% amino acid identity to NCBI Reference Sequence: NM_003179.2.

(SEQ ID NO: 41)
MLLLADMDVVNQLVAGGQFRVVKEPLGFVKVLQWVFAIFAFATCGSYSGE
LQLSVDCANKTESDLSIEVEFEYPFRLHQVYFDAPTCRGGTTKVFLVGDY
SSSAEFFVTVAVFAFLYSMGALATYIFLQNKYRENNKGPMLDFLATAVFA
FMWLVSSSAWAKGLSDVKMATDPENIIKEMPVCRQTGNTCKELRDPVTSG
LNTSVVFGFLNLVLWVGNLWFVFKETGWAAPFLRAPPGAPEKQPAPGDAY
GDAGYGQGPGGYGPQDSYGPQGGYQPDYGQPAGSGGSGYGPQGDYGQQGY
GPQGAPTSFSNQM

By “SYP nucleic acid molecule” (or synaptophysin gene) is meant a polynucleotide encoding an SYN1polypeptide. An exemplary SYP nucleic acid molecule (e.g., mRNA) is provided at NCBI Reference Sequence: NM_003179.2.

| (SEQ ID NO: 42) | |
|---|---|
| 1 | gccccctgca ttgctgatgc tgctgctggc ggacatggac gtggtgaatc agctggtggc |
| 61 | tgggggtcag ttccgggtgg tcaaggagcc cctcggcttt gtgaaggtgc tgcaatgggt |
| 121 | cttcgccatc ttcgcctttg ccacatgcgg cagctacagt ggggagctcc agctgagcgt |
| 181 | ggattgtgcc aacaagaccg agagtgacct cagcatcgag gtcgagttcg agtacccctt |
| 241 | caggctgcac caagtgtact ttgatgcacc cacctgccga gggggcacca ccaaggtctt |
| 301 | cttagttggg gactactcct cgtcagccga attctttgtc accgtggccg tgtttgcctt |
| 361 | cctctactcc atgggggctc tggccaccta catcttcctg cagaacaagt accgagagaa |
| 421 | taacaaaggg cccatgctgg actttctggc cacggctgtg ttcgccttca tgtggctagt |
| 481 | tagctcatcg gcatgggcca aggggctgtc agatgtgaag atggccacag acccagagaa |
| 541 | cattatcaag gagatgcctg tctgccgcca gacagggaac acatgcaagg agctgagaga |
| 601 | ccctgtgacc tcgggactca acacctcggt ggtgttcggc ttcctgaacc tggtgctctg |
| 661 | ggtcggcaac ctgtggttcg tgtttaagga gacaggctgg gccgccccgt tcctgcgcgc |
| 721 | gcctcccggc gcccccgaga aacaaccggc acccggggac gcctacggcg atgcaggcta |
| 781 | cgggcagggc cccggcgggt acgggcccca ggattcctac gggcctcagg gcggctacca |
| 841 | gcctgactat ggtcaaccag ccggcagcgg tggcagtggc tacgggcctc agggcgacta |
| 901 | tgggcagcaa ggctacggcc cgcagggtgc acccacctcc ttctccaatc agatgtagtc |
| 961 | tggtcagtga agcccaggag gacctggggg gggcaagagc tcaggagaag gcctgccccc |
| 1021 | cttcccaccc ctatacccta ggtctccacc cctcaagcca ggagaccctg tctttgctgt |
| 1081 | ttatatatat atatattata tataaatatc tatttatctg tctgagccct gccctcactc |
| 1141 | cactcccctc atccactagg tgcccagtct tgagtgggcc ccctctctta ccccgtccct |
| 1201 | ttccctgcat cccttggccc ctctctgttt accctccctg tcccctgagg ttaaggggat |
| 1261 | ctaaaaggag gacagggagg gaacagacct cggctgtgtg gggagggtgg gcgtgacttc |
| 1321 | agactctctc ctctctctcc ctccactcct cccaactctg gccttggttc ctccagcaat |
| 1381 | gcctgcctga acaaaggccg ttagggaaat ccaactccag ggttaaagaa aggcagagat |
| 1441 | tgggggggct tggggtagag aggacagttt aggacccaag gtggtcttgg agaggaggtg |
| 1501 | tggagtggag gggtcagcag gggggttggg ttccagacag agtggatctg gagtctgaag |
| 1561 | gagaggagtg cgctagagca ttctggggtg gggcttggaa gggcgctgag ggcagggttc |
| 1621 | tagaaggggc gaggctttaa gcgaggcaga atggtgggct ccagagtagg tgggtcttgg |
| 1681 | attggtacca gagcctatgg aaagggtgtg gcttggaaca tttgggagac tgagcttgat |
| 1741 | tctaaagggg acagatcttg agcaaggcaa gaagtgggat tcaggaatgg gccaagccag |
| 1801 | ggttccagac agggtggggc ttagaatggg gcttccatgg tggtttcaga aagggcagcc |
| 1861 | cctccccatg gtgcagtgaa gaaaatgttt tacaatggct gggtttgggc agtggagagg |
| 1921 | ggacttggat aggagcttcc agatgggttt tgttaggggt gggggagaat ggctctggct |
| 1981 | acgacttggg acggaagtgg cctgagaaga gtcgagtgat atggcttgta gggtgaggcg |
| 2041 | tgggatccag agagaagcac cccaccacac acacccttcc ccactcccgt gatgaaacag |
| 2101 | ctaggttaat aggaggacag aaccaacggg tctgtgggac tggcccaccc ctcttccccc |
| 2161 | ttcccctgcg ccctccctcc ctccacacct ccacccgtcc tggggtggtt ggaggcctgg |
| 2221 | tctggagccc ctatcctgca ccctctgcta tgtctgtgat gtcagtagtg cctgtgatcg |
| 2281 | tgtgttgcca ttttgtctgg ctgtggcccc tccttctccc ctccagaccc ctaccctttc |
| 2341 | ccaaaccctt cggtattgtt caaagaaccc ccctccccaa ggaagaacaa atatgattct |
| 2401 | cctctcccaa ataaactcct taaccaccta gtcaaaaaaa aaaaaaaaa |

By “NOGOA polypeptide” (or neurite outgrowth inhibitor A: neurite outgrowth inhibitor isoform A: human reticulon-4; human reticulon-4 isoform A) is meant a polypeptide or fragment thereof having at least about 85% amino acid identity to NCBI Accession No. NP_065393.

| (SEQ ID NO: 43) | |
|---|---|
| 1 | medldqsplv sssdspprpq pafkyqfvre pedeeeeeee eeedededle elevlerkpa |
| 61 | aglsaapvpt apaagaplmd fgndfvppap rgplpaappv aperqpswdp spvsstvpap |
| 121 | splsaaavsp sklpeddepp arppppppas vspqaepvwt ppapapaapp stpaapkrrg |
| 181 | ssgsvdetlf alpaasepvi rssaenmdlk eqpgntisag qedfpsvlle taaslpslsp |
| 241 | lsaasfkehe ylgnlstvlp tegtlqenvs easkevseka ktllidrdlt efseleysem |
| 301 | gssfsvspka esavivanpr eeiivknkde eeklvsnnil hnqqelptal tklvkedevv |
| 361 | ssekakdsfn ekrvaveapm reeyadfkpf ervwevkdsk edsdmlaagg kiesnleskv |
| 421 | dkkcfadsle qtnhekdses snddtsfpst pegikdrsga yitcapfnpa atesiatnif |
| 481 | pllgdptsen ktdekkieek kaqivteknt stktsnpflv aaqdsetdyv ttdnltkvte |
| 541 | evvanmpegl tpdlvqeace selnevtgtk iayetkmdlv qtsevmqesl ypaaqlcpsf |
| 601 | eeseatpspv lpdivmeapl nsavpsagas viqpsssple assvnyesik hepenpppye |
| 661 | eamsvslkkv sgikeeikep eninaalqet eapyisiacd liketklsae papdfsdyse |
| 721 | makveqpvpd hselvedssp dsepvdlfsd dsipdvpqkq detvmlvkes ltetsfesmi |
| 781 | eyenkeklsa lppeggkpyl esfklsldnt kdtllpdevs tlskkekipl qmeelstavy |
| 841 | snddlfiske aqiretetfs dsspieiide fptlissktd sfsklareyt dlevshksei |
| 901 | anapdgagsl pctelphdls lkniqpkvee kisfsddfsk ngsatskvll lppdvsalat |
| 961 | qaeiesivkp kvlvkeaekk lpsdtekedr spsaifsael sktsvvdlly wrdikktgvv |
| 1021 | fgaslfllls ltvfsivsvt ayialallsv tisfriykgv iqaiqksdeg hpfraylese |
| 1081 | vaiseelvqk ysnsalghvn ctikelrrlf lvddlvdslk favlmwvfty vgalfngltl |
| 1141 | lilalislfs vpviyerhqa qidhylglan knvkdamaki qakipglkrk ae |

By “NOGOA nucleic acid molecule” (or neurite outgrowth inhibitor A; neurite outgrowth inhibitor isoform A; human reticulon-4; human reticulon-4 isoform A) is meant a polynucleotide encoding an NOGOA polypeptide. An exemplary NOGOA nucleic acid molecule (e.g., mRNA) is provided at NCBI Accession No. NM_020532.

| (SEQ ID NO: 44) | |
|---|---|
| 1 | agtccctgcc ctcccctggg gagggtgagt cacgccaaac tgggcggaga gtccgctggc |
| 61 | ctcactccta gctcatctgg gcggcggcgg caagtgggga cagggcgggt ggcgcatcac |
| 121 | cggcgcggag gcaggaggag cagtctcatt gttccgggag ccgtcaccac agtaggtccc |
| 181 | tcggctcagt cggcccagcc cctctcagtc ctccccaacc cccacaaccg cccgcggctc |
| 241 | tgagacgcgg ccccggcggc ggcggcagca gctgcagcat catctccacc ctccagccat |
| 301 | ggaagacctg gaccagtctc ctctggtctc gtcctcggac agcccacccc ggccgcagcc |
| 361 | cgcgttcaag taccagttcg tgagggagcc cgaggacgag gaggaagaag aggaggagga |
| 421 | agaggaggac gaggacgaag acctggagga gctggaggtg ctggagagga agcccgccgc |
| 481 | cgggctgtcc gcggccccag tgcccaccgc ccctgccgcc ggcgcgcccc tgatggactt |
| 541 | cggaaatgac ttcgtgccgc cggcgccccg gggacccctg ccggccgctc cccccgtcgc |
| 601 | cccggagcgg cagccgtctt gggacccgag cccggtgtcg tcgaccgtgc ccgcgccatc |
| 661 | cccgctgtct gctgccgcag tctcgccctc caagctccct gaggacgacg agcctccggc |
| 721 | ccggcctccc cctcctcccc cggccagcgt gagcccccag gcagagcccg tgtggacccc |
| 781 | gccagccccg gctcccgccg cgcccccctc caccccggcc gcgcccaagc gcaggggctc |
| 841 | ctcgggctca gtggatgaga ccctttttgc tcttcctgct gcatctgagc ctgtgatacg |
| 901 | ctcctctgca gaaaatatgg acttgaagga gcagccaggt aacactattt cggctggtca |
| 961 | agaggatttc ccatctgtcc tgcttgaaac tgctgcttct cttccttctc tgtctcctct |
| 1021 | ctcagccgct tctttcaaag aacatgaata ccttggtaat ttgtcaacag tattacccac |
| 1081 | tgaaggaaca cttcaagaaa atgtcagtga agcttctaaa gaggtctcag agaaggcaaa |
| 1141 | aactctactc atagatagag atttaacaga gttttcagaa ttagaatact cagaaatggg |
| 1201 | atcatcgttc agtgtctctc caaaagcaga atctgccgta atagtagcaa atcctaggga |
| 1261 | agaaataatc gtgaaaaata aagatgaaga agagaagtta gttagtaata acatccttca |
| 1321 | taatcaacaa gagttaccta cagctcttac taaattggtt aaagaggatg aagttgtgtc |
| 1381 | ttcagaaaaa gcaaaagaca gttttaatga aaagagagtt gcagtggaag ctcctatgag |
| 1441 | ggaggaatat gcagacttca aaccatttga gcgagtatgg gaagtgaaag atagtaagga |
| 1501 | agatagtgat atgttggctg ctggaggtaa aatcgagagc aacttggaaa gtaaagtgga |
| 1561 | taaaaaatgt tttgcagata gccttgagca aactaatcac gaaaaagata gtgagagtag |
| 1621 | taatgatgat acttctttcc ccagtacgcc agaaggtata aaggatcgtt caggagcata |
| 1681 | tatcacatgt gctcccttta acccagcagc aactgagagc attgcaacaa acatttttcc |
| 1741 | tttgttagga gatcctactt cagaaaataa gaccgatgaa aaaaaaatag aagaaaagaa |
| 1801 | ggcccaaata gtaacagaga agaatactag caccaaaaca tcaaaccctt ttcttgtagc |
| 1861 | agcacaggat tctgagacag attatgtcac aacagataat ttaacaaagg tgactgagga |
| 1921 | agtcgtggca aacatgcctg aaggcctgac tccagattta gtacaggaag catgtgaaag |
| 1981 | tgaattgaat gaagttactg gtacaaagat tgcttatgaa acaaaaatgg acttggttca |
| 2041 | aacatcagaa gttatgcaag agtcactcta tcctgcagca cagctttgcc catcatttga |
| 2101 | agagtcagaa gctactcctt caccagtttt gcctgacatt gttatggaag caccattgaa |
| 2161 | ttctgcagtt cctagtgctg gtgcttccgt gatacagccc agctcatcac cattagaagc |
| 2221 | ttcttcagtt aattatgaaa gcataaaaca tgagcctgaa aaccccccac catatgaaga |
| 2281 | ggccatgagt gtatcactaa aaaaagtatc aggaataaag gaagaaatta aagagcctga |
| 2341 | aaatattaat gcagctcttc aagaaacaga agctccttat atatctattg catgtgattt |
| 2401 | aattaaagaa acaaagcttt ctgctgaacc agctccggat ttctctgatt attcagaaat |
| 2461 | ggcaaaagtt gaacagccag tgcctgatca ttctgagcta gttgaagatt cctcacctga |
| 2521 | ttctgaacca gttgacttat ttagtgatga ttcaatacct gacgttccac aaaaacaaga |
| 2581 | tgaaactgtg atgcttgtga aagaaagtct cactgagact tcatttgagt caatgataga |
| 2641 | atatgaaaat aaggaaaaac tcagtgcttt gccacctgag ggaggaaagc catatttgga |
| 2701 | atcttttaag ctcagtttag ataacacaaa agataccctg ttacctgatg aagtttcaac |
| 2761 | attgagcaaa aaggagaaaa ttcctttgca gatggaggag ctcagtactg cagtttattc |
| 2821 | aaatgatgac ttatttattt ctaaggaagc acagataaga gaaactgaaa cgttttcaga |
| 2881 | ttcatctcca attgaaatta tagatgagtt ccctacattg atcagttcta aaactgattc |
| 2941 | attttctaaa ttagccaggg aatatactga cctagaagta tcccacaaaa gtgaaattgc |
| 3001 | taatgccccg gatggagctg ggtcattgcc ttgcacagaa ttgccccatg acctttcttt |
| 3061 | gaagaacata caacccaaag ttgaagagaa aatcagtttc tcagatgact tttctaaaaa |
| 3121 | tgggtctgct acatcaaagg tgctcttatt gcctccagat gtttctgctt tggccactca |
| 3181 | agcagagata gagagcatag ttaaacccaa agttcttgtg aaagaagctg agaaaaaact |
| 3241 | tccttccgat acagaaaaag aggacagatc accatctgct atattttcag cagagctgag |
| 3301 | taaaacttca gttgttgacc tcctgtactg gagagacatt aagaagactg gagtggtgtt |
| 3361 | tggtgccagc ctattcctgc tgctttcatt gacagtattc agcattgtga gcgtaacagc |
| 3421 | ctacattgcc ttggccctgc tctctgtgac catcagcttt aggatataca agggtgtgat |
| 3481 | ccaagctatc cagaaatcag atgaaggcca cccattcagg gcatatctgg aatctgaagt |
| 3541 | tgctatatct gaggagttgg ttcagaagta cagtaattct gctcttggtc atgtgaactg |
| 3601 | cacgataaag gaactcaggc gcctcttctt agttgatgat ttagttgatt ctctgaagtt |
| 3661 | tgcagtgttg atgtgggtat ttacctatgt tggtgccttg tttaatggtc tgacactact |
| 3721 | gattttggct ctcatttcac tcttcagtgt tcctgttatt tatgaacggc atcaggcaca |
| 3781 | gatagatcat tatctaggac ttgcaaataa gaatgttaaa gatgctatgg ctaaaatcca |
| 3841 | agcaaaaatc cctggattga agcgcaaagc tgaatgaaaa cgcccaaaat aattagtagg |
| 3901 | agttcatctt taaaggggat attcatttga ttatacgggg gagggtcagg gaagaacgaa |
| 3961 | ccttgacgtt gcagtgcagt ttcacagatc gttgttagat ctttattttt agccatgcac |
| 4021 | tgttgtgagg aaaaattacc tgtcttgact gccatgtgtt catcatctta agtattgtaa |
| 4081 | gctgctatgt atggatttaa accgtaatca tatctttttc ctatctatct gaggcactgg |
| 4141 | tggaataaaa aacctgtata ttttactttg ttgcagatag tcttgccgca tcttggcaag |
| 4201 | ttgcagagat ggtggagcta gaaaaaaaaa aaaaaaagcc cttttcagtt tgtgcactgt |
| 4261 | gtatggtccg tgtagattga tgcagatttt ctgaaatgaa atgtttgttt agacgagatc |
| 4321 | ataccggtaa agcaggaatg acaaagcttg cttttctggt atgttctagg tgtattgtga |
| 4381 | cttttactgt tatattaatt gccaatataa gtaaatatag attatatatg tatagtgttt |
| 4441 | cacaaagctt agacctttac cttccagcca ccccacagtg cttgatattt cagagtcagt |
| 4501 | cattggttat acatgtgtag ttccaaagca cataagctag aagaagaaat atttctagga |
| 4561 | gcactaccat ctgttttcaa catgaaatgc cacacacata gaactccaac atcaatttca |
| 4621 | ttgcacagac tgactgtagt taattttgtc acagaatcta tggactgaat ctaatgcttc |
| 4681 | caaaaatgtt gtttgtttgc aaatatcaaa cattgttatg caagaaatta ttaattacaa |
| 4741 | aatgaagatt tataccattg tggtttaagc tgtactgaac taaatctgtg gaatgcattg |
| 4801 | tgaactgtaa aagcaaagta tcaataaagc ttatagactt aaaaaaaaaa aaaaaaaaaa |
| 4861 | aaaaaaaaaa a |

By “GFAP” (or Glial fibrillary acidic protein) is meant a polypeptide or fragment thereof having at least about 85% amino acid identity to NCBI Accession No. P14136.

| (SEQ ID NO: 45) | |
|---|---|
| 1 | merrritsaa rrsyvssgem mvgglapgrr lgpgtrlsla rmppplptrv dfslagalna |
| 61 | gfketraser aemmelndrf asyiekvrfl eqqnkalaae lnqlrakept kladvyqael |
| 121 | relrlrldql tansarleve rdnlaqdlat vrqklqdetn lrleaennla ayrqeadeat |
| 181 | larldlerki esleeeirfl rkiheeevre lqeqlarqqv hveldvakpd ltaalkeirt |
| 241 | qyeamassnm heaeewyrsk fadltdaaar naellrqakh eandyrrqlq sltcdleslr |
| 301 | gtneslerqm reqeerhvre aasyqealar leeegqslkd emarhlqeyq dllnvklald |
| 361 | ieiatyrkll egeenritip vqtfsnlqir etsldtksvs eghlkrnivv ktvemrdgev |
| 421 | ikeskqehkd vm |

By “GFAP nucleic acid molecule” (or Glial fibrillary acidic protein) is meant a polynucleotide encoding an GFAP polypeptide. An exemplary GFAP nucleic acid molecule (e.g., mRNA) is provided at NCBI Accession No. NM_002055.

| (SEQ ID NO: 46) | |
|---|---|
| 1 | atcgccagtc tagcccactc cttcataaag ccctcgcatc ccaggagcga gcagagccag |
| 61 | agcaggatgg agaggagacg catcacctcc gctgctcgcc gctcctacgt ctcctcaggg |
| 121 | gagatgatgg tggggggcct ggctcctggc cgccgtctgg gtcctggcac ccgcctctcc |
| 181 | ctggctcgaa tgccccctcc actcccgacc cgggtggatt tctccctggc tggggcactc |
| 241 | aatgctggct tcaaggagac ccgggccagt gagcgggcag agatgatgga gctcaatgac |
| 301 | cgctttgcca gctacatcga gaaggttcgc ttcctggaac agcaaaacaa ggcgctggct |
| 361 | gctgagctga accagctgcg ggccaaggag cccaccaagc tggcagacgt ctaccaggct |
| 421 | gagctgcgag agctgcggct gcggctcgat caactcaccg ccaacagcgc ccggctggag |
| 481 | gttgagaggg acaatctggc acaggacctg gccactgtga ggcagaagct ccaggatgaa |
| 541 | accaacctga ggctggaagc cgagaacaac ctggctgcct atagacagga agcagatgaa |
| 601 | gccaccctgg cccgtctgga tctggagagg aagattgagt cgctggagga ggagatccgg |
| 661 | ttcttgagga agatccacga ggaggaggtt cgggaactcc aggagcagct ggcccgacag |
| 721 | caggtccatg tggagcttga cgtggccaag ccagacctca ccgcagccct gaaagagatc |
| 781 | cgcacgcagt atgaggcaat ggcgtccagc aacatgcatg aagccgaaga gtggtaccgc |
| 841 | tccaagtttg cagacctgac agacgctgct gcccgcaacg cggagctgct ccgccaggcc |
| 901 | aagcacgaag ccaacgacta ccggcgccag ttgcagtcct tgacctgcga cctggagtct |
| 961 | ctgcgcggca cgaacgagtc cctggagagg cagatgcgcg agcaggagga gcggcacgtg |
| 1021 | cgggaggcgg ccagttatca ggaggcgctg gcgcggctgg aggaagaggg gcagagcctc |
| 1081 | aaggacgaga tggcccgcca cttgcaggag taccaggacc tgctcaatgt caagctggcc |
| 1141 | ctggacatcg agatcgccac ctacaggaag ctgctagagg gcgaggagaa ccggatcacc |
| 1201 | attcccgtgc agaccttctc caacctgcag attcgagaaa ccagcctgga caccaagtct |
| 1261 | gtgtcagaag gccacctcaa gaggaacatc gtggtgaaga ccgtggagat gcgggatgga |
| 1321 | gaggtcatta aggagtccaa gcaggagcac aaggatgtga tgtgaggcag gacccacctg |
| 1381 | gtggcctctg ccccgtctca tgaggggccc gagcagaagc aggatagttg ctccgcctct |
| 1441 | gctggcacat ttccccagac ctgagctccc caccacccca gctgctcccc tccctcctct |
| 1501 | gtccctaggt cagcttgctg ccctaggctc cgtcagtatc aggcctgcca gacggcaccc |
| 1561 | acccagcacc cagcaactcc aactaacaag aaactcaccc ccaaggggca gtctggaggg |
| 1621 | gcatggccag cagcttgcgt tagaatgagg aggaaggaga gaaggggagg agggcggggg |
| 1681 | gcacctacta catcgccctc cacatccctg attcctgttg ttatggaaac tgttgccaga |
| 1741 | gatggaggtt ctctcggagt atctgggaac tgtgcctttg agtttcctca ggctgctgga |
| 1801 | ggaaaactga gactcagaca ggaaagggaa ggccccacag acaaggtagc cctggccaga |
| 1861 | ggcttgtttt gtcttttggt ttttatgagg tgggatatcc ctatgctgcc taggctgacc |
| 1921 | ttgaactcct gggctcaagc agtctaccca cctcagcctc ctgtgtagct gggattatag |
| 1981 | attggagcca ccatgcccag ctcagagggt tgttctccta gactgaccct gatcagtcta |
| 2041 | agatgggtgg ggacgtcctg ccacctgggg cagtcacctg cccagatccc agaaggacct |
| 2101 | cctgagcgat gactcaagtg tctcagtcca cctgagctgc catccaggga tgccatctgt |
| 2161 | gggcacgctg tgggcaggtg ggagcttgat tctcagcact tgggggatct gttgtgtacg |
| 2221 | tggagaggga tgaggtgctg ggagggatag aggggggctg cctggccccc agctgtgggt |
| 2281 | acagagaggt caagcccagg aggactgccc cgtgcagact ggaggggacg ctggtagaga |
| 2341 | tggaggagga ggcaattggg atggcgctag gcatacaagt aggggttgtg ggtgaccagt |
| 2401 | tgcacttggc ctctggattg tgggaattaa ggaagtgact catcctcttg aagatgctga |
| 2461 | aacaggagag aaaggggatg tatccatggg ggcagggcat gactttgtcc catttctaaa |
| 2521 | ggcctcttcc ttgctgtgtc ataccaggcc gccccagcct ctgagcccct gggactgctg |
| 2581 | cttcttaacc ccagtaagcc actgccacac gtctgaccct ctccacccca tagtgaccgg |
| 2641 | ctgcttttcc ctaagccaag ggcctcttgc ggtcccttct tactcacaca caaaatgtac |
| 2701 | ccagtattct aggtagtgcc ctattttaca attgtaaaac tgaggcacga gcaaagtgaa |
| 2761 | gacactggct catattcctg cagcctggag gccgggtgct cagggctgac acgtccaccc |
| 2821 | cagtgcaccc actctgcttt gactgagcag actggtgagc agactggtgg gatctgtgcc |
| 2881 | cagagatggg actgggaggg cccacttcag ggttctcctc tcccctctaa ggccgaagaa |
| 2941 | gggtccttcc ctctccccaa gacttggtgt cctttccctc cactccttcc tgccacctgc |
| 3001 | tgctgctgct gctgctaatc ttcagggcac tgctgctgcc tttagtcgct gaggaaaaat |
| 3061 | aaagacaaat gctgcgccct tccccaaaaa aaaaaaa |

By “s100b” (or S-100 protein beta chain; S-100 protein subunit beta; $100 calcium-binding protein B) is meant a polypeptide or fragment thereof having at least about 85% amino acid identity to NCBI Accession No. P04271.

| (SEQ ID NO: 47) | |
|---|---|
| 1 | mselekamva lidvfhqysg regdkhklkk selkelinne lshfleeike qevvdkvmet |
| 61 | ldndgdgecd fqefmafvam vttacheffe he |

By “s100b nucleic acid molecule” (or S-100 protein beta chain; S-100 protein subunit beta; S100 calcium-binding protein B) is meant a polynucleotide encoding an s100b polypeptide. An exemplary s100b nucleic acid molecule (e.g., mRNA) is provided at NCBI Accession No. NM_006272.

| (SEQ ID NO: 48) | |
|---|---|
| 1 | gggcagaggg aataagaggc tgcctctgcc caccagtcct gccgcccagg acccgcagca |
| 61 | gagacgacgc ctgcagcaag gagaccagga aggggtgaga caaggaagag gatgtctgag |
| 121 | ctggagaagg ccatggtggc cctcatcgac gttttccacc aatattctgg aagggaggga |
| 181 | gacaagcaca agctgaagaa atccgaactg aaggagctca tcaacaatga gctttcccat |
| 241 | ttcttagagg aaatcaaaga gcaggaggtt gtggacaaag tcatggaaac actggacaat |
| 301 | gatggagacg gcgaatgtga cttccaggaa ttcatggcct ttgttgccat ggttactact |
| 361 | gcctgccacg agttctttga acatgagtga gattagaaag cagccaaacc tttcctgtaa |
| 421 | cagagacggt catgcaagaa agcagacagc aagggcttgc agcctagtag gagctgagct |
| 481 | ttccagccgt gttgtagcta attaggaagc ttgatttgct ttgtgattga aaaattgaaa |
| 541 | acctctttcc aaaggctgtt ttaacggcct gcatcattct ttctgctata ttaggcctgt |
| 601 | gtgtaagctg actggcccca gggactcttg ttaacagtaa cttaggagtc aggtctcagt |
| 661 | gataaagcgt gcaccgtgca gcccgccatg gccgtgtaga ccctaacccg gagggaaccc |
| 721 | tgactacaga aattaccccg gggcaccctt aaaacttcca ctacctttaa aaaacaaagc |
| 781 | cttatccagc attatttgaa aacactgctg ttctttaaat gcgttcctca tccatgcaga |
| 841 | taacagctgg ttggccggtg tggccctgca agggcgtggt ggcttcggcc tgcttcccgg |
| 901 | gatgcgcctg atcaccaggt gaacgctcag cgctggcagc gctcctggaa aaagcaactc |
| 961 | catcagaact cgcaatccga gccagctctg ggggctccag cgtggcctcc gtgacccatg |
| 1021 | cgattcaagt cgcggctgca ggatccttgc ctccaacgtg cctccagcac atgcggcttc |
| 1081 | cgagggcact accgggggct ctgagccacc gcgagggcct gcgttcaata aaaag |

By “PAX6 polypeptide” (or paired box protein PAX6) is meant a polypeptide or fragment thereof having at least about 85% amino acid identity to NCBI Accession No. AAK95849.1.

(SEQ ID NO: 49)
MQNSHSGVNQLGGVFVNGRPLPDSTRQKIVELAHSGARPCDISRILQVSN
GCVSKILGRYYETGSIRPRAIGGSKPRVATPEVVSKIAQYKRECPSIFAW
EIRDRLLSEGVCTNDNIPSVSSINRVLRNLASEKQQMGADGMYDKLRMLN
GQTGSWGTRPGWYPGTSVPGQPTQDGCQQQEGGGENTNSISSNGEDSDEA
QMRLQLKRKLQRNRTSFTQEQIEALEKEFERTHYPDVFARERLAAKIDLP
EARIQVWFSNRRAKWRREEKLRNQRRQASNTPSHIPISSSFSTSVYQPIP
QPTTPVSSFTSGSMLGRTDTALTNTYSALPPMPSFTMANNLPMQPPVPSQ
TSSYSCMLPTSPSVNGRSYDTYTPPHMQTHMNSQPMGTSGTTSTGLISPG
VSVPVQVPGSEPDMSQYWPRLQ

By “PAX6 polynucleotide” (or paired box protein PAX6) is meant a polynucleotide encoding an PAX6 polypeptide. An exemplary PAX6 nucleic acid molecule (e.g., mRNA) is provided at NCBI Accession No. AY047583.

| (SEQ ID NO: 50) | |
|---|---|
| 1 | agggggaaga ctttaactag gggcgcgcag atgtgtgagg ccttttattg tgagagtgga |
| 61 | cagacatccg agatttcaga gccccatatt cgagccccgt ggaatcccgc ggcccccagc |
| 121 | cagagccagc atgcagaaca gtcacagcgg agtgaatcag ctcggtggtg tctttgtcaa |
| 181 | cgggcggcca ctgccggact ccacccggca gaagattgta gagctagctc acagcggggc |
| 241 | ccggccgtgc gacatttccc gaattctgca ggtgtccaac ggatgtgtga gtaaaattct |
| 301 | gggcaggtat tacgagactg gctccatcag acccagggca atcggtggta gtaaaccgag |
| 361 | agtagcgact ccagaagttg taagcaaaat agcccagtat aagcgggagt gcccgtccat |
| 421 | ctttgcttgg gaaatccgag acagattact gtccgagggg gtctgtacca acgataacat |
| 481 | accaagcgtg tcatcaataa acagagttct tcgcaacctg gctagcgaaa agcaacagat |
| 541 | gggcgcagac ggcatgtatg ataaactaag gatgttgaac gggcagaccg gaagctgggg |
| 601 | cacccgccct ggttggtatc cggggacttc ggtgccaggg caacctacgc aagatggctg |
| 661 | ccagcaacag gaaggagggg gagagaatac caactccatc agttccaacg gagaagattc |
| 721 | agatgaggct caaatgcgac ttcagctgaa gcggaagctg caaagaaata gaacatcctt |
| 781 | tacccaagag caaattgagg ccctggagaa agagtttgag agaacccatt atccagatgt |
| 841 | gtttgcccga gaaagactag cagccaaaat agatctacct gaagcaagaa tacaggtatg |
| 901 | gttttctaat cgaagggcca aatggagaag agaagaaaaa ctgaggaatc agagaagaca |
| 961 | ggccagcaac acacctagtc atattcctat cagcagtagt ttcagcacca gtgtctacca |
| 1021 | accaattcca caacccacca caccggtttc ctccttcaca tctggctcca tgttgggccg |
| 1081 | aacagacaca gccctcacaa acacctacag cgctctgccg cctatgccca gcttcaccat |
| 1141 | ggcaaataac ctgcctatgc aacccccagt ccccagccag acctcctcat actcctgcat |
| 1201 | gctgcccacc agcccttcgg tgaatgggcg gagttatgat acctacaccc ccccacatat |
| 1261 | gcagacacac atgaacagtc agccaatggg cacctcgggc accacttcaa caggactcat |
| 1321 | ttcccctggt gtgtcagttc cagttcaagt tcccggaagt gaacctgata tgtctcaata |
| 1381 | ctggccaaga ttacagtaa |

By “Nestin polypeptide” is meant a polypeptide or fragment thereof having at least about 85% amino acid identity to NCBI Accession No. NP_006608.1.

(SEQ ID NO: 51)
MEGCMGEESFQMWELNRRLEAYLARVKALEEQNELLSAELGGLRAQSADT
SWRAHADDELAALRALVDQRWREKHAAEVARDNLAEELEGVAGRCQQLRL
ARERTTEEVARNRRAVEAEKCARAWLSSQVAELERELEALRVAHEEERVG
LNAQAACAPRCPAPPRGPPAPAPEVEELARRLGEAWRGAVRGYQERVAHM
ETSLGQARERLGRAVQGAREGRLELQQLQAERGGLLERRAALEQRLEGRW
QERLRATEKFQLAVEALEQEKQGLQSQIAQVLEGRQQLAHLKMSLSLEVA
TYRTLLEAENSRLQTPGGGSKTSLSFQDPKLELQFPRTPEGRRLGSLLPV
LSPTSLPSPLPATLETPVPAFLKNQEFLQARTPTLASTPIPPTPQAPSPA
VDAEIRAQDAPLSLLQTQGGRKQAPEPLRAEARVAIPASVLPGPEEPGGQ
RQEASTGQSPEDHASLAPPLSPDHSSLEAKDGESGGSRVFSICRGEGEGQ
IWGLVEKETAIEGKVVSSLQQEIWEEEDLNRKEIQDSQVPLEKETLKSLG
EEIQESLKTLENQSHETLERENQECPRSLEEDLETLKSLEKENKELLKDV
EVVRPLEKEAVGQLKPTGKEDTQTLQSLQKENQELMKSLEGNLETFLFPG
TENQELVSSLQENLESLTALEKENQEPLRSPEVGDEEALRPLTKENQEPL
RSLEDENKEAFRSLEKENQEPLKTLEEEDQSIVRPLETENHKSLRSLEEQ
DQETLRTLEKETQQRRRSLGEQDQMTLRPPEKVDLEPLKSLDQEIARPLE
NENQEFLKSLKEESVEAVKSLETEILESLKSAGQENLETLKSPETQAPLW
TPEEINQGAMNPLEKEIQEPLESVEVNQETFRLLEEENQESLRSLGAWNL
ENLRSPEEVDKESQRNLEEEENLGKGEYQESLRSLEEEGQELPQSADVQR
WEDTVEKDQELAQESPPGMAGVENEDEAELNLREQDGFTGKEEVVEQGEL
NATEEVWIPGEGHPESPEPKEQRGLVEGASVKGGAEGLQDPEGQSQQVGA
PGLQAPQGLPEAIEPLVEDDVAPGGDQASPEVMLGSEPAMGESAAGAEPG
PGQGVGGLGDPGHLTREEVMEPPLEEESLEAKRVQGLEGPRKDLEEAGGL
GTEFSELPGKSRDPWEPPREGREESEAEAPRGAEEAFPAETLGHTGSDAP
SPWPLGSEEAEEDVPPVLVSPSPTYTPILEDAPGPQPQAEGSQEASWGVQ
GRAEALGKVESEQEELGSGEIPEGPQEEGEESREESEEDELGETLPDSTP
LGFYLRSPTSPRWDPTGEQRPPPQGETGKEGWDPAVLASEGLEAPPSEKE
EGEEGEEECGRDSDLSEEFEDLGTEAPFLPGVPGEVAEPLGQVPQLLLDP
AAWDRDGESDGFADEEESGEEGEEDQEEGREPGAGRWGPGSSVGSLQALS
SSQRGEFLESDSVSVSVPWDDSLRGAVAGAPKTALETESQDSAEPSGSEE
ESDPVSLEREDKVPGPLEIPSGMEDAGPGADIIGVNGQGPNLEGKSQHVN
GGVMNGLEQSEEVGQGMPLVSEGDRGSPFQEEEGSALKTSWAGAPVHLGQ
GQFLKFTQREGDRESWSSGED

By “Nestin polynucleotide” is meant a polynucleotide encoding an Nestin polypeptide. An exemplary Nestin nucleic acid molecule (e.g., mRNA) is provided at NCBI Accession No. NM_006617.

| (SEQ ID NO: 52) | |
|---|---|
| 1 | gctactccca ccccgccccg ccccgtcatt gtccccgtcg gtctcttttc tcttccgtcc |
| 61 | taaaagctct gcgagccgct cccttctccc ggtgccccgc gtctgtccat cctcagtggg |
| 121 | tcagacgagc aggatggagg gctgcatggg ggaggagtcg tttcagatgt gggagctcaa |
| 181 | tcggcgcctg gaggcctacc tggcccgggt caaggcgctg gaggagcaga atgagctgct |
| 241 | cagcgcggag ctcggggggc tccgggcaca atccgcggac acctcctggc gggcgcatgc |
| 301 | cgacgacgag ctggcggccc tgcgggccct cgttgaccaa cgctggcggg agaagcacgc |
| 361 | ggccgaggtg gcgcgcgaca acctggctga agagctggag ggcgtggcag gccgatgcca |
| 421 | gcagctgcgg ctggcccggg agcggacgac ggaggaggta gcccgcaacc ggcgcgccgt |
| 481 | cgaggcagag aaatgcgccc gggcctggct gagtagccag gtggcagagc tggagcgcga |
| 541 | gctagaggct ctacgcgtgg cgcacgagga ggagcgcgtc ggcctgaacg cgcaggctgc |
| 601 | ctgtgccccc cgctgccccg cgccgccccg cgggcctccc gcgccggccc cggaggtaga |
| 661 | ggagctggca aggcgactgg gcgaggcgtg gcgcggggca gtgcgcggct accaggagcg |
| 721 | cgtggcacac atggagacgt cgctgggcca ggcccgcgag cggctgggcc gggcggtgca |
| 781 | gggtgcccgc gagggccgcc tggagctgca gcagctccag gctgagcgcg gaggcctcct |
| 841 | ggagcgcagg gcagcgttgg aacagaggtt ggagggccgc tggcaggagc ggctgcgggc |
| 901 | tactgaaaag ttccagctgg ctgtggaggc cctggagcag gagaaacagg gcctacagag |
| 961 | ccagatcgct caggtcctgg aaggtcggca gcagctggcg cacctcaaga tgtccctcag |
| 1021 | cctggaggtg gccacgtaca ggaccctcct ggaggctgag aactcccggc tgcaaacacc |
| 1081 | tggcggtggc tccaagactt ccctcagctt tcaggacccc aagctggagc tgcaattccc |
| 1141 | taggacccca gagggccggc gtcttggatc tttgctccca gtcctgagcc caacttccct |
| 1201 | cccctcaccc ttgcctgcta cccttgagac acctgtgcca gcctttctta agaaccaaga |
| 1261 | attcctccag gcccgtaccc ctaccttggc cagcaccccc atccccccca cacctcaggc |
| 1321 | accctctcct gctgtagatg cagagatcag agcccaggat gctcctctct ctctgctcca |
| 1381 | gacacagggt gggaggaaac aggctccaga gcccctgcgg gctgaagcca gggtggccat |
| 1441 | tcctgccagc gtcctgcctg gaccagagga gcctgggggc cagcggcaag aggccagtac |
| 1501 | aggccagtcc ccagaggacc atgcctcctt ggcaccaccc ctcagccctg accactccag |
| 1561 | tttagaggct aaggatggag aatccggtgg gtctagagtg ttcagcatat gccgagggga |
| 1621 | aggtgaaggg caaatctggg ggttggtaga gaaagaaaca gccatagagg gcaaagtggt |
| 1681 | aagcagcttg cagcaggaaa tatgggaaga agaggatcta aacaggaagg aaatccagga |
| 1741 | ctcccaggtt cctttggaaa aagaaaccct gaagtctctg ggagaggaga ttcaagagtc |
| 1801 | actgaagact ctggaaaacc agagccatga gacactagaa agggagaatc aagaatgtcc |
| 1861 | gaggtcttta gaagaagact tagaaacact aaaaagtcta gaaaaggaaa ataaagagct |
| 1921 | attaaaggat gtggaggtag tgagacctct agaaaaagag gctgtaggcc aacttaagcc |
| 1981 | tacaggaaaa gaggacacac agacattgca atccctgcaa aaggagaatc aagaactaat |
| 2041 | gaaatctctt gaaggtaatc tagagacatt tttatttcca ggaacggaaa atcaagaatt |
| 2101 | agtaagttct ctgcaagaga acttagagtc attgacagct ctggaaaagg agaatcaaga |
| 2161 | gccactgaga tctccagaag taggggatga ggaggcactg agacctctga caaaggagaa |
| 2221 | tcaggaaccc ctgaggtctc ttgaagatga gaacaaagag gcctttagat ctctagaaaa |
| 2281 | agagaaccag gagccactga agactctaga agaagaggac cagagtattg tgagacctct |
| 2341 | agaaacagag aatcacaaat cactgaggtc tttagaagaa caggaccaag agacattgag |
| 2401 | aactcttgaa aaagagactc aacagcgacg gaggtctcta ggggaacagg atcagatgac |
| 2461 | attaagaccc ccagaaaaag tggatctaga accactgaag tctcttgacc aggagatagc |
| 2521 | tagacctctt gaaaatgaga atcaagagtt cttaaagtca ctcaaagaag agagcgtaga |
| 2581 | ggcagtaaaa tctttagaaa cagagatcct agaatcactg aagtctgcgg gacaagagaa |
| 2641 | cctggaaaca ctgaaatctc cagaaactca agcaccactg tggactccag aagaaataaa |
| 2701 | tcagggggca atgaatcctc tagaaaagga aattcaagaa ccactggagt ctgtggaagt |
| 2761 | gaaccaagag acattcagac tcctggaaga ggagaatcag gaatcattga gatctctggg |
| 2821 | agcatggaac ctggagaatt tgagatctcc agaggaggta gacaaggaaa gtcaaaggaa |
| 2881 | tctggaagag gaagagaacc tgggaaaggg agagtaccaa gagtcactga ggtctctgga |
| 2941 | ggaggaggga caggagctgc cgcagtctgc agatgtgcag aggtgggaag atacggtgga |
| 3001 | gaaggaccaa gaactggctc aggaaagccc tcctgggatg gctggagtgg aaaatgagga |
| 3061 | tgaggcagag ctgaatctga gggagcagga tggcttcact gggaaggagg aggtggtaga |
| 3121 | gcagggagag ctgaatgcca cagaggaggt ctggatccca ggcgaggggc acccagagag |
| 3181 | ccctgagccc aaagagcaga gaggcctggt tgagggagcc agtgtgaagg gaggggctga |
| 3241 | gggcctccag gaccctgaag ggcaatcaca acaggtgggg gccccaggcc tccaggctcc |
| 3301 | ccaggggctg ccagaggcga tagagcccct ggtggaagat gatgtggccc cagggggtga |
| 3361 | ccaagcctcc ccagaggtca tgttggggtc agagcctgcc atgggtgagt ctgctgcggg |
| 3421 | agctgagcca ggcccggggc agggggtggg agggctgggg gacccaggcc atctgaccag |
| 3481 | ggaagaggtg atggaaccac ccctggaaga ggagagtttg gaggcaaaga gggttcaggg |
| 3541 | cttggaaggg cctagaaagg acctagagga ggcaggtggt ctggggacag agttctccga |
| 3601 | gctgcctggg aagagcagag acccttggga gcctcccagg gagggtaggg aggagtcaga |
| 3661 | ggctgaggcc cccaggggag cagaggaggc gttccctgct gagaccctgg gccacactgg |
| 3721 | aagtgatgcc ccttcacctt ggcctctggg gtcagaggaa gctgaggagg atgtaccacc |
| 3781 | agtgctggtc tcccccagcc caacgtacac cccgatcctg gaagatgccc ctgggcctca |
| 3841 | gcctcaggct gaagggagtc aggaggctag ctggggggtg caggggaggg ctgaagccct |
| 3901 | ggggaaagta gagagcgagc aggaggagtt gggttctggg gagatccccg agggccccca |
| 3961 | ggaggaaggg gaggagagca gagaagagag cgaggaggat gagctcgggg agacccttcc |
| 4021 | agactccact cccctgggct tctacctcag gtcccccacc tcccccaggt gggaccccac |
| 4081 | tggagagcag aggccacccc ctcaagggga gactggaaag gagggctggg atcctgctgt |
| 4141 | cctggcttcc gagggccttg aggccccacc ctcagaaaag gaggaggggg aggagggaga |
| 4201 | agaggagtgt ggccgtgact ctgacctgtc agaagaattt gaggacctgg ggactgaggc |
| 4261 | accttttctt cctggggtcc ctggggaggt ggcagaacct ctgggccagg tgccccagct |
| 4321 | gctactggat cctgcagcct gggatcgaga tggggagtcc gatgggtttg cagatgagga |
| 4381 | agaaagtggg gaggagggag aggaggatca ggaggagggg agggagccag gggctgggcg |
| 4441 | gtgggggcca gggtcttctg ttggcagcct ccaggccctg agtagctccc agagagggga |
| 4501 | attcctggag tctgattctg tgagtgtcag tgtcccctgg gatgacagct tgaggggtgc |
| 4561 | agtggctggt gcccccaaga ctgccctgga aacggagtcc caggacagtg ctgagccttc |
| 4621 | tggctcagag gaagagtctg accctgtttc cttggagagg gaggacaaag tccctggccc |
| 4681 | tctagagatc cccagtggga tggaggatgc aggcccaggg gcagacatca ttggtgttaa |
| 4741 | tggccagggt cccaacttgg aggggaagtc acagcatgtg aatgggggag tgatgaacgg |
| 4801 | gctggagcag tctgaggaag tggggcaagg aatgccgcta gtctctgagg gagaccgagg |
| 4861 | gagccccttt caggaggagg aggggagtgc tctgaagacc tcttgggcag gggctcctgt |
| 4921 | tcacctgggc cagggtcagt tcctgaagtt cactcagagg gaaggagata gagagtcctg |
| 4981 | gtcctcaggg gaggactagg aaaagaccat ctgcccggca ctggggactt aggggtgcgg |
| 5041 | ggaggggaag gacgcctcca agcccgctcc ctgctcagga gcagcactct taacttacga |
| 5101 | tctcttgaca tatggtttct ggctgagagg cctggcccgc taaggtgaaa aggggtgtgg |
| 5161 | caaaggagcc tactccaaga atggaggctg taggaatata acctcccacc ctgcaaaggg |
| 5221 | aatctcttgc ctgctccatc tcataggcta agtcagctga atcccgatag tactaggtcc |
| 5281 | ccttccctcc gcatcccgtc agctggaaaa ggcctgtggc ccagaggctt ctccaaaggg |
| 5341 | agggtgacat gctggctttt gtgcccaagc tcaccagccc tgcgccacct cactgcagta |
| 5401 | gtgcaccatc tcactgcagt agcacgccct cctgggccgt ctggcctgtg gctaatggag |
| 5461 | gtgacggcac tcccatgtgc tgactccccc catccctgcc acgctgtggc cctgcctggc |
| 5521 | tagtccctgc ctgaataaag taatgcctcc gcttcaaaaa aaaaaaaaaa aaaaaaaaaa |
| 5581 | aaaaaaaaaa a |

By “LHX6 polypeptide” (or LIM homeobox 6) is meant a polypeptide or fragment thereof having at least about 85% amino acid identity to NCBI Accession No. AAI03937.1.

(SEQ ID NO: 53)
MAQPGSGCKATTRCLEGTAPPAMAQSDAEALAGALDKDEGQASPCTPSTP
SVCSPPSAASSVPSAGKNICSSCGLEILDRYLLKVNNLIWHVRCLECSVC
RTSLRQQNSCYIKNKEIFCKMDYFSRFGTKCARCGRQIYASDWVRRARGN
AYHLACFACFSCKRQLSTGEEFGLVEEKVLCRIHYDTMIENLKRAAENGN
GLTLEGAVPSEQDSQPKPAKRARTSFTAEQLQVMQAQFAQDNNPDAQTLQ
KLADMTGLSRRVIQVWFQNCRARHKKHTPQHPVPPSGAPPSRLPSALSDD
IHYTPFSSPERARMVTLHGYIESHPFSVLTLPALPHLPVGAPQLPLSR

By “LHX6 polynucleotide” (or LIM homeobox 6) is meant a polynucleotide encoding an LHX6 polypeptide. An exemplary LHX6 nucleic acid molecule (e.g., mRNA) is provided at NCBI Accession No. BC103936.

| (SEQ ID NO: 54) | |
|---|---|
| 1 | cccgccaccg accaggtgat ggcccagcca gggtccggct gcaaagcgac cacccgctgt |
| 61 | cttgaaggga ccgcgccgcc cgccatggct cagtctgacg ccgaggccct ggcaggagct |
| 121 | ctggacaagg acgagggtca ggcctcccca tgtacgccca gcacgccatc tgtctgctca |
| 181 | ccgccctctg ccgcctcctc cgtgccgtct gcaggcaaga acatctgctc cagctgcggc |
| 241 | ctcgagatcc tggaccgata tctgctcaag gtcaacaacc tcatctggca cgtgcggtgc |
| 301 | ctcgagtgct ccgtgtgtcg cacgtcgctg aggcagcaga acagctgcta catcaagaac |
| 361 | aaggagatct tctgcaagat ggactacttc agccgattcg ggaccaagtg tgcccggtgc |
| 421 | ggccgacaga tctacgccag cgactgggtg cggagagctc gcggcaacgc ctaccacctg |
| 481 | gcctgcttcg cctgcttctc gtgcaagcgc cagctgtcca ctggtgagga gttcggcctg |
| 541 | gtcgaggaga aggtgctctg ccgcatccac tacgacacca tgattgagaa cctcaagagg |
| 601 | gccgccgaga acgggaacgg cctcacgttg gagggggcag tgccctcgga acaggacagt |
| 661 | caacccaagc cggccaagcg cgcgcggacg tccttcaccg cggaacagct gcaggttatg |
| 721 | caggcgcagt tcgcgcagga caacaacccc gacgctcaga cgctgcagaa gctggcggac |
| 781 | atgacgggcc tcagccggag agtcatccag gtgtggtttc aaaactgccg ggcgcgtcat |
| 841 | aaaaagcaca cgccgcaaca cccagtgccg ccctcggggg cgcccccgtc ccgccttccc |
| 901 | tccgccctgt ccgacgacat ccactacacc ccgttcagca gccccgagcg ggcgcgcatg |
| 961 | gtcaccctgc acggctacat tgagagtcat cctttttcag tactaacgct gccggcactt |
| 1021 | ccgcatctgc ccgtgggcgc cccacagctg cccctcagcc gctgagatcc agtgtccaag |
| 1081 | ctgcggccag gagtccaccc acctccgcat ccacccccgt ccgccatcct gcccaccacc |
| 1141 | aggtcggttc ccgaggcctg gcctttccct ctcctgctga gaaccagaac ccaccaggag |
| 1201 | caccacagag tcctcctctt ggaaggcaga actccctgaa atctggaatc agggtggaaa |
| 1261 | cagcctgttt ttcccattta aacaggagtc ctcttcaact tcagctgatt acaataacaa |
| 1321 | aaggcggaat tgaattgtgc gatgccaacg gccttctcat ttacaggttt ttttccccca |
| 1381 | cattggcctt tatttactac ttccttggaa ccatctctga attctgaata gctgacaacc |
| 1441 | cccaatgtta tccactctgt tgcttttgtc tggaaaactc tacagtgttt gtgggatgtc |
| 1501 | cccaaaggta agctatgttc taattttatc atttccatct gtctggttat gtcaagttaa |
| 1561 | ttcagaaaga gaagagacag tgaccaaccc tgagaggcct aatagggcag agatggaggc |
| 1621 | ctgcccagac taggaggcag cggggataga cagggaatgg ggagaagaaa gacccccatt |
| 1681 | ggtttggaaa tcaaggagag ggcggtgaca tattggacca gaagaggcac tagccatttt |
| 1741 | aaggagagga aagagaaaac tctggggtca gggagagacc ctacccccac ctaattatcc |
| 1801 | agcatatatg taagaaacat agcagcgatg gtattcgatc tgtgccatga ctcttctgaa |
| 1861 | tgtttggaca ggttagagtt ggggacccct gttggccact tgttgacctc tcatagtggt |
| 1921 | gcttgggcca ggtcttctca atggaagggg aatcccttat aggggagagg gaacagagcc |
| 1981 | cagtgaaatg gcagtcagaa tgttaaccct ggatccatct ctaagtagag agagggtgcc |
| 2041 | cattgcctag gtgagtgtgc caagctcagg attccaactg gtgcctctga gcttcccaat |
| 2101 | caatacttcc tggagccagc cccacccacc cctgagaaca gaggtcagac acagctgcgt |
| 2161 | aacatccatc ctgctacaac tcttccaccc caaacaaaag ggctcaggct acacacgacc |
| 2221 | atgatttatg ttttcagggg atgcccattt gtcccaagct tatcctgtaa ttctagaatt |
| 2281 | acctggtgtc ctgatgcatt ttccactaga ggttgctaat cagcatgttt tagcccaagt |
| 2341 | ccaccttcct gctgtggtta acctgttatg ttgcttttgg aaggagactc taagacaggg |
| 2401 | aaagcaagtt catggtacat acgcagccat tgtctctgtt tttacccatg gcagacattg |
| 2461 | ctaatcaatg gcagctctat ttcactgagt ctggataagg tttcagagtt caaatgcttg |
| 2521 | acgttggcac ttaacatgaa agcctatagg tcattcttgc tctgggatct acaggcaggg |
| 2581 | taggcacagg tgcagcctaa gaagggaacc tgcttcctct cccttccaaa gacagtgaca |
| 2641 | gctgactgag ggcaaagagc aggcaccact cagaacgtgg tgagtacagc tcagctcagc |
| 2701 | actcagtcag tggtaacttg tgcccagccc tgtgctaggc gctgacatta acaggagcaa |
| 2761 | ccagggccca attcctggcc ttggagctca aatctttcct ttgatttttg ctcctgatca |
| 2821 | tcaaggcccc agtgg |

By “LHX8 polypeptide” (or LIM homeobox 8) is meant a polypeptide or fragment thereof having at least about 85% amino acid identity to NCBI Accession No. AAH40321.1.

(SEQ ID NO: 55)
MQILSRCQGLMSEECGRTTALAAGRTRKGAGEEGLVSPEGAGDEDSCSSS
APLSPSSSPRSMASGSGCPPGKCVCNSCGLEIVDKYLLKVNDLCWHVRCL
SCSVCRTSLGRHTSCYIKDKDIFCKLDYFRRYGTRCSRCGRHIHSTDWVR
RAKGNVYHLACFACFSCKRQLSTGEEFALVEEKVLCRVHYDCMLDNLKRE
VENGNGISVEGALLTEQDVNHPKPAKRARTSFTADQLQVMQAQFAQDNNP
DAQTLQKLAERTGLSRRVIQVWFQNCRARHKKHVSPNHSSSTPVTAAPPS
RLSPPMLEEMAYSAYVPQDGTMLTALHSYMDAHSPTTLGLQPLLPHSMTQ
LPISHT

By “LHX8 polynucleotide” (or LIM homeobox 8) is meant a polynucleotide encoding an LHX8 polypeptide. An exemplary LHX8 nucleic acid molecule (e.g., mRNA) is provided at NCBI Accession No. BC040321.

| (SEQ ID NO: 56) | |
|---|---|
| 1 | agcggcaaga ggctagcggc tggaccactt gtgctggagt ggtaaagaac tatcatgaat |
| 61 | ccatttactg aaagtgtcca tttctgaact caccctaaag aggacaaaca ccgcaaagta |
| 121 | gttaaaagtc aggcattcgc gtcggacgtc tgggtttgaa ttctgccctg gcttgactgg |
| 181 | aaacgcttcc cctatttctt ccgtagcgga ccgggagagc ttactggcgc tctgcgaacc |
| 241 | ggctggaaag aaacaccgag tcactcgtac agactcttgg tcgcagaact tggctttccg |
| 301 | ctattggtcc tccagaaccg cttgaaacaa ctggccccag ctggcgcatc agaccgcagt |
| 361 | gaggaatgcc gcggggcggg tggcgaaggc agggtctgcc cgccagtgga ttcccgggtg |
| 421 | tcccgcgtgg agcaggcttg cccagctggg aagcccatca aacctcagtc ttggcccaca |
| 481 | gtgggagaga gaccagtggg tcccagacgg aggccatcgc ccgcttttgg cgacctccac |
| 541 | tggcgtgaat aaaagcaccc ctctcttacc ctcagaaact gtgggtagca aggtataaaa |
| 601 | cggagtctgg gaccggtaag tcccaaggtg agcccgtata cagctctgcc atctctgagg |
| 661 | ggttatgcag attctgagca ggtgtcaggg gctcatgtca gaggagtgcg ggcggactac |
| 721 | agccctggcg gccgggagga ctcgcaaagg cgccggggaa gagggactgg tgagccccga |
| 781 | gggagcgggg gacgaggact cgtgctcctc ctcggccccg ctgtccccgt cgtcctcgcc |
| 841 | ccggtccatg gcctcgggct ccggctgccc tcctggcaag tgtgtgtgca acagttgcgg |
| 901 | cctggagatc gtggacaaat accttctcaa ggtgaatgac ctatgctggc atgtccggtg |
| 961 | tctctcctgc agtgtttgca gaacctccct aggaaggcac accagctgtt atattaaaga |
| 1021 | caaagacatt ttctgcaaac ttgattattt cagaaggtat ggaactcgct gctctcgatg |
| 1081 | tgggagacac atccattcta ctgactgggt ccggagagcc aaggggaatg tctatcactt |
| 1141 | ggcatgcttt gcctgctttt cctgcaaaag gcaactttcc acaggagagg agtttgcttt |
| 1201 | ggtggaagag aaagtcctct gcagagtaca ttatgactgc atgctggata atttaaaaag |
| 1261 | agaagtagaa aatgggaatg ggattagtgt ggaaggtgcc ctcctcacag agcaagatgt |
| 1321 | taaccatcca aaaccagcaa aaagagctcg gaccagcttt acagcagatc agcttcaggt |
| 1381 | tatgcaagca caatttgctc aggacaacaa cccagatgca cagacactcc agaaattggc |
| 1441 | agaaaggaca ggcttgagca gacgtgtgat acaggtgtgg tttcagaatt gtagagcacg |
| 1501 | ccacaagaaa cacgtcagtc ctaatcactc atcctccacc ccagtcacag cagccccacc |
| 1561 | ctccaggctg tctccaccca tgttagaaga aatggcttat tctgcctacg tgccccaaga |
| 1621 | tggaacgatg ttaactgcgc tgcatagtta tatggatgct cattcaccaa caactcttgg |
| 1681 | actccagccc ttgttacccc attcaatgac acaactgcca ataagtcata cctaattctt |
| 1741 | ttttcaggga tagacttgat taaggatata aatttgtcat ttattatgta taaaatacca |
| 1801 | ttgaaaagat attactgtta attttttatt taacacctaa agcatttcca acatcacttt |
| 1861 | gctgcccagg tatgtatcta tagttggcct gcaagacact tttattaatt cttcattttt |
| 1921 | tgtaaaactt atgtttacaa gaagaaaaca aatcaaaaca ttttttgtat tgtctggaaa |
| 1981 | tagttcactc tagtgtgtat ctgttaattt atttgtcatc aaaagagcac tttgcctaaa |
| 2041 | agaaaggact gacaagtgtg caaaatgttt acaatctttt gtgaaattgt agtttatcat |
| 2101 | tagtttgtat ctgtaagtta ttgtaataaa tattacctgt attttttgtt atatacaact |
| 2161 | ttatactttg aagcttgtat ctgtgaattt gcaactgaaa tttattttgc caatgttttc |
| 2221 | tgaatgaact gaataaagct tctgttgtag catgccatgc aaacacatta ttgtgtttgt |
| 2281 | ggttgatgaa ttatggctgt aaataacact atagtttaat aagcccacca ttctgagttt |
| 2341 | attaaacatt ttccattctt gtgaaaattt caaaaaaaaa aaaaaaaaaa aaagaaaaaa |
| 2401 | aaaaaaaaaa a |

By “TBR1 polypeptide” (or T-box, brain 1 (TBR1)) is meant a polypeptide or fragment thereof having at least about 85% amino acid identity to NCBI Accession No. NP_006584.1.

(SEQ ID NO: 57)
MQLEHCLSPSIMLSKKFLNVSSSYPHSGGSELVLHDHPIISTTDNLERSS
PLKKITRGMTNQSDTDNFPDSKDSPGDVQRSKLSPVLDGVSELRHSFDGS
AADRYLLSQSSQPQSAATAPSAMFPYPGQHGPAHPAFSIGSPSRYMAHHP
VITNGAYNSLLSNSSPQGYPTAGYPYPQQYGHSYQGAPFYQFSSTQPGLV
PGKAQVYLCNRPLWLKFHRHQTEMIITKQGRRMFPFLSFNISGLDPTAHY
NIFVDVILADPNHWRFQGGKWVPCGKADTNVQGNRVYMHPDSPNTGAHWM
RQEISFGKLKLTNNKGASNNNGQMVVLQSLHKYQPRLHVVEVNEDGTEDT
SQPGRVQTFTFPETQFIAVTAYQNTDITQLKIDHNPFAKGFRDNYDTIYT
GCDMDRLTPSPNDSPRSQIVPGARYAMAGSFLQDQFVSNYAKARFHPGAG
AGPGPGTDRSVPHTNGLLSPQQAEDPGAPSPQRWFVTPANNRLDFAASAY
DTATDFAGNAATLLSYAAAGVKALPLQAAGCTGRPLGYYADPSGWGARSP
PQYCGTKSGSVLPCWPNSAAAAARMAGANPYLGEEAEGLAAERSPLPPGA
AEDAKPKDLSDSSWIETPSSIKSIDSSDSGIYEQAKRRRISPADTPVSES
SSPLKSEVLAQRDCEKNCAKDISGYYGFYSHS

By “TBR1 polynucleotide” (or T-box, brain 1 (TBR1)) is meant a polynucleotide encoding an TBR1 polypeptide. An exemplary TBR1 nucleic acid molecule (e.g., mRNA) is provided at NCBI Accession No. NM_006593.

| (SEQ ID NO: 58) | |
|---|---|
| 1 | gtcgctacca ggagccaggt gattatccta attaatgtct atctaattaa attactgtca |
| 61 | gcagctaacc aatggcagga gccgtttcat cggctgcaca agcagcaaga tcaaaagtga |
| 121 | gccttttctg attgctgcat agtgtcaatt ggccaatctc ttctcccagg gaaaaaaaaa |
| 181 | agtaaatcaa acctttgaga agcatttgct ggttgaagtg ctttctgtct agtgaggggg |
| 241 | tctgtggatt tctagtttat gataaatagg actttaaaaa ccagggacgg gagggcgagt |
| 301 | gttcaggttc tagagctatg cagctggagc actgcctttc tccttctatc atgctctcca |
| 361 | agaaatttct caatgtgagc agcagctacc cacattcagg cggatccgag cttgtcttgc |
| 421 | acgatcatcc cattatctcg accactgaca acctggagag aagttcacct ttgaaaaaaa |
| 481 | ttaccagggg gatgacgaat cagtcagata cagacaattt tcctgactcc aaggactcac |
| 541 | caggggacgt ccagagaagt aaactctctc ctgtcttgga cggggtctct gagcttcgtc |
| 601 | acagtttcga tggctctgct gcagatcgct acctcctctc tcagtccagc cagccacagt |
| 661 | ctgcggccac tgctcccagt gccatgttcc cgtaccccgg ccagcacgga ccggcgcacc |
| 721 | ccgccttctc catcggcagc cctagccgct acatggccca ccacccggtc atcaccaacg |
| 781 | gagcctacaa cagcctcctg tccaactcct cgccgcaggg ataccccacg gccggctacc |
| 841 | cctacccaca gcagtacggc cactcctacc aaggagctcc gttctaccag ttctcctcca |
| 901 | cccagccggg gctggtgccc ggcaaagcac aggtgtacct gtgcaacagg cccctttggc |
| 961 | tgaaatttca ccggcaccaa acggagatga tcatcaccaa acagggaagg cgcatgtttc |
| 1021 | cttttttaag ttttaacatt tctggtctcg atcccacggc tcattacaat atttttgtgg |
| 1081 | atgtgatttt ggcggatccc aatcactgga ggtttcaagg aggcaaatgg gttccttgcg |
| 1141 | gcaaagcgga caccaatgtg caaggaaatc gggtctatat gcatccggat tcccccaaca |
| 1201 | ctggggctca ctggatgcgc caagaaatct cttttggaaa attaaaactt acgaacaaca |
| 1261 | aaggagcttc aaataacaat gggcagatgg tggttttaca gtccttgcac aagtaccagc |
| 1321 | cccgcctgca tgtggtggaa gtgaacgagg acggcacgga ggacactagc cagcccggcc |
| 1381 | gcgtgcagac gttcactttc cctgagactc agttcatcgc cgtcaccgcc taccagaaca |
| 1441 | cggatattac acaactgaaa atagatcaca acccttttgc aaaaggattt cgggataatt |
| 1501 | atgacacgat ctacaccggc tgtgacatgg accgcctgac cccctcgccc aacgactcgc |
| 1561 | cgcgctcgca gatcgtgccc ggggcccgct acgccatggc cggctctttc ctgcaggacc |
| 1621 | agttcgtgag caactacgcc aaggcccgct tccacccggg cgcgggcgcg ggccccgggc |
| 1681 | cgggtacgga ccgcagcgtg ccgcacacca acgggctgct gtcgccgcag caggccgagg |
| 1741 | acccgggcgc gccctcgccg caacgctggt ttgtgacgcc ggccaacaac cggctggact |
| 1801 | tcgcggcctc ggcctatgac acggccacgg acttcgcggg caacgcggcc acgctgctct |
| 1861 | cttacgcggc ggcgggcgtg aaggcgctgc cgctgcaggc tgcaggctgc actggccgcc |
| 1921 | cgctcggcta ctacgccgac ccgtcgggct ggggcgcccg cagtcccccg cagtactgcg |
| 1981 | gcaccaagtc gggctcggtg ctgccctgct ggcccaacag cgccgcggcc gccgcgcgca |
| 2041 | tggccggcgc caatccctac ctgggcgagg aggccgaggg cctggccgcc gagcgctcgc |
| 2101 | cgctgccgcc cggcgccgcc gaggacgcca agcccaagga cctgtccgat tccagctgga |
| 2161 | tcgagacgcc ctcctcgatc aagtccatcg actccagcga ctcggggatt tacgagcagg |
| 2221 | ccaagcggag gcggatctcg ccggccgaca cgcccgtgtc cgagagttcg tccccgctca |
| 2281 | agagcgaggt gctggcccag cgggactgcg agaagaactg cgccaaggac attagcggct |
| 2341 | actatggctt ctactcgcac agctaggccg cccctgcccg cccggccccg ccgcggcccg |
| 2401 | gacccccagc cagcccctca cagctcttcc ccagctccgc ctccccacac tcctccttgc |
| 2461 | gcacccactc attttatttg accctcgatg gccgtctgca gcgaataagt gcaggtctcc |
| 2521 | gagcgtgatt ttaacctttt ttgcacagca gtctctgcaa ttagctcacc gaccttcaac |
| 2581 | tttgctgtaa accttttggt tttcctactt actcttcttc tgtggagtta tcctcctaca |
| 2641 | attcccctcc ccctcgtctt tctcttacct cctacttctc tttcttgtaa tgaaactctt |
| 2701 | cacctttagg agacctgggc agtcctgtca ggcagcagcg attccgaccc gccaagtctc |
| 2761 | ggcctccaca ttaaccatag gatgttgact ctagaacctg gacccaccca gcgcgtcctt |
| 2821 | tcttatcccc gagtggatgg atggatggat ggatggtagg gatgttaata attttagtgg |
| 2881 | aacaaagcct gtgaaatgat tgtacatagt gttaatttat tgtaacgaat ggctagtttt |
| 2941 | tattctcgtc aaggcacaaa accagttcat gcttaacctt tttttccttt cctttctttg |
| 3001 | cttttctttc tctcctctca tactttctct tctctctctt ttaattttct tgtgagataa |
| 3061 | tattctaaga ggctctagaa acatgaaata ctcagtagtg atgggtttcc cacttctcct |
| 3121 | caatccgttg catgaaataa ttactatgtg ccctaatgca cacaaatagc taaggagaat |
| 3181 | ccacccaaac acctttaaag gataggtgtc tgttcatagg caagtcgatt aagtggcatg |
| 3241 | atgcctgcaa agcaaagtca actggagttg tatgttcccc ccaccttcta aatagaatag |
| 3301 | ctcgacatca gcaatattat tttgccttat ttgtttttcc ccaaagtgcc aaatccatta |
| 3361 | ctggtctgtg caggtgccaa atatgctgac aaactgtttc tgaatatctt tcagtacccc |
| 3421 | ttcaccttta tatgctgtaa atctttgtaa tgaatactct attaatgata tagatgactg |
| 3481 | aattgttggt aactatagtg tagtctagtg aagatgaatt gtgtgagttg tatattttac |
| 3541 | tgcattttag ttttgaaaat gacttcccca ccacctagaa acagctgaaa tttgacttcc |
| 3601 | ttgggagaac actagcatta atgcaagtaa gactgatttt cccctaagtc ttgttatatt |
| 3661 | tgataaggag cattaatccc cctggaaata gattagtagg atttctaatg ttgtgtagca |
| 3721 | aacctatact tttttgtatt taaaaattaa tgtgaaatat gcatcataca caatattcaa |
| 3781 | tctagattcc agtccatggg gggatttttc ctaataggaa ttcagggtct aaacgtgtgt |
| 3841 | atattttggc tcttctgtaa atctaatgtt gtgattttta tatttgtttc gttttgtctg |
| 3901 | tgaactgaat aatttataca agaacacact ccattgagaa acgttttgtt ttttgctcgt |
| 3961 | ttgtatcgtc tgtgtataac aagtaaaata aacctggtaa aaacgc |

By “SLC1A3 polypeptide” (or solute carrier family 1; glial high affinity glutamate transporter member 3 (SLC1A3)) is meant a polypeptide or fragment thereof having at least about 85% amino acid identity to NCBI Accession No. BAG35230.1.

(SEQ ID NO: 59)
MTKSNGEEPKMGGRMERFQQGVRKRTLLAKKKVQNITKEDVKSYLFRNAF
VLLTVTAVIVGTILGFTLRPYRMSYREVKYFSFPGELLMRMLQMLVLPLI
ISSLVTGMAALDSKASGKMGMRAVVYYMTTTIIAVVIGIIIVIIIHPGKG
TKENMHREGKIVRVTAADAFLDLIRNMFPPNLVEACFKQFKTNYEKRSFK
VPIQANETLVGAVINNVSEAMETLTRITEELVPVPGSVNGVNALGLVVFS
MCFGFVIGNMKEQGQALREFFDSLNEAIMRLVAVIMWYAPVGILFLIAGK
IVEMEDMGVIGGQLAMYTVTVIVGLLIHAVIVLPLLYFLVTRKNPWVFIG
GLLQALITALGTSSSSATLPITFKCLEENNGVDKRVTRFVLPVGATINMD
GTALYEALAAIFIAQVNNFELNFGQIITISITATAASIGAAGIPQAGLVT
MVIVLTSVGLPTDDITLIIAVDWFLDRLRTTTNVLGDSLGAGIVEHLSRH
ELKNRDVEMGNSVIEENEMKKPYQLIAQDNETEKPIDSETKM

By “SLC1A3 polynucleotide” (or solute carrier family 1; glial high affinity glutamate transporter member 3 (SLC1A3)) is meant a polynucleotide encoding an SLC1A3 polypeptide. An exemplary SLC1A3 nucleic acid molecule (e.g., mRNA) is provided at NCBI Accession No. AK312304.

| (SEQ ID NO: 60) | |
|---|---|
| 1 | gatagtaact tgcagtttca gagcacatgc acactgtcag ggctagcctg cctgcttacg |
| 61 | cgcgctgcgg attgttgctc cgttgtacct gctggggaat tcacctcgtt actgcttgat |
| 121 | atcttccacc ccttacaaaa tcagaaaagt tgtgttttct aataccaaag aggaggtttg |
| 181 | gctttctgtg ggtgattccc agacactgaa gtgcaaagaa gagaccctcc tagaaaagta |
| 241 | aaatatgact aaaagcaatg gagaagagcc caagatgggg ggcaggatgg agagattcca |
| 301 | gcagggagtc cgtaaacgca cacttttggc caagaagaaa gtgcagaaca ttacaaagga |
| 361 | ggatgttaaa agttacctgt ttcggaatgc ttttgtgctg ctcacagtca ccgctgtcat |
| 421 | tgtgggtaca atccttggat ttaccctccg accatacaga atgagctacc gggaagtcaa |
| 481 | gtacttctcc tttcctgggg aacttctgat gaggatgtta cagatgctgg tcttaccact |
| 541 | tatcatctcc agtcttgtca caggaatggc ggcgctagat agtaaggcat cagggaagat |
| 601 | gggaatgcga gctgtagtct attatatgac taccaccatc attgctgtgg tgattggcat |
| 661 | aatcattgtc atcatcatcc atcctgggaa gggcacaaag gaaaacatgc acagagaagg |
| 721 | caaaattgta cgagtgacag ctgcagatgc cttcctggac ttgatcagga acatgttccc |
| 781 | tccaaatctg gtagaagcct gctttaaaca gtttaaaacc aactatgaga agagaagctt |
| 841 | taaagtgccc atccaggcca acgaaacgct tgtgggtgct gtgataaaca atgtgtctga |
| 901 | ggccatggag actcttaccc gaatcacaga ggagctggtc ccagttccag gatctgtgaa |
| 961 | tggagtcaat gccctgggtc tagttgtctt ctccatgtgc ttcggttttg tgattggaaa |
| 1021 | catgaaggaa caggggcagg ccctgagaga gttctttgat tctcttaacg aagccatcat |
| 1081 | gagactggta gcagtaataa tgtggtatgc ccccgtgggt attctcttcc tgattgctgg |
| 1141 | gaagattgtg gagatggaag acatgggtgt gattgggggg cagcttgcca tgtacaccgt |
| 1201 | gactgtcatt gttggcttac tcattcacgc agtcatcgtc ttgccactcc tctacttctt |
| 1261 | ggtaacacgg aaaaaccctt gggtttttat tggagggttg ctgcaagcac tcatcaccgc |
| 1321 | tctggggacc tcttcaagtt ctgccaccct acccatcacc ttcaagtgcc tggaagagaa |
| 1381 | caatggcgtg gacaagcgcg tcaccagatt cgtgctcccc gtaggagcca ccattaacat |
| 1441 | ggatgggact gccctctatg aggctttggc tgccattttc attgctcaag ttaacaactt |
| 1501 | tgaactgaac ttcggacaaa ttattacaat cagcatcaca gccacagctg ccagtattgg |
| 1561 | ggcagctgga attcctcagg cgggcctggt cactatggtc attgtgctga catctgtcgg |
| 1621 | cctgcccact gacgacatca cgctcatcat cgcggtggac tggttcctgg atcgcctccg |
| 1681 | gaccaccacc aacgtactgg gagactccct gggagctggg attgtggagc acttgtcacg |
| 1741 | acatgaactg aagaacagag atgttgaaat gggtaactca gtgattgaag agaatgaaat |
| 1801 | gaagaaacca tatcaactga ttgcacagga caatgaaact gagaaaccca tcgacagtga |
| 1861 | aaccaagatg tag |

By “TH polypeptide” (or tyrosine hydroxylase) is meant a polypeptide or fragment thereof having at least about 85% amino acid identity to NCBI Accession No. AAI43612.1.

(SEQ ID NO: 61)
MPTPDATTPQAKGFRRAVSELDAKQAEAIMSPRFIGRRQSLIEDARKERE
AAVAAAAAAVPSEPGDPLEAVAFEEKEGKAVLNLLFSPRATKPSALSRAV
KVFETFEAKIHHLETRPAQRPRAGGPHLEYFVRLEVRRGDLAALLSGVRQ
VSEDVRSPAGPKVPWFPRKVSELDKCHHLVTKFDPDLDLDHPGFSDQVYR
QRRKLIAEIAFQYRHGDPIPRVEYTAEEIATWKEVYTTLKGLYATHACGE
HLEAFALLERFSGYREDNIPQLEDVSRFLKERTGFQLRPVAGLLSARDFL
ASLAFRVFQCTQYIRHASSPMHSPEPDCCHELLGHVPMLADRTFAQFSQD
IGLASLGASDEEIEKLSTLYWFTVEFGLCKQNGEVKAYGAGLLSSYGELL
HCLSEEPEIRAFDPEAAAVQPYQDQTYQSVYFVSESFSDAKDKLRSYASR
IQRPFSVKFDPYTLAIDVLDSPQAVRRSLEGVQDELDTLAHALSAIG

By “TH polynucleotide” (or tyrosine hydroxylase) is meant a polynucleotide encoding an TH polypeptide. An exemplary TH nucleic acid molecule (e.g., mRNA) is provided at NCBI Accession No. BC143611.

| (SEQ ID NO: 62) | |
|---|---|
| 1 | acccagaggg ggctttgacg tcagctcagc ttataagagg ctgctgggcc agggctgtgg |
| 61 | agacggagcc cggacctcca cactgagcca tgcccacccc cgacgccacc acgccacagg |
| 121 | ccaagggctt ccgcagggcc gtgtctgagc tggacgccaa gcaggcagag gccatcatgt |
| 181 | ccccgcggtt cattgggcgc aggcagagcc tcatcgagga cgcccgcaag gagcgggagg |
| 241 | cggcggtggc agcagcggcc gctgcagtcc cctcggagcc cggggacccc ctggaggctg |
| 301 | tggcctttga ggagaaggag gggaaggccg tgctaaacct gctcttctcc ccgagggcca |
| 361 | ccaagccctc ggcgctgtcc cgagctgtga aggtgtttga gacgtttgaa gccaaaatcc |
| 421 | accatctaga gacccggccc gcccagaggc cgcgagctgg gggcccccac ctggagtact |
| 481 | tcgtgcgcct cgaggtgcgc cgaggggacc tggccgccct gctcagtggt gtgcgccagg |
| 541 | tgtcagagga cgtgcgcagc cccgcggggc ccaaggtccc ctggttccca agaaaagtgt |
| 601 | cagagctgga caagtgtcat cacctggtca ccaagttcga ccctgacctg gacttggacc |
| 661 | acccgggctt ctcggaccag gtgtaccgcc agcgcaggaa gctgattgct gagatcgcct |
| 721 | tccagtacag gcacggcgac ccgattcccc gtgtggagta caccgccgag gagattgcca |
| 781 | cctggaagga ggtctacacc acgctgaagg gcctctacgc cacgcacgcc tgcggggagc |
| 841 | acctggaggc ctttgctttg ctggagcgct tcagcggcta ccgggaagac aatatccccc |
| 901 | agctggagga cgtctcccgc ttcctgaagg agcgcacggg cttccagctg cggcctgtgg |
| 961 | ccggcctgct gtccgcccgg gacttcctgg ccagcctggc cttccgcgtg ttccagtgca |
| 1021 | cccagtatat ccgccacgcg tcctcgccca tgcactcccc tgagccggac tgctgccacg |
| 1081 | agctgctggg gcacgtgccc atgctggccg accgcacctt cgcgcagttc tcgcaggaca |
| 1141 | ttggcctggc gtccctgggg gcctcggatg aggaaattga gaagctgtcc acgctgtact |
| 1201 | ggttcacggt ggagttcggg ctgtgtaagc agaacgggga ggtgaaggcc tatggtgccg |
| 1261 | ggctgctgtc ctcctacggg gagctcctgc actgcctgtc tgaggagcct gagattcggg |
| 1321 | ccttcgaccc tgaggctgcg gccgtgcagc cctaccaaga ccagacgtac cagtcagtct |
| 1381 | acttcgtgtc tgagagcttc agtgacgcca aggacaagct caggagctat gcctcacgca |
| 1441 | tccagcgccc cttctccgtg aagttcgacc cgtacacgct ggccatcgac gtgctggaca |
| 1501 | gcccccaggc cgtgcggcgc tccctggagg gtgtccagga tgagctggac acccttgccc |
| 1561 | atgcgctgag tgccattggc taggtgcacg gcgtccctga gggcccttcc caacctcccc |
| 1621 | tggtcctgc |

By “Neurofilament 200 polypeptide” (or neurofilament heavy (NEFH)) is meant a polypeptide or fragment thereof having at least about 85% amino acid identity to NCBI Accession No. NP_066554.2.

(SEQ ID NO: 63)
MMSFGGADALLGAPFAPLHGGGSLHYALARKGGAGGTRSAAGSSSGFHSW
TRTSVSSVSASPSRFRGAGAASSTDSLDTLSNGPEGCMVAVATSRSEKEQ
LQALNDRFAGYIDKVRQLEAHNRSLEGEAAALRQQQAGRSAMGELYEREV
REMRGAVLRLGAARGQLRLEQEHLLEDIAHVRQRLDDEARQREEAEAAAR
ALARFAQEAEAARVDLQKKAQALQEECGYLRRHHQEEVGELLGQIQGSGA
AQAQMQAETRDALKCDVTSALREIRAQLEGHAVQSTLQSEEWFRVRLDRL
SEAAKVNTDAMRSAQEEITEYRRQLQARTTELEALKSTKDSLERQRSELE
DRHQADIASYQEAIQQLDAELRNTKWEMAAQLREYQDLLNVKMALDIEIA
AYRKLLEGEECRIGFGPIPFSLPEGLPKIPSVSTHIKVKSEEKIKVVEKS
EKETVIVEEQTEETQVTEEVTEEEEKEAKEEEGKEEEGGEEEEAEGGEEE
TKSPPAEEAASPEKEAKSPVKEEAKSPAEAKSPEKEEAKSPAEVKSPEKA
KSPAKEEAKSPPEAKSPEKEEAKSPAEVKSPEKAKSPAKEEAKSPAEAKS
PEKAKSPVKEEAKSPAEAKSPVKEEAKSPAEVKSPEKAKSPTKEEAKSPE
KAKSPEKEEAKSPEKAKSPVKAEAKSPEKAKSPVKAEAKSPEKAKSPVKE
EAKSPEKAKSPVKEEAKSPEKAKSPVKEEAKTPEKAKSPVKEEAKSPEKA
KSPEKAKTLDVKSPEAKTPAKEEARSPADKFPEKAKSPVKEEVKSPEKAK
SPLKEDAKAPEKEIPKKEEVKSPVKEEEKPQEVKVKEPPKKAEEEKAPAT
PKTEEKKDSKKEEAPKKEAPKPKVEEKKEPAVEKPKESKVEAKKEEAEDK
KKVPTPEKEAPAKVEVKEDAKPKEKTEVAKKEPDDAKAKEPSKPAEKKEA
APEKKDTKEEKAKKPEEKPKTEAKAKEDDKTLSKEPSKPKAEKAEKSSST
DQKDSKPPEKATEDKAAKGK

By “Neurofilament 200 polynucleotide” (or neurofilament heavy (NEFH)) is meant a polynucleotide encoding an Neurofilament 200 polypeptide. An exemplary Neurofilament 200 nucleic acid molecule (e.g., mRNA) is provided at NCBI Accession No. NM_021076.

| (SEQ ID NO: 64) | |
|---|---|
| 1 | aaaagggccg gcgccctggt gctgccgcag tgcctcccgc cccgtcccgg cctcgcgcac |
| 61 | ctgctcaggc catgatgagc ttcggcggcg cggacgcgct gctgggcgcc ccgttcgcgc |
| 121 | cgctgcatgg cggcggcagc ctccactacg cgctagcccg aaagggtggc gcaggcggga |
| 181 | cgcgctccgc cgctggctcc tccagcggct tccactcgtg gacacggacg tccgtgagct |
| 241 | ccgtgtccgc ctcgcccagc cgcttccgtg gcgcaggcgc cgcctcaagc accgactcgc |
| 301 | tggacacgct gagcaacggg ccggagggct gcatggtggc ggtggccacc tcacgcagtg |
| 361 | agaaggagca gctgcaggcg ctgaacgacc gcttcgccgg gtacatcgac aaggtgcggc |
| 421 | agctggaggc gcacaaccgc agcctggagg gcgaggctgc ggcgctgcgg cagcagcagg |
| 481 | cgggccgctc cgctatgggc gagctgtacg agcgcgaggt ccgcgagatg cgcggcgcgg |
| 541 | tgctgcgcct gggcgcggcg cgcggtcagc tacgcctgga gcaggagcac ctgctcgagg |
| 601 | acatcgcgca cgtgcgccag cgcctagacg acgaggcccg gcagcgagag gaggccgagg |
| 661 | cggcggcccg cgcgctggcg cgcttcgcgc aggaggccga ggcggcgcgc gtggacctgc |
| 721 | agaagaaggc gcaggcgctg caggaggagt gcggctacct gcggcgccac caccaggaag |
| 781 | aggtgggcga gctgctcggc cagatccagg gctccggcgc cgcgcaggcg cagatgcagg |
| 841 | ccgagacgcg cgacgccctg aagtgcgacg tgacgtcggc gctgcgcgag attcgcgcgc |
| 901 | agcttgaagg ccacgcggtg cagagcacgc tgcagtccga ggagtggttc cgagtgaggc |
| 961 | tggaccgact gtcggaggca gccaaggtga acacagacgc tatgcgctca gcgcaggagg |
| 1021 | agataactga gtaccggcgt cagctgcagg ccaggaccac agagctggag gcactgaaaa |
| 1081 | gcaccaagga ctcactggag aggcagcgct ctgagctgga ggaccgtcat caggccgaca |
| 1141 | ttgcctccta ccaggaagcc attcagcagc tggacgctga gctgaggaac accaagtggg |
| 1201 | agatggccgc ccagctgcga gaataccagg acctgctcaa tgtcaagatg gctctggata |
| 1261 | tagagatagc cgcttacaga aaactcctgg aaggtgaaga gtgtcggatt ggctttggcc |
| 1321 | caattccttt ctcgcttcca gaaggactcc ccaaaattcc ctctgtgtcc actcacataa |
| 1381 | aggtgaaaag cgaagagaag atcaaagtgg tggagaagtc tgagaaagaa actgtgattg |
| 1441 | tggaggaaca gacagaggag acccaagtga ctgaagaagt gactgaagaa gaggagaaag |
| 1501 | aggccaaaga ggaggagggc aaggaggaag aagggggtga agaagaggag gcagaagggg |
| 1561 | gagaagaaga aacaaagtct cccccagcag aagaggctgc atccccagag aaggaagcca |
| 1621 | agtcaccagt aaaggaagag gcaaagtcac cggctgaggc caagtcccca gagaaggagg |
| 1681 | aagcaaaatc cccagccgaa gtcaagtccc ctgagaaggc caagtctcca gcaaaggaag |
| 1741 | aggcaaagtc accgcctgag gccaagtccc cagagaagga ggaagcaaaa tctccagctg |
| 1801 | aggtcaagtc ccccgagaag gccaagtccc cagcaaagga agaggcaaag tcaccggctg |
| 1861 | aggccaagtc tccagagaag gccaagtccc cagtgaagga agaagcaaag tcaccggctg |
| 1921 | aggccaagtc cccagtgaag gaagaagcaa aatctccagc tgaggtcaag tccccggaaa |
| 1981 | aggccaagtc tccaacgaag gaggaagcaa agtcccctga gaaggccaag tccccagaga |
| 2041 | aggaagaggc caagtcccct gagaaggcca agtccccagt gaaggcagaa gcaaagtccc |
| 2101 | ctgagaaggc caagtcccca gtgaaggcag aagcaaagtc ccctgagaag gccaagtccc |
| 2161 | cagtgaagga agaagcaaag tcccctgaga aggccaagtc cccagtgaag gaagaagcaa |
| 2221 | agtcccctga gaaggccaag tccccagtga aggaagaagc aaagaccccc gagaaggcca |
| 2281 | agtccccagt gaaggaagaa gctaagtccc cagagaaggc caagtcccca gagaaggcca |
| 2341 | agactcttga tgtgaagtct ccagaagcca agactccagc gaaggaggaa gcaaggtccc |
| 2401 | ctgcagacaa attccctgaa aaggccaaaa gccctgtcaa ggaggaggtc aagtccccag |
| 2461 | agaaggcgaa atctcccctg aaggaggatg ccaaggcccc tgagaaggag atcccaaaaa |
| 2521 | aggaagaggt gaagtcccca gtgaaggagg aggagaagcc ccaggaggtg aaagtcaaag |
| 2581 | agcccccaaa gaaggcagag gaagagaaag cccctgccac accaaaaaca gaggagaaga |
| 2641 | aggacagcaa gaaagaggag gcacccaaga aggaggctcc aaagcccaag gtggaggaga |
| 2701 | agaaggaacc tgctgtcgaa aagcccaaag aatccaaagt tgaagccaag aaggaagagg |
| 2761 | ctgaagataa gaaaaaagtc cccaccccag agaaggaggc tcctgccaag gtggaggtga |
| 2821 | aggaagacgc taaacccaaa gaaaagacag aggtagccaa gaaggaacca gatgatgcca |
| 2881 | aggccaagga acccagcaaa ccagcagaga agaaggaggc agcaccggag aaaaaagaca |
| 2941 | ccaaggagga gaaggccaag aagcctgagg agaaacccaa gacagaggcc aaagccaagg |
| 3001 | aagatgacaa gaccctctca aaagagccta gcaagcctaa ggcagaaaag gctgaaaaat |
| 3061 | cctccagcac agaccaaaaa gacagcaagc ctccagagaa ggccacagaa gacaaggccg |
| 3121 | ccaaggggaa gtaaggcagg gagaaaggaa catccggaac agccaaagaa actcagaaga |
| 3181 | gtcccggagc tcaaggatca gagtaacaca attttcactt tttctgtctt tatgtaagaa |
| 3241 | gaaactgctt agatgacggg gcctccttct tcaaacagga atttctgtta gcaatatgtt |
| 3301 | agcaagagag ggcactccca ggcccctgcc cccaggccct ccccaggcga tggacaatta |
| 3361 | tgatagctta tgtagctgaa tgtgatacat gccgaatgcc acacgtaaac acttgactat |
| 3421 | aaaaactgcc cccctccttt ccaaataagt gcatttattg cctctatgtg caactgacag |
| 3481 | atgaccgcaa taatgaatga gcagttagaa atacattatg cttgagatgt cttaacctat |
| 3541 | tcccaaatgc cttctgtttt ccaaaggagt ggtcaagccc ttgcccagag ctctctattc |
| 3601 | tggaagagcg gtccaggtgg ggccggggac tggccactga attatgccag ggcgcacttt |
| 3661 | ccactggagt tcactttcaa ttgcttctgt gcaataaaac caagtgctta taaaatgaaa |
| 3721 | a |

By “Map2” (or microtubule-associated protein 2) is meant a polypeptide or fragment thereof having at least about 85% amino acid identity to NCBI Accession No. AAH38857.1.

(SEQ ID NO: 65)
MADERKDEAKAPHWTSAPLTEASAHSHPPEIKDQGGAGEGLVRSANGFPY
REDEEGAFGEHGSQGTYSNTKENGINGELTSADRETAEEVSARIVQVVTA
EAVAVLKGEQEKEAQHKDQTAALPLAAEETANLPPSPPPSPASEQTVTVE
EAAGGESALAPSVFKQAKDKVSNSTLSKIPALQGSTKSPRYSSACPSTTK
RATFSDSLLIQPTSAGSTDRLPYSKSGNKDGVTKSPEKRSSLPRPSSILP
PRRGVSGDRDENSFSLNSSISSSARRTTRSEPIRRAGKSGTSTPTTPGST
AITPGTPPSYSSRTPGTPGTPSYPRTPHTPGTPKSAILVPSEKKVAIIRT
PPKSPATPKQLRLINQPLPDLKNVKSKIGSTDNIKYQPKGGQVRILNKKI
DFSKVQSRCGSKDNIKHSAGGGNVQIVTKKIDLSHVTSKCGSLKNIRHRP
GGGRVKIESVKLDFKEKAQAKVGSLDNAHHVPGGGNVKIDSQKLNFREHA
KARVDHGAEIITQSPGRSSVASPRRLSNVSSSGSINLLESPQLATLAEDV
TAALAKQGL

By “Map2 polynucleotide” (or microtubule-associated protein 2) is meant a polynucleotide encoding an Map2 polypeptide. An exemplary Map2 nucleic acid molecule (e.g., mRNA) is provided at NCBI Accession No. BC038857.

| (SEQ ID NO: 66) | |
|---|---|
| 1 | ggcgctcggg ctgcgcgggc tctgggcagc agcagcagca gcagcagcat cctctcttcc |
| 61 | tttacttccc ttccgcttct ttctcttcct tctccttctt tttccccccc ctccccttct |
| 121 | tcccctaacc cttctacccc tctccttttt ctccggaggg cgctaagtcc gtgagcggtg |
| 181 | gcagtcgcga ccgcgggtgc atccagtttc tgcgcccaga ttttattgat ctaatccaaa |
| 241 | gtatcttata acttctggct ggaattaaga ttcttcagct tgtctctaac cgaggaagca |
| 301 | ttgattggga gctactcatt cagaaaatta aaagaaagaa gccagaaaat attatcaacc |
| 361 | ctttgagaac acgacacaac gaactttata ttttaccact tccttgaata gttgcaggag |
| 421 | aaataacaag gcattgaaga atggcagatg aacggaaaga cgaagcaaag gcacctcact |
| 481 | ggacctcagc accgctaaca gaggcatctg cacactcaca tccacctgag attaaggatc |
| 541 | aaggcggagc aggggaagga cttgtccgaa gcgccaatgg attcccatac agggaggatg |
| 601 | aagagggtgc ctttggagag catgggtcac agggcaccta ttcaaatacc aaagagaatg |
| 661 | ggatcaacgg agagctgacc tcagctgaca gagaaacagc agaggaggtg tctgcaagga |
| 721 | tagttcaagt agtcactgct gaggctgtag cagtcctgaa aggtgaacaa gagaaagaag |
| 781 | ctcaacataa agaccagact gcagctctgc ctttagcagc tgaagaaaca gctaatctgc |
| 841 | ctccttctcc acccccatca cctgcctcag aacagactgt cacagtggag gaagcagcag |
| 901 | gtggggaatc agctctggct cccagtgtat ttaaacaggc aaaggacaaa gtctctaatt |
| 961 | ctaccttgtc aaagattcct gctttacagg gtagcacaaa gtccccaaga tacagctcag |
| 1021 | cctgccctag cacgactaaa agggctacat tttctgacag tttattaata cagcccacct |
| 1081 | cagcaggctc cacagaccgt ttgccatact caaaatcagg gaacaaggac ggagtaacca |
| 1141 | agagcccaga aaagcgctct tctctcccaa gaccttcctc cattctccct cctcggcgag |
| 1201 | gtgtgtcagg agacagagat gagaattcct tctctctcaa cagttctatc tcttcttcag |
| 1261 | cacggcggac caccaggtca gagccaattc gcagagcagg gaagagtggt acctcaacac |
| 1321 | ccactacccc tgggtctact gccatcactc ctggcacccc accaagttat tcttcacgca |
| 1381 | caccaggcac tcctggaacc cctagctatc ccaggacccc tcacacacca ggaaccccca |
| 1441 | agtctgccat cttggtgccg agtgagaaga aggtcgccat catacgtact cctccaaaat |
| 1501 | ctcctgcgac tcccaagcag cttcggctta ttaaccaacc actgccagac ctgaagaatg |
| 1561 | tcaaatccaa aatcggatca acagacaaca tcaaatacca gcctaaaggg gggcaggtta |
| 1621 | ggattttaaa caagaagatc gattttagca aagttcagtc cagatgtggt tccaaggata |
| 1681 | acatcaaaca ttcggctggg ggcggaaatg tacaaattgt taccaagaaa atagacctaa |
| 1741 | gccatgtgac atccaaatgt ggctctctga agaacatccg ccacaggcca ggtggcggac |
| 1801 | gtgtgaaaat tgagagtgta aaactagatt tcaaagaaaa ggcccaagct aaagttggtt |
| 1861 | ctcttgataa tgctcatcat gtacctggag gtggtaatgt caagattgac agccaaaagt |
| 1921 | tgaacttcag agagcatgct aaagcccgtg tggaccatgg ggctgagatc attacacagt |
| 1981 | ccccaggcag atccagcgtg gcatcacccc gacgactcag caatgtctcc tcgtctggaa |
| 2041 | gcatcaacct gctcgaatct cctcagcttg ccactttggc tgaggatgtc actgctgcac |
| 2101 | tcgctaagca gggcttgtga atatttctca tttagcattg aaataataat atttaggcat |
| 2161 | gagctcttgg caggagtggg ctctgagcag ttgttatatt cattctttat aaaccataaa |
| 2221 | ataaataatc tcatccccaa aaaaaaaaaa aaaaaaaaaa aaaaaaaaaa aaaaaaaaaa |
| 2281 | aaaaaa |

By “DCX” (or doublecortin) is meant a polypeptide or fragment thereof having at least about 85% amino acid identity to NCBI Accession No. NP_835366.1.

(SEQ ID NO: 67)
MELDFGHFDERDKTSRNMRGSRMNGLPSPTHSAHCSFYRTRTLQALSNEK
KAKKVRFYRNGDRYFKGIVYAVSSDRFRSFDALLADLTRSLSDNINLPQG
VRYIYTIDGSRKIGSMDELEEGESYVCSSDNFFKKVEYTKNVNPNWSVNV
KTSANMKAPQSLASSNSAQARENKDFVRPKLVTIIRSGVKPRKAVRVLLN
KKTAHSFEQVLTDITEAIKLETGVVKKLYTLDGKQVTCLHDFFGDDDVFI
ACGPEKFRYAQDDFSLDENECRVMKGNPSATAGPKASPTPQKTSAKSPGP
MRRSKSPADSANGTSSSQLSTPKSKQSPISTPTSPGSLRKHKDLYLPLSL
DDSDSLGDSM

By “DCX polynucleotide” (or doublecortin) is meant a polynucleotide encoding an DCX polypeptide. An exemplary DCX nucleic acid molecule (e.g., mRNA) is provided at NCBI Accession No. NM_178153.

| (SEQ ID NO: 68) | |
|---|---|
| 1 | ctggcaggaa tttcttgctt ggagctcaga caacaaaggc atagagagat tggttttctt |
| 61 | tctctcagca tctccaccca accagcagaa aaccggtctc tgaggttcca ccaaaatatg |
| 121 | gaacttgatt ttggacactt tgacgaaaga gataagacat ccaggaacat gcgaggctcc |
| 181 | cggatgaatg ggttgcctag ccccactcac agcgcccact gtagcttcta ccgaaccaga |
| 241 | accttgcagg cactgagtaa tgagaagaaa gccaagaagg tacgtttcta ccgcaatggg |
| 301 | gaccgctact tcaaggggat tgtgtacgct gtgtcctctg accgttttcg cagctttgac |
| 361 | gccttgctgg ctgacctgac gcgatctctg tctgacaaca tcaacctgcc tcagggagtg |
| 421 | cgttacattt acaccattga tggatccagg aagatcggaa gcatggatga actggaggaa |
| 481 | ggggaaagct atgtctgttc ctcagacaac ttctttaaaa aggtggagta caccaagaat |
| 541 | gtcaatccca actggtctgt caacgtaaaa acatctgcca atatgaaagc cccccagtcc |
| 601 | ttggctagca gcaacagtgc acaggccagg gagaacaagg actttgtgcg ccccaagctg |
| 661 | gttaccatca tccgcagtgg ggtgaagcct cggaaggctg tgcgtgtgct tctgaacaag |
| 721 | aagacagccc actcttttga gcaagtcctc actgatatca cagaagccat caaactggag |
| 781 | accggggttg tcaaaaaact ctacactctg gatggaaaac aggtaacttg tctccatgat |
| 841 | ttctttggtg atgatgatgt gtttattgcc tgtggtcctg aaaaatttcg ctatgctcag |
| 901 | gatgattttt ctctggatga aaatgaatgc cgagtcatga agggaaaccc atcagccaca |
| 961 | gctggcccaa aggcatcccc aacacctcag aagacttcag ccaagagccc tggtcctatg |
| 1021 | cgccgaagca agtctccagc tgactcagca aacggaacct ccagcagcca gctctctacc |
| 1081 | cccaagtcta agcagtctcc catctctacg cccaccagtc ctggcagcct ccggaagcac |
| 1141 | aaggacctgt acctgcctct gtccttggat gactcggact cgcttggtga ttccatgtaa |
| 1201 | aggaggggag agtgctcaga gtccagagta caaatccaag cctatcattg tagtagggta |
| 1261 | cttctgctca agtgtccaac agggctattg gtgctttcaa gtttttattt tgttgttgtt |
| 1321 | gttattttga aaaacacatt gtaatatgtt gggtttattt tcctgtgatt tctcctctgg |
| 1381 | gccactgatc cacagttacc aattatgaga gatagattga taaccatcct ttggggcagc |
| 1441 | attccaggga tgcaaaatgt gctagtccat gacctttcaa tggaaagctt aggtgcctgc |
| 1501 | gttatatttg ccctgtctaa ttttgcccat acagtcttcc ttctgtagag ggctgtttac |
| 1561 | atatacagca cttaaaatgt ttgtgtggga aaaaaaaaac tcattggcag atccaagaat |
| 1621 | gacaaacaca agtgcccctt ttctctggat ctcaagaatg gtggaggacc ctggaaggac |
| 1681 | agcaaggcag ctccccagcc tcactcttca ctcctgattg aggcccgggt ttgttgtcca |
| 1741 | gcaccaattc tggctgtcaa tggggagaaa taaaccaaca acttataatt gtgacaccag |
| 1801 | atgcttagga tcctggtgct gggttagcta agagaataga cagaattgga aaatactgca |
| 1861 | gacatttccg aagagtttat aaagcacagt gaattcctgg tcaatctctc cactgaggca |
| 1921 | atttggaatc aataagcaat tgataatagt ttggagtaag ggacttcata tacctgattc |
| 1981 | ctctagaagg ctgtctaaca taccacatga ttacatgaac tgtatggtat ccatctatct |
| 2041 | ctgttctatt gaatgccttg ttaacagcca acactgaaaa cactgtgaga atttgttttc |
| 2101 | aggtctgaca cctttcagtc tctttttata gcaagaaatc aatatccttt ttataaaaat |
| 2161 | tcatgtctgt atttcaggag caaactcttc aggctccttt tttataaact ggtgattttt |
| 2221 | cttttgtcta aaaaacacat gaagaaaatt taccaaaaaa aaaaaaaaaa gcagaagaat |
| 2281 | aatgtagttt agaaattatg ctgtcactgc caaacagtaa cctccaggag aaaacaagat |
| 2341 | gaatagcaga ggccaattca atagaatcag ttttttgata gctttttaac agttatgctt |
| 2401 | gcattaataa tttcaatgtg gaccagacat tctaattata ttttaaatga aatgttacag |
| 2461 | catattttaa gcaactcttt ttatctataa tcctaatatt tcatactgaa gacacagaaa |
| 2521 | tctttcactt gtctttaaca ttagaaagga tttctcttta ctaaggactg atcatttgaa |
| 2581 | atagttttca gtcttttgag atacaggttt ataacactgc tttttttttt ctgtaatcat |
| 2641 | agcccataat ggcaaagaca actaaattta agtgaaggtc atgcatgcca attctgtgtt |
| 2701 | tgcttttagc agatatgaag atttccttat ttctttgtaa ttgtgcagat attttgaaag |
| 2761 | gcacagcatt cgaagccaag ctgctgtttg gctactgaat ggcttgcagt tgttcctcca |
| 2821 | ctctaaatgg aatgagcttg ctgtgtgtgt gtgtggtggt ggtgggaggg ggtggtgcat |
| 2881 | gtgtgtgtgt gtgtgtgcat ctgcagctgc ttcaaaatta ggaaatacta caggacaccc |
| 2941 | ctgtaatgga ttggtggcaa ctgggtggca ctgctgatgt gcactgtgta ggggggaacc |
| 3001 | cagtggtggt ggggtagctc agatgcccct agacaagctt cagatgtctg tagctaccag |
| 3061 | aaacattttc ggttcaggaa aagtgagatg atggtagtac tggtttctgg tgaaattgaa |
| 3121 | gaaccccaaa tgatgaggat ctctttttgc cccctctcct ttttttgtag acccattcaa |
| 3181 | aaccattaat aagcccattt tactaagccc ctatttcttt ctagaagctc agggttttct |
| 3241 | tagtgcctcc cagaacattt tgtagttaat tgggaaaaag tgatacttgg attagggggt |
| 3301 | gtgggcataa agaatggtgg gaggcctgat tttaaacttc aggccagaac ccccaatgac |
| 3361 | tccacccata gtctcacttt aggtctcatt tagtccatca cctttatttt aagttgagga |
| 3421 | agtggaggct ggtaaagagc aggaccagag gaagaatcca gatttcctta tgcttgggcc |
| 3481 | tcacactagc tctctgagta tttccttgat tgcggtatat gtactactag aaaataccaa |
| 3541 | atggatatat tttctttagg ataacctttg aaccaacaat cttcaataac aatagtacat |
| 3601 | cttccatctt acttttaatc gagtataagg aaatgtttct ttatggccat tttggaggga |
| 3661 | gcaggggatg aggcttggca tagtccaaaa tttaagtctc caataattaa ttgcatttta |
| 3721 | aattggccca ctttcaaggc aatttttttt gtgtgtctgt aactgagctc ctccacccct |
| 3781 | gtcattcact tccaatttta cccaatccaa ttttagcact caagttccat tgtgttaatt |
| 3841 | tctgcacggt caacaaacat caagtcagca agcatttgcc accactccct atacttctcc |
| 3901 | ctccttctta cacacacaca cacacacaca cacacaatcc atctcttgct tgttcctacc |
| 3961 | tcctgatttt tcttccctac agaaatagaa atagggacaa agaaggggaa aatgtatata |
| 4021 | ttggggctgg gctgaacaac taacttcata agtagtatta actaggggta aattgagaga |
| 4081 | aaagctcctt ttctcttcac tgttttggaa aggatagcca ttagcatgac tgctttgtgt |
| 4141 | ccttatggac tttagtatta gcctagattg aattatagcg ttttctagct ggaaggaacc |
| 4201 | ttaagatcac atcatctact cctctactcc aaatttctca ttcttcaggc caggaaaccg |
| 4261 | agacacagag gtaaagtaat ttccccaagg tcacacagct ggctggggca ggattgggtt |
| 4321 | tacaacccac atctcctggc tcttattcca gggccttttc ccactaagta gtattgcctt |
| 4381 | ccattaggct cctgagagtt atttctcagg gtcatgttgc atcttggagc cacatgctgc |
| 4441 | tgccctgatc tcagtgggaa atccacccag caacctaata cagccccttt tccctgcatt |
| 4501 | cacctggttc ccatccacat gggttgcaga tgtccttgaa gagagtgagg cattgagggc |
| 4561 | caataggagc aatggggtcc ctggccttgt ccatctgatt caggagatca ctgctccatc |
| 4621 | gtgaggagcc ctctgaatag ccccccactg aatgcttgcc ttgcccaaat ggaatggagg |
| 4681 | aagattgatt ttctccatca gttcaccttg tgtcatctca taatggttgg tctttccagg |
| 4741 | ctgagggaaa tgtttcttgt ttccagagta gaaaaagaaa gagtggaaca atagctttgt |
| 4801 | tcatcctaac tttctgagat ggcttttcaa cattttaaaa aaactagtgt ggctaccatt |
| 4861 | cactggcaat gatttctttt agaatatggg agtaagatga gctagagaaa ataacctggt |
| 4921 | ctcactgtgg ttgccctcat ccacaatgtc cccaaagcca tcctgctctg atgaggacaa |
| 4981 | tttccaggta taagcaaggg gctttgtgac aaaaatgtac cctggctgat gttaaacatt |
| 5041 | ggctcctgtg tttgcaccaa aatagcaagc tgtgtgctct atacactctt cccatcgtct |
| 5101 | tgtgtacact gctcctgtgg ccttccacag cagaaaccag ggcaaaaggg tccaaacaca |
| 5161 | tggttttcct tgctgcaagg ctcttcctgg gaactaaggg ggtatttatt agttcagttc |
| 5221 | taagagacct ccttctgggc ttaccccact cctcaggtac ttctctctcc ttcctccttc |
| 5281 | tcctccacag tcacaagtaa ccaaggaacc tgaaagtgga tgtgtagcta tttgaagaag |
| 5341 | gcaaggaacc ctgagattct tctttgaatc ctctagtcca agtcttagac cagtgattgg |
| 5401 | tgcttacctt gaacaaaatt ttgtctgtgt tcctaatccc ttcaatactc tgggtacaat |
| 5461 | gctcccaatc accctgcaca tttgattcta aatggctttt attttttaaa aatccatatc |
| 5521 | cctaggacaa gagaacagga tgcctatatc cccaaaatga gctccaggac actgatggga |
| 5581 | atgatcccaa agatcacccc acctcagaaa cgtctgtgcc aagagacttc cccagataga |
| 5641 | aacactggga cagtggtttg aacgacttct tttatggttg tccagtttgc tatggaaata |
| 5701 | aaaggcattg attttttaaa aagatgattg gaacctgtct ttggccacat agggccactt |
| 5761 | ggatccattt ccaggcctta ctcatatatt gccttcactg aagggctttg gctttaagtc |
| 5821 | ccagactggt ctcccaagtg aaccataagt gttttggagc tcatctgggg tgaggcatga |
| 5881 | gaatgttgcc ccatctatcc cttcaggaaa aggtgccttc cctccctttc tcctaaagcc |
| 5941 | tggtccccag aaattgtttt tgtctccaaa agtctagtat ggtctttata cacccagact |
| 6001 | cttagtgttg cgtcctgcct tgtttccttg ttaaggatct atgcagacct cccgctttgg |
| 6061 | cttagctagc gtgacattgg ctatcatttg acaagactaa cttttttttt tttttttttt |
| 6121 | tgactgagtc tccctctgtc acctaggctg gagtgcagtg gcacaatctt ggctcgctgc |
| 6181 | aaccttcacc cttcacctcc caggtcgaag cgattctcct gcctcagtct cccgagtagc |
| 6241 | tgggattaca ggcgtgcgcc accaaatctg gctatttttt tattattatt atttttagta |
| 6301 | gagatggggt ttcaccatgt tggccagact ggtcttgaac tcttggcctc aaattatctg |
| 6361 | cccacctcgg cctcccaaag tgctgggatt acaggcatga gccaccatgc ccagctgaca |
| 6421 | agactaattt tttatccctt ggtttattgg cttcaacatc ttctggaatc agaggtgatt |
| 6481 | ttttcttacc ttggatgcct gagactaggg gagtatagaa ttccaattgg taattaaggc |
| 6541 | atctttctgc tcctgatcag aagggcaggt tagttgggag aggtcagatg gcacaacaga |
| 6601 | agtcaccttg taagtaaggc aaagacttga aggcattagc gtttctcatt actaggtcaa |
| 6661 | taacctgagg gaatcaatgg ctttttgccg ctctacctct tgtgtatctc tttgactttt |
| 6721 | ctttctctgt ctagtttcct ctgttctcag tttatattct atgttatcag tctctctttc |
| 6781 | cacagtacaa acatccatcc tttctcctgt gcaattctgt ctctccctct tattatcttt |
| 6841 | atttgtactt tttccttcct ccctgtctag gcattgggca tgtgcctctt cttagcctgt |
| 6901 | gattttgcct tgggactgat gataaattat ttccagattc aatcagccct ggtcctaccc |
| 6961 | cagtccaatc agaagtatgt tggtgggaat caacctgatc ctggcccttt cttcttctcc |
| 7021 | attttcattc gtaatccccc tcagcagatc tttacaagca gtttccttat agctcatgta |
| 7081 | tctttaggtc tttgccttcc aagcactgta cagaatactt tgtggttcct ttttagtctg |
| 7141 | acattttgtg gagcagtgaa gcgtgctcag agacataatc agctgaagag aaaaaatcca |
| 7201 | cccatggatt tatatcagct aaatactaat aattgatttt gtttgatgtg cccataattt |
| 7261 | ttaaagctgc aatataatat aatgagggac cacaggtaat ttctcctgtc atttgttttg |
| 7321 | gctggatggg ggtgggggag taattgctta aagttttacc attacacatt aaactctcta |
| 7381 | taataatctt gtttggggct tgctaactgt tgagctgttt taactaaact ggtaggcaat |
| 7441 | cggagttgat ttaaatgaaa agataattta acaaatctat actataaaaa gagacatttg |
| 7501 | cttaattgac atgtattttt tccttctgag tcacctaaac atttactctt gacaccaact |
| 7561 | gttcatgata ctgaatagac agtccatata agagaaatta gtggacctaa agaagccaga |
| 7621 | ttgtaggtgt taatttatta aacagagtgc aaagcccttg gaaatgtcac tgcttggcaa |
| 7681 | taccatatgg aatgccaaaa tttacaatga cttttcttta taagttatcc aaaagggatt |
| 7741 | tgaacaagta agaggttatg ccaaaatgtc tccaatgtat ggtcctgtaa tatattgcag |
| 7801 | cttgaagcca atgatccctt atgacttgta tacaactaat gcatgtttta ttgaattttg |
| 7861 | catttcccac gtgtggtaag ttctttaaaa tgtttttgat cacctttttg tgccattaaa |
| 7921 | cttgtacaga aaatgttttt atggccattt tcaaagggag aaagtttaaa atggaaacag |
| 7981 | cccacccttt ctgccctata gctgtagtta gaattgagta cctgtagcaa aacagctgta |
| 8041 | attggtggtt gtagtgttag aggtgttagc ttgctagtga ctagctttgg agagtaaatg |
| 8101 | catggtattg tacatcacat ttcttaactc gttttaacct ctgaaaagaa tatattcttc |
| 8161 | tttgtagtcc ttcttcccac ccccttgccc tctccctctc cctgctccca gttgtcttac |
| 8221 | agttgtaaat atctgatttg aggcccaata actcttgcca agtaaagtca gcaaacaaca |
| 8281 | aacaaaccaa aatgtgggga aaaggcattt ctcaaccatc tctcagcagt tattgatcat |
| 8341 | ttcttaagga acagcattgt gatcaaagac tcaactttac gtaaaaatca gtggtaaatt |
| 8401 | ggggttgtat ttggccattt gattacattt caggattgaa tagttttcag aatcacatgt |
| 8461 | aatccaaaga cagtaggtag tgatgtccct tatccctgca gctgttttaa gatagagacc |
| 8521 | tcagaagact ctgcttgacc gatgaccaat aattatttga aaaaaaaaga aaaaatgaga |
| 8581 | gaaataaaac agatatttaa gaactttagc cacctattta gaatagttat agccagaaaa |
| 8641 | aaaaacaagg gcatgagttc aaatgcatta ctatcagtgt cctaggcaat acctaaccta |
| 8701 | ctctgaaatt gtgattcaaa agcagtattt caagaggcat tctccttttt tggtttgctg |
| 8761 | accccacttg gactggtagg tttggtgagg cccccataaa ccagctggag cagacccttt |
| 8821 | tcatctcctg tgcctgtaac acccctcttc ccccaccccc tccgcaattc aatgagggct |
| 8881 | ttcttgggtc agaggacttc aaggttgtct agagaagttt gccatgtgtg taaggtgctg |
| 8941 | tgaactgtga gtgctgaaga ttcgcagcat tcaataccag gcagccaaag agctgctctt |
| 9001 | gcaattattt tggctctcaa gctctgttct tcatcgcatt ctcatttctg tgtacatttg |
| 9061 | caagatgtgt gtaatgtcat tttccaaaaa taaaatttga tttcaataaa aaaaaaaaaa |
| 9121 | aaaaaaaaaa aaaaa |

By “GABRA1” (or gamma-aminobutyric acid (GABA) A receptor) is meant a polypeptide or fragment thereof having at least about 85% amino acid identity to NCBI Accession No. AAH30696.1.

(SEQ ID NO: 69)
MRKSPGLSDCLWAWILLLSTLTGRSYGQPSLQDELKDNTTVFTRILDRLL
DGYDNRLRPGLGERVTEVKTDIFVTSFGPVSDHDMEYTIDVFFRQSWKDE
RLKFKGPMTVLRLNNLMASKIWTPDTFFHNGKKSVAHNMTMPNKLLRITE
DGTLLYTMRLTVRAECPMHLEDFPMDAHACPLKFGSYAYTRAEVVYEWTR
EPARSVVVAEDGSRLNQYDLLGQTVDSGIVQSSTGEYVVMTTHFHLKRKI
GYFVIQTYLPCIMTVILSQVSFWLNRESVPARTVFGVTTVLTMTTLSISA
RNSLPKVAYATAMDWFIAVCYAFVFSALIEFATVNYFTKRGYAWDGKSVV
PEKPKKVKDPLIKKNNTYAPTATSYTPNLARGDPGLATIAKSATIEPKEV
KPETKPPEPKKTFNSVSKIDRLSRIAFPLLFGIFNLVYWATYLNREPQLK
APTPHQ

By “GABRA1 polynucleotide” (or gamma-aminobutyric acid (GABA) A receptor) is meant a polynucleotide encoding an GABRA1 polypeptide. An exemplary GABRA1 nucleic acid molecule (e.g., mRNA) is provided at NCBI Accession No. BC030696.

| (SEQ ID NO: 70) | |
|---|---|
| 1 | agcggagcgg gcgagcaagg gagcgagcag gacaggagcc tgatcccaca gctgctgctc |
| 61 | cagcccgcga tgaggaaaag tccaggtctg tctgactgtc tttgggcctg gatcctcctt |
| 121 | ctgagcacac tgactggaag aagctatgga cagccgtcat tacaagatga acttaaagac |
| 181 | aataccactg tcttcaccag gattttggac agactcctag atggttatga caatcgcctg |
| 241 | agaccaggat tgggagagcg tgtaaccgaa gtgaagactg atatcttcgt caccagtttc |
| 301 | ggacccgttt cagaccatga tatggaatat acaatagatg tatttttccg tcaaagctgg |
| 361 | aaggatgaaa ggttaaaatt taaaggacct atgacagtcc tccggttaaa taacctaatg |
| 421 | gcaagtaaaa tctggactcc ggacacattt ttccacaatg gaaagaagtc agtggcccac |
| 481 | aacatgacca tgcccaacaa actcctgcgg atcacagagg atggcacctt gctgtacacc |
| 541 | atgaggctga cagtgagagc tgaatgtccg atgcatttgg aggacttccc tatggatgcc |
| 601 | catgcttgcc cactaaaatt tggaagttat gcttatacaa gagcagaagt tgtttatgaa |
| 661 | tggaccagag agccagcacg ctcagtggtt gtagcagaag atggatcacg tctaaaccag |
| 721 | tatgaccttc ttggacaaac agtagactct ggaattgtcc agtcaagtac aggagaatat |
| 781 | gttgttatga ccactcattt ccacttgaag agaaagattg gctactttgt tattcaaaca |
| 841 | tacctgccat gcataatgac agtgattctc tcacaagtct ccttctggct caacagagag |
| 901 | tctgtaccag caagaactgt ctttggagta acaactgtgc tcaccatgac aacattgagc |
| 961 | atcagtgcca gaaactccct ccctaaggtg gcttatgcaa cagctatgga ttggtttatt |
| 1021 | gccgtgtgct atgcctttgt gttctcagct ctgattgagt ttgccacagt aaactatttc |
| 1081 | actaagagag gttatgcatg ggatggcaaa agtgtggttc cagaaaagcc aaagaaagta |
| 1141 | aaggatcctc ttattaagaa aaacaacact tacgctccaa cagcaaccag ctacacccct |
| 1201 | aatttggcca ggggcgaccc gggcttagcc accattgcta aaagtgcaac catagaacct |
| 1261 | aaagaggtca agcccgaaac aaaaccacca gaacccaaga aaacctttaa cagtgtcagc |
| 1321 | aaaattgacc gactgtcaag aatagccttc ccgctgctat ttggaatctt taacttagtc |
| 1381 | tactgggcta cgtatttaaa cagagagcct cagctaaaag cccccacacc acatcaatag |
| 1441 | atcttttact cacattctgt tgttcagtcc tctgcactgg gaatttattt atgttctcaa |
| 1501 | cgcagtaatt cccatctgct ttattgcctc tgtcttaaag aatttgaaag tttccttatt |
| 1561 | ttcataattc atttaagaac aagagacccc tgtctggcag tctggagcaa agcagactat |
| 1621 | gcagcttgga gacaggattc tgacagagca agcgaaagag caaagtcatg tcagaaggag |
| 1681 | acagaatgag agagaaaaga gggggaagat ggttcaaaga tacaagaaaa agtagaaaaa |
| 1741 | aaaataacac ttaactaaaa cccctaggtc atttgtagat atatatttcc aaatattcta |
| 1801 | aaaaagatac tgtatatgtc aaaaatattt ttatgtgaag gtgtttcaaa gggtaaatta |
| 1861 | taaatgtttc atgaagaaaa aattttaaaa atctacgtct ttattacaca aactatggtg |
| 1921 | tgcttatgtt tttgttttgc tttttaaact gatgtatagc tttaacattt tgtttccaaa |
| 1981 | gctgaagatc cccattcttt ctctttgaaa aaaaaaaagg cctaatgcat tattttgtca |
| 2041 | taaaatgcta ttttaaaatt catggaactt tcatacgtaa aggtgcagtt gctcattgta |
| 2101 | gagcacattt agtccaatga agataaatgc tttaaatagt ttacttcact ttcatctgag |
| 2161 | cttttaccac tagactcaag gaagaataat tttaacagac atgtatactc catagaaact |
| 2221 | aaattaaaat agtttaaaaa tattcccttt ttcaccctat tttcagatag cacatgagcc |
| 2281 | caacactcac ttaattctca ttatgaagat gtttttagag gggcaaaaat attttgcaag |
| 2341 | ctctggaatt gttgaatgta ttcttttata taactacatt aaaagcttta gattgaaatt |
| 2401 | tatgactagc aaacaaaaat agaatatata aacgatatat gtaaatatac agcatgagat |
| 2461 | tgtacatttt ttactttttt aaaattgtgt tcttaaaata ttgtgtaaga atcactgcac |
| 2521 | ttagctgttg gaatgttgtt aaatgctatg gaaatacatt tagaacctgc atttaagaac |
| 2581 | agaacagcaa gtatgaacca catggaactt aaaacatatg ggtgtgaagt ccacttatgt |
| 2641 | agacaaaact tataatttcc aaactgttgt ctagtataca gtgatcagtt gctctctgtt |
| 2701 | caagtcattc cacacatttc cctattttag gctattataa tatagaaaga aaatgggaag |
| 2761 | cattagttgg agctagaaaa tgaactgtat attattgcta tatttgctaa taccaactat |
| 2821 | ttcaataagt gttgtaccat atgtagcatt aaatataaaa tacataaaag aatgtacaga |
| 2881 | aaatagcttt tattgagtaa tattacattt catttatact gtagcaatat atttgtaggt |
| 2941 | atactatgta agggctttaa ataaaagagg tccattaata cttccttata aaaattctag |
| 3001 | tctgtttcat tactgcccag atgttttaga gataaatatt tatgcagaag gtatttttga |
| 3061 | agtctccttt tgtctgatag agtttaacag atatttaaat ttagtgctca gaatccacaa |
| 3121 | gtcacggtct aaacacactt agaatactac agcataaatc tgttagcatt attgccaaat |
| 3181 | aagacagttg ggatccaaac ccaagtcttg agcaatgttt ttctcaaaaa gctgctatcc |
| 3241 | aatgatatag gaaaatacat tgtgttttcc taaacacact tttcttttta aatgtgcttc |
| 3301 | attgtttgat ttggtcctgc ctaaatttca caagctaggc caatgaaggc tgaatcaaag |
| 3361 | acatttcatc caccaatatc atgtgtagat attatgtata gaaaataaaa taaattatgg |
| 3421 | ctccaaaaaa aaaaaaaaaa |

Other features and advantages of the invention will be apparent to those skilled in the art from the following detailed description and claims.

### BRIEF DESCRIPTION OF THE DRAWINGS

FIGS. 1A-1D depict characterization of BMPS during differentiation. FIG. 1A depicts a diagram of a differentiation protocol. FIG. 1B depicts size of aggregates measured during the 3D neuronal differentiation. Negative days on the x-axis represent 3D cells cultured in NPC medium while positive days represent 3D cells cultured in differentiation medium. FIG. 1C1-C5 depicts BMPS mRNA and miRNA expression of different markers during differentiation. FIG. 1D depicts flow cytometry population analysis of BMPS at different stages of differentiation.

FIGS. 2A-2C depict morphological characterization of BMPS. FIG. 2A depicts co-immunostaining of neurons with markers. MAP2+ neurons were co-immunostained with the maturation marker Nestin at 2, 4, and 8 weeks of differentiation, which showed progressive increase of MAP2+ neurons and decrease of Nestin+ cells over time (panels a, b, c), demonstrating neuronal maturation. Co-immunostaining of neurons (NF-H) with the myelin marker MBP at 2, 4, and 8 weeks of differentiation (d,e,f, respectively) showed progressive increase of MBP+ cells in association with axonal processes. An increasing number of MBP+ cells (oligodendrocytes) was observed in association with axons (panels d, e, f). FIG. 2B depicts neuronal and glial cell diversity was evaluated at 8 weeks. Neurons (MAP2, NF, SYP and SMI32) were visualized interacting with glia (GFAP and NOGOA). Neurons disclosed characteristic perykaria, dendrites (MAP2, panels a, b) and axons (NF, SMI32, panels c-f) associated with glia. Neurons exhibited diverse neurotransmitter identities shown by identification of glutamatergic VGLUT1+ (panels g, h), GABAergic CALB+ (panels i, j) and dopaminergic TH (panels k, 1) neurons. FIG. 2C depicts that GFAP+ astroglia and CNPase+, O1+ and MBP+ oligodendroglia were identified. Oligodendroglia appeared mixed among astrocytes (panels a, b). 01+(panels c, d) and MBP+(panels e, f) oligodendrocytes were associated with axonal processes. Astrocytes established relationships with oligodendrocytes and exhibited characteristic multipolar processes (panels g, h). MBP+ oligodendrocytes issued processes in association with axons (panel i) 3D-reconstruction demonstrated myelinating processes resembling human myelination (panels j, k). Electron microscopy analysis of BMPS at 4 and 8 weeks of differentiation identified morphology of axonal structures and cells (e.g., oligodendrocytes) (panel 1). Myelinating-like processes, which closely resembled cross-sections of myelinated axons of the CNS were identified at 8 weeks of differentiation (panel m). FIG. 2D depicts MBP+ oligodendrocytes issued processes in close association with axons and seemed to enwrap them at 8 weeks (a,b,c). Myelination calculated as the mean percentage MBP positive oligodendrocyte processes coverage of NF-H-positive axons (a,b,c) at 2, 4 and 8 weeks in at least 2 independent experiments showed significant increase of myelination observed with time of differentiation (p<0.001) (d). FIG. 2E depicts 3D-reconstruction based on confocal z-stacks at 8 weeks demonstrating a “wrapping” myelinating process, which resembled the myelination of axons in human CNS. FIG. 2F depicts a comparison of expression of neuronal and glial markers at 2 and 8 weeks. At 2 weeks, oligodendrocytes (O1, CNPase, NOGOA) were identified without a preferential localization (a,b,e,f,i,j), later they resemble human oligodendrocytes and localize in close proximity with axons (c,d, g,h, k,l). At 2 weeks there are few MAP2-positive cells without identifiable neuronal shape (I,j) whereas at 8 weeks, the MAP2+ cells acquire a well-defined dendritic network (k,l). The amount of astrocytes and density of the astroglial network increases with time of differentiation (GFAP, g,h). FIG. 2G depicts variation in the nuclear morphology. Co-immunostaining of neurons (MAP2) with cell-division marker KI67 showed that some cells are dividing (a,b), there was also a small degree of apoptosis demonstrated by positive staining with CASP3 (c). CASP 3-positive nuclei did not co-localize with mature neurons (d). FIG. 2H depicts ultrastructure analysis by electron microscopy of 4 week BMPS showed evidence of cell to cell junctions demonstrating functional interactions between the cells (arrows, a,b). Nuclear variation was confirmed by the presence of a few apoptotic nuclei (c) and normal healthy nuclei (d). NF: Neurofilament-heavy-chain, MAP2: Microtubule-associated-protein 2, MBP: myelin-basic-protein, VGLUT1: Vesicular-glutamate-transporter 1, GFAP: Glial-fibrillary-acidic-protein, CALB: Calbindin, NOGOA: Neurite-outgrowth-inhibitor, SYP: Synaptophysin, SMI32: Nonphosphorylated-neurofilament, TH: Tyrosine-hydroxylase, O1: Olig1, CNPase: 2′,3′-Cyclic-nucleotide-3′-phosphodiesterase. Scale Bar: 10 μm.

FIGS. 3A-3F depict electrical activity of BMPS. Cells were cultured in 3D for 8 weeks and then cultured in 12-well and 48-well MEA plates for 4 more weeks. FIG. 3A depicts heat map recordings from a 48-well plate. FIG. 3B depicts illustration of an active well showing spike morphology and FIG. 3C depicts spike activity. FIGS. 3D and 3E depicts phase-contrast imaging of the mini-brains on MEAs, electrode diameter is 40-50 μm and inter-electrode space is 350 μm. FIG. 3F depicts activity pattern recordings over 0.05 spikes/sec of the electrode over 10 min.

FIGS. 4A-4G depict Parkinson's disease (PD) application of BMPS. BMPS were differentiated for 4 weeks and exposed to rotenone and MPP+ for 12 and 24 hours. FIG. 4A depicts viability (resazurin assay) of BMPS after 24 hours rotenone exposure. FIG. 4B depicts ROS (OxiSelect™ In Vitro ROS/RNS Assay Kit) production of BMPS after 12 and 24 hours rotenone exposure. FIG. 4C depicts viability (resazurin assay) of BMPS after 24 hours MPP+ exposure. FIG. 4D depicts ROS (OxiSelect™ In Vitro ROS/RNS Assay Kit) production of BMPS after 12 and 24 hours MPP+ exposure. FIGS. 4E and 4F depict confocal images of BMPS exposed to different concentrations of rotenone and MPP+ for NF200 (Red), TH (Green) and Hoechst nucleus staining (Blue). FIG. 4G depicts expression of genes associated with oxidative stress and PD by real time RT-PCR. Graphs represent the relative expression of different markers compared to control (cells not treated) after 24 hours exposure to 5 μM rotenone and 1 mM MPP+. Genes of interest: mitochondrial complex 5 (ATP50, ATP5C1), mitochondrial complex 1 (NDUFB1), oxidative stress (KEAP1) and genes related to PD (TH, SNCA, TBR1, CASP1). Data are presented as mean±SD, of 3 independent experiments performed in 3 replicates. *P<0.05 comparing to control (untreated).

FIGS. 5A-5D depict Down's Syndrome application of BMPS. BMPS were produced with iPSCs derived from a patient with Down's Syndrome. FIG. 5A depicts morphological characterization with immunostaining of neurons (MAP2, Syn1, TH, SYP), neural precursor cells (nestin) and glial cells (GFAP) at 8 weeks of differentiation. FIG. 5B depicts expression of genes in healthy BMPS vs. Down's Syndrome BMPS before and after treatment with 5 μM rotenone, after 24 hours exposure. Genes of interest include CNS markers (TH, OLIG2, NEFH), mitochondrial markers (ATP5C1, ATPSJ, ATP50) and ROS markers (NFE2L2, SOD1) which were measured by comparing control with exposed cells to rotenone on both healthy and Down syndrome derived mini-brains. FIGS. 5C and 5D depict karyotyping of iPSCs derived from the patient with Down's Syndrome. aCGH+SNP results for Down syndrome iPSC line are shown.

FIG. 6 depicts viability of pre-frozen NT2 human teratocarcinoma cell line and iPSC derived mini-brains. Fmedium corresponds to 95% FBS and 5% DMSO. NPC fmedium corresponds to STEMdiff™ Neural Progenitor Freezing Medium. Viability was measured by resazurin cell viability assay. Non-frozen cells at the same stage of differentiation were used as control aggregates.

FIG. 7 depicts an example of a BMPS covered with other cell types. LUHMES fluorescent cells (red) were incorporated to a BMP using gravity systems to cover the surface of the aggregate.

FIGS. 8A-8E depict morphologic characterization of mature human BMPS. FIG. 8A shows at 8 weeks, neuronal populations exhibited a diversity of neurotransmitter identities as shown by identification of dopaminergic TH+ (a,b), glutamatergic VGLUT1+ (c,d) and gabaergic calbindin+ (e,f) neurons. Neurons disclosed characteristic axons (NF) and synaptic proteins (SYN) (g,h). FIG. 8B depicts two distinctive glial populations were identified in close interaction with neuronal populations, GFAP+ astroglia and CNPase+, O1+, NOGOA+ oligodendroglia. O1+ oligodendrocytes were closely associated with axonal processes (NF) (a,b), CNPase+ oligodendroglia appeared mixed among GFAP+ astroglia (c,d) and exhibited the characteristic multipolar glial processes, which extended from the perykaria (e,f). NOGOA+ cells were associated with MAP+ neurons (g,h). FIG. 8C depicts example of custom algorithm created using the Cellomics Target Activation image-analysis software package to study astrocytes and oligodendrocytes (a,b,c,d). Quantification of cell populations as a percentage of the total nuclei count showed 3% NOGOA+ positive cells, 9% CNPase+ cells and 19% GFAP+ cells at 8 weeks (e). FIG. 8D shows Co-expression of mature oligodendroglia markers (MBP and 02). FIG. 8E shows expression of neuronal markers (VGLUT, TUJ1, SYN). Scale Bar: 10 μm.

### DETAILED DESCRIPTION OF THE INVENTION

The present invention is based, at least in part, upon the discovery that brain microphysiological systems (BMPS) can be produced from induced pluripotent stem cells (iPSCs). Furthermore, the invention provides for reproducible BMPS that differentiate into mature neurons and glial cells (astrocytes and oligodendrocytes) in the central nervous system. This model is spontaneously electrophysiological active and may be reproduced with patient or genetically modified cells. The derivation of 3D BMPS from iPSCs has applications in the study and treatment of neurological and neurodevelopmental diseases. In some embodiments, the present disclosure provides for compositions and methods to study and/or treat neurodevelopmental and neurodegenerative disorders. In some cases, the neurodevelopmental and neurodegenerative disorders treated and/or studied by the present disclosure include, but are not limited to, autism, encephalitis, trauma, brain cancer, stroke, Amyotrophic lateral sclerosis, Huntington's Disease, muscular dystrophy, neurodegenerative disorder, neurodevelopmental disorder, Multiple Sclerosis, infection, Parkinson's Disease and Alzheimer's Disease.

As described herein, the present disclosure provides for the derivation of a multitude of identical brain microphysiological systems (BMPS) from stem cells, preferably of human origin, but including stem cells from animal origin. The preferred starting material are human induced pluripotent stem cells or embryonic stem cells, although other pluripotent stem cells such as, for example, neuronal precursor cells and mesenchymal stem cells may also be employed. Human in-vitro models of brain neurophysiology are needed to investigate molecular and cellular mechanisms associated with neurological disorders and neurotoxicity. The techniques herein provide a reproducible iPSC-derived human 3D BMPS that includes differentiated mature neurons and glial cells (astrocytes and oligodendrocytes) that reproduce neuronal-glial interactions and connectivity. BMPS mature over about eight weeks and show the critical elements of neuronal function including, but not limited to, synaptogenesis and neuron-to-neuron (e.g. spontaneous electric field potentials) and neuronal-glial interactions (e.g. myelination). Advantageously, the BMPS described herein include mature neurons (e.g., glutamatergic, dopaminergic and GABAergic neurons) and glial cells (e.g., astrocytes and oligodendrocytes). Quantification of the different cell types exhibited high reproducibility between experiments. Moreover, the BMPS disclosed herein present neuron and glial functions such as spontaneous electrical activity and axon myelination. The BMPS described herein are able to mimic the microenvironment of the central nervous system, which is a significant advance in the field of neurobiology as this ability has not been achieved at this level of functionality, reproducibility, and consistency in prior art in vitro systems.

In particular, the high amount of myelination of axons (up to 40%) in the disclosed BMPS represents a significant improvement over the prior art. Myelin pathology is a rather frequent condition in demyelinating and inflammatory disorders such as multiple sclerosis and post-infection diseases as well as other neurological diseases such as acute and post-traumatic brain injury, stroke and neurodegenerative disorders (see e.g., Fumagalli et al., 2016; Tse and Herrup, 2016). Moreover, the myelination process can be perturbed by exposure to chemicals and drugs (see e.g., Garcia et al., 2005; Brubaker et al., 2009; Creeley et al., 2013) during brain development and adulthood. For example, the BMPS disclosed herein show 40% overall myelination after 8 weeks of differentiation. Myelin was observed by immunohistochemistry and confirmed by confocal microscopy 3D reconstruction and electron microscopy. These findings are of particular relevance since myelin is crucial for proper neuronal function and development. The ability to assess oligodendroglia function and mechanisms associated with myelination in this BMPS model provide an excellent tool for future studies of neurological disorders such as multiple sclerosis and other demyelinating diseases. Thus, the BMPS provides a suitable and reliable model to investigate neuron-neuroglia function in neurotoxicology or other pathogenic mechanisms that has heretofore not been available in the prior art.

The method disclosed combines gyratory shaking or regular stirring and the addition of growth factors to obtain the basic model. Suitable conditions as to how to achieve reproducible brain composition are disclosed herein. In contrast to earlier models, identical units of BMPS are produced, which allow comparative testing for the purpose of product development or safety assessments.

According to the techniques herein, a number of additional measures complement the basic BMPS to increase their completeness in modeling the human brain and improve its usefulness for such testing, for example:

1. The addition of microglia: All stem-cell-derived brain models described so far lack micro-glia. The techniques herein provide that the addition of micro-glia precursor cells and suitable growth factors may allow microglia to be added to the model. Suitable cells may be monocytes (e.g., human monocytes), hematopoetic stem cells, respective (pro-)monocyte cell lines, and isolated microglia.

2. The addition of a blood-brain-barrier: The human brain is protected by a tight blood-brain-barrier that excludes many substances from the brain. For the first time, the techniques herein provide a method to form a blood-brain-barrier to the BMPS via cells such as, for example, human endothelial cells.

3. Addition of reporter and reporter cells: During the generation of the BMPS, cells carrying reporter for testing purposes may be used or added. These include, but are not limited to, fluorescent or luminescent markers to indicate a certain cell lineage or cell response. Genetic transient or permanent transfections are the primary, but not only, method of choice.

4. The BMPS may also be produced, entirely or in its components, from cells from a specific genetic background, e.g. from patients with a specific disease or after selective genetic manipulation of the cells.

5. The versatility of the BMPS may be improved by combining it with electrodes including, but not limited to, micro-electrode arrays (MEA).

6. The versatility of the BMPS may be improved by combining it with other MPS (organ models) platforms such as, for example, microfluidic human-on-chip systems, perfusion chambers and others.

7. Transportability of BMPS: Methods to cryopreserve BMPS were developed, which allow transport to other laboratories and testing or integration into multi-MPS platforms.

Simplified neural in vitro systems do not reflect physiology, interactions between different cell types, or human genetics. Induced pluripotent stem cells (iPSC)-derived human-relevant microphysiological systems (MPS) better mimic the organ level, but are too complex for chemical and drug screening. As described herein, a reproducible 3D brain MPS (BMPS) that differentiates into mature neurons and glial cells (astrocytes and oligodendrocytes), which reproduces the topology of neuronal-glial interactions and connectivity in the central nervous system was developed. BMPS from healthy donors or patients evolve from a period of differentiation to maturity over about 8 weeks, including synaptogenesis, neuron-neuron interactions (e.g. spontaneous electric field potentials) and neuronal-glial interactions (e.g. myelination of axons), which mimic the microenvironment of the central nervous system. Effects of substances on neurodevelopment may be studied during this phase of BMPS development. In an exemplary embodiment, the techniques herein were used to study Parkinson's disease (PD) by evaluating neurotoxicants with a link to PD pathogenesis. Exposure to 5 μM rotenone or 100 μM 1-methyl-4-phenylpyridinium (MPP+) (or 1 mM 1-methyl-4-phenylpyridinium (MPP+) for gene expression studies) disrupted dopaminergic neurons, as observed by immunohistochemistry and altered expression of PD-related genes (TH, TBR1, SNCA, KEAP1, NDUFB1, ATP5C1, ATP50 and CASP1), thus recapitulating hallmarks of PD pathogenesis linked to toxicant compounds in the respective animal models. The BMPS, as described herein, provide a suitable and reliable model to investigate neuron-neuroglia function in neurotoxicity or other pathogenic mechanisms.

There is growing concern about the continuing increase in neurodevelopmental and -degenerative disorders such as autism [1, 2], Parkinson's [3] and Alzheimer disease [4]. Although genetic factors play an important role, environmental factors such as pesticides, air pollution, cigarette smoke, and dietary toxicants appear to contribute [5, 6, 7]. Due to a lack of mechanistic understanding, it is difficult to study their contributions and interactions with respect to neurotoxicity and neurological disorders. The complexity of the CNS makes it challenging to find appropriate in vitro human-relevant models, ideally from different genetic backgrounds, that are able to recapitulate the relevant pathophysiology. The poor predictive ability of animal-based models for human health, which may fail to mimic human pathology as outlined in the costly and time-consuming current developmental neurotoxicity (DNT) guidelines, contributes to the lack of reliable information on DNT mechanisms [8]. At the same time, more than 90% of all drugs fail clinical trials after extensive animal testing [9] due, in part, to the fact that animal studies often do not reflect human physiology and inter-individual differences. Simple in vitro systems do not represent physiology and organ function [10], which creates a critical demand for better models in drug development, study of disease mechanisms and progression, bioengineering and toxicological testing.

Attempts to generate more complex organotypic cultures or microphysiological systems (MPS) [11, 12, 13, 14] have resulted in more physiological multicellular 3D co-culture models able to simulate a functional part of the brain [15, 16]. 3D MPS have shown increased cell survival, differentiation, cell-cell interactions and can reproduce the complexity of the organ more closely [18]. Recent US research programs by NIH, FDA, DARPA, and DTRA have initiated the systematic development of MPS, including the model presented here, and their combinations to human-on-a-chip technologies to assess the safety and efficacy of countermeasures to biological and chemical terrorism and warfare [19].

The discovery of induced pluripotent stem cells (iPSC) and new protocols to differentiate them into various cell types have boosted the development of human in vitro models [20, 21]. iPSC from healthy or patient donors with a specific disease [22, 23, 24, 12] used in MPS promise more human-representative models, e.g. the brain organoids by Lancaster et al. and Kadoshima et al., have been able to recapitulate features of human cortical development [15, 16]. These complex systems present novel tools to study biological mechanisms in the CNS, however, they have certain limitations: 1) an elaborate and complex protocol, 2) size differences between organoids, 3) necrosis in the center of the organoid, 4) low reproducibility in cell differentiation. The human BMPS described herein overcomes these limitations. The reproducible in vitro iPSC-derived human 3D brain microphysiological system (BMPS) is comprised of differentiated and mature neurons and glial cells (astrocytes and oligodendrocytes).

The techniques herein provide a reproducible BMPS that contains several different cell types of the human brain, such as glutamatergic, dopaminergic and GABAergic neurons, astrocytes and oligodendrocytes. Moreover, the system has shown neural functionality as observed by spontaneous electrical activity and myelination of axons. Furthermore, the BMPS is reproducible from batch to batch and displays differences between healthy and patient donors. In addition, the obtained results demonstrate the application of such BMPS to the study of neurological disorders such as, for example, Parkinson's Disease (PD).

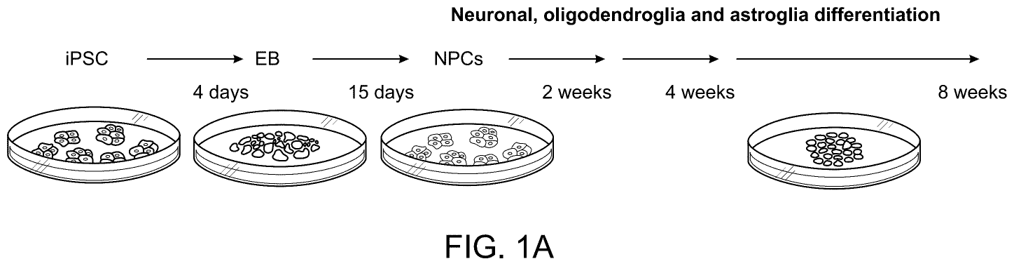

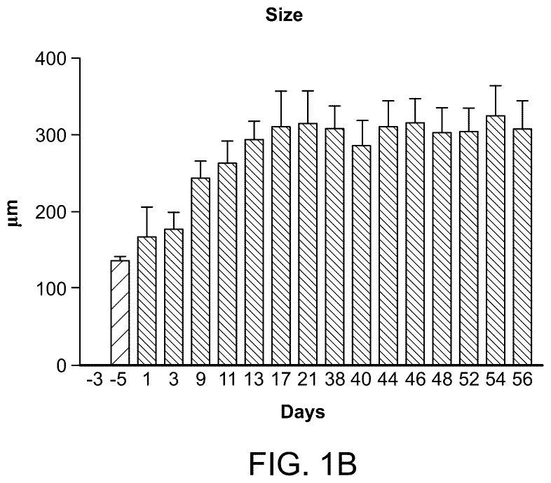

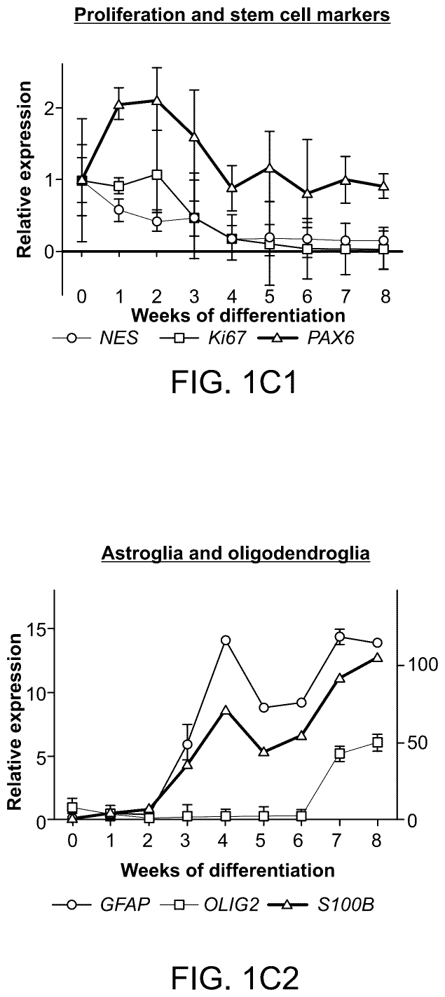

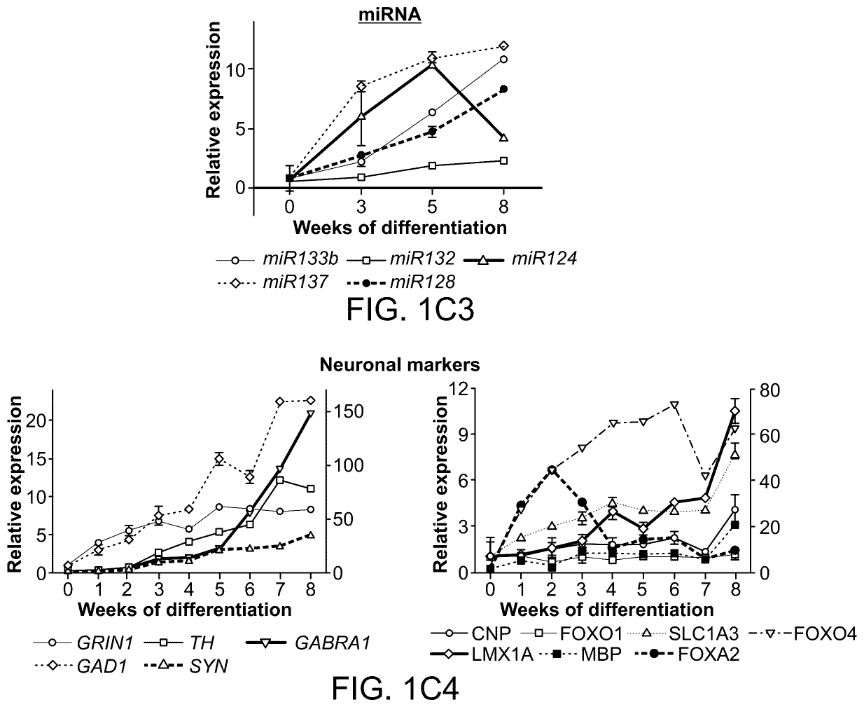

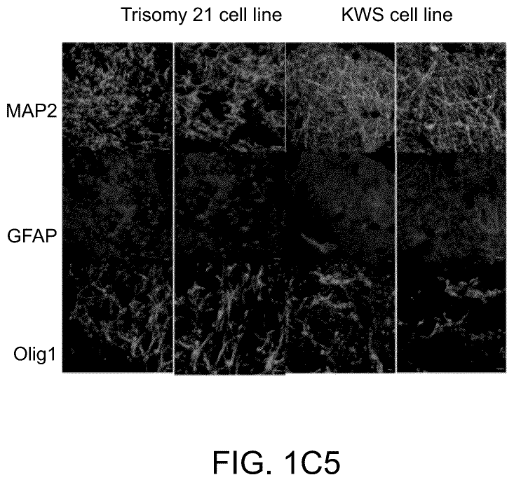

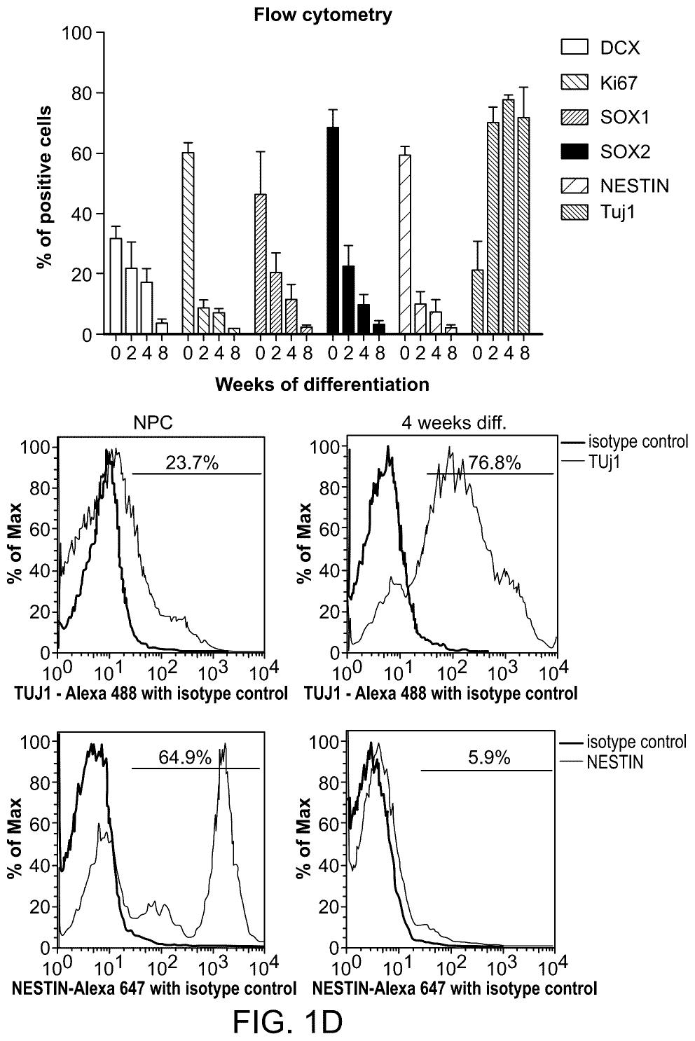

The brain MPS described herein is a versatile tool for more complex testing platforms and strategies as well as research into neurotoxicity (e.g., developmental), CNS physiology and pathology. Some stem cell-derived brain microphysiological systems have been developed in the latest years showing the capability to recapitulate some of the in vivo biological process [36, 37, 38]. These models have an enormous advantage over the classical in vitro models to study various differentiation mechanisms, developmental processes and diseases [15]. However, they are mostly based on human embryonic stem cells raising ethical concerns and not allowing the use of patient cells. Moreover, they require complicated protocols that may reduce the reproducibility of the system and make it difficult to use in other fields such as chemical and drug screening. Some of these complex organoids have a large diameter, which can lead to extensive cell death, visible in the core of these tissues [15]. This may be due to insufficient diffusion of nutrients and oxygen in these non-vascularized systems, which may generate artifacts in toxicological and disease measurements and make it difficult to study different endpoints in a medium- to high-throughput manner. In addition, it will be challenging to adapt endpoints, established for relative simple 2D cultures, to such complex models. In the study described herein, the ability to generate a high number of viable (about 800 per batch), BMPS that are homogeneous in size (e.g., about 300 μm) and shape using iPSC by applying a constant or regular gyratory shaking or stirring technique as described earlier for rat re-aggregating brain cell cultures [40] is shown. Control of the size using specific shaker speed allowed the aggregates to be maintained below 350 μM in diameter (FIG. 1B) and avoid disparate morphology and/or necrosis in the middle of the organoids. Moreover, a spherical homogeneous shape facilitates fluorescent quantification and further imaging-based endpoints as well as reproducibility between aggregates. The BMPS had reproducible cell composition by immunomorphological quantification, assessment of imaging-based endpoints and neurophysiological testing.

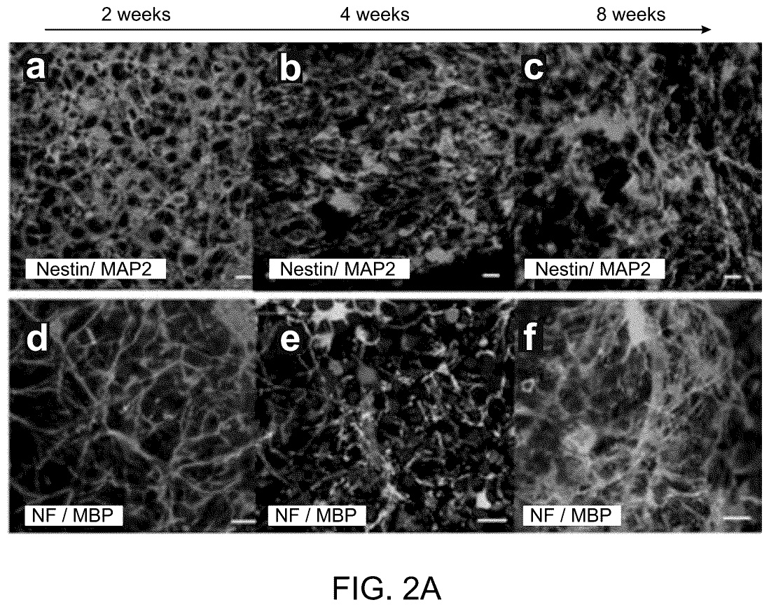

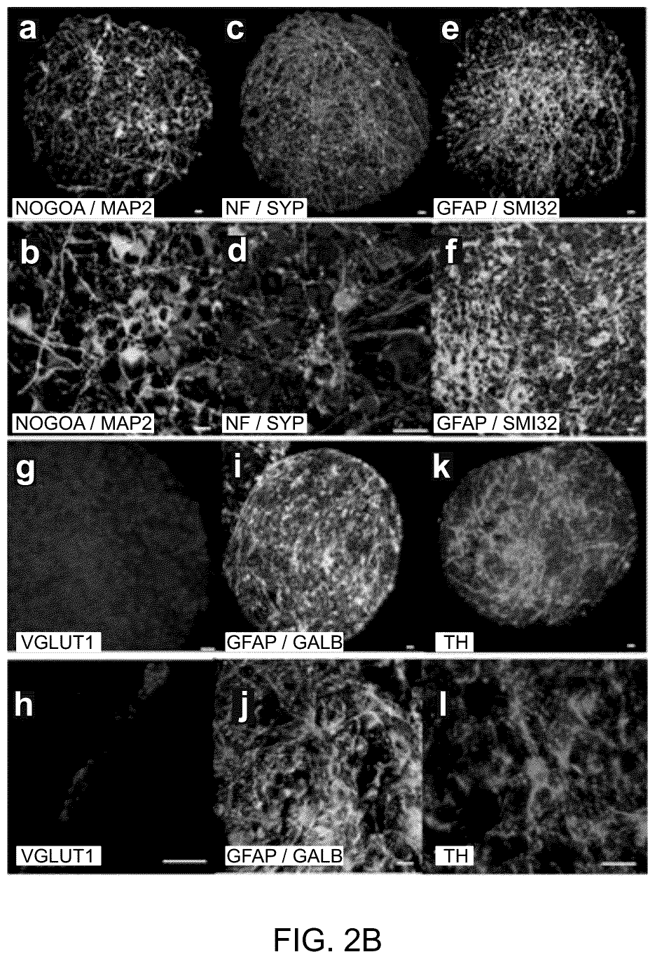

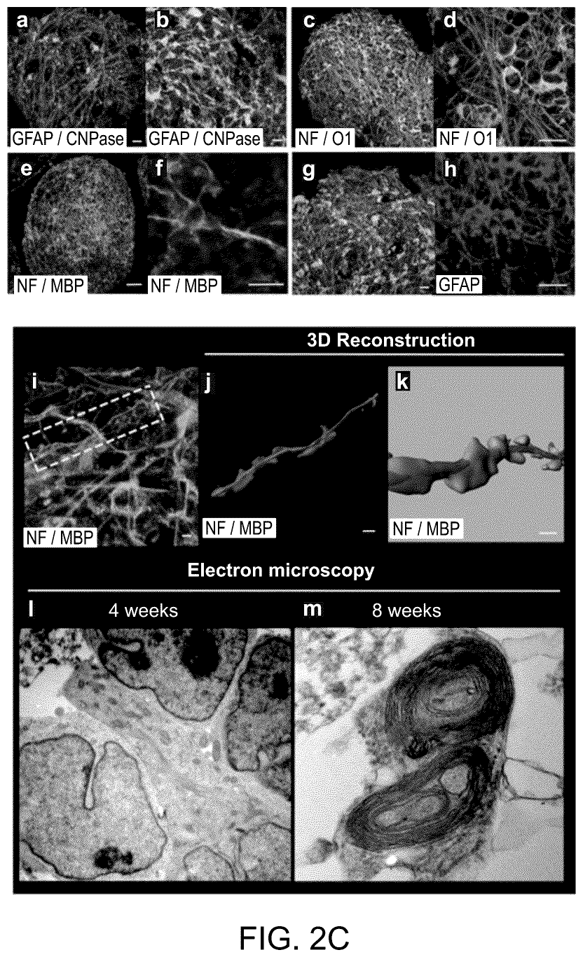

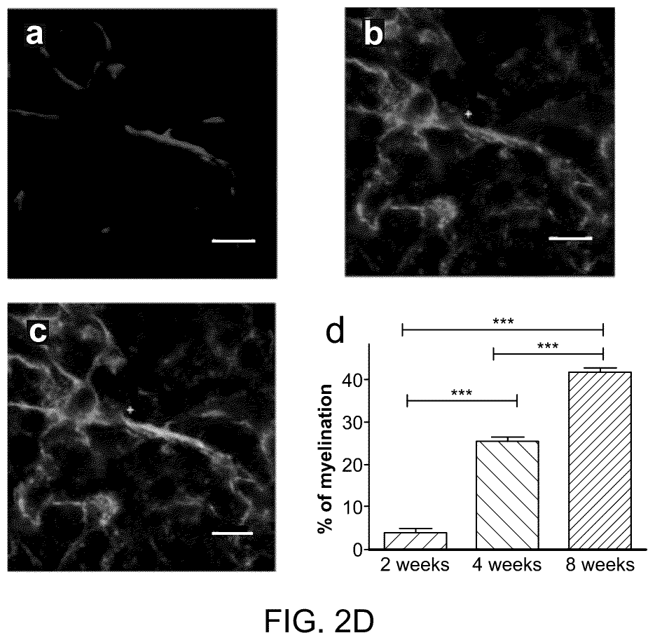

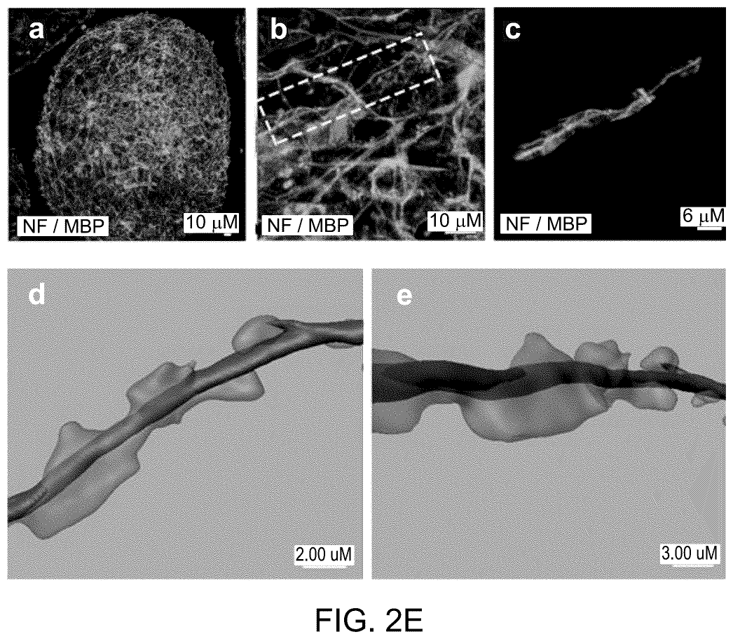

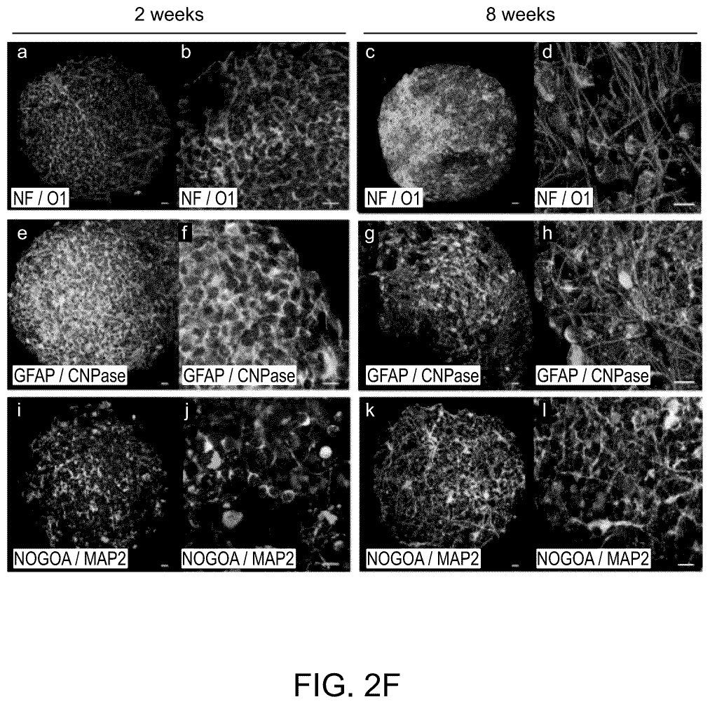

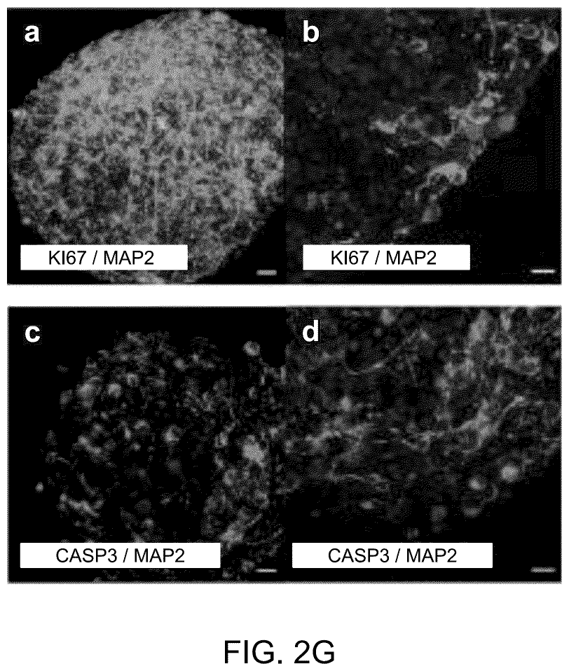

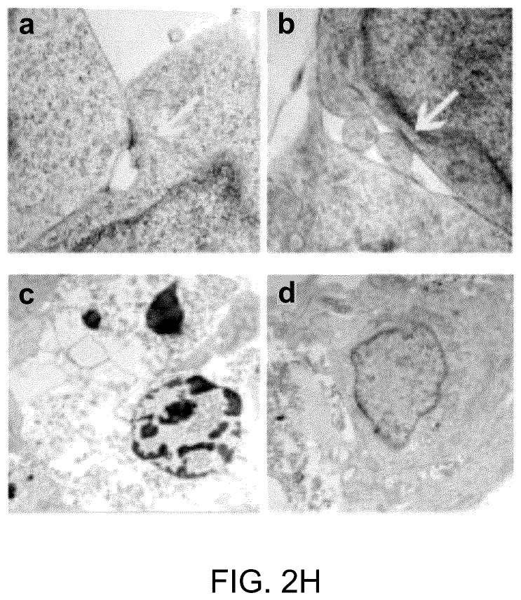

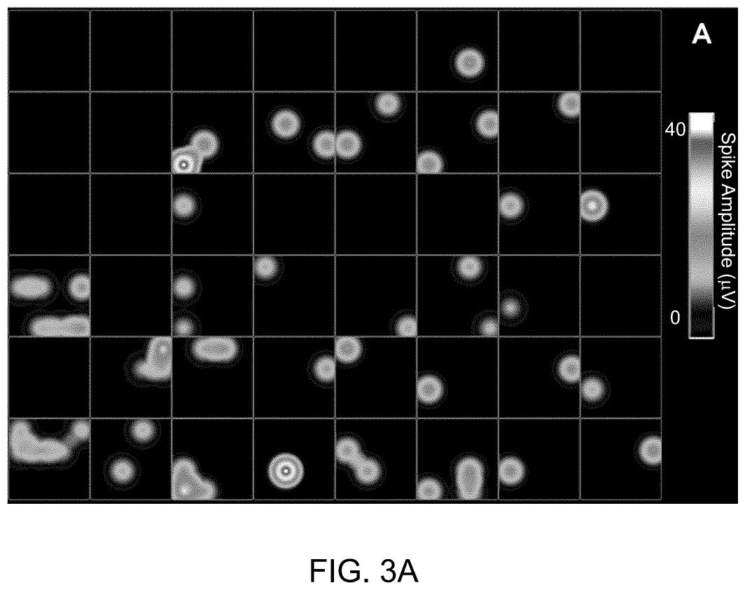

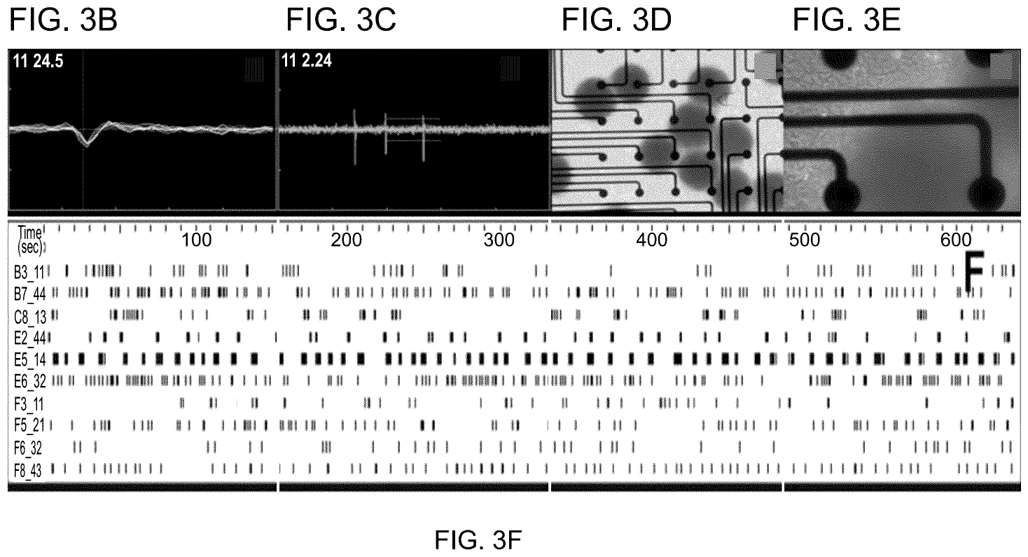

The 3D differentiation protocol described herein covered stages from neuronal precursors to different cell types of the mature CNS. After 2 weeks, BMPS consisted of an immature population of cells, showing minimal neuronal networks, low percentage of mature astrocytes and oligodendrocytes, with no myelin basic protein expression (FIG. 1C). Cell populations in the BMPS were further differentiated and matured over time (FIG. 2A). Evidence of iPSC differentiation into mature BMPS was supported by decreased Nestin expression over time. Nestin is normally expressed in embryonic tissue and its expression decreases with age in humans, therefore its decrement is a sign of maturation towards the adult phenotype [41, 42]. Also, the increasing presence of mature neuronal and glial markers such as MAP2, GFAP, Olig1 and MBP corroborate differentiation of the system. Different markers of pluripotency and proliferation decreased during the differentiation process, indicating maturing of the in vitro system (FIGS. 1C and 1D). Neuronal precursor markers such as Nestin, SOX1, SOX2 and the proliferation marker Ki67 decreased at the gene expression level and in flow cytometry measurements during the differentiation process (FIGS. 1C and 1D). Gene expression studies, flow cytometry, image analysis, immunostaining and miRNA studies have demonstrated an increase of cell maturation markers, which follows the BMPS differentiation (FIGS. 1A-1D, 2A-2H and 9A-9C). Obtained data demonstrate that this simple protocol is sufficient to generate representative CNS cell phenotypes that can reproduce various stages of differentiation. The presence of GABAergic neurons, dopaminergic neurons and glutamatergic neurons was observed by immunohistochemistry and real-time-PCR data (FIG. 1C and FIG. 2B). In addition, miRNAs such as mir-124, mir-132, mir-128, mir-137 and mir133b with a role in nervous system differentiation and neuronal degeneration [43, 44] increased during differentiation in patterns consistent with the in vivo situation. Moreover, the BMPS described herein produced spontaneous electrical activity (FIG. 3) confirming neuronal functionality of the system. However, further optimizations of the electrophysiological measurements using MEAs in 3D systems are needed.

Most of the brain MPS published so far are entirely focused on neurons and not glia populations [45, 46]; the brain MPS described herein is the first 3D model with fully characterized mature human oligodendrocytes, astrocytes and neurons, derived from iPSC. Astrocytes and oligodendrocytes play an important role during neuronal development, plasticity and neuronal injury. Astrocytes have a role in protecting neurons, increasing neuronal viability and mitochondrial biogenesis from both exogenous (e.g. chemicals) or endogenous (such as glutamate-induced excitotoxicity or the Alzheimer related Aβ1-42) toxicity [47, 48, 49, 50]. Astrocytes have an especially important role in neuroprotection from oxidative stress. Oxidative stress is known to be involved in a number of neuropathological conditions (such as neurodegenerative diseases) [51, 52, 53]. Thus, the presence of astrocytes in a biological system to study disease is crucial due to their role in detoxification and neuronal protection. Immunochemistry results from the iPSC-derived BMPS showed low numbers of astrocytes (GFAP-positive cells) at 2 weeks of differentiation, which increased continuously throughout differentiation (FIG. 2F-2H, and FIG. 2A). Real-time RT-PCR data supports these findings, as a continuous increase in both s100b and GFAP mRNA levels could be observed from 2 weeks up to 8 weeks old BMPS. Immunohistochemistry and RT-PCR data results showed increasing numbers of astrocytes (GFAP-positive cells) in the BMPS model, reaching 19% astrocytes of the total cell population at 8 weeks. After 4 weeks of differentiation, astrocytes demonstrated increased positive staining for GFAP and the presence of glial network was observed (FIG. 2C, panels g, h). At the same time, the presence of oligodendrocytes and myelination of axons could be observed in the system described herein. This process is highly important, since it is known to be involved in many degenerative diseases such as multiple sclerosis [54], congenital hypomyelination [55], progressive multifocal leukoencephalopathy caused by JC virus infection [56], periventricular leukomalacia (PVL) [57] and Alzheimer's disease [58]. Moreover, several chemicals such as ethanol [59], tellurium [60] and lead [(61, 62, 63, 64, 65] have shown to have an effect on the myelination process.

The presence of astroglia and oligodendroglia in the model described herein brings the system closer to the in vivo brain physiology, which is a crucial component to study neurodegeneration and neurotoxicity. In addition, the system has shown functionality as seen by imaging of cell-cell junctions, myelination, a rich astroglial network and electrical activity (FIG. 3). These characteristics make the BMPS described herein a promising tool to study interactions between human neuronal cells in neurological diseases. The use of iPSCs makes it possible to study genetic factors and gene/environment interactions.

An assessment of the myelination process by quantification of MBP immunostaining along axons showed an increase over time reaching 42% of myelinated axons at 8 weeks (FIG. 2D). 3D reconstruction of confocal z-stacks images (FIGS. 2C and 2E) and electron microscopy confirmed the wrapping of axonal structures after 8 weeks of differentiation (FIG. 2C). These findings are of particular relevance since myelin is a critical element for proper neuronal function and development, the ensheathment of axons by myelin allows faster action potential transmission, reduces axonal energy consumption and protects the axons from degeneration[79]. Furthermore, recent evidence suggests that oligodendrocytes and myelin have a role in the metabolic support of axons independent of their role in action potential conduction, highlighting their importance in neuronal survival[80]. The ability of assessing oligodendroglia function and mechanisms associated with myelination in the BMPS model provide an excellent tool for future studies of neurological disorders such as multiple sclerosis and other demyelinating disorders.

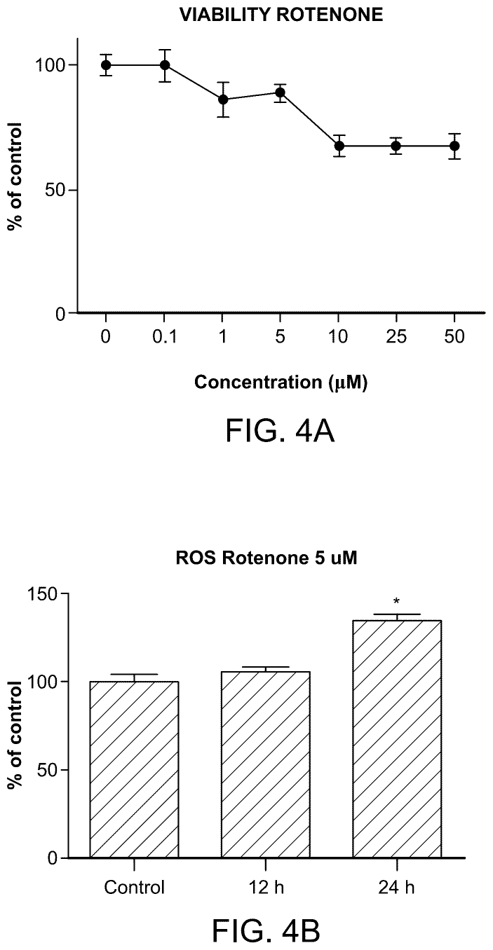

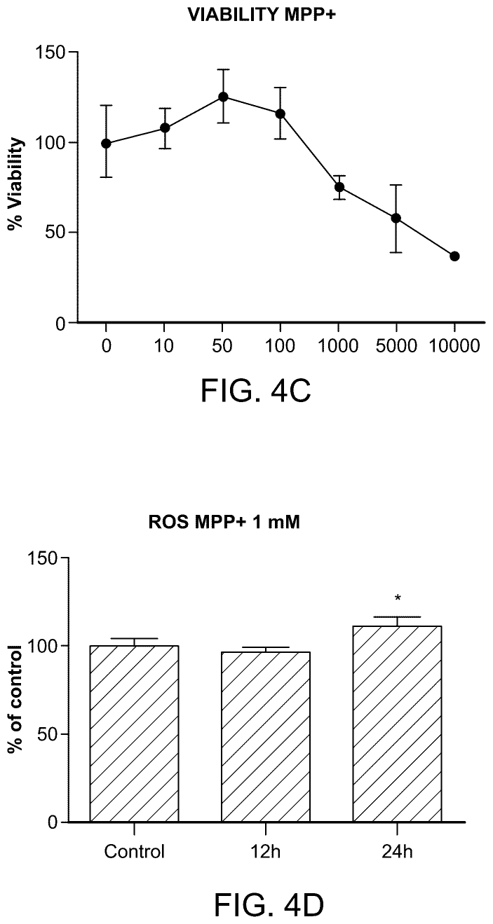

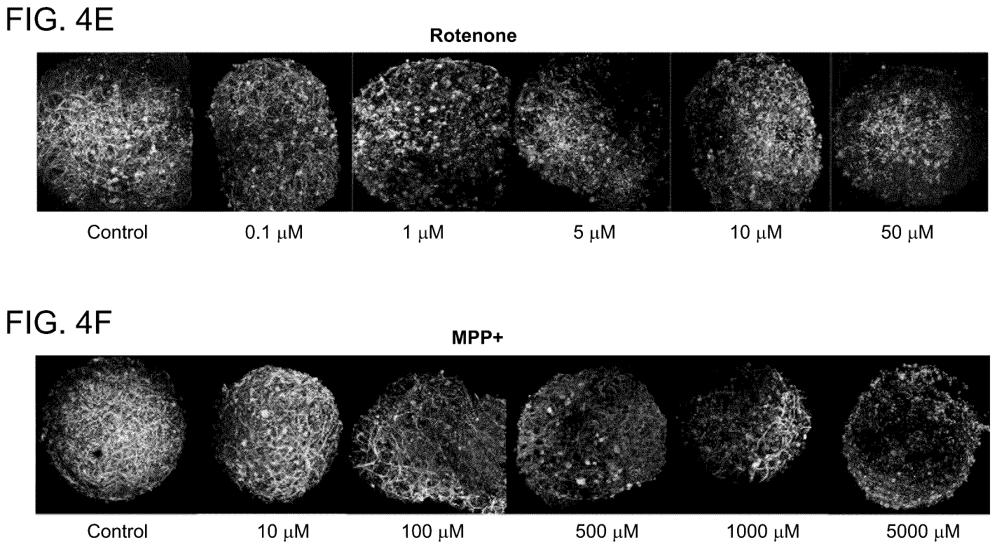

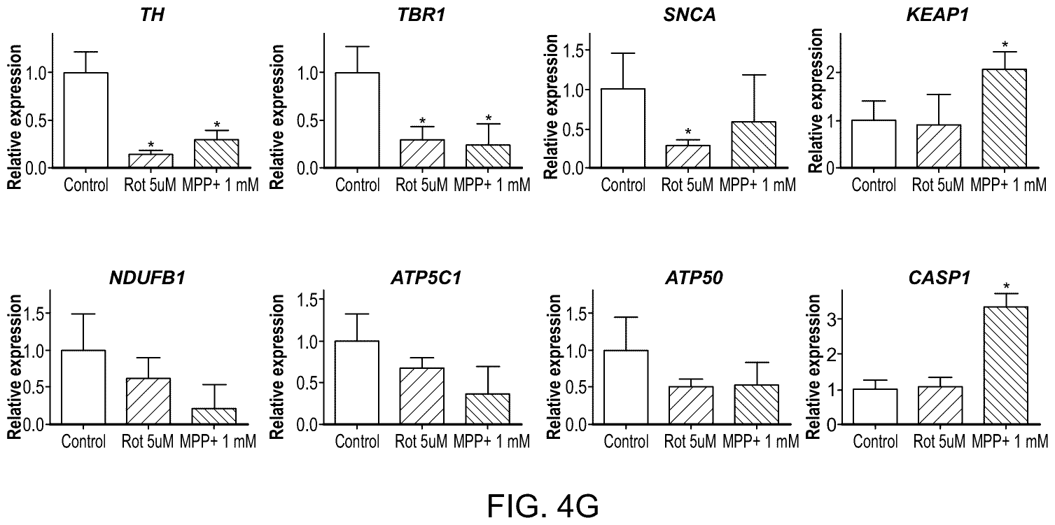

In one embodiment, the model described herein is useful for studying Parkinson's disease (PD). Traditionally, PD has been described as a pre-synaptic degenerative process that affects dopaminergic neurons and induces a fundamental motor disorder [66], however, non-motor symptoms can also be present [67]. Research in Parkinson's disease is experiencing an upswing at the moment, owing to a lack of curative drugs for the large number of patients. Drug testing is nearly exclusively performed in vivo in the so-called MPTP (the parent compound to the metabolite MPP+ used here), rotenone, methamphetamine and 6-hydroxydopamine models requiring tens of thousands of animals [68, 69, 70]. These model toxins are mainly used in mice and primates (and less in cell cultures) to model a disease state resembling PD. Human neurons, which would be most relevant, are not usually available and existing cell lines are only very poor substitutes. The model described herein shows that treatment with MPP+ or rotenone induced specific degeneration of dopaminergic neurons in agreement with Parkinson patients and current animal models of the disease (FIGS. 4E and 4F). The BMPS PD model has shown to recapitulate some of the molecular mechanisms of the human disease, e.g. increase in ROS production (FIGS. 4B and 3D) and changes in genes related to PD (FIG. 4G). BMPS treated with rotenone or MPP+ had decreased TH gene expression compared to controls, supporting the results presented in FIGS. 4E and 4F where the dopaminergic neuronal phenotype is altered after treatment with the two chemicals. TBR1 encodes a transcription factor involved in the regulation of developmental processes. It also plays a role in major neurological diseases such as Alzheimer Disease and PD [71]. This gene was down-regulated after treatment with non-cytotoxic concentrations of MPP+ and rotenone. At the same time, mRNA levels of SNAC were altered. α-Synucleinopathy (common in Parkinson) is a neurodegenerative disease, which consists of the abnormal accumulation of aggregates of alpha-synuclein protein in neurons, nerve fibers or glial cells [72]. Alpha-synuclein plays regulatory roles such as synaptic maintenance, mitochondrial homeostasis, proteasome function, dopamine metabolism [73]. Reduction of SNCA (the alpha-synuclein encoding gene) after treatment with 5 μM rotenone and to a lesser extent after 1 mM MPP+ exposure could be explained by the alteration of alpha-synuclein protein metabolism. However, it may be that longer exposure times are required to produce an increase in gene expression. Caspase-1 (CASP1) expression increased significantly after 24 h exposure to 1 μM MPP+. Recently, some studies have identified human enzyme caspase-1 as the protease that cleaves α-synuclein in vivo [74]. This cleavage generates α-synuclein fragments that are prone to toxic aggregate formation. Finally, effects upon genes related with mitochondrial function (such as NDUFB1, ATP5C1 and ATP50) were down-regulated, more strongly in BMPS treated with MPP+ than rotenone. Changes in NDUFB1, indicate an alteration in mitochondrial function, agreeing with the phenomena already described in Parkinson's disease. This downregulation is linked to the increase in KEAP1 expression (oxidative stress marker) after 24 h exposure to 1 mM MPP+. The high variability in some of the genes may be explained by the selective effects of these chemicals (especially MPP+) to dopaminergic neurons, which represent only a subpopulation within the BMPS. While rotenone and MPP+ alter gene expression of this cell population, the other populations presented in BMPS appear not to be affected. Further studies using cell sorting could identify cell-specific effects.

This disclosure provides for a description of a brain microphysiological system aiming to study various aspects of brain development, pathophysiology and disturbance by genetic and environmental factors. The possibilities to study developmental and neurodegenerative disorders, infections, toxicity and trauma are emerging with such a system. Furthermore, the potential to use iPSC from different donors adds a personalized component to these studies. The high reproducibility and relatively easy protocol, enables future higher throughput testing of chemicals, and drugs and their potential to induce or treat diseases.

### Autism

Autism is a highly variable neurodevelopmental disorder that first appears during infancy or childhood, and generally follows a steady course without remission. Patients with autism may be severely impaired in some respects but normal, or even superior, in others. Overt symptoms gradually begin after the age of six months, become established by age two or three years, and tend to continue through adulthood, although often in more muted form. It is distinguished not by a single symptom, but by a characteristic triad of symptoms: impairments in social interaction; impairments in communication; and restricted interests and repetitive behavior. Other aspects, such as atypical eating, are also common but are not essential for diagnosis. Autism's individual symptoms occur in the general population and appear not to associate highly, without a sharp line separating pathologically severe from common traits.

While autism is highly heritable, researchers suspect both environmental and genetic factors as causes. In rare cases, autism is strongly associated with agents that cause birth defects. Controversies surround other proposed environmental causes; for example, the vaccine hypotheses have been disproven. Autism affects information processing in the brain by altering how nerve cells and their synapses connect and organize; how this occurs is not well understood. It is one of three recognized disorders in the autism spectrum (ASDs), the other two being Asperger syndrome, which lacks delays in cognitive development and language, and pervasive developmental disorder, not otherwise specified (commonly abbreviated as PDD-NOS), which is diagnosed when the full set of criteria for autism or Asperger syndrome are not met.

Globally, autism is estimated to affect 21.7 million people as of 2013. As of 2010, the number of people affected is estimated at about 1-2 per 1,000 worldwide. It occurs four to five times more often in boys than girls. About 1.5% of children in the United States (one in 68) are diagnosed with ASD as of 2014, a 30% increase from one in 88 in 2012. The rate of autism among adults aged 18 years and over in the United Kingdom is 1.1%. The number of people diagnosed has been increasing dramatically since the 1980s, partly due to changes in diagnostic practice and government-subsidized financial incentives for named diagnoses; the question of whether actual rates have increased is unresolved.

Autism has a strong genetic basis, although the genetics of autism are complex and it is unclear whether ASD is explained more by rare mutations with major effects, or by rare multigene interactions of common genetic variants. Complexity arises due to interactions among multiple genes, the environment, and epigenetic factors which do not change DNA but are heritable and influence gene expression. Studies of twins suggest that heritability is 0.7 for autism and as high as 0.9 for ASD, and siblings of those with autism are about 25 times more likely to be autistic than the general population. However, most of the mutations that increase autism risk have not been identified. Typically, autism cannot be traced to a Mendelian (single-gene) mutation or to a single chromosome abnormality, and none of the genetic syndromes associated with ASDs have been shown to selectively cause ASD. Numerous candidate genes have been located, with only small effects attributable to any particular gene. The large number of autistic individuals with unaffected family members may result from copy number variations-spontaneous deletions or duplications in genetic material during meiosis. Hence, a substantial fraction of autism cases may be traceable to genetic causes that are highly heritable but not inherited: that is, the mutation that causes the autism is not present in the parental genome.

Several lines of evidence point to synaptic dysfunction as a cause of autism. Some rare mutations may lead to autism by disrupting some synaptic pathways, such as those involved with cell adhesion. Gene replacement studies in mice suggest that autistic symptoms are closely related to later developmental steps that depend on activity in synapses and on activity-dependent changes. All known teratogens (agents that cause birth defects) related to the risk of autism appear to act during the first eight weeks from conception, and though this does not exclude the possibility that autism can be initiated or affected later, there is strong evidence that autism arises very early in development.

Exposure to air pollution during pregnancy, especially heavy metals and particulates, may increase the risk of autism. Environmental factors that have been claimed to contribute to or exacerbate autism, or may be important in future research, include certain foods, infectious diseases, solvents, diesel exhaust, PCBs, phthalates and phenols used in plastic products, pesticides, brominated flame retardants, alcohol, smoking, illicit drugs, vaccines, and prenatal stress, although no links have been found, and some have been completely disproven.

Autism does not have a clear unifying mechanism at either the molecular, cellular, or systems level; it is not known whether autism is a few disorders caused by mutations converging on a few common molecular pathways, or is (like intellectual disability) a large set of disorders with diverse mechanisms. Autism appears to result from developmental factors that affect many or all functional brain systems, and to disturb the timing of brain development more than the final product. Neuroanatomical studies and the associations with teratogens strongly suggest that autism's mechanism includes alteration of brain development soon after conception. This anomaly appears to start a cascade of pathological events in the brain that are significantly influenced by environmental factors. Just after birth, the brains of children with autism tend to grow faster than usual, followed by normal or relatively slower growth in childhood. It is not known whether early overgrowth occurs in all children with autism. It seems to be most prominent in brain areas underlying the development of higher cognitive specialization. Hypotheses for the cellular and molecular bases of pathological early overgrowth include the following: an excess of neurons that causes local over connectivity in key brain regions, disturbed neuronal migration during early gestation, unbalanced excitatory-inhibitory networks, and abnormal formation of synapses and dendritic spines, for example, by modulation of the neurexin-neuroligin cell-adhesion system, or by poorly regulated synthesis of synaptic proteins.

The immune system is thought to play an important role in autism. Children with autism have been found by researchers to have inflammation of both the peripheral and central immune systems as indicated by increased levels of pro-inflammatory cytokines and significant activation of microglia. Biomarkers of abnormal immune function have also been associated with increased impairments in behaviors that are characteristic of the core features of autism such as deficits in social interactions and communication. Interactions between the immune system and the nervous system begin early during the embryonic stage of life, and successful neurodevelopment depends on a balanced immune response. It is thought that activation of a pregnant mother's immune system such as from environmental toxicants or infection can contribute to causing autism through causing a disruption of brain development. This is supported by recent studies that have found that infection during pregnancy is associated with an increased risk of autism.

The relationship of neurochemicals to autism is not well understood; several have been investigated, with the most evidence for the role of serotonin and of genetic differences in its transport. The role of group I metabotropic glutamate receptors (mGluR) in the pathogenesis of fragile X syndrome, the most common identified genetic cause of autism, has led to interest in the possible implications for future autism research into this pathway. Some data suggests neuronal overgrowth potentially related to an increase in several growth hormones or to impaired regulation of growth factor receptors. Also, some inborn errors of metabolism are associated with autism, but probably account for less than 5% of cases.

The mirror neuron system (MNS) theory of autism hypothesizes that distortion in the development of the MNS interferes with imitation and leads to autism's core features of social impairment and communication difficulties. The MNS operates when an animal performs an action or observes another animal perform the same action. The MNS may contribute to an individual's understanding of other people by enabling the modeling of their behavior via embodied simulation of their actions, intentions, and emotions. Several studies have tested this hypothesis by demonstrating structural abnormalities in MNS regions of individuals with ASD, delay in the activation in the core circuit for imitation in individuals with Asperger syndrome, and a correlation between reduced MNS activity and severity of the syndrome in children with ASD. However, individuals with autism also have abnormal brain activation in many circuits outside the MNS and the MNS theory does not explain the normal performance of children with autism on imitation tasks that involve a goal or object.

The under connectivity theory of autism hypothesizes that autism is marked by under functioning high-level neural connections and synchronization, along with an excess of low-level processes. Evidence for this theory has been found in functional neuroimaging studies on autistic individuals and by a brainwave study that suggested that adults with ASD have local over connectivity in the cortex and weak functional connections between the frontal lobe and the rest of the cortex. Other evidence suggests the under connectivity is mainly within each hemisphere of the cortex and that autism is a disorder of the association cortex.

From studies based on event-related potentials, transient changes to the brain's electrical activity in response to stimuli, there is considerable evidence for differences in autistic individuals with respect to attention, orientation to auditory and visual stimuli, novelty detection, language and face processing, and information storage; several studies have found a preference for nonsocial stimuli. For example, magnetoencephalography studies have found evidence in children with autism of delayed responses in the brain's processing of auditory signals.

Relations have been found between autism and schizophrenia based on duplications and deletions of chromosomes; research showed that schizophrenia and autism are significantly more common in combination with 1q21.1 deletion syndrome. Research on autism/schizophrenia relations for chromosome 15 (15q13.3), chromosome 16 (16p13.1) and chromosome 17 (17p12) are inconclusive.

Diagnosis is based on behavior, not cause or mechanism. Under the DSM-5, autism is characterized by persistent deficits in social communication and interaction across multiple contexts, as well as restricted, repetitive patterns of behavior, interests, or activities. These deficits are present in early childhood, typically before age three, and lead to clinically significant functional impairment. Sample symptoms include lack of social or emotional reciprocity, stereotyped and repetitive use of language or idiosyncratic language, and persistent preoccupation with unusual objects. The disturbance must not be better accounted for by Rett syndrome, intellectual disability or global developmental delay. ICD-10 uses essentially the same definition. A pediatrician commonly performs a preliminary investigation by taking developmental history and physically examining the child. If warranted, diagnosis and evaluations are conducted with help from ASD specialists, observing and assessing cognitive, communication, family, and other factors using standardized tools, and taking into account any associated medical conditions. A pediatric neuropsychologist is often asked to assess behavior and cognitive skills, both to aid diagnosis and to help recommend educational interventions.

Clinical genetics evaluations are often done once ASD is diagnosed, particularly when other symptoms already suggest a genetic cause. Although genetic technology allows clinical geneticists to link an estimated 40% of cases to genetic causes, consensus guidelines in the US and UK are limited to high-resolution chromosome and fragile X testing. Metabolic and neuroimaging tests are sometimes helpful, but are not routine.

Many medications are used to treat ASD symptoms that interfere with integrating a child into home or school when behavioral treatment fails. More than half of US children diagnosed with ASD are prescribed psychoactive drugs or anticonvulsants, with the most common drug classes being antidepressants, stimulants, and antipsychotics. Antipsychotics, such as risperidone and aripiprazole, have been found to be useful for treating some conditions associated with autism, including irritability, repetitive behavior, and sleeplessness. A person with ASD may respond atypically to medications, the medications can have adverse effects, and no known medication relieves autism's core symptoms of social and communication impairments. Experiments in mice have reversed or reduced some symptoms related to autism by replacing or modulating gene function, suggesting the possibility of targeting therapies to specific rare mutations known to cause autism. Although many alternative therapies and interventions are available, few are supported by scientific studies. Some alternative treatments may place the child at risk. A 2008 study found that compared to their peers, autistic boys have significantly thinner bones if on casein-free diets; in 2005, botched chelation therapy killed a five-year-old child with autism. There has been early research looking at hyperbaric treatments in children with autism.

### Parkinson's Disease

Parkinson's disease (PD, also known as idiopathic or primary parkinsonism, hypokinetic rigid syndrome (HRS), or paralysis agitans) is a degenerative disorder of the central nervous system mainly affecting the motor system. The motor symptoms of Parkinson's disease result from the death of dopamine-generating cells in the substantia nigra, a region of the midbrain. The causes of this cell death are poorly understood. Early in the course of the disease, the most obvious symptoms are movement-related; these include shaking, rigidity, slowness of movement and difficulty with walking and gait. Later, thinking and behavioral problems may arise, with dementia commonly occurring in the advanced stages of the disease, and depression is the most common psychiatric symptom. Other symptoms include sensory, sleep and emotional problems. Parkinson's disease is more common in older people, with most cases occurring after the age of 50; when it is seen in young adults, it is called young onset PD (YOPD).

The main motor symptoms are collectively called “parkinsonism,” or a “parkinsonian syndrome.” The disease can be either primary or secondary. Primary Parkinson's disease is referred to as idiopathic (having no known cause), although some atypical cases have a genetic origin, while secondary parkinsonism is due to known causes like toxins. The pathology of the disease is characterized by the accumulation of a protein into Lewy bodies in neurons, and insufficient formation and activity of dopamine in certain parts of the midbrain. Where the Lewy bodies are located is often related to the expression and degree of the symptoms of an individual. Diagnosis of typical cases is mainly based on symptoms, with tests such as neuroimaging being used for confirmation.

Diagnosis of Parkinson's disease involves a physician taking a medical history and performing a neurological examination. There is no lab test that will clearly identify the disease, but brain scans are sometimes used to rule out disorders that could give rise to similar symptoms. People may be given levodopa and resulting relief of motor impairment tends to confirm diagnosis. The finding of Lewy bodies in the midbrain on autopsy is usually considered proof that the person had Parkinson's disease. The progress of the illness over time may reveal it is not Parkinson's disease, and some authorities recommend that the diagnosis be periodically reviewed. Other causes that can secondarily produce a parkinsonian syndrome are Alzheimer's disease, multiple cerebral infarction and drug-induced parkinsonism. Parkinson plus syndromes such as progressive supranuclear palsy and multiple system atrophy must be ruled out. Anti-Parkinson's medications are typically less effective at controlling symptoms in Parkinson plus syndromes. Faster progression rates, early cognitive dysfunction or postural instability, minimal tremor or symmetry at onset may indicate a Parkinson plus disease rather than PD itself. Genetic forms are usually classified as PD, although the terms familial Parkinson's disease and familial parkinsonism are used for disease entities with an autosomal dominant or recessive pattern of inheritance.

The PD Society Brain Bank criteria require slowness of movement (bradykinesia) plus either rigidity, resting tremor, or postural instability. Other possible causes for these symptoms need to be ruled out prior to diagnosis with PD. Finally, three or more of the following features are required during onset or evolution: unilateral onset, tremor at rest, progression in time, asymmetry of motor symptoms, response to levodopa for at least five years, clinical course of at least ten years and appearance of dyskinesias induced by the intake of excessive levodopa. Accuracy of diagnostic criteria evaluated at autopsy is 75-90%, with specialists such as neurologists having the highest rates. Computed tomography (CT) and conventional magnetic resonance imaging (MRI) brain scans of people with PD usually appear normal. These techniques are nevertheless useful to rule out other diseases that can be secondary causes of parkinsonism, such as basal ganglia tumors, vascular pathology and hydrocephalus. A specific technique of MRI, diffusion MRI, has been reported to be useful at discriminating between typical and atypical parkinsonism, although its exact diagnostic value is still under investigation. Dopaminergic function in the basal ganglia can be measured with different PET and SPECT radiotracers. Examples are ioflupane (123I) (trade name DaTSCAN) and iometopane (Dopascan) for SPECT or fluorodeoxyglucose (18F) and DTBZ for PET. A pattern of reduced dopaminergic activity in the basal ganglia can aid in diagnosing PD.

Treatments, typically the medications L-DOPA and dopamine agonists, improve the early symptoms of the disease. As the disease progresses and dopaminergic neurons continue to be lost, these drugs eventually become ineffective at treating the symptoms and at the same time produce a complication marked by involuntary writhing movements. Surgery and deep brain stimulation have been used to reduce motor symptoms as a last resort in severe cases where drugs are ineffective. Although dopamine replacement alleviates the symptomatic motor dysfunction, its effectiveness is reduced as the disease progresses, leading to unacceptable side effects such as severe motor fluctuations and dyskinesias. Furthermore, there is no therapy that will halt the progress of the disease. Moreover, this palliative therapeutic approach does not address the underlying mechanisms of the disease.

The term parkinsonism is used for a motor syndrome whose main symptoms are tremor at rest, stiffness, slowing of movement and postural instability. Parkinsonian syndromes can be divided into four subtypes according to their origin: primary or idiopathic, secondary or acquired, hereditary parkinsonism, and Parkinson plus syndromes or multiple system degeneration. Usually classified as a movement disorder, PD also gives rise to several non-motor types of symptoms such as sensory deficits, cognitive difficulties or sleep problems. Parkinson plus diseases are primary parkinsonisms which present additional features. They include multiple system atrophy, progressive supranuclear palsy, corticobasal degeneration and dementia with Lewy bodies.

In terms of pathophysiology, PD is considered a synucleiopathy due to an abnormal accumulation of alpha-synuclein protein in the brain in the form of Lewy bodies, as opposed to other diseases such as Alzheimer's disease where the brain accumulates tau protein in the form of neurofibrillary tangles. Nevertheless, there is clinical and pathological overlap between tauopathies and synucleinopathies. The most typical symptom of Alzheimer's disease, dementia, occurs in advanced stages of PD, while it is common to find neurofibrillary tangles in brains affected by PD. Dementia with Lewy bodies (DLB) is another synucleinopathy that has similarities with PD, and especially with the subset of PD cases with dementia. However, the relationship between PD and DLB is complex and still has to be clarified. They may represent parts of a continuum or they may be separate diseases.

Mutations in specific genes have been conclusively shown to cause PD. These genes encode alpha-synuclein (SNCA), parkin (PRKN), leucine-rich repeat kinase 2 (LRRK2 or dardarin), PTEN-induced putative kinase 1 (PINK1), DJ-1 and ATP13A2. In most cases, people with these mutations will develop PD. With the exception of LRRK2, however, they account for only a small minority of cases of PD. The most extensively studied PD-related genes are SNCA and LRRK2. Mutations in genes including SNCA, LRRK2 and glucocerebrosidase (GBA) have been found to be risk factors for sporadic PD. Mutations in GBA are known to cause Gaucher's disease. Genome-wide association studies, which search for mutated alleles with low penetrance in sporadic cases, have now yielded many positive results.

The role of the SNCA gene is important in PD because the alpha-synuclein protein is the main component of Lewy bodies. The histopathology (microscopic anatomy) of the substantia nigra and several other brain regions shows neuronal loss and Lewy bodies in many of the remaining nerve cells. Neuronal loss is accompanied by death of astrocytes (star-shaped glial cells) and activation of the microglia (another type of glial cell). Lewy bodies are a key pathological feature of PD.

### Alzheimer's Disease

Alzheimer's disease (AD) accounts for 60% to 70% of cases of dementia. It is a chronic neurodegenerative disease that often starts slowly, but progressively worsens over time. The most common early symptom is short-term memory loss. As the disease advances, symptoms include problems with language, mood swings, loss of motivation, disorientation, behavioral issues, and poorly managed self-care. Gradually, bodily functions are lost, ultimately leading to death. Although the speed of progression can vary, the average life expectancy following diagnosis is three to nine years. The cause of Alzheimer's disease is poorly understood. About 70% of the risk is believed to be genetic with many genes involved. Other risk factors include a history of head injuries, hypertension, or depression. The disease process is associated with plaques and tangles in the brain.

Alzheimer's disease is characterized by loss of neurons and synapses in the cerebral cortex and certain subcortical regions. This loss results in gross atrophy of the affected regions, including degeneration in the temporal lobe and parietal lobe, and parts of the frontal cortex and cingulate gyrus. Alzheimer's disease has been hypothesized to be a protein misfolding disease (proteopathy), caused by accumulation of abnormally folded A-beta and tau proteins in the brain. Plaques are made up of small peptides, 39-43 amino acids in length, called beta-amyloid (also written as A-beta or Aβ). Beta-amyloid is a fragment from a larger protein called amyloid precursor protein (APP), a transmembrane protein that penetrates through the neuron's membrane. APP is critical to neuron growth, survival and post-injury repair. In Alzheimer's disease, an unknown process causes APP to be divided into smaller fragments by enzymes through proteolysis. One of these fragments gives rise to fibrils of beta-amyloid, which form clumps that deposit outside neurons in dense formations known as senile plaques.

A probable diagnosis is based on the history of the illness and cognitive testing with medical imaging and blood tests to rule out other possible causes. Initial symptoms are often mistaken for normal ageing. Examination of brain tissue is needed for a definite diagnosis. Alzheimer's disease is diagnosed through a complete medical assessment. There is no one clinical test that can determine whether a person has Alzheimer's. Usually several tests are performed to rule out any other cause of dementia. The only definitive method of diagnosis is examination of brain tissue obtained from a biopsy or autopsy. Tests (such as blood tests and brain imaging) are used to rule out other causes of dementia-like symptoms. Laboratory tests and screening include: complete blood cell count; electrolyte panel; screening metabolic panel; thyroid gland function tests; vitamin B-12 folate levels; tests for syphilis and, depending on history, for human immunodeficiency antibodies; urinalysis; electrocardiogram (ECG); chest X-ray; computerized tomography (CT) head scan; and an electroencephalogram (EEG). A lumbar puncture may also be informative in the overall diagnosis.

There are no known medications or supplements that decrease risk of Alzheimer's. Additionally, no known treatments stop or reverse Alzheimer's progression, although some may temporarily improve symptoms.

This invention is further illustrated by the following examples, which should not be construed as limiting. The contents of all references, patents, and published patent applications cited throughout this application, as well as the figures, are incorporated herein by reference.

### EXAMPLES

### Example 1: Characterization of BMPS by Expression of Neural Specific Genes During Differentiation

According to the techniques herein, the BMPS model established herein follows a stepwise differentiation protocol (FIG. 1A). In the final step, cells were differentiated into various neuronal and glial cell types during constant gyratory shaking. Briefly, the BMPS were established as follows: cells were differentiated, by addition of B27, GDNF and BDNF and withdrawal of stempro, basic FGF and EGF, into different neuronal and glial cell types with CNS functions during constant gyratory shaking. Advantageously, the techniques herein provide that the BMPS that were produced were of a spherical shape and a consistent size. For example, the BMPS showed spherical shapes and controlled sizes that were below 350 μm after 17 days in culture, a size that avoids necrosis in the center of the aggregate (FIG. 1B) that occurs in larger spheroids (e.g., >350 μm) due to nutrient and oxygen deprivation. Nutrient and oxygen deprivation-induced necrosis could produce artifacts in the different endpoints measured, especially in disease and toxicity studies. Five days after initiation of aggregation in NPC medium, spheres were on average 130±5 μm in diameter; the size increased to 300±40 μm during the first 17 days in differentiation medium. From day 17 onwards size remained constant around 310 μm. Advantageously, this technique significantly increases throughput of BMPS production by allowing simultaneous production of several batches with different conditions. Without the shaking condition, aggregates tend to stick together, grow in different shapes, attach to the bottom and in some point get necrotic in the middle of the sphere. Thus, constant gyratory shaking technology is a suitable method to control the shape and size of BMPS.

In order to characterize different stages of the differentiation and maturation process, BMPS were collected every week up to 8 weeks of differentiation (FIGS. 1C1-C5). Analysis of different neuronal and glial cell-specific genes by real-time reverse transcription polymerase chain reaction (RT-PCR) was performed to characterize the presence of neurons, astrocytes, oligodendrocytes and neural precursor cells (NPC). NPC are self-renewing and proliferating multi-potent cells able to generate different cell types of the central nervous system. The differentiation of NPC in 3D was initiated by changing the medium to differentiation medium. Gene expression of the cell proliferation marker Ki67 decreased 95% after 2 weeks of differentiation (FIG. 1C1, proliferation and stem cell markers). The remaining Ki67 expression appears to be due to the presence of a small population of NPC and other proliferating cell types such as oligodendrocytes and astrocytes (FIG. 1C2, astroglia and oligodendroglia). Astrocyte-specific genes (S100B and GFAP) showed a constant increase after two weeks, while, differentiation of oligodendrocytes was induced later, after six weeks of differentiation as shown by OLIG2 gene expression (FIG. 1C2).

Gene expression of specific neurotransmitters or their receptors was used to characterize the identity of different neuronal populations and the differentiation patterns of the human iPSC derived BMPS (FIG. 1C4, neuronal markers; right y-axis relative quantification of GRIN1 and GABRA1; MBP, FOXA2, and SLC1A3). GRIN1 encodes the essential Glutamate [NMDA] receptor subunit zeta-1 [25] was increased at very early stages of differentiation (one week after induction of differentiation) and continued to increase up to 5 weeks when it reached a plateau (FIG. 1C4). Similarly, GAD1, a GABAergic neuronal gene marker which encodes the Glutamate decarboxylase 1, and catalyzes decarboxylation of glutamate to GABA, showed an increase in expression during the first 4 weeks of differentiation, reaching a plateau thereafter (FIG. 1C4). The expression of tyrosine hydroxylase (TH) a gene, which identifies dopaminergic neurons, was observed first after three weeks, showing delayed differentiation compared to glutamatergic neurons. The expression of TH increased constantly thereafter reaching an 86-fold increase at seven weeks compared to NPC (week 0; FIG. 1C4). GABRA1, which encodes the gamma-aminobutyric acid (GABA) receptor, showed a steady increase of expression after 2 weeks and reached its maximum increase of a 150-fold change at 8 weeks compared to week 0 (FIG. 1C4). Moreover other markers for specific part of the brain, such as ventral midbrain neuron marker LMX1A, FOXO1 and FOXA2 (Hedlund et al., 2016; Stott et al., 2013), cerebral cortex marker FOXO4, or markers for myelination CNP and MBP (Li and Richardson, 2008; Agrawal et al., 1994) and L-glutamate transport SLC1A6 (Sery et al., 2015) has been studied (FIG. 1D d). Based on the patterns of expression of neuronal genes, the iPSC-derived BMPS model closely represents the different neuronal populations of different cortical and subcortical areas of the human CNS, suggesting that some of the mechanisms implicated in the early stages of nervous system development are reflected.

To prove that BMPS can be generated from different IPCs, another healthy line (IPS IMR90) and Down syndrome line (DYP0730) were used (FIG. 1C5). Both lines were able to generate BMPS and differentiated to neurons (MAP2 marker), astrocytes (GFAP marker) and oligodendrocytes (OLIG1 marker).

### Example 2: Characterization of BMPS by Flow Cytometry Analysis Shows Neuronal Maturation of the Human Induced Pluripotent Stem Cells Over Time

In order to quantify cell populations in the iPSC-derived BMPS and verify the reproducibility between experiments and batches of the cell line (C1, CRL-2097), flow cytometry was performed using CNS-specific antibodies for identification of neural markers (Table 1). Flow cytometry allowed quantifying 60% of cells with proliferation marker (Ki67) at the NPCs stage (week 0), which was reduced during differentiation down to 9% at 2 weeks, 7% at 4 weeks and 1% at 8 weeks (FIG. 1D), indicating a fast reduction of proliferating cells after induction of differentiation. This confirms the gene expression data and indicates a fast reduction of proliferating cells after induction of differentiation. This result was confirmed by further analysis of NPC markers such as SOX1, SOX2 and Nestin. SOX1 and SOX2 are known to be involved in the maintenance of neural progenitor cell identity. The number of SOX1-, SOX2- and NES-positive (NPC marker) cells in the NPC population (week 0) was 46%, 68% and 60%, respectively. SOX1, SOX2 and NES expression was reduced dramatically with differentiation, showing very low positive populations at eight weeks (2%, 3% and 2%, respectively). This loss in the NPC population during differentiation was corroborated by Doublecortin (DCX), a microtubule-associated protein expressed in neuroblasts and immature neurons: the number of DCX-positive cells in NPC (week o) was around 30%, which reduced to 22% at two, 17% at four and 4% at eight weeks, respectively. On the other hand, the marker for mature neurons, Tuj1 (Neuron-specific class III beta-tubulin) presented the opposite pattern. Analysis showed low levels of Tuj1-positive cells at the NPC stage (week 0). The expression of this marker in the cell population increased to 70% after 2 weeks of differentiation and remained constant up to 8 weeks. These flow cytometry experiments indicate differentiation and maturation of the BMPS over time.

Quantification of the cell population in at least three independent experiments showed low variability between cultures, demonstrating the reproducibility of the system. The variation (standard deviation, SD) between experiments decreased with the cell differentiation process and was very small at the latest maturation stage (eight weeks); DCX SD 0.9%, Ki67 SD 0.2%, SOX1 SD 0.7%, SOX2 SD 1.2%, NES SD 0.7% and Tuj1 SD 9.8% (FIG. 1E). These results indicate that after eight weeks of differentiation the cellular composition is similar and shows high reproducibility between different BMPS experiments.

TABLE 1
Gene and miRNAs Taqman Assays. List of the primers used for the experiments.

| | | | | |
|---|---|---|---|---|
| Assay ID | Assay Type | Availability | Catalog Number | Assay Name |

Gene Expression Taqman Primers

| | | | | |
|---|---|---|---|---|
| Hs01060665 | TaqMan ® Gene Expression Assay | Inventoried | 4331182 | BACT |
| Hs99999901 | TaqMan ® Gene Expression Assay | Inventoried | 4331182 | 18S |
| Hs04187831 | TaqMan ® Gene Expression Assay | Inventoried | 4331182 | NES |
| Hs01032443 | TaqMan ® Gene Expression Assay | Inventoried | 4331182 | Ki67 |
| Hs01088112 | TaqMan ® Gene Expression Assay | Inventoried | 4331182 | PAX6 |
| Hs00909233 | TaqMan ® Gene Expression Assay | Inventoried | 4331182 | GFAP |
| Hs00300164 | TaqMan ® Gene Expression Assay | Inventoried | 4331182 | OLIG2 |
| Hs00902901 | TaqMan ® Gene Expression Assay | Inventoried | 4331182 | S100B |
| Hs00609557 | TaqMan ® Gene Expression Assay | Inventoried | 4331182 | GRIN1 |
| Hs00165941 | TaqMan ® Gene Expression Assay | Inventoried | 4331182 | TH |
| Hs00971228 | TaqMan ® Gene Expression Assay | Inventoried | 4331182 | GABRA1 |
| Hs01065893 | TaqMan ® Gene Expression Assay | Inventoried | 4331182 | GAD1 |
| Hs00199577 | TaqMan ® Gene Expression Assay | Inventoried | 4331182 | SYN1 |
| Hs00232429 | TaqMan ® Gene Expression Assay | Inventoried | 4331182 | TBR1 |
| Hs01003383 | TaqMan ® Gene Expression Assay | Inventoried | 4331182 | SNCA |
| Hs01003430 | TaqMan ® Gene Expression Assay | Inventoried | 4331182 | KEAP1 |
| Hs00929425 | TaqMan ® Gene Expression Assay | Inventoried | 4331182 | NDUFB1 |
| Hs01101219 | TaqMan ® Gene Expression Assay | Inventoried | 4331182 | ATP5C1 |
| Hs00919163 | TaqMan ® Gene Expression Assay | Inventoried | 4331182 | ATP50 |
| Hs00354836 | TaqMan ® Gene Expression Assay | Inventoried | 4331182 | CASP1 |
| Hs00263981 | TaqMan ® Gene Expression Assay | Inventoried | 4331182 | CNP |
| Hs01054576 | TaqMan ® Gene Expression Assay | Inventoried | 4331182 | FOXO1 |
| Hs00188193 | TaqMan ® Gene Expression Assay | Inventoried | 4331182 | SLC1A3 |
| Hs00936217 | TaqMan ® Gene Expression Assay | Inventoried | 4331182 | FOXO4 |
| Hs00892663 | TaqMan ® Gene Expression Assay | Inventoried | 4331182 | LMX1A |
| Hs00232764 | TaqMan ® Gene Expression Assay | Inventoried | 4331182 | FOXA2 |

miRNA Taqman Assays

| | | | | |
|---|---|---|---|---|
| 1182 | TaqMan ® microRNA Assay | Inventoried | 4427975 | mmu-miR-124a |
| 2216 | TaqMan ® microRNA Assay | Inventoried | 4427975 | hsa-miR-128a |
| 457 | TaqMan ® microRNA Assay | Inventoried | 4427975 | hsa-miR-132 |
| 2247 | TaqMan ® microRNA Assay | Inventoried | 4427975 | hsa-miR-133b |
| 1129 | TaqMan ® microRNA Assay | Inventoried | 4427975 | mmu-miR-137 |
| 1094 | Control miRNA Assay | Inventoried | 4427975 | RNU44 |

### Example 3: MicroRNAs as Neuronal Differentiation Markers in Human iPSC-Derived BMPS

MicroRNAs (miRNA), known as posttranscriptional regulators of developmental timing, have recently been established as markers to study the differentiation process [26]. Expression of neural-specific miRNAs showed strong induction of miRNAs involved in neurogenesis (FIG. 1C3, miRNA). mir-124, the most abundant brain miRNA, was strongly induced in the earlier stages of differentiation, then slightly down-regulated at eight weeks of differentiation. This finding correlates with previous studies, where mir-124 was shown to promote neuronal lineage commitment at earlier stages of neural stem cells specification by targeting anti-neuronal factors [26]. mir-128, a modulator of late neural differentiation, was strongly up-regulated after 5 weeks of differentiation. mir-137, the most induced miRNA over time in the system described herein, is known as a regulator of neural differentiation of embryonic stem cells (ESCs) [27]. mir-132 and mir-133b which are involved in regulation of dopaminergic neuron maturation and function, were induced in week three of differentiation, a finding which correlates with the expression pattern of TH. Moreover, mir-132 is involved in dendritic spine formation [28]. These results support the view of a coordinated mechanism of neuronal differentiation as reflected by the patterns of neuronal gene and miRNA expression and neuronal and neurotransmitter identity.

### Example 4: Characterization of Human BMPS by Immunohistochemistry and Electron Microscopy Shows Evidence of Differentiation into Mature Brain Cell Types

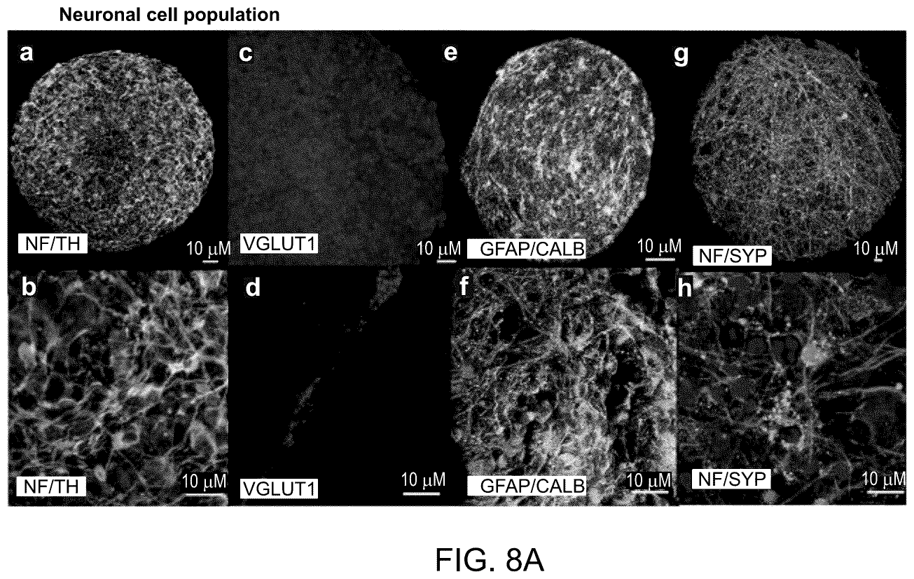

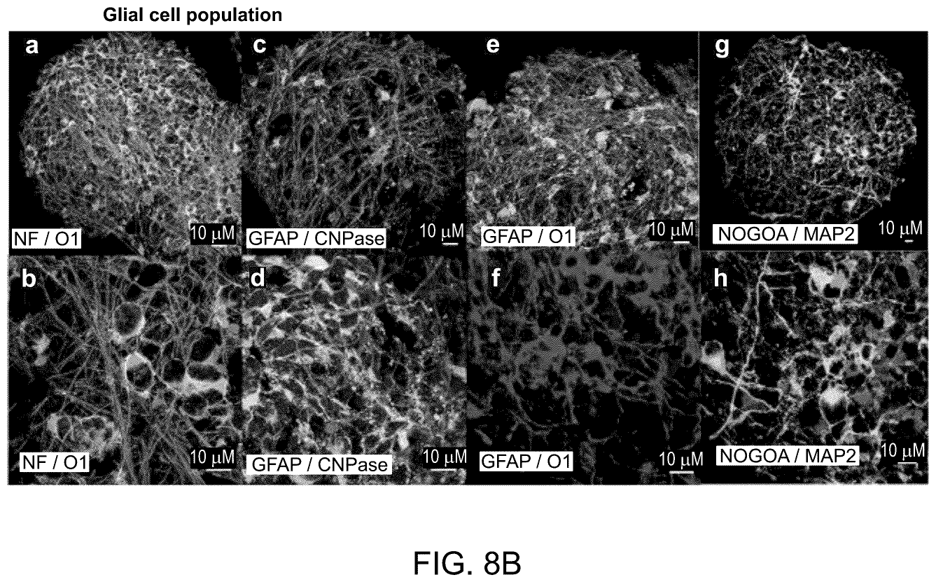

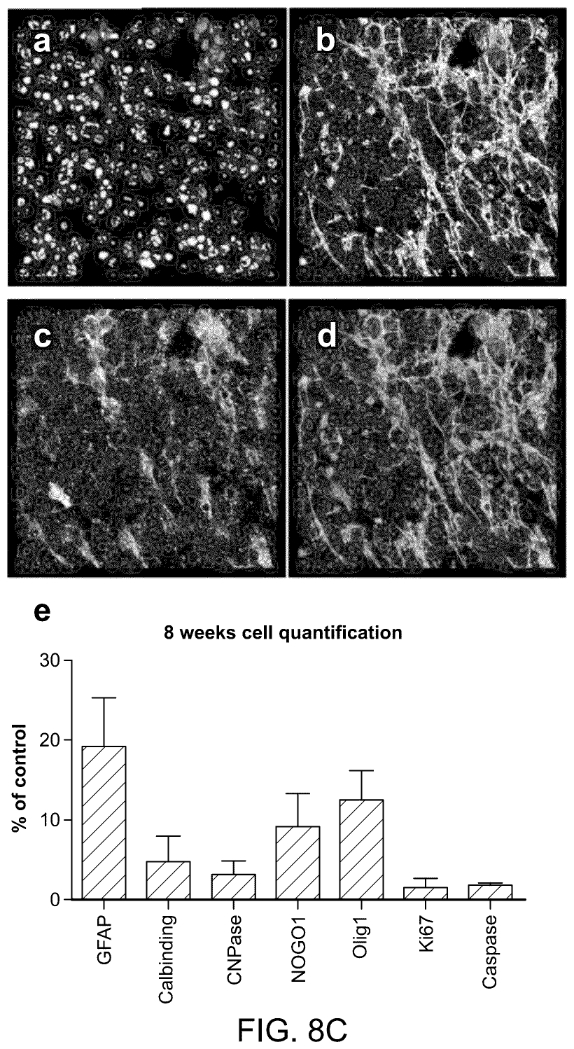

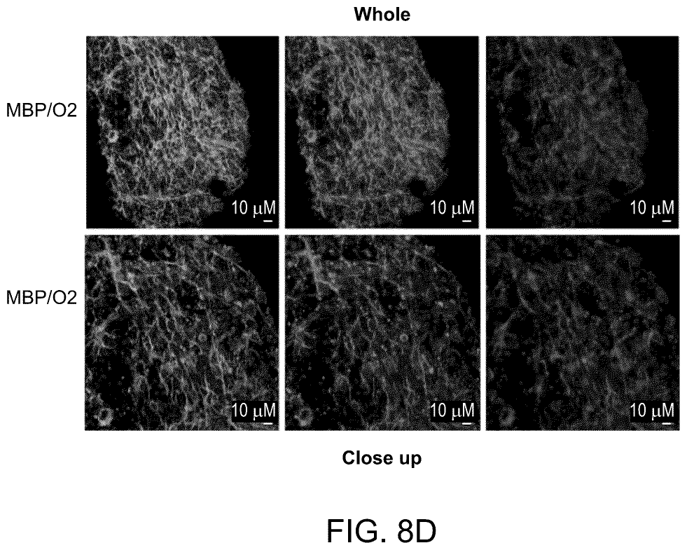

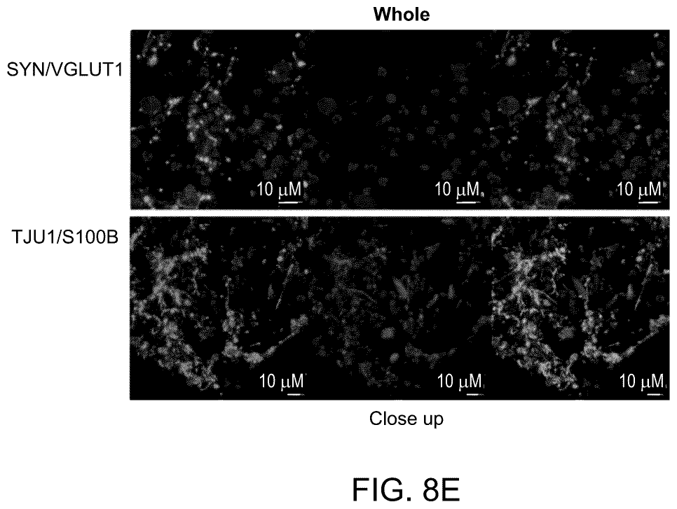

In order to assess the cellular composition and the process of maturation of the cells within the human BMPS, the expression of markers for different CNS cell populations including neurons and glial cells at 2, 4 and 8 weeks of differentiation were evaluated using immunohistochemistry and electron microscopy techniques. A reproducible pattern of expression consistent with maturation of the BMPS towards mature neural phenotypes was found. After 4 weeks of differentiation, the BMPS showed positive staining for mature neuronal markers such as microtubule-associated protein 2 (MAP2), neurofilament-heavy chain (NF, SMI32) and synaptophysin (FIG. 2A, 2B). Furthermore, different neuronal subtypes in the BMPS including dopaminergic (TH-positive neurons), glutamatergic (VGLUT1-positive neurons) and GABAergic interneurons (calbindin-positive neurons) (FIG. 2B, FIG. 8A) were observed. Moreover, the BMPS matured over time of differentiation as seen by decreased NES-positive cells (FIG. 2A) and increased cell-cell interactions (neuron-neuron and neuron glia) as subsets of neurons showed several processes, which resembled dendritic and axonal projections (FIG. 8A).

A subset of neuronal cells exhibited immunoreactivity for markers such as NOGOA, O1, O2, and CNPase (FIG. 8B, panels a-j; FIG. 1C5), which identifies the presence of mature oligodendrocytes in the BMPS [31, 33]. Automatic image quantification showed that oligodendrocytes (CNPase, NOGOA, and Olig1) comprised 3, 9, and 11% of the total cell population, respectively, at 8 weeks of differentiation (FIG. 8C; FIG. 1C5). Similar to the in vivo physiology, these cells were immunoreactive for myelin basic protein (MBP) (FIG. 2), which characterizes myelinating oligodendrocytes [32]. Moreover, they had morphological features of normal human oligodendrocytes in vivo and appeared in close contact with neuronal processes (FIG. 8a-b, FIG. 2C, 2D)

Similarly, populations of neuroglia such as astrocytes and oligodendrocytes were identified using specific antibody markers. A subset of neuroglial cells exhibit immunoreactivity for markers such as NOGOA, Olig1 and CNPase (FIG. 2C, panels a-f and 2C, panel i), which identify the presence of mature oligodendrocytes in the BMPS [29, 30, 31, 32]. This pattern of immunostaining suggests that oligodendrocytes within the BMPS are functional and myelinate axons. Similar to the in vivo physiology, these cells were also immunoreactive for myelin basic protein (MBP) (FIG. 2C panel i and 2C panel j), which characterizes myelinating oligodendrocytes [33, 30]. These cells had morphological features of normal human oligodendrocytes and appeared in close contact with neuron processes, which resemble axonal structures (FIG. 2C, panels j-m). In addition, a high number of mature astrocytes (FIG. 2Ca, 2Cb, 2Cg, 2Ch and 2F) at 4 and 8 weeks of differentiation were observed. Morphometric studies of neuronal processes identified by immunostaining with NF antibodies and MBP markers were used to estimate the percentage of myelinated axons within the BMPS with an average of 4% at 2 weeks, 25% at 4 weeks and 42% at 8 weeks of differentiation (p<0.001) (FIG. 2D). All analyzed BMPS showed similar extent of myelination at the same differentiation window. Percentages were calculated as the mean of at least 18 microscopy fields from at least 3 individual BMPS in 2 different experiments. Ultrastructural analysis by electron microscopy demonstrated cell projections, which enwrapped cell processes resembling axons after 8 weeks of differentiation (FIG. 2C).

GFAP-positive cells formed numerous cell processes organized in a network typical for human astrocyte glial processes in vivo, which established contacts with other glial cells and neurons (FIG. 2Cg, 2Ch, 2F, and FIG. 8B). Image quantification revealed 19% of astrocytes in the total population (FIG. 8C). Altogether, the patterns of cell morphology, immunostaining and cell-cell interactions shown by neuronal and glial cell populations demonstrates that the BMPS recapitulates the cellular types and pattern of interactions seen in the human CNS and is, therefore, considered organotypic.

The morphology of cell nuclei observed by immunocytochemistry and electron microscopy showed some variation in nuclear morphology attributed to (i) cell proliferation as seen by positive staining for Ki67 and Nestin markers, and (ii) nuclear fragmentation likely associated with apoptosis as indicated by caspase 3 staining (FIG. 2G, 2H) was observed. These observations were also confirmed by electron microscopy studies at 4 and 8 weeks of differentiation (FIG. 2H). The variation of nuclei morphology likely reflects the active stages of cell differentiation that BMPS exhibited during all stages of development. The presence of apoptotic nuclei likely resemble stages of cell death seen in normal neurodevelopment [34, 35]. Importantly, Caspase 3-positive nuclei did not concentrate in the center of the spheres and BMPS did not present necrosis in the center of the 3D structures (FIG. 2G). Thus, Caspase3-positive nuclei do not appear linked to deprivation of oxygen or nutrients. Caspase has been quantified at eight weeks in BMPS (FIG. 8C). Additionally, FIGS. 8D and 8E depict co-expression of mature oligodendroglia markers (MBP and O2) and expression of neuronal markers (VGLUT, TUJ1, SYN), respectively.

Further analysis of neuronal cell populations and morphology presented a pattern of evolution that suggests BMPS maturation as seen by Nestin-positive cells decreasing over time of differentiation while MBP expressing cells increased (FIG. 2A). There was also evidence of cell-cell interactions as subsets of neurons showed several processes, which resemble dendritic and axonal projections that interact with other neurons as well as glial cells (FIG. 2B, FIG. 2H). Furthermore, cells immunostained with myelin binding protein (MBP) antibodies issued projections, which appear to enwrap neuronal processes, which resemble axons (FIG. 2C, panels i-k, 2C, panel m). The pattern of immunostaining with MBP and its association with neuronal processes suggests that oligodendrocytes within the BMPS exhibit myelinating properties such as in the human CNS in vivo. Ultrastructural analysis by electron microscopy demonstrated cell projections, which enwrapped cell processes resembling axons (FIG. 2C, panel m).

### Example 5: Microelectrode Array Recording of Spontaneous Electrical Activity of BMPS

To test the neurophysiological properties of the cells within the BMPS model, spontaneous electrical activity in BMPS was analyzed by micro-electrode array (MEA) (see FIG. 3 generally). BMPS were plated in 12-well or 48-well MEA plates at 8 weeks of differentiation. The aggregates were attached to the MEAs using Matrigel coating. Spontaneous electrical activity was measured starting one week after plating up to two weeks. The activity was measured for 20 minutes on 7 different days. Electrodes were considered active when the recorded activity was above 0.05 spikes/sec. FIG. 3A shows a representative heatmap of a 48-well MEA plate measurement from one 20 minute recording. The heatmap represents the spike amplitude (V) with a minimum of 0 μV and maximum of 40 μV (FIG. 3A). The spikes showed a common waveform between different electrodes and measurements (FIG. 3B) and neurons were repeatedly firing. 25 electrodes, distributed over 19 wells, were included after the first step of data analysis. 20 to 40% of these 25 electrodes reached the threshold of 0.05 spikes/sec during each recording. FIG. 3F shows the spike events of active electrodes from one representative 20 minutes recording. These data show potential for the use of MEA to measure electrical activity of the 3D BMPS. Further optimization of the protocol may increase the measurement of the neuronal activity on the electrodes.

### Example 6: A Human 3D Model to Study Parkinson's Disease

Due to the presence of TH-positive dopaminergic neurons in the iPSC-derived BMPS (FIG. 2B, panels k, l, and FIG. 8), the possibility of using this model to study Parkinson's Disease (PD), a neurodegenerative disorder known to specifically affect dopaminergic neurons, was further explored. Two well-known neurotoxicants, which induce pathogenic processes resembling the mechanism associated with neurodegeneration in PD: the illicit drug MPTP's toxic metabolite MPP+ and the broadly used pesticide rotenone, were selected. Both MPP+ and rotenone interfere with oxidative phosphorylation in mitochondria by inhibiting complex I [36]. Initially, cytotoxicity experiments were performed to estimate sub-cytotoxic concentrations of these two compounds affecting only dopaminergic neurons (FIGS. 4A and 4C). Selective disruption of dopaminergic neurons but not of any other cell types in the systems described herein were observed with immunohistochemistry after exposure to 1 μM rotenone and 100 μM MPP+ for 24 h (FIGS. 4E and 4F). This effect was likely selective even at cytotoxic concentrations of 10 μM rotenone and 1000 μM MPP+as these concentrations did not show any alterations in other neurofilament 200-positive neurons. Lower concentrations of these compounds may induce effects in dopaminergic neurons, however, the effect was not as obvious by immunocytochemistry. Higher concentrations of rotenone and MPP+(up to 50 μM and 5000 μM, respectively) led to general cytotoxicity and affected also other neuronal types stained positive for neurofilament 200 (FIGS. 4E and F). 5 μM of rotenone and 1000 μM of MPP+ were selected for further studies as these concentrations induced clear and selective dopaminergic effects. Reactive oxygen species (ROS) were measured in the cellular medium using the OxiSelect™ In Vitro ROS/RNS Assay Kit (Cellbiolabs, San Diego, CA) as an indication of oxidative stress. Exposure to rotenone at 5 μM and MPP+ at 1000 μM showed an increase in ROS production after 24 hours exposure, while 12 hours showed no statistically significant changes. Real time RT-PCR was performed in order to determine effects of both chemicals on genes related to PD, mitochondrial dysfunction and oxidative stress. Tyrosine hydroxylase (TH, Dopaminergic neuronal marker) mRNA expression decreased by 84%±11 after exposure to 5 μM rotenone and 70%±9 after exposure to 1000 μM MPP+ for 24 hours. Additional genes related to PD also showed changes at sub-cytotoxic concentrations of MPP+ and rotenone. The expression of genes that encode T-box brain 1 (TBR1) and Alpha-synuclein (SNCA) protein decreased after 24 hours exposure. The reduction of TBR1 was 70±13% (rotenone) and 76±22% (MPP+) and the reduction of SNCA was 72±6% (rotenone) and 41±40% (MPP, however, BMPS exposed to 1 mM MPP+ led to no statistically significant changes in SNCA expression). Expression of genes related to mitochondrial function complex I (NDUFB1) or complex 0 (ATP5C1 or ATP50) tended to decrease in expression but these changes were not statistically significant. Caspase-1 gene expression, which has been related to SNCA, increased after 24 hours exposure to MPP+. These results demonstrate the potential of BMPS for studies elucidating molecular mechanisms of PD, lending itself to PD drug and neurotoxicity screening.

### Example 7: Addition of Microglia

Peripheral blood mononuclear cells (PBMCs) are isolated from fresh or commercially available cryo-preserved whole blood of pooled healthy donors by Ficoll or Percoll gradient centrifugation. Monocyte populations are obtained by negative magnet-antibody selection after Ficoll or Percoll gradient and then re-suspend in RPMI 1640. Monocytes are cultured in macrophage serum-free medium, stimulated with a cocktail of cytokines, GM-CSF and IL-34. Monocytes may also be obtained by differentiation of iPSCs, hematopoetic or other stem cells. The microglia-like cells are combined with neuronal precursor cells in shaker cultures to preferably arrive at a final concentration of 5-8% microglia.

Primary monocytes or iPSC-derived monocytes may be incorporated into the system, both at early and later stages of BMPS differentiation. For the early stages, a number of 2×10⁶ NPCs mixed with 2×10⁴ monocytes are plated per 1 well (6 well-plate). Gyratory shaking is used at 88 rpms to generate spheres. After 2 days media are replaced with ½ CNS differentiation medial (Neurobasal® electro Medium (Gibco) supplemented with 5% B-27® Electrophysiology (Gibco), 1% glutamax (Gibco), 10 μg human recombinant GDNF (Gemini), 10 μg human recombinant BDNF (Gemini)) and ½ macrophage differentiation media (Dulbecco's modified Eagle's medium (Invitrogen) supplemented with 10% FCS, 0.055 mM β-mercaptoethanol, M-CSF (50 ng/ml), and IL-3 (25 ng/ml) (R&D Systems). The medium is replaced every 3 days.

Monocytes can also be incorporated after BMPS differentiation. For that, BMPS are differentiated up to 8 weeks. BMPS spheres are separated in 500 μl Eppendorf tubes. 2×10⁴ monocytes are added to the Eppendorf with the BMPS. Tubes are shaking manually every hour, up to 8 hours. After that, BMPS-monocytes are collected and plated in 6 well plates. Cells are kept on constant shaking until use.

The characterization of the immune-competent human organoids can be carried out by immunocytochemically assessing the presence of markers such as HLA-DR, and the ionized calcium-binding adapter molecule 1 (Iba1), specific microglial markers. Measures of cytokines and chemokines release and expression of receptors associated with microglia function (e.g., CCL2 and CX3CL) demonstrates successful engrafting of the microglia cells. This modified model is more suitable to investigate the neuroimmunological component associated with many substance exposures and diseases.

### Example 8: Addition of a Blood Brain Barrier

The blood brain barrier (BBB) has a crucial role in neurotoxicity, being the last barrier for substances before reaching the brain. Moreover, the BBB is the bottleneck in brain drug development and is the single most important factor limiting the future growth of neurotherapeutics [81]. Most of the in vitro models do not incorporate BBB.

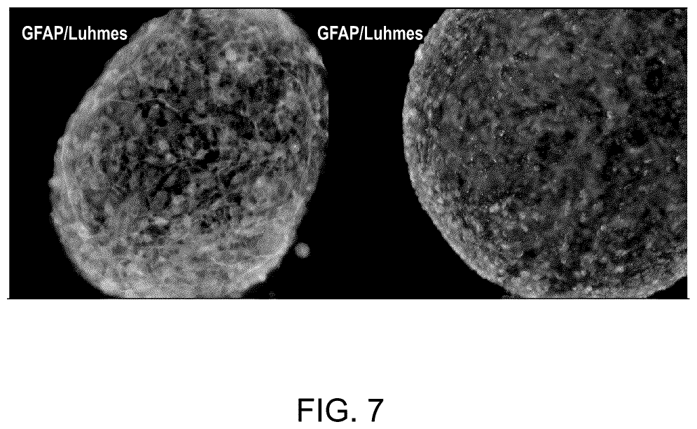

Human brain microvascular endothelial cells (hBMECs) from human iPSCs are incorporated into the BMPS by two techniques. In the first approach, mature BBB endothelial cells and neuronal precursors cells (NPCs) are combined in a single cells suspension in a ratio of 1:5, gyratory shaking or stirring are used to generated spheroids and aggregates are cultured up to 8 weeks. In the second technique, mature BMPS (8 weeks of differentiation) are covered by BBB endothelial cells using gravity systems (aggrewell, gravity well or hanging drops). Cells may be covered as well with other cell types, such as fluorescent LUHMES cells (FIG. 7).

### Example 9: Addition of Reporters

The BMPS gives the opportunity to develop cell-based assays allowing for high-content imaging (HCI) that can be adapted to high-throughput platforms, to evaluate the effects of toxicants on key cellular processes of neural development and physiology in the culture system.

Example of establishing fluorescent iPSC cell line: Creation of reporter cells lines greatly assists imaging efforts by allowing us to avoid complications associated with staining 3D cultures, to image subsets of cells, and to perform functional assays. Differentiated 3D aggregates from iPSC cultures spiked with 1-2% of iPSCs ubiquitously expressing fluorescent protein allow visualizing individual cells within the aggregates aiding quantification of phenotypic parameters, including neurite outgrowth and migration. Lines expressing markers allow measurement of synapse formation (PSD95, Synapsin 1), proliferation (Ki67), glial maturation (GFAP), and calcium signaling (GCaMP). Clustered Regularly Interspaced Short Palindromic Repeats/Cas (CRISPR) were used to create the various lines. Similar in function to the well-established zinc-finger (ZFNs) and TALEN nucleases, the Cas9-CRISPR system is a new entrant into the rapidly emerging field of genome engineering and has been quickly adopted and validated across a wide array of human stem cells. Gene-editing in hiPSCs has traditionally been a technically difficult task but with these advances it is now possible to generate reporter and mutant cell lines with genetically matched controls [83, 84, 85, 86]; essential tools not only for this project but also for the future success of using human iPSC-derived cells in quantitative live-cell phenotypic assays of toxicant testing.

Using the CRISPR-Cas9 system, fluorescent protein (FxP) reporter cell lines were generated by generating gRNAs targeting the gene of interested. In this system as described herein, an RNA guided Cas9 endonuclease is used in conjunction with customizable small guide RNAs (gRNAs) to target and cleave any DNA template with a GN21GG sequence; the first G is for the U6 polymerase promoter while the N21GG is for the protospacer adjacent motif (PAM) sequence requirement of Cas9 [86, 87, 89].

For reporter cell generation, homology-directed repair (HDR) guides the insertion of the appropriate DNA donor fragment into a target site at regions of homology between the donor fragment and the genomic DNA target. An ES line that ubiquitously expresses GFP was created by introducing CAG promoter-driven GFP into the AAVS1 safe harbor locus, and can use these constructs to transfect iPSC cells. For other reporters, constructs may be created that will direct the integration of a self-cleaving P2A peptide sequence [90] targeted fluorescent protein cassette in frame at the stop codon of the gene of interest. The P2A sequence engineered between the C-terminus of the endogenous protein and the fluorescent protein may minimize possible fusion protein functional defects. Plasmids encoding the Cas9 nuclease, the targeting gRNA, and appropriate donor DNA will be introduced by electroporation, recombinant hiPSC clones will be manually selected and screened for the desired insertion by PCR, and the genotype may be verified by sequencing. Reporter hiPSCs will be subjected to a differentiation protocol and expression of the reporter validated by examining expression patterns and through immunohistochemistry experiments where it may be determined whether the FxP expressing cells co-label with known markers.

### Example 10: Using Cells with Specific Genetic Backgrounds

The use of iPSCs, as described herein, has created new opportunities to study human diseases and gene/environment interaction [20, 21]. Fibroblasts or other somatic cells from healthy and diseased individuals can be reprogrammed into iPSCs, and subsequently be differentiated into all neural cell types. Similarly, iPSC can be genetically modified before creating the BMPS. As a proof-of-principle, iPSCs were obtained from patients with Down's syndrome (FIGS. 1C5 and 5A-D), Rett Syndrome and from individuals with mutations in disrupted in schizophrenia 1 (DISC1). DISC1 may have some functional overlap with TSC-iPSCs as both are involved in the mTOR cell signaling pathway.

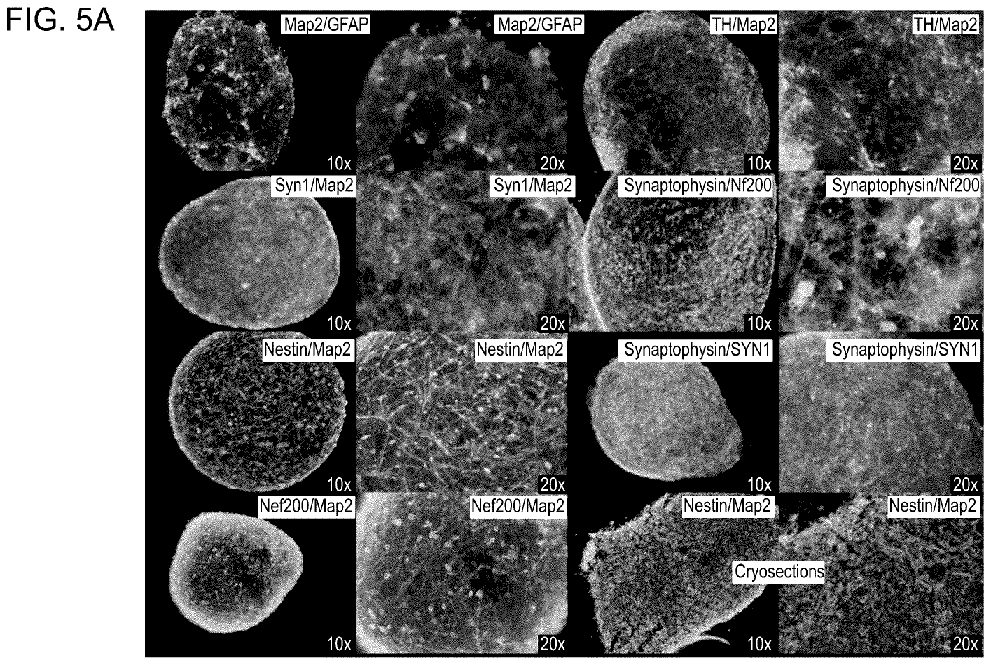

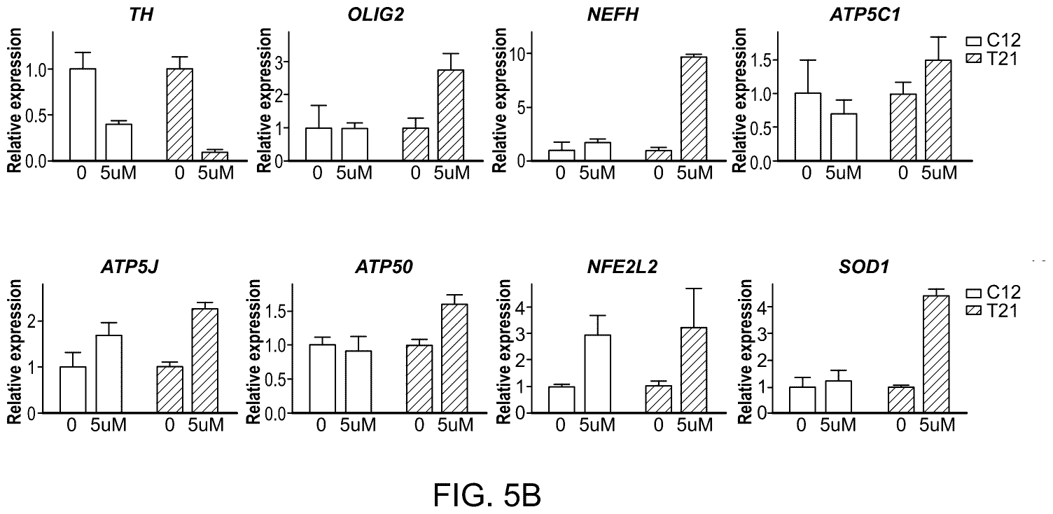

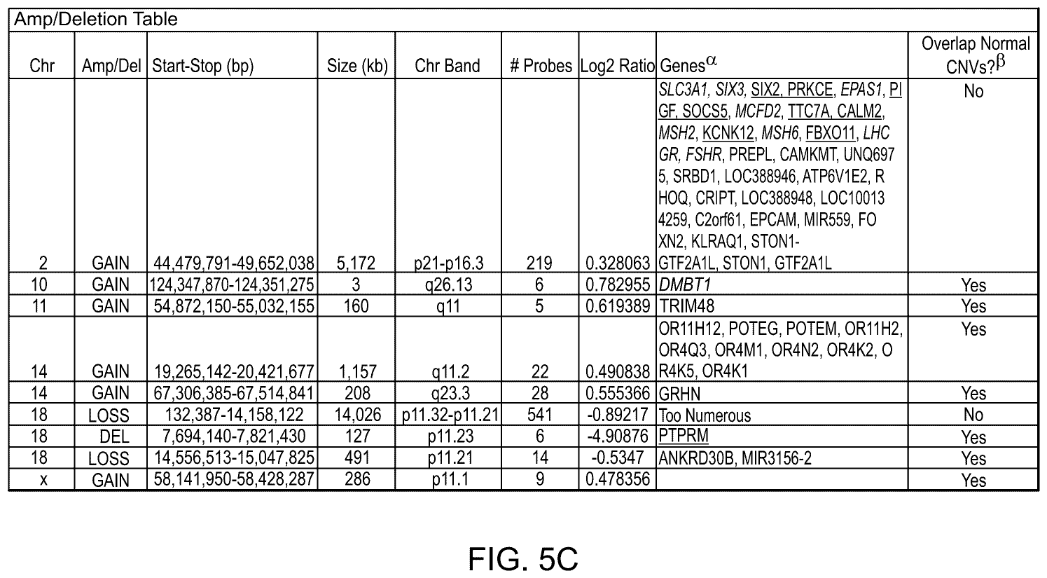

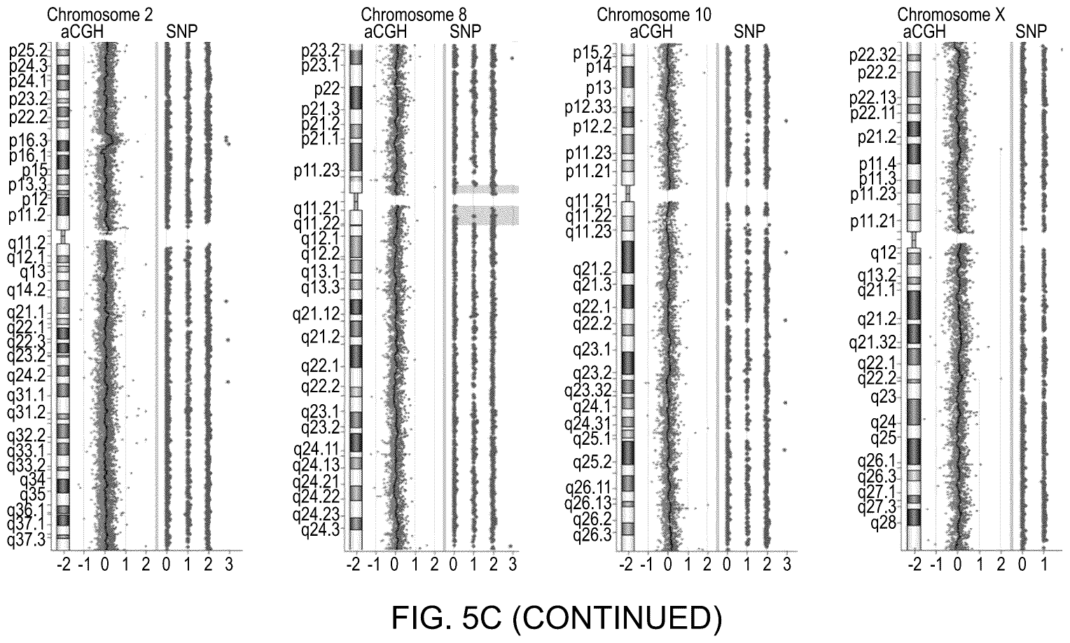

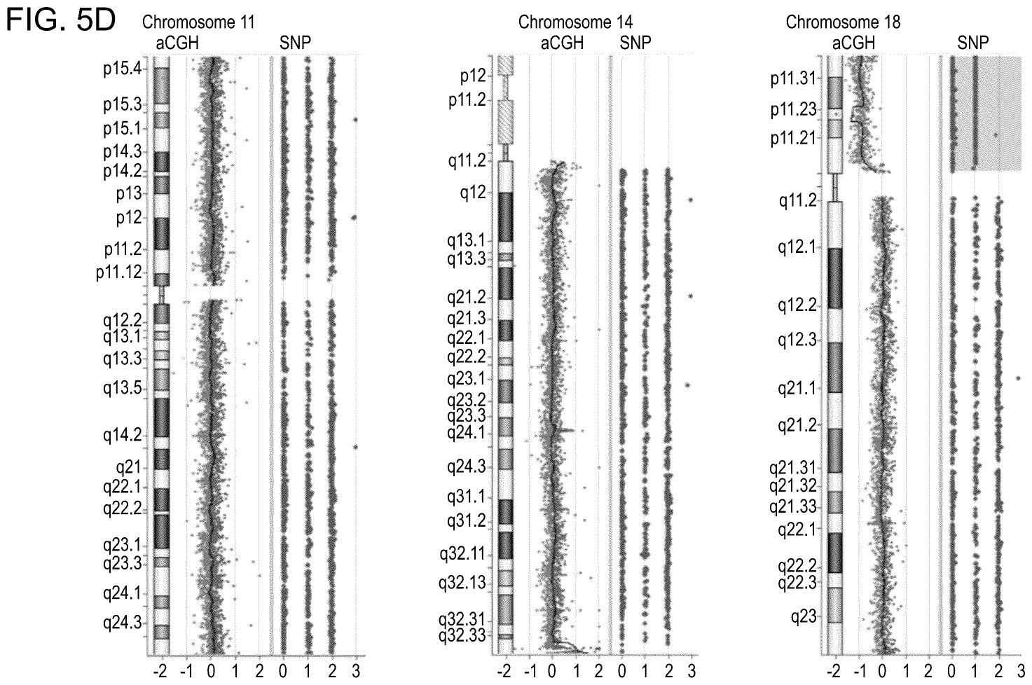

The Down's syndrome model is further characterized (see FIGS. 5A-5D). Down's syndrome iPSCs have been successfully differentiated into neural precursor cells (NPCs). Currently the cells are differentiated in 3D and characterization by gene expression and immunohistochemistry is being performed. The Down's syndrome model has been exposed to compounds that induce oxidative stress (rotenone and paraquat). The response was compared to the model from healthy donors, which were more sensitive to these compounds than the healthy model.

### Example 11: Combining the BMPS with Other Organoids

In some embodiments, BMPS may be combined with other organs and/or organ model systems. Several groups have been developing organ-on-a-chip platforms for different organs by using microfluidic techniques. Those platforms are designed to mimic in-vivo fluidic flows in the organs by separating cell culture chambers and perfusion channels, and successfully demonstrate recapitulation of iPSC-based organ functions. Together with other organ models on these platforms, the BMPS can be integrated, which allow us to untwine the complex toxicology from organ interactions. Such platforms allow (1) in-situ and high-throughput production of mini-brains on chip, (2) in-vivo like fluidic flow around mini-brains with enough supply of nutrient and small molecule through diffusion, (3) a large number of parallel test of toxic materials, and (4) a real-time monitoring of electrophysiological activities from BMPS with integrated electrodes. Companies such as TissUse GmbH have designed microfluidics platform that allow culture of floating spheres like the BMPS as described herein.

### Example 12: Cryopreservation and Other Modes of Transportability

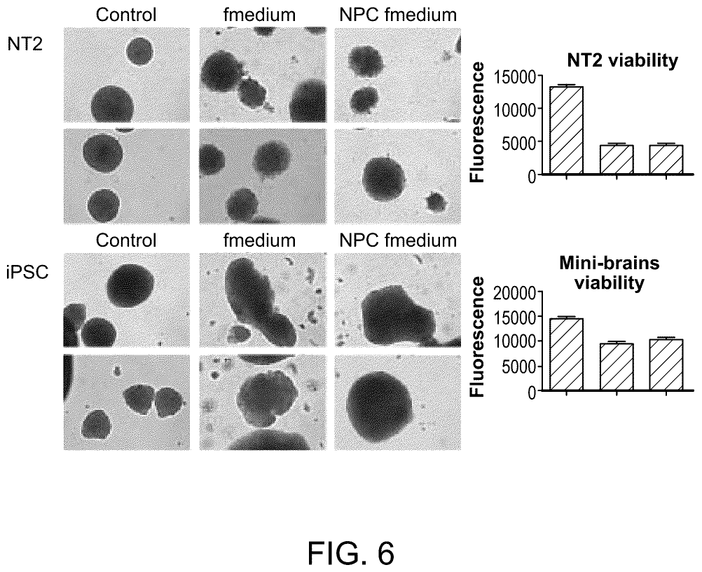

In order to e.g. incorporate the BMPS into platforms or enable any use in other laboratories, transportability of the system was optimized. Preliminary studies have shown possible recovery of the neuronal 3D aggregates after cryopreservation (FIG. 6). A human embryonal carcinoma stem cell line, (hNT2), and iPSC derived-aggregates were differentiated into mature neurons (8 weeks of differentiation for each cell line) and then cryopreserved with regular cryopreservation medium (95% FBS and 5% DMSO) or STEMdiff™ Neural Progenitor Freezing Medium (Stem cells technologies). After 2 days in liquid nitrogen, cells were thawed. Freezing media was removed and fresh media was added. One day later, viability was measured using the resazurin cell viability assay. hNT2 aggregates presented a 70% decrease in viability in both freezing medias while iPSC derived mini-brains showed a 20%-35% reduction in viability (FIG. 6). However, viability recovery of the 3D aggregates is currently optimized using other viability and functional assays. Optimization of this protocol will vary additives (DMSO, HES, glycerol, serum etc.), the cooling temperature gradient as well as thawing protocol.

Human iPSC derived mini-brains are kept in culture at 37° C. In order to transport the live mini-brains, temperature must be controlled. Different methods can be used to control temperature during transport. Heating pads combined with an insulated box have been used to transport live biological material. Disposable chemical pads employ a one-time exothermic chemical reaction such as catalyzed rusting of iron, or dissolving calcium chloride. The most common reusable heat pads are based on a chemical reaction that transforms a liquid into a solid thus releasing energy. Some new heating pads (such as Deltaphase Isothermal Pad 3SET, from Braintree Scientific, Inc.) have been able to maintain 37° C. for more than 6 hours. 3D mini-brains cultured up to 8 weeks are sent in an insulated material box with heating pads. After transport, viability may be measured.

### Example 13: Overview

The techniques herein provide a human BMPS model that is a versatile tool for more complex testing platforms, as well as for research into CNS physiology, mechanisms associated with (developmental) neurotoxicity, and pathogenesis of neurological disorders. Prior art stem cell-derived brain model systems developed in the past few years have shown the capability to recapitulate some of the in vivo biological processes (Juraver-Geslin and Durand, 2015; Nakano et al., 2012; Krug et al., 2014) and have an advantage over other classical in vitro models as they facilitate the study of various differentiation mechanisms, developmental processes and diseases (Lancaster et al., 2013). Unfortunately, these prior art systems require complicated protocols that reduce the reproducibility of the system and make it difficult to use in other fields such as chemical toxicity and drug screening. Additionally, these prior art models are also limited by large diameters, which lead to extensive cell death in the interior regions due to insufficient diffusion of oxygen and nutrients (Lancaster et al., 2013) and other artifacts.

The techniques herein overcome the limitations of the prior art by developing a human in vitro model by the gyratory shaking technique that enables reliably generation of a high number (about 500 per six-well plate) of viable BMPS that are homogeneous in size and shape. Control of size makes it possible to keep cell aggregates below 350 μM in diameter (FIG. 1C) and thereby avoid disparate morphology and/or necrosis in the center of the spheres. Moreover, the BMPS showed reproducible cell composition by immunomorphological quantification, assessment of imaging-based endpoints and flow cytometry analysis.

As described herein, the 3D differentiation protocol for the BMPS covers stages from neuronal precursors to different cell types of the mature CNS. As discussed in detail above, at two weeks, BMPS consisted of an immature population of cells, showing minimal neuronal networks, a low percentage of mature astrocytes and oligodendrocytes, and minimal but early stages of myelin basic protein (MBP) expression. iPSC differentiation into mature BMPS was indicated by decreasing NES expression over time and a progressive expression of mature neuronal and glial markers such as MAP2, GFAP, O1 and MBP. Gene expression studies, flow cytometry, image analysis, immunostaining and miRNA studies have shown increase of cell maturation markers, which follow the BMPS differentiation. The presence of GABAergic neurons, dopaminergic neurons and glutamatergic neurons was documented by immunohistochemistry and real-time PCR data. Moreover, the BMPS showed spontaneous electrical activity, indicating neuronal functionality of the system.

Since astrocytes and oligodendrocytes play important roles during neuronal development, plasticity and injury, the presence of glial cell populations in the presently disclosed BMPS model provides an excellent opportunity for the evaluation of neuronal-glial interactions and the role of glia in pathogenesis and toxicity processes. Astrocytes have an important role in protecting neurons, increasing neuronal viability and mitochondrial biogenesis from both exogenous (e.g. chemicals) and endogenous toxicity (Shinozaki et al., 2014; Aguirre-Rueda et al., 2015), especially against oxidative stress (Shao et al., 1997; Schwab and McGeer, 2008). Thus, their presence in a biological system to study disease and neurotoxicity is crucial. Immunohistochemistry and RT-PCR results showed increasing numbers of astrocytes (GFAP-positive cells) in the BMPS model reaching 19% astrocytes of the total cell population at eight weeks, which is earlier than in previously described cortical spheroids, where similar proportions of GFAP-positive cells were observed first at day 181, at day 86 the number of GFAP+ cells was below 10% (Pasca et al., 2015).

The most novel element of this BMPS is the presence of mature human oligodendrocytes with myelination properties, which has not been achieved in the prior art. Immunocytochemical and ultrastructural studies confirmed the morphological identity of these cells (FIG. 2D) as multiple markers for mature oligodendrocytes were expressed by rounded cells with branching processes and membrane sheaths that are similar to the ones found in humans in vivo. The structure and morphology was further confirmed by electron microscopy. Quantitative assessment of the myelination process of MBP immunostaining along axons showed an increase over time of differentiation reaching 42% of myelinated axons at eight weeks (FIG. 2D). 3D reconstruction of confocal z-stacks images (FIG. 2A) and electron microscopy confirmed the wrapping of axonal structures after eight weeks of differentiation (FIG. 2C). These findings are of particular relevance since myelin is a critical element for proper neuronal function and development, and the covering of axons by myelin allows faster action potential transmission, reduces axonal energy consumption and protects the axons from degeneration (Nave, 2010). Furthermore, recent evidence suggests that oligodendrocytes and myelin have a role in the metabolic support of axons independent of their role in action potential conduction, highlighting their importance in neuronal survival (Saab et al., 2013). This is the first time that a 3D human microphysiological system, consisting of different types of neurons and glial cells, has achieved such a high percentage of myelination. The ability to assess oligodendroglia function and mechanisms associated with myelination in this BMPS model provides an excellent tool for future studies of neurological disorders such as multiple sclerosis and other demyelinating disorders. As an illustration it was recently discovered that astroglia cells could promote oligodendrogenesis via secreted molecules (Jiang et al., 2016). A human BMPS that consist of neurons, astrocytes and oligodendrocytes is essential to evaluate this mechanism further and to develop a potential therapy for demyelinating disorders.

In conclusion, the techniques herein provide a BMPS that replicates crucial aspects of brain physiology and functionality. The potential for studying developmental and neurodegenerative disorders, brain infections, toxicity and trauma with such a system is growing. Furthermore, the potential to use iPSCs from different donors adds a personalized component to these studies. The high reproducibility and relatively simple protocol, enables future medium-throughput (96-well format) testing of chemicals, drugs and their potential to induce or treat diseases.

### Methods and Materials

### Chemicals

Rotenone and MPP+ were supplied from Sigma-Aldrich (St. Louis, MO). A 10 mM rotenone stock was prepared in DMSO Hybri-Max (Sigma) while MPP+ was diluted in water to a concentration of 30 mM.

iPSC Generation

CCD1079Sk (ATCC® CRL2097™), IPS IMR90 (WiCELL) and ATCCDYP0730 Human (IPS) Cells (ATCC® ACS1003™) fibroblasts were originally purchased from ATCC. All studies followed institutional IRB protocols approved by the Johns Hopkins University School of Medicine. Human fibroblasts and mouse embryonic fibroblasts (MEFs) were cultured in Dulbecco's modified Eagle's medium (DMEM, Mediatech Inc.) supplemented with 10% fetal bovine serum (FBS, HyClone) and 2 mM L-glutamine (Invitrogen). MEFs were derived from E13.5 CF-1 mouse embryos. Human iPCS cells were generated with the EBV-based vectors as previously described [75]. iPSC from other sources were used as well. Colonies of iPSCs were manually picked after 3-6 weeks for further expansion and characterization. iPSCs (passage ≤20) were cultured on irradiated MEFs in human embryonic stem cell (hESC) medium comprising D-MEM/F12 (Invitrogen), 20% Knockout Serum Replacement (KSR, Invitrogen), 2 mM L-glutamine (Invitrogen), 100 μM MEM NEAA (Invitrogen), 100 μM β-mercaptoethanol (Invitrogen), and 10 ng/mL human basic FGF (bFGF, PeproTech). Media were changed daily and iPSC lines were passaged using collagenase (Invitrogen, 1 mg/ml in D-MEM/F12 for 1 hr at 37° C.). These iPSC lines have been previously fully characterized [75].

### Neuronal Progenitor Cells (NPC) Production

NPC generated followed the previous published protocol [75]. Briefly, iPSCs colonies were detached from the feeder layer with collagenase (1 mg/ml) treatment for 1 hr and suspended in EB medium, comprising of FGF-2-free hESC medium supplemented with Dorsomorphin (2 μM) and A-83 (2 μM), in non-treated polystyrene plates for 4 days with a daily medium change. After 4 days, EB medium was replaced by neural induction medium (hNPC medium) comprising of DMEM/F12, N2 supplement, NEAA, heparin (2 μg/ml) for 15 more days. The floating neurospheres were then dissociated to single cells in Accutase and plated in 175 mm flasks and were allowed to expand for 7 days. NPCs were expanded in poly-1-ornithine and laminin-coated 175 mm flask on StemPro® NSC SFM (Life Technologies). Half of the media was changed every day. Cultures were maintained at 37° C. in an atmosphere of 5% CO₂. After NPC generation, iPSCs colonies were detached and NPCs were expanded in poly-1-ornithine and laminin-coated 175 mm flask in StemPro® NSC SFM (Life Technologies). Half of the media was changed every day. Cultures were maintained at 37° C. in an atmosphere of 5% CO2.

### BMPS Differentiation

At 100% confluence NPCs were detached mechanically and counted. 2×10⁶ cells per well were plated in 2 ml of medium in non-treated 6 well-plates. Cells were grown in NPC media for two days under constant gyratory shaking. Subsequently, medium was changed to differentiation medium (Neurobasal® electro Medium (Gibco) supplemented with 5% B-27® Electrophysiology (Gibco), 1% glutamax (Gibco), 0.02 μg/ml human recombinant GDNF (Gemini), 0.02 μg/ml human recombinant BDNF (Gemini)). Cultures were maintained at 37° C., 5% CO₂ under constant gyratory shaking for up to 8 weeks. Differentiation medium was routinely changed every 2 days.

### Size Measurement

Aggregates (n=20) from 3 independent experiments were randomly selected per time point for obtaining pictures and measuring size using SPOT software 5.0. Results were expressed as mean±SD. Cells were kept two days in NPC medium, indicated as NPC med. 2d in FIG. 1B.

RNA and miRNA Extraction

Total RNA was extracted from aggregates every week up to 8 weeks of differentiation using Tripure (Roche, Switzerland) according to Chomczynski and Sacchi (1987) [76]. The same RNA extraction method was used to isolate RNA after toxicant treatment. RNA quantity and purity was determined using NanoDrop 2000c (Thermo Scientific). One microgram of RNA was reverse-transcribed using the M-MI V Promega Reverse Transcriptase (Promega) according to the manufacturer's recommendations. For miRNA reverse-transcription 60 ng of RNA were reverse transcribed using TaqMan microRNA Reverse transcription kit in combination with miRNA specific stem-loop primers, which are a part of TaqMAn microRNA expression assay. Upto eight stem-loop primers were multiplexed in one reaction.

### Quantitative RT-PCR

The expression of genes was evaluated using specific Taqman® gene expression assays (Life Technologies). miRNA expression was analyzed using TaqMAn microRNA expression assay in combination with TaqMan miRNA Reverse Transcription kit using protocol described in [77]. Table 1 shows a summary of the genes assayed. Real time RT-PCRs were performed using a 7500 Fast Real Time system machine (Applied Biosystems). Fold changes were calculated using the 2(−ΔΔCt) method [78]. β-actin and 18 s were used as a housekeeping genes for mRNA and RNU44 for miRNA. There were no statistically significant differences in expression for β-actin, 18 s, and RNU44. Data were presented as mean±SD, normalized to housekeeping genes and week 0.

### Immunocytochemistry of the BMPS

BMPS aggregates were collected at 2, 4 and 8 weeks. BMPS were fixed in 4% paraformaldehyde for 1 hour, washed 3 times in PBS, then incubated for 1 hour in blocking solution consisting of 5% normal goat serum (NGS) in PBS with 0.4% TritonX (Sigma). BMPS were then incubated at 4° C. for 48 hours with a combination of primary antibodies (Table 2) diluted in PBS containing 3% NGS and 0.1% TritonX. BMPS were washed in PBS 3 times after which they were incubated with the appropriate fluorophore-tagged secondary antibody for 1 hour in PBS with 3% NGS at room temperature. Double immunostaining was visualized using the proper combination of secondary antibodies (e.g., goat anti-rabbit secondary antibody conjugated with Alexa 594 and goat ant-mouse secondary antibody conjugated with Alexa 488 (Molecular Probes). Nuclei were counterstained with DRAQ5 dye (Cell Signaling; 1:5000 in 1×PBS) or NucRed Live (Molecular Probes) for 15 minutes before mounted on slides with coverslips and Prolong Gold antifade reagent (Molecular Probes); BMPS used as negative controls for immunostaining were processed omitting the primary antibody. Images were taken using a Zeiss UV-LSM 510 confocal microscope. The experiments were performed in duplicates; at least three different fields of view were analyzed for each combination of antibodies. 3D reconstruction was done using Imaris 7.6.4 software for scientific imaging.

TABLE 2
Primary Antibodies.

| Antibody | Host | Type | Source | Dilution |
|---|---|---|---|---|
| NF-H | Rabbit | Polyclonal | Enzo | 1:1000 |
| GFAP | Rabbit | Polyclonal | Dako | 1:500 |
| Olig 1 | Mouse | Monoclonal | Millipore | 1:500 |
| CNPase | Mouse | Monoclonal | Millipore | 1:500 |
| Calbindin | Mouse | Monoclonal | SIGMA | 1:500 |
| NOGO-A | Rabbit | Polyclonal | Santa Cruz | 1:500 |
| Map2 | Mouse | Monoclonal | Chemicon | 1:1000 |
| MBP/SMI99 | Mouse | Monoclonal | COVANCE | 1:1000 |
| SMI-32 | Mouse | Monoclonal | Stenberger | 1:2000 |
| | | | Monoclonals | |
| Synaptophysin | Mouse | Monoclonal | SIGMA | 1:500 |
| VGLUT1 | Rabbit | Polyclonal | Alpha Diagnostic | 1:500 |
| TH | Mouse | Monoclonal | Millipore | 1:250 |
| Nestin | Rabbit | Polyclonal | Millipore | 1:200 |
| Ki67 | Rabbit | Polyclonal | abcam | 1:100 |
| Caspase3 | Rabbit | Polyclonal | R&D | 0.2 μg/ml |
| OLIG1 | Mouse | Monoclonal | Millipore | 1:200 |
| TUJ1 | Mouse | Monoclonal | Stemcell | 1:200 |
| | | | technologies | |
| S100B | Rabbit | Polyclonal | Santa Cruz | 1:200 |

### Automated Quantitation of Cell Types

BMPS was differentiated for 8 weeks. Randomly selected pictures from three experiments were acquired by confocal imaging and then analyzed with a custom algorithm created with the Cellomics TargetActivation (Thermo Fisher Scientific, Pittsburgh, PA) image-analysis software package. With this algorithm, cells were identified based on DRAQ5 stained nucleus and quantified oligodendrocytes and astrocytes based on staining of CNPase, NOGO1 and GFAP.

### Myelination Assessment and Quantification

To calculate the percentage of axonal myelination, a semi-automated computer platform was used, termed computer-assisted evaluation of myelin formation (CEM) [82], which uses NIH Image J built-in tools as well as a Math lab processing functions. The results were generated as pixel counts and percent values. The percent of myelinated axons was calculated by dividing the pixel count for myelin by the pixel count for axons after cell body removal and multiplying by 100. For each time point at least 18 fields from at least two independent experiments were analyzed.

### Electron Microscopy

BMPS aggregates were collected at 2, 4 and 8 weeks and were fixed in 2% glutaraldehyde and 4% formaldehyde in 0.1M Sodium Cacodylate buffer (EMS, electron microscopy sciences) pH 7.4 with 3% sucrose and 3 mM CaCl₂. Post-fixation was done with 2% osmium for 2 hours. The BMPS aggregates were then stained en bloc with 2% uranyl acetate in distilled water for 30 min and subsequently dehydrated in graded ethanol. Embed 812 (EMS) was used as the embedding media. Thin sections (70-80 nm) were cut on a Reichert Jung Ultracut E microtome and placed on formvar coated 100 mesh copper grids. The grids were stained with uranyl acetate and followed by lead citrate. All imaging was performed on a Zeiss Libra 120 electron microscope with a Veleta (Olympus) camera.

### Treatment and Cytotoxicity Assay

BMPS was exposed to different concentrations of rotenone and MPP+ for 24 and 48 hours after 4 weeks of differentiation. Rotenone working solutions were prepared in differentiation medium from 10 nM or 100 μM stocks to reach final concentrations of 0.1, 1, 10, 25 and 50 μM. DMSO was used as vehicle control. MPP+ working solutions were prepared in differentiation medium from 30 mM stocks to reach final concentrations of 10, 50, 100, 500, 1,000, 5,000 and 10,000 μM. Four independent experiments in 3 replicates were performed for each experimental condition (control and toxicant exposure for the different time points). Resazurin reduction assay was performed in order to determine cell viability after rotenone and MPP+ treatment. Resazurin (7-Hydroxy-3H-phenoxazin-3-one 10-oxide) is a blue dye that is reduced into red fluorescent resorufin by redox reactions in viable cells. 100 μl Resazurin (2 mg/ml stock) were added directly to the 6 well plates (2 ml/well). Plates were incubated for 3 h at 37° C., 5% CO₂. Subsequently, 50 μl of medium were transferred from each well in triplicates to a 96-well plate and fluorescence was measured at 530 nm/590 nm (excitation/emission) using a multi-well fluorometric reader CytoFluor series 4000 (PerSeptive Biosystems, Inc). Data were presented as mean±SD. Statistical analysis was performed using Dunnett's test.

### Reactive Oxygen Species Measurement

Reactive oxygen species (ROS) were measured in cell media collected 24 hours after treatment with 5 μM rotenone or 1,000 μM MPP+ using the OxiSelect™ In Vitro ROS/RNS Assay Kit (Cell Biolabs, San Diego, CA). This is a fluorescence-based assay measuring the presence of total free radicals within a sample and was used according to the manufacturer's protocol. The quenched fluorogenic dye dichlorodihydrofluorescin-DiOxyQ (DCFH-DiOxyQ) which is similar to the popular 2′,7′-dichlorodihydrofluorescein diacetate (DCFH-DA) is first primed with a quench removal reagent. The resulted highly reactive non-fluorescent DCFH can react with present ROS species in the cell supernatant and is then oxidized to the highly fluorescent DCF (2′,7′-dichlorodihydroxyfluorescein). At every time point, 50 μl of the cell supernatant was added to a 96-well plate in triplicates and was mixed and incubated with the DCFH-DiOxyQ for 45 minutes. The fluorescence intensity was measured with a fluorescence microplate reader at 480 nm/530 nm (excitation/emission) and was proportional to the total ROS/RNS levels within the sample.

### Flow Cytometry

In order to quantify percentage of NPCs, and neurons within the aggregates, flow cytometry with NPC and neuronal markers was performed. Flow cytometry was performed according to previously published protocol [77] with some optimization steps for 3D cultures. Aggregates were washed once with PBS/1 mM EDTA and trypsinized directly in the well using TrypLE Express containing 4 units/ml DNAse for 30 min at 37° C. on the shaker. Pipetting the aggregates up and down with a 1 ml syringe and a 26G3/8 needle ensured generation of single cell suspension. Cells were counted, washed once with PBS/1 mM EDTA, fixed with 2% PFA for 20 min at 4° C., washed twice with PBS/1% BSA (wash solution I, WS I) and blocked for 30 min in blocking solution (PBS/1% BSA/0.15% saponin/10% NGS). 1×10⁶ cells were stained for one hour at 4° C. with fluorochrome-conjugated antibodies dissolved in blocking solution (Table 3). Unstained cells as well as cells incubated with isotype controls were used as negative controls to set the gates for measurements. Cells were washed twice with PBS/1% BSA/0.15% saponin, once with PBS/1% BSA. Flow cytometry was performed using a Becton Dickinson FACSCalibur system by measuring 10⁴ gating events per measurement. Data was analyzed using FlowJo v10 software.

TABLE 3
Antibodies for flow cytometry analysis

| Antibodies | Host | type | Source | Dilution |
|---|---|---|---|---|
| Alexa Fluor ® 647 Nestin | Mouse | Monoclonal, clone 25 | BD Pharmingen | 1:05 |
| Alexa Fluor ® 488 β-III-Tubulin | Mouse | Monoclonal, clone TUJ1 | BD Pharmingen | 1:05 |
| PerCP-Cy ™ 5.5 Sox2 | Mouse | Monoclonal, clone 030-678 | BD Pharmingen | 1:20 |
| PerCP-Cy ™ 5.5 Sox1 | Mouse | Monoclonal, clone N23-844 | BD Pharmingen | 1:20 |
| PE Doublecortin | Mouse | Monoclonal, clone 30 | BD Pharmingen | 1:20 |
| Alexa Fluor ® 647 Ki67 | Mouse | Monoclonal, clone B56 | BD Pharmingen | 1:20 |

### Microelectrode Array (MEA) Recordings

After 8 weeks of differentiation, BMPS were plated on 48-well MEA plates previously coated with Matrigel. During two weeks spontaneous electrical activity was recorded using the ‘Maestro’ MEA platform and Axion's Integraded Studio (AXIS) software [Axion Biosystems inc.; Atlanta, US]. Each well of the 48-well MEA plate contains 16 individual microelectrodes (˜40-50 μm diameter, center-to-center spacing 350 μm) with integrated ground electrodes, resulting in a total of 768 electrodes/plate. The ‘Maestro’ MEA platform has an integrated heating system, which can be controlled by AXIS software. All recordings were performed at a constant temperature of 37° C. Prior to a twenty minutes recording, the MEA plates were placed in the Maestro MEA platform and equilibrated for five min. AXIS software was used to control heating system and monitor the recordings, which includes simultaneously sampling of the channels at 12.5 kHz/channel with a gain of 1200× and a band pass filter of 200-5000 Hz. The recordings were converted into RAW files. After a recording the RAW-files were re-recorded with AXIS to convert the data into a spike file, which includes spike timing and profile information. A variable threshold spike detector was used for the spike-file, it was set at 6 times standard deviations of the rms-noise on each channel. The spike file was later used for data analysis with NeuroExplorer® [Nex Technologies, Madison (AL), US] to convert data into Microsoft Excel files. Using the function rate histogram, a summary of the spikes of all electrodes of one plate was put into one Excel sheet. Only electrodes that recorded activity higher than 0.05 spikes/sec at least once over the time measured were included for data analysis.

### Statistical Analysis

Statistical analysis was performed using GraphPad InStat 3. The Dunnett's test was applied to all the experiments shown here that compare to a control group. Statistically significant values (p<0.01) are marked with an asterisk (*). For myelination quantification at the different time points, a Kruskal-Wallis test was employed, statistical significance was considered for p values <0.05.

### REFERENCES

1. K. Y. Liu, M. King, P. S. Bearman. Social influence and the autism epidemic. AJS 115, 1387-434 (2010).
2. M. Rutter. Incidence of autism spectrum disorders: changes over time and their meaning. Acta. Paediatr. 94, 2-15 (2005).
3. S. K. Van Den Eeden, C. M. Tanner, A. L. Bernstein, R. D. Fross, A. Leimpeter, D. A. Bloch, L. M. Nelson. Incidence of Parkinson's disease: variation by age, gender, and race/ethnicity. Am. J. Epidemiol.157, 1015-22 (2003).
4. W, A. Kukull, R. Higdon, J. D. Bowen, W. C. McCormick, L. Teri, G. D. Schellenberg, G. van Belle, L. Jolley, E. B. Larson. Dementia and Alzheimer disease incidence: a prospective cohort study. Arch. Neurol. 59, 1737-46 (2002).
5. C. Mo, A. J. Hannan, T. Renoir. Environmental factors as modulators of neurodegeneration: Insights from gene-environment interactions in Huntington's disease. Neurosci. Biobehav. Rev. 52, 178-192 (2015).
6. S. Karama, S. Ducharme, J. Corley, F. Chouinard-Decorte, J. M. Starr, J. M. Wardlaw, M. E. Bastin, I. J. Deary. Cigarette smoking and thinning of the brain's cortex. Mol. Psychiatry. In press (2015).
7. O. van de Rest, A. A. Berendsen, A. Haveman-Nies, L. C. de Groot. Dietary Patterns, Cognitive Decline, and Dementia: A Systematic Review. Adv. Nutr. 13, 154-168. (2015)
8. L. Smirnova H. T. Hogberg, M. Leist and T. Hartung. Developmental neurotoxicity-challenges in the 21st century and in vitro opportunities. ALTEX 31, 129-156 (2014),
9. T. Hartung. Look back in anger—what clinical studies tell us about preclinical work. ALTEX. 30, 275-91 (2013)
10. T. Hartung. Food for thought . . . on cell culture. ALTEX. 24, 143-52 (2007)
11. D. Huh, D. C. Leslie, B. D. Matthews, J. P. Fraser, S. Jurek, G. A. Hamilton, K. S. Thorneloe, M. A. McAlexander, D. E. Ingber. A human disease model of drug toxicity-induced pulmonary edema in a lung-on-a-chip microdevice. Sci. Transl. Med. 7, 4(159):159ra147 (2012).
12. D. Pamies, T. Hartung, H. T. Hogberg. Biological and medical applications of a brain-on-a-chip. Exp. Biol. Med. 239, 1096-107 (2014)
13. A. Agarwal, J. A. Goss, A. Cho, M. L. McCain, K. K. Parker. Microfluidic heart on a chip for higher throughput pharmacological studies. Lab on a chip 13, 3599-3608 (2013).
14. H. T. Hogberg, J. Bressler, K. M. Christian, G. Harris, G. Makri, C. O'Driscol, D. Pamies, L. Smirnova, Z. Wen, T. Hartung. Toward a 3D model of human brain development for studying gene/environment interactions. Stem. Cell. Res. Ther. 4 Suppl 1:54 (2013).
15. M. A. Lancaster, M. Renner, C. A. Martin, D. Wenzel, L. S. Bicknell, M. E. Hurles, T. Homfray, J. M. Penninger, A. P. Jackson, J. A. Knoblich. Cerebral organoids model human brain development and microcephaly. Nature 19, 373-9 (2013).
16. Kadoshima T, Sakaguchi H, Nakano T, Soen M, Ando S, Eiraku M, Sasai Y. Self-organization of axial polarity, inside-out layer pattern, and species-specific progenitor dynamics in human ES cell-derived neocortex. Proc. Natl. Acad. Sci. USA. 110, 20284-9 (2013).
17. D. Huh, H. J. Kim, J. P. Fraser, D. E. Shea, M. Khan, A. Bahinski, G. A. Hamilton, D. E. Ingber. Microfabrication of human organs-on-chips. Nat. Protoc. 8, 2135-57 (2013).
18. N. Aldpde, A. Bahinski, M. Daneshian, B. De Wever, E. Fritsche, A. Goldberg, J. Hansmann, T. Hartung, J. Haycock, H. Hogberg, L. Hoelting, J. M. Kelm, S. Kadereit, E. McVey, R. Landsiedel, M. Leist, M. Liibberstedt, F. Noor, C. Pellevoisin, D. Petersohn, U. Pfannenbecker, K. Reisinger, T. Ramirez, B. Rothen-Rutishauser, M. Schafer-Korting, K. Zeilinger, M. G. Zurich. State-of-the-art of 3D cultures (organs-on-a-chip) in safety testing and pathophysiology. ALTEX. 31, 441-77 (2014).
19. T. Hartung, J. Zurlo. Alternative approaches for medical countermeasures to biological and chemical terrorism and warfare. ALTEX. 29, 251-60 (2012).
20. K. Takahashi, K. Tanabe, M. Ohnuki, M. Narita, T. Ichisaka, K. Tomoda, S. Yamanaka. Induction of pluripotent stem cells from adult human fibroblasts by defined factors. Cell 131: 861-872 (2007).
21. J. Yu, M. A. Vodyanik, K. Smuga-Otto, J. Antosiewicz-Bourget, J. L. Frane, S. Tian, J. Nie, G. A. Jonsdottir, V. Ruotti, R. Stewart, I. I. Slukvin, J. A. Thomson. Induced pluripotent stem cell lines derived from human somatic cells. Science 318: 1917-1920 (2007)
22. Y. Tsai, B. Lu, B. Bakondi, S. Girman, A. Sahabian, D. Sareen, C. N. Svendsen, S. Wang. Human iPSC-Derived Neural Progenitors Preserve Vision in an AMD-like Model. Stem. Cells. In press (2015).
23. K. Nieweg, A. Andreyeva, B. van Stegen, G. Tanriöver, K. Gottmann. Alzheimer's disease-related amyloid-β induces synaptotoxicity in human iPS cell-derived neurons. Cell. Death. Dis. In press. (2015)
24. S. Raitano, L. Ordovas, L. De Muynck, W. Guo, I. Espuny-Camacho, M. Geraerts, S. Khurana, K. Vanuytsel, B. I. Tóth, T. Voets, R. Vandenberghe, T. Cathomen, L. Van Den Bosch, P. Vanderhaeghen, P. Van Damme, C. M. Verfaillie. Restoration of progranulin expression rescues cortical neuron generation in an induced pluripotent stem cell model of frontotemporal dementia. Stem Cell Reports. 13, 16-24 (2015).
25. H. Monyer, R. Sprengel, R. Schoepfer, A. Herb, M. Higuchi, H. Lomeli, N. Burnashev, B. Sakmann, P. H. Seeburg. Heteromeric NMDA receptors: molecular and functional distinction of subtypes. Science 256, 1217-1221 (1992).
26. X. Li, P. Jin. Roles of small regulatory RNAs in determining neuronal identity. Nat. Rev. Neurosci. 11, 329-38 (2010).
27. C. Tarantino, G. Paolella, L. Cozzuto, G. Minopoli, L. Pastore, S. Parisi, T. Russo. miRNA 34a, 100, and 137 modulate differentiation of mouse embryonic stem cells. FASEB Journal 24, 3255-3263 (2010).
28. D. Yang, T. Li, Y. Wang, Y. Tang, H. Cui, X. Zhang, D. Chen, N. Shen, W. Le W. miR-132 regulates the differentiation of dopamine neurons by directly targeting Nurr1 expression. Journal of Cell Science 125, 1673-1682 (2012).
29. D. Edbauer, J. R. Neilson, K. A. Foster, C. F. Wang, D. P. Seeburg, M. N. Batterton, T. Tada, B. M. Dolan, P. A. Sharp, M. Sheng. Regulation of synaptic structure and function by FMRP-associated microRNAs miR-125b and miR-132. Neuron 65, 373-384 (2010).
30. S. U. Kim, F. A. McMorris, T. J. Sprinkle. Immunofluorescence demonstration of 2′:3′-cyclic-nucleotide 3′-phosphodiesterase in cultured oligodendrocytes of mouse, rat, calf and human. Brain Res. 300, 195-9 (1984).
31. W. Deng, R. D. Poretz. Oligodendroglia in developmental neurotoxicity. Neurotoxicology. 24, 161-78 (2003).
32. S. P. Fancy, J. R. Chan, S. E. Baranzini, R. J. Franklin, D. H. Rowitch. Myelin Regeneration: A recapitulation of development? Annu. Rev. Neurosci. 34, 21-43 (2011)
33. M. E. Schwab. Functions of Nogo proteins and their receptors in the nervous system. Nat. Rev. Neurosci. 11, 799-811 (2010).
34. D. H. Meijer, M. F. Kane, S. Mentha, H. Liu, E. Harrington, C. M. Taylor, C. D. Stiles, D. H. Rowitch. Separated at birth? The functional and molecular divergence of OLIG1 and OLIG2. Nat. Rev. Neurosci. 13, 819-31 (2012).
35. Y. Yamaguchi, M. Miura. Programmed cell death in neurodevelopment. Dev. Cell. 32, 478-90(2015).
36. H. A. Juraver-Geslin, B. C. Durand. Early development of the neural plate: new roles for apoptosis and for one of its main effectors caspase-3. Genesis. 53, 203-24 (2015).
37. A. K. Krug, S. Gutbier, L. Zhao, D. Pöltl, C. Kullmann, V. Ivanova, S. Förster, S. Jagtap, J. Meiser, G. Leparc, S. Schildknecht, M. Adam, K. Hiller, H. Farhan, T. Brunner, T. Hartung, A. Sachinidis, M. Leist. Transcriptional and metabolic adaptation of human neurons to the mitochondrial toxicant MPP(+). Cell Death Dis.5, e1222 (2014).
38. T. Nakano, S. Ando, N. Takata, M. Kawada, K. Muguruma, K. Sekiguchi, K. Saito, S. Yonemura, M. Eiraku, Y. Sasai. Self-formation of optic cups and storable stratified neural retina from human ESCs. Cell Stem Cell. 14, 771-85 (2012)
39. M. Eiraku, N. Takata, H. Ishibashi, M. Kawada, E. Sakakura, S. Okuda, K. Sekiguchi, T. Adachi, Y. Sasai. Self-organizing optic-cup morphogenesis in three-dimensional culture. Nature. 472, 51-6 (2012).
40. H. Suga, T. Kadoshima, M. Minaguchi, M. Ohgushi, M. Soen, T. Nakano, N. Takata, T. Wataya, K. Muguruma, H. Miyoshi, S. Yonemura, Y. Oiso, Y. Sasai. Self-formation of functional adenohypophysis in three-dimensional culture. Nature. 480, 57-62 (2011).
41. E. van Vliet, S. Morath, C. Eskes, J. Linge, J. Rappsilber, P. Honegger, T. Hartung, S. Coecke. A novel in vitro metabolomics approach for neurotoxicity testing, proof of principle for methyl mercury chloride and caffeine. Neurotoxicology. 29, 1-12 (2008).
42. J. Kim, K. Inoue, J. Ishii, W. B. Vanti, S. V. Voronov, E. Murchison, G. Hannon, A. Abeliovich. A MicroRNA feedback circuit in midbrain dopamine neurons. Science 317, 1220-1224(2007).
43. C. Wiese, A. Rolletschek, G. Kania, P. Blyszczuk, K. V. Tarasov, Y. Tarasova, R. P. Wersto, K. R. Boheler, A. M. Wobus. Nestin expression—a property of multi-lineage progenitor cells? Cell. Mol. Life. Sci. 61, 2510-22 (2004).
44. C. Lépinoux-Chambaud, J. Eyer. Review on intermediate filaments of the nervous system and their pathological alterations. Histochem. Cell. Biol. 140, 13-22 (2013).
45. J. Park, B. K. Lee, G. S. Jeong, J. K. Hyun, C. J. Lee, S. H. Lee. Three-dimensional brain-on-a-chip with an interstitial level of flow and its application as an in vitro model of Alzheimer's disease. Lab Chip. 15, 141-50 (2015).
46. J. P. Dollé, B. Morrison, R. S. Schloss, M. L. Yarmush. Brain-on-a-chip microsystem for investigating traumatic brain injury: Axon diameter and mitochondrial membrane changes play a significant role in axonal response to strain injuries. Technology (Singap. World. Sci.). 2,106 (2014).
47. S. J. Mullett, D. A. Hinkle. DJ-1 knock-down in astrocytes impairs astrocyte-mediated neuroprotection against rotenone. Neurobiol. Dis. 33, 28-36 (2009).
48. Y. Shinozaki, M. Nomura, K. Iwatsuki, Y. Moriyama, C. Gachet, S. Koizumi. Microglia trigger astrocyte-mediated neuroprotection via purinergic gliotransmission. Sci. Rep. 4, 4329 (2014).
49. D. Aguirre-Rueda, S, Guerra-Ojeda, M. Aldasoro, A. Iradi, E. Obrador, A. Ortega, M. D. Mauricio, J. M. Vila, S. L. Valles. Astrocytes protect neurons from Aβ1-42 peptide-induced neurotoxicity increasing TFAM and PGC-1 and decreasing PPAR-γ and SIRT-1. Int. J. Med. Sci.12, 48-56. (2015).
50. R. Sattler, M. Tymianski. Molecular mechanisms of glutamate receptormediated excitotoxic neuronal cell death. Mol. Neurobiol. 24, 107-129 (2001).
51. Y. Shao, M. Gearing, S. S. Mirra. Astrocyte-apolipoprotein E associations in senile plaques in Alzheimer disease and vascular lesions: a regional immunohistochemical study. J. Neuropathol. Exp. Neurol. 56, 376-381 (1997)
52. C. Schwab, P. L. McGeer. Inflammatory aspects of Alzheimer disease and other neurodegenerative disorders. J. Alzheimers. Dis. 13, 359-369 (2008).
53. P. Damier, E. C. Hirsch, P. Zhang, Y. Agid, F. Javoy-Agid. Glutathione peroxidase, glial cells and Parkinson's disease. Neuroscience 52, 1-6 (1993).
54. C. Xie, Y. Q. Liu, Y. T. Guan, G. X. Zhang. Induced Stem Cells as a Novel Multiple Sclerosis Therapy. Curr Stem Cell Res Ther. In press (2015).
55. S. Wang, J. Bates, X. Li, S. Schanz, D. Chandler-Militello, C. Levine, N. Maherali, L. Studer, K. Hochedlinger, M. Windrem, S. A. Goldman. Human iPSC-derived oligodendrocyte progenitor cells can myelinate and rescue a mouse model of congenital hypomyelination. Cell Stem Cell. 12, 252-64 (2013)
56. M. Pinto, S. Dobson. B K and J C virus: a review. J. Infect. 68 Suppl 1:S2-8 (2014).
57. X. B. Liu, Y. Shen, J. M. Plane, W. Deng. Vulnerability of premyelinating oligodendrocytes to white-matter damage in neonatal brain injury. Neurosci. Bull. 29, 229-38 (2013).
58. G. Bartzokis. Age-related myelin breakdown: a developmental model of cognitive decline and Alzheimer's disease. Neurobiol. Aging. 25, 5-18 (2004).
59. H. Okamoto, T. Miki, K. Y. Lee, T. Yokoyama, H. Kuma, Z. Y. Wang, H. Gu, H. P. Li, Y. Matsumoto, S. Irawan, K. S. Bedi, Y. Nakamura, Y. Takeuchi. Oligodendrocyte myelin glycoprotein (OMgp) in rat hippocampus is depleted by chronic ethanol consumption. Neurosci. Lett. 406, 76-80 (2006).
60. T. W. Bouldin, G. Samsa, T. S. Earnhardt, M. R. Krigman. Schwann cell vulnerability to demyelination is associated with internodal length in tellurium neuropathy. J. Neuropathol. Exp. Neurol. 47, 41-47 (1988).
61. P. David, K. Subramaniam. Prenatal alcohol exposure and early postnatal changes in the developing nerve-muscle system. Birth Defects Res. A. Clin. Mol. Teratol. 73, 897-903 (2005).
62. G. J. Harry, A. D. Toews, M. R. Krigman, P. Morell. The effect of lead toxicity and milk deprivation of myelination in the rat. Toxicol. Appl. Pharmacol. 77, 458-464 (1985).
63. S. J. Rothenberg, A. Poblano, S. Garza-Morales. Prenatal and perinatal low level lead exposure alters brainstem auditory evoked responses in infants. Neurotoxicology 15, 695-699 (1994).
64. E. Tiffany-Castiglioni, J. Zmudzki, G. R. Bratton. Cellular targets of lead neurotoxicity: in vitro models. Toxicology 42, 303-315 (1986).
65. E. Tiffany-Castiglioni. Cell culture models for lead toxicity in neuronal and glial cells. Neurotoxicology. 14, 513-36 (1993).
66. J. Parkinson. An essay on the shaking palsy. 1817. J. Neuropsychiatry Clin. Neurosci. 14,: 223-36 (2002)
67. K. R. Chaudhuri, P. Odin. The challenge of non-motor symptoms in Parkinson's disease. Prog. Brain. Res.184, 325-41 (2010).
68. P. McGonigle. Animal models of CNS disorders. Biochem. Pharmacol. 87, 140-9 (2014).
69. K. Tieu. A guide to neurotoxic animal models of Parkinson's disease. Cold. Spring. Harb. Perspect. Med. In press (2011).
70. S. E. Cavanaugh, J. J. Pippin, N. D. Barnard. Animal models of Alzheimer disease: historical pitfalls and a path forward. ALTEX. 31, 279-302 (2014).
71. A. Pombero, C. Bueno, L. Saglietti, M. Rodenas, J. Guimera, A. Bulfone, S. Martinez. Pallial origin of basal forebrain cholinergic neurons in the nucleus basalis of Meynert and horizontal limb of the diagonal band nucleus. Development. 138, 4315-26 (2011).
72. H. McCann, C. H. Stevens, H. Cartwright, G. M. Halliday. α-Synucleinopathy phenotypes. Parkinsonism. Relat. Disord. 20 Suppl 1, S62-7 (2014).
73. V. N. Uversky. A protein-chameleon: conformational plasticity of alphasynuclein, a disordered protein involved in neurodegenerative disorders. J. Biomol. Struct. Dyn. 21, 211-34 (2003)
74. G. A. Petsko and D. Ringer. Ice cleaved alpha-synuclein a biomarker. Patent WO2012061786 A1. (2012)
75. Wen Z, Nguyen H N, Guo Z, Lalli M A, Wang X, Su Y, Kim N S, Yoon K J, Shin J, Zhang C, Makri G, Nauen D, Yu H, Guzman E, Chiang C H, Yoritomo N, Kaibuchi K, Zou J, Christian K M, Cheng L, Ross C A, Margolis R L, Chen G, Kosik K S, Song H, Ming G L. Synaptic dysregulation in a human iPS cell model of mental disorders. Nature. 2014 Nov. 20; 515(7527):414-8.
76. P. Chomczynski, N. Sacchi N. Single-step method of RNA isolation by acid guanidinium thiocyanate-phenol-chloroform extraction. Ana.l Biochem. 162, 156-9 (1987).
77. L. Smirnova, A. E. M. Seiler, A. Luch. microRNA profiling as tool for developmental neurotoxicity testing (DNT). Toxicol. 64:20.9.1-20.9.22, 2015
78. K. J. Livak, T. D. Schmittgen. Analysis of relative gene expression data using real-time quantitative PCR and the 2(−Delta Delta C(T)) Method. Methods. 25, 402-8 (2001).
79. Nave, K. A. Myelination and support of axonal integrity by glia. Nature 468, 244-252, (2010).
80. Saab, A. S., Tzvetanova, I. D. & Nave, K. A. The role of myelin and oligodendrocytes in axonal energy metabolism. Current opinion in neurobiology 23, 1065-1072, (2013).
81. Pardridge, W. M. Crossing the blood brain barrier: are we getting it right? Drug Disc. Today January 1; 6(1): 1-2, (2001).
82. Kerman, B. E. et al. In vitro myelin formation using embryonic stem cells. Development 142, 2213-2225, (2015)
83. Gaj T, Gersbach C A, Barbas C F 3rd. ZFN, TALEN, and CRISPR/Cas-based methods for genome engineering. Trends Biotechnol. July; 31(7):397-405, (2013).
84. Hockemeyer D, Soldner F, Beard C, Gao Q, Mitalipova M, DeKelver R C, Katibah G E, Amora R, Boydston E A, Zeitler B, Meng X, Miller J C, Zhang L, Rebar E J, Gregory P D, Urnov F D, Jaenisch R. Efficient targeting of expressed and silent genes in human ESCs and iPSCs using zinc-finger nucleases. Nat Biotechnol. September; 27(9):851-7, (2009).
85. Hockemeyer D, Wang H, Kiani S, Lai C S, Gao Q, Cassady J P, Cost G J, Zhang L, Santiago Y, Miller J C, Zeitler B, Cherone J M, Meng X, Hinkley S J, Rebar E J, Gregory P D, Urnov F D, Jaenisch R. Genetic engineering of human pluripotent cells using TALE nucleases. Nat Biotechnol. July 7; 29(8):731-4, (2011).
86. Mali P, Aach J, Stranges P B, Esvelt K M, Moosburner M, Kosuri S, Yang L, Church G M. CAS9 transcriptional activators for target specificity screening and paired nickases for cooperative genome engineering. Nat Biotechnol. September; 31(9):833-8, (2013).
87. Chang N, Sun C, Gao L, Zhu D, Xu X, Zhu X, Xiong J W, Xi J J. Genome editing with RNA-guided Cas9 nuclease in zebrafish embryos. Cell Res. April; 23(4):465-72, (2013).
88. Cong L, Ran F A, Cox D, Lin S, Barretto R, Habib N, Hsu P D, Wu X, Jiang W, Marraffini L A, Zhang F. Multiplex genome engineering using CRISPR/Cas systems. Science. February 15; 339(6121):819-23, (2013).
89. Ran F A, Hsu P D, Wright J, Agarwala V, Scott D A, Zhang F. Genome engineering using the CRISPR-Cas9 system. Nat Protoc. November; 8(11):2281-308, (2013).
90. Kim W R, Sun W. 2011. Programmed cell death during postnatal development of the rodent nervous system. Development, growth & differentiation 53: 225-235, (2011).

### EQUIVALENTS

Those skilled in the art will recognize, or be able to ascertain using no more than routine experimentation, many equivalents to the specific embodiments of the invention described herein. Such equivalents are intended to be encompassed by the following claims.

1-47. (canceled)

48. An in vitro brain microphysiological system (BMPS), comprising:
at least two neural cell types aggregated into a spheroid mass and endothelial cells capable of forming a blood brain barrier, wherein the spheroid mass has a diameter that is less than about 500 μm and the in vitro BMPS is electrophysiologically active in a spontaneous manner.

49. The BMPS of claim 48, further comprising one or more microglia-like cells.

50. The BMPS of claim 49, wherein the micro-glia like cells comprise microglia, microglia precursor cells, or a combination thereof.

51. The BMPS of claim 48, wherein the in vitro BMPS has neural characteristics selected from the group consisting of synaptogenesis, neuron-neuron interactions, neuronal-glial interactions, axon myelination, and combinations thereof.

52. The BMPS of claim 48, wherein at least one neural cell type comprises a mature neuron, a glial cell, or a combination thereof.

53. The BMPS of claim 48, wherein at least one neural cell type comprises astrocytes, polydendrocytes, oligodendrocytes, or combinations thereof.

54. The BMPS of claim 481, wherein the BMPS mimics the microenvironment of the central nervous system (CNS).

55. A synthetic neurological organ comprising a mature neuron, at least one glial cell aggregated into a spheroid mass, and a population of microglia-like cells, wherein the spheroid mass has a diameter that is less than 500 μm and the synthetic neurological organ is electrophysiologically active in a spontaneous manner.

56. The synthetic neurological organ of claim 55, further comprising one or more endothelial cells capable of forming a blood-brain-barrier.

57. The synthetic neurological organ of claim 55, wherein the micro-glia like cells comprise microglia, microglia precursor cells, or a combination thereof.

58. The synthetic neurological organ of claim 55, wherein the mature neuron and glial cells further comprise cells selected from the group consisting of astrocytes, polydendrocytes, oligodendrocytes, and combinations thereof.

59. The synthetic neurological organ of claim 55, wherein synthetic neurological organ further comprises neural characteristics selected from the group consisting of synaptogenesis, neuron-neuron interactions, neuronal-glial interactions, axon myelination, and combinations thereof.

60. The synthetic neurological organ of claim 55, wherein the synthetic neurological organ mimics the microenvironment of the central nervous system (CNS).

61. A method of reproducibly producing an in vitro brain microphysiological system (BMPS) that is electrophysiologically active in a spontaneous manner, comprising:
exposing one or more NPC types to gyratory shaking or stirring; and
differentiating the one or more NPC types into one or more neural cell types aggregated into a spheroid mass.

62. The method of claim 61, wherein the spheroid mass has a diameter that is less than about 450 μm, less than about 400 μm, less than about 350 μm, or less than about 300 μm.

63. The method of claim 61, wherein gyratory shaking comprises constant or regular gyratory shaking or stirring for 2 or more, 3 or more, 4 or more, 5 or more, 6 or more, 7 or more, or 8 or more weeks.

64. The method of claim 61, further comprising adding one or more microglia-like cells.

65. The method of claim 64, wherein the micro-glia like cells comprise microglia, microglia precursor cells, or a combination thereof.

66. The method of claim 61, wherein at least one neural cell type comprises a mature neuron, at least one neuronal cell type comprises a glial cell, or a combination thereof.

67. The method of claim 61, further comprising adding one or one or more endothelial cells capable of forming a blood-brain-barrier.


SEQUENCE LISTING (70 sequences)

SEQ ID NO: 1 - protein, 938 aa, Homo sapiens
MSTMRLLTLALLFSCSVARAACDPKIVNIGAVLSTRKHEQMFREAVNQANKRHGSWKIQLNATSVTHKPNAIQMALSVCEDLISSQVYAILVSHPPTPNDHFTPTPVSYTAGFYRIPVLGLTTRMSIYSDKSIHLSFLRTVPPYSHQSSVWFEMMRVYSWNHIILLVSDDHEGRAAQKRLETLLEERESKAEKVLQFDPGTKNVTALLMEAKELEARVIILSASEDDAATVYRAAAMLNMTGSGYVWLVGEREISGNALRYAPDGILGLQLINGKNESAHISDAVGVVAQAVHELLEKENITDPPRGCVGNTNIWKTGPLFKRVLMSSKYADGVTGRVEFNEDGDRKFANYSIMNLQNRKLVQVGIYNGTHVIPNDRKIIWPGGETEKPRGYQMSTRLKIVTIHQEPFVYVKPTLSDGTCKEEFTVNGDPVKKVICTGPNDTSPGSPRHTVPQCCYGFCIDLLIKLARTMNFTYEVHLVADGKFGTQERVNNSNKKEWNGMMGELLSGQADMIVAPLTINNERAQYIEFSKPFKYQGLTILVKKEIPRSTLDSFMQPFQSTLWLLVGLSVHVVAVMLYLLDRFSPFGRFKVNSEEEEEDALTLSSAMWFSWGVLLNSGIGEGAPRSFSARILGMVWAGFAMIIVASYTANLAAFLVLDRPEERITGINDPRLRNPSDKFIYATVKQSSVDIYFRRQVELSTMYRHMEKHNYESAAEAIQAVRDNKLHAFIWDSAVLEFEASQKCDLVTTGELFFRSGFGIGMRKDSPWKQNVSLSILKSHENGFMEDLDKTWVRYQECDSRSNAPATLTFENMAGVFMLVAGGIVAGIFLIFIEIAYKRHKDARRKQMQLAFAAVNVWRKNLQDRKSGRAEPDPKKKATFRAITSTLASSFKRRRSSKDTSTGGGRGALQNQKDTVLPRRAIEREEGQLQLCSRHRES

SEQ ID NO: 2 - DNA, 4400 bp, Homo sapiens
  source 1..4400: mol_type = unassigned DNA
gtcgccgcagcgtccggaccggaaccagcgccgtccgcggagccgccgccgccgccgccgggccctttccaagccgggcgctcggagctgtgcccggccccgcttcagcaccgcggacagcgccggccgcgtggggctgagccccgagcccccgcgcacgcttcagcgccccttccctcggccgacgtcccgggaccgccgctccgggggagacgtggcgtccgcagcccgcggggccgggcgagcgcaggacggcccggaagccccgcgggggatgcgccgagggccccgcgttcgcgccgcgcagagccaggcccgcggcccgagcccatgagcaccatgcgcctgctgacgctcgccctgctgttctcctgctccgtcgcccgtgccgcgtgcgaccccaagatcgtcaacattggcgcggtgctgagcacgcggaagcacgagcagatgttccgcgaggccgtgaaccaggccaacaagcggcacggctcctggaagattcagctcaatgccacctccgtcacgcacaagcccaacgccatccagatggctctgtcggtgtgcgaggacctcatctccagccaggtctacgccatcctagttagccatccacctacccccaacgaccacttcactcccacccctgtctcctacacagccggcttctaccgcatacccgtgctggggctgaccacccgcatgtccatctactcggacaagagcatccacctgagcttcctgcgcaccgtgccgccctactcccaccagtccagcgtgtggtttgagatgatgcgtgtctacagctggaaccacatcatcctgctggtcagcgacgaccacgagggccgggcggctcagaaacgcctggagacgctgctggaggagcgtgagtccaaggcagagaaggtgctgcagtttgacccagggaccaagaacgtgacggccctgctgatggaggcgaaagagctggaggcccgggtcatcatcctttctgccagcgaggacgatgctgccactgtataccgcgcagccgcgatgctgaacatgacgggctccgggtacgtgtggctggtcggcgagcgcgagatctcggggaacgccctgcgctacgccccagacggcatcctcgggctgcagctcatcaacggcaagaacgagtcggcccacatcagcgacgccgtgggcgtggtggcccaggccgtgcacgagctcctcgagaaggagaacatcaccgacccgccgcggggctgcgtgggcaacaccaacatctggaagaccgggccgctcttcaagagagtgctgatgtcttccaagtatgcggatggggtgactggtcgcgtggagttcaatgaggatggggaccggaagttcgccaactacagcatcatgaacctgcagaaccgcaagctggtgcaagtgggcatctacaatggcacccacgtcatccctaatgacaggaagatcatctggccaggcggagagacagagaagcctcgagggtaccagatgtccaccagactgaagattgtgacgatccaccaggagcccttcgtgtacgtcaagcccacgctgagtgatgggacatgcaaggaggagttcacagtcaacggcgacccagtcaagaaggtgatctgcaccgggcccaacgacacgtcgccgggcagcccccgccacacggtgcctcagtgttgctacggcttttgcatcgacctgctcatcaagctggcacggaccatgaacttcacctacgaggtgcacctggtggcagatggcaagttcggcacacaggagcgggtgaacaacagcaacaagaaggagtggaatgggatgatgggcgagctgctcagcgggcaggcagacatgatcgtggcgccgctaaccataaacaacgagcgcgcgcagtacatcgagttttccaagcccttcaagtaccagggcctgactattctggtcaagaaggagattccccggagcacgctggactcgttcatgcagccgttccagagcacactgtggctgctggtggggctgtcggtgcacgtggtggccgtgatgctgtacctgctggaccgcttcagccccttcggccggttcaaggtgaacagcgaggaggaggaggaggacgcactgaccctgtcctcggccatgtggttctcctggggcgtcctgctcaactccggcatcggggaaggcgcccccagaagcttctcagcgcgcatcctgggcatggtgtgggccggctttgccatgatcatcgtggcctcctacaccgccaacctggcggccttcctggtgctggaccggccggaggagcgcatcacgggcatcaacgaccctcggctgaggaacccctcggacaagtttatctacgccacggtgaagcagagctccgtggatatctacttccggcgccaggtggagctgagcaccatgtaccggcatatggagaagcacaactacgagagtgcggcggaggccatccaggccgtgagagacaacaagctgcatgccttcatctgggactcggcggtgctggagttcgaggcctcgcagaagtgcgacctggtgacgactggagagctgtttttccgctcgggcttcggcataggcatgcgcaaagacagcccctggaagcagaacgtctccctgtccatcctcaagtcccacgagaatggcttcatggaagacctggacaagacgtgggttcggtatcaggaatgtgactcgcgcagcaacgcccctgcgacccttacttttgagaacatggccggggtcttcatgctggtagctgggggcatcgtggccgggatcttcctgattttcatcgagattgcctacaagcggcacaaggatgctcgccggaagcagatgcagctggcctttgccgccgttaacgtgtggcggaagaacctgcaggatagaaagagtggtagagcagagcctgaccctaaaaagaaagccacatttagggctatcacctccaccctggcttccagcttcaagaggcgtaggtcctccaaagacacgagcaccgggggtggacgcggcgctttgcaaaaccaaaaagacacagtgctgccgcgacgcgctattgagagggaggagggccagctgcagctgtgttcccgtcatagggagagctgagactccccgcccgccctcctctgccccctcccccgcagacagacagacagacggacgggacagcggcccggcccacgcagagccccggagcaccacggggtcgggggaggagcacccccagcctcccccaggctgcgcctgcccgcccgccggttggccggctggccggtccaccccgtcccggccccgcgcgtgcccccagcgtggggctaacgggcgccttgtctgtgtatttctattttgcagcagtaccatcccactgatatcacgggcccgctcaacctctcagatccctcggtcagcaccgtggtgtgaggcccccggaggcgcccacctgcccagttagcccggccaaggacactgatgggtcctgctgctcgggaaggcctgagggaagcccacccgccccagagactgcccaccctgggcctcccgtccgtccgcccgcccaccccgctgcctggcgggcagcccctgctggaccaaggtgcggaccggagcggctgaggacggggcagagctgagtcggctgggcagggccgcagggcgctccggcagaggcagggccctggggtctctgagcagtggggagcgggggctaactggccccaggcggaggggcttggagcagagacggcagccccatccttcccgcagcaccagcctgagccacagtggggcccatggccccagctggctgggtcgcccctcctcgggcgcctgcgctcctctgcagcctgagctccaccctcccctcttcttgcggcaccgcccacccacaccccgtctgccccttgaccccacacgccggggctggccctgccctcccccacggccgtccctgacttcccagctggcagcgcctcccgccgcctcgggccgcctcctccagactcgagagggctgagcccctcctctcctcgtccggcctgcagcccagaacgggcctccccgggggtccccggacgctggctcgggactgtcttcaaccctgccctgcaccttgggcacgggagagcgccacccgcccgcccccgccctcgctccgggtgcgtgaccggcccgccaccttgtacagaaccagcactcccagggcccgagcgcgtgccttccccgtgcggcccgtgcgcagccgcgctctgcccctccgtccccagggtgcaggcgcgcaccgcccaacccccacctcccggtgtatgcagtggtgatgcctaaaggaatgtcacgcagttttcaaaaaaaaaaaaaaaaaa

SEQ ID NO: 3 - protein, 594 aa, Homo sapiens
MASSTPSSSATSSNAGADPNTTNLRPTTYDTWCGVAHGCTRKLGLKICGFLQRTNSLEEKSRLVSAFKERQSSKNLLSCENSDRDARFRRTETDFSNLFARDLLPAKNGEEQTVQFLLEVVDILLNYVRKTFDRSTKVLDFHHPHQLLEGMEGFNLELSDHPESLEQILVDCRDTLKYGVRTGHPRFFNQLSTGLDIIGLAGEWLTSTANTNMFTYEIAPVFVLMEQITLKKMREIVGWSSKDGDGIFSPGGAISNMYSIMAARYKYFPEVKTKGMAAVPKLVLFTSEQSHYSIKKAGAALGFGTDNVILIKCNERGKIIPADFEAKILEAKQKGYVPFYVNATAGTTVYGAFDPIQEIADICEKYNLWLHVDAAWGGGLLMSRKHRHKLNGIERANSVTWNPHKMMGVLLQCSAILVKEKGILQGCNQMCAGYLFQPDKQYDVSYDTGDKAIQCGRHVDIFKFWLMWKAKGTVGFENQINKCLELAEYLYAKIKNREEFEMVFNGEPEHTNVCFWYIPQSLRGVPDSPQRREKLHKVAPKIKALMMESGTTMVGYQPQGDKANFFRMVISNPAATQSDIDFLIEEIERLGQDL

SEQ ID NO: 4 - DNA, 4007 bp, Homo sapiens
  source 1..4007: mol_type = unassigned DNA
agcgtgtggtagaggagaaacgctgaaaccggaccgaaacctcgccctaggcttagcgatggctaaaaaccggctgggacaagagggaggcaagcaacattccgactcgctgctttctggctgtctggagtgcaaggtgactgtggttcttctctggccaagtccgagggagaacgtaaagatatgggcctttttccccctctcaccttgtctcaccaaagtccctagtccccggagcagttagcctctttctttccagggaattagccagacacaacaacgggaaccagacaccgaaccagacatgcccgccccgtgcgccctccccccgctggcccacacgccggctgctgagtgcccaatggggcttgtagcggctcggctggaaaatcgctcactgagcgctcccctgtgctcctagcccagtcccccacacccttgcgtcttgtactggccttggacccccaccccgaccccgaccccgcctcgtctcggcgcttcactccaggtcgcgccgatgcaccgccagactcgagagcggcccagggctacgctccctgcgccccagtaccggagctagcgcgcacgtctcctccgctgcccccacccctgcgcacccctaccaggcaggctcgctgcctttcctccctcttgtctctccagagccggatcttcaaggggagcctccgtgcccccggctgctcagtccctccggtgtgcaggaccccggaagtcctccccgcacagctctcgcttctctttgcagcctgtttctgcgccggaccagtcgaggactctggacagtagaggccccgggacgaccgagctgatggcgtcttcgaccccatcttcgtccgcaacctcctcgaacgcgggagcggaccccaataccactaacctgcgccccacaacgtacgatacctggtgcggcgtggcccatggatgcaccagaaaactggggctcaagatctgcggcttcttgcaaaggaccaacagcctggaagagaagagtcgccttgtgagtgccttcaaggagaggcaatcctccaagaacctgctttcctgtgaaaacagcgaccgggatgcccgcttccggcgcacagagactgacttctctaatctgtttgctagagatctgcttccggctaagaacggtgaggagcaaaccgtgcaattcctcctggaagtggtggacatactcctcaactatgtccgcaagacatttgatcgctccaccaaggtgctggactttcatcacccacaccagttgctggaaggcatggagggcttcaacttggagctctctgaccaccccgagtccctggagcagatcctggttgactgcagagacaccttgaagtatggggttcgcacaggtcatcctcgatttttcaaccagctctccactggattggatattattggcctagctggagaatggctgacatcaacggccaataccaacatgtttacatatgaaattgcaccagtgtttgtcctcatggaacaaataacacttaagaagatgagagagatagttggatggtcaagtaaagatggtgatgggatattttctcctgggggcgccatatccaacatgtacagcatcatggctgctcgctacaagtacttcccggaagttaagacaaagggcatggcggctgtgcctaaactggtcctcttcacctcagaacagagtcactattccataaagaaagctggggctgcacttggctttggaactgacaatgtgattttgataaagtgcaatgaaagggggaaaataattccagctgattttgaggcaaaaattcttgaagccaaacagaagggatatgttcccttttatgtcaatgcaactgctggcacgactgtttatggagcttttgatccgatacaagagattgcagatatatgtgagaaatataacctttggttgcatgtcgatggatttaacttctcacaattggccaataggatcatctgccttgctactgaactaatgactaacaaaggctgtgtcacgtggcatcccaactattcagtaaacatgcatcatggctgcctggggaggtgggctgctcatgtccaggaagcaccaccataaactcaacggcatagaaagggccaactcagtcacctggaaccctcacaagatgatgggcgtgctgttgcagtgctctgccattctcgtcaaggaaaagggtatactccaaggatgcaaccagatgtgtgcaggatacctcttccagccagacaagcagtatgatgtctcctacgacaccggggacaaggcaattcagtgtggccgccacgtggatatcttcaagttctggctgatgtggaaagcaaagggcacagtgggatttgaaaaccagatcaacaaatgcctggaactggctgaatacctctatgccaagattaaaaacagagaagaatttgagatggttttcaatggcgagcctgagcacacaaacgtctgtttttggtatattccacaaagcctcaggggtgtgccagacagccctcaacgacgggaaaagctacacaaggtggctccaaaaatcaaagccctgatgatggagtcaggtacgaccatggttggctaccagccccaaggggacaaggccaacttcttccggatggtcatctccaacccagccgctacccagtctgacattgacttcctcattgaggagatagaaagactgggccaggatctgtaatcatccttcgcagaacatgagtttatgggaatgccttttccctctggcactccagaacaaacctctatatgttgctgaaacacacaggccatttcattgagggaaaacataatatcttgaagaatattgttaaaaccttacttaaagcttgtttgttctagttagcaggaaatagtgttctttttaaaaagttgcacattaggaacagagtatatatgtacagttatacatacctctctctatatatacatgtatagtgagtgtggcttagtaatagatcacggcatgtttcccgctccaagagaattcactttaccttcagcagttaccgaggagctaaacatgctgccaaccagcttgtccaacaactccaggaaaactgtttttcaaaacgccatgtcctaggggccaagggaaatgctgttggtgagaatcgacctcactgtcagcgtttctccacctgaagtgatgatggatgagaaaaaacaccaccaaatgacaagtcacaccctccccattagtatcctgttaggggaaaatagtagcagagtcattgttacaggtgtactatggctgtatttttagagattaatttgtgtagattgtgtaaattcctgttgtctgaccttggtggtgggagggggagactatgtgtcatgatttcaatgattgtttaattgtaggtcaatgaaatatttgcttatttatattcagagatgtaccatgttaaagaggcgtcttgtattttcttcccatttgtaatgtatcttatttatatatgaagtaagttctgaaaactgtttatggtattttcgtgcatttgtgagccaaagagaaaagattaaaattagtgagatttgtatttatattagagtgcccttaaaataatgatttaagcattttactgtctgtaagagaattctaagattgtacataaagtcatatatatggaaatcctgttacttaaatagcatctgctcttctcttacgctctctgtctggctgtacgtctggtgttctcaatgcttttctagcaactgttggataataactagatctcctgtaattttgtagtagttgatgaccaatctctgttactcgcttagctgaaacctaaggcaacatttccgaagaccttctgaagatctcagataaagtgaccaggctcacaactgtttttgaagaagggaaattcacactgtgcgttttagagtatgcaagaagaatataaataaataaaaatattctccatggagaatttgaacaaaaaaaaaaaaaaaa

SEQ ID NO: 5 - protein, 599 aa, Homo sapiens [sequence omitted]

SEQ ID NO: 6 - DNA, 1981 bp, Homo sapiens
  misc_difference 68: note = a, c, t, g, unknown or other
  source 1..1981: mol_type = unassigned DNA
gtagcttcactaaggtgggatggatagcagggtctcaggcacaaccagtaatggagagacaaaaccantgtatcacaagatggagtttgtgctgtcagtggctggggagatcattggcttaggcaacgtctggaggtttccctatctctgctacaaaaatgggggaggtgccttcttcatcccctacctcgtcttcctctttacctgtggcattcctgtcttccttctggagacagcactaggccagtacactagccagggaggcgtcacagcctggaggaagatctgccccatctttgagggcattggctatgcctcccagatgatcgtcatcctcctcaacgtctactacatcattgtgttggcctgggccctgttctacctcttcagcagcttcaccatcgacctgccctggggcggctgctaccatgagtggaacacagaacactgtatggagttccagaagaccaacggctccctgaatggtacctctgagaatgccacctctcctgtcatcgagttctgggagcggcgggtcttgaagatctctgatgggatccagcacctgggggccctgcgctgggagctggctctgtgcctcctgctggcctgggtcatctgctacttctgcatctggaagggggtgaagtccacaggcaaggtggtgtacttcacggccacatttccttacctcatgctggtggtcctgttaattcgaggggtgacgttgcctggggcagcccaaggaattcagttttacctgtacccaaacctcacgcgtctgtgggatccccaggtgtggatggatgcaggcacccagatattcttctccttcgccatctgtcttgggtgcctgacagccctgggcagctacaacaagtaccacaacaactgctacagcggcaccagctttgtggccggctttgccatcttctccatcctgggcttcatgtctcaggagcagggggtgcccatttctgaggtggccgagtcaggccctggcctggctttcatcgcttacccgcgggctgtggtgatgctgcccttctctcctctctgggcctgctgtttcttcttcatggtcgttctcctgggactggatagccagtttgtgtgtgtagaaagcctggtgacagcgctggtggacatgtaccctcacgtgttccgcaagaagaaccggagggaagtcctcatccttggagtatctgtcgtctccttccctgtggggctgatcatgctcacagagggcggaatgtacgtgttccagctctttgactactatgcggccagtggcatgtgcctcctgttcgtggccatcttcgagtccctctgtgtggcttgggtttacggagccaagcgcttctacgacaacatcgaagacatgattgggtacaggccatggcctcttatcaaatactgttggctcttcctcacaccagctgtgtgcacagccacctttctcttctccctgataaagtacactccgctgacctacaacaagaagtacacgtacccgtggtggggcgatgccctgggctggctcctggctctgtcctcctggtctgcattcctgcctggagcctctacagactcggaaccctcaagggccccttcagagagagaatccgtcagctcatgtgcccagccgaggacctgccccagcggaacccagcaggaccctcggctcccgccacccccaggacctcactgctcagactcacagagctagagtctcactgctagggggcaggcccttggatggtgcctgtgtgcctggccttggggatggctgtggagggaacgtggcagaagcagccccatgtgttccctgcccccgacctggagtggataagacaagaggggtattttggagtccacctgctgagctggaggcctcccactgcaacttttcagctcaggggttgttgaacagatgtgaaaaggccagtgccaagagtgtccctcggagacccttgaaggc

SEQ ID NO: 7 - protein, 528 aa, Homo sapiens
MPTPDATTPQAKGFRRAVSELDAKQAEAIMVRGQGAPGPSLTGSPWPGTAAPAASYTPTPRSPRFIGRRQSLIEDARKEREAAVAAAAAAVPSEPGDPLEAVAFEEKEGKAVLNLLFSPRATKPSALSRAVKVFETFEAKIHHLETRPAQRPRAGGPHLEYFVRLEVRRGDLAALLSGVRQVSEDVRSPAGPKVPWFPRKVSELDKCHHLVTKFDPDLDLDHPGFSDQVYRQRRKLIAEIAFQYRHGDPIPRVEYTAEEIATWKEVYTTLKGLYATHACGEHLEAFALLERFSGYREDNIPQLEDVSRFLKERTGFQLRPVAGLLSARDFLASLAFRVFQCTQYIRHASSPMHSPEPDCCHELLGHVPMLADRTFAQFSQDIGLASLGASDEEIEKLSTLYWFTVEFGLCKQNGEVKAYGAGLLSSYGELLHCLSEEPEIRAFDPEAAAVQPYQDQTYQSVYFVSESFSDAKDKLRSYASRIQRPFSVKFDPYTLAIDVLDSPQAVRRSLEGVQDELDTLAHALSAIG

SEQ ID NO: 8 - DNA, 14877 bp, Homo sapiens
  source 1..14877: mol_type = unassigned DNA
gcgggggggcagtgtgtgctccagcatgtgtgtgtgtgtgtgcatgtacacgtgtgcacctgtatcgcctgtgtgtgtgcatgtgatgtgtacacgtgtcatgcatgcacgcacatgtgtagtgtgtgctcgtgtgtggtgtgtgcctgtgtcatgtatgagcacacttgtatatgttgtgtgtactgtgtcatatatgagtgtgtttgcctgtgtagtgcatgcacatccgtgtgtgcatctggtgtgtccgtgggtcattacgagtgcatcgtatgtgtatcgtgtacatgagtacacttgtatgtgtggtgtgtacaggtgccatgtaagtgtgcttgtacatatatgcatgcatgtgtcatatgcatctgtgtgtgcatgtgtgtggtgcacacatgtgttatgtctgagtgtgcctgtatgtgtgctatgtacacgtcatgtgtgagtgtgcttgcatgtgcagtgtgtggatgctgcttgtacctgtggtgtgtacctgtgtcatgggtgctcacacgtgcatggagtgttgtgtgtgtgcttgtgtgccccatgtgtgcatgtgtgtgtgcctcacacagatgcctgcatttgcctaggcacttgcaagaggacaccatgctggctctcaaagatcacagggccacctgagccctgtgcacaccacagccaggccatggctagaccctgcagagccacagggcgatgcctgtcagccaggggacccagaacacctcctgggctcctccccagcacatggctgggctcctccagcaggcctggatttgggaagggcccgtggtgggcaaggctggtgctggggagcaggcctggtggcctcagagactcgccctgtgggcggagcagcctcacagccaggtcgaagtcagcactctgaccctgccccacgcggggagtgggcaccagtcccagggcacagacgtgctgggtgattaatctgggtgattaagcctcgggctgagaggctgttgagagagaacacgctccattgtggagctggctcagcattccttacggccatggtggcaggggctgtaaccacagggacggcggaagtggtggagggtggtggggtatggagggaagcccagagggctccgtgcaggaaggtggagcctggtgcaatggaggggacagcaagggctcctcagacctctgcggggcccccactcccctggtcacctgttttgtctctgatctggcctgggtcggccctcactcctggccccacctcatagccccccctggtggggctccgctccagcccttctccttcccaggggccagtatgctggccccaggggtctcttggggcgtgacctcggcctccagagaaccctgtcccagctctgcccttccctctggggtctctgtagatgggacgctggtcacagcagcctgtctgatttgttccctgtggcctaggttcctgagccccacagtgccaggggatggatgccaccggatctttgaaagaccagtgtcaggccgggcgcagtggctcacgcctgtaatcccagcactttgggaggccgaggtgggcggatcacgaagtcaggagatcgagaccatcctggctaacacagtgaaaccccgtctccactaaaaatacaaaaagttagctgggcgtggtggtgggcgcctgtagtcccagctacttgggaggctgaggcaggagaatggcgtgaaccggggaggcggagcttgcagtgagccgagatcgcgccattgcactccagcctgggtgacagagcgagactcggtctcaaaaaaaaagaaaaaaaggaaagaccagtgtcttgggagttgggaaacctgggctggagactcactgcatgacccctgagaagttgcacctcagaacctcagtcctcgcatctgcagaatgggtctgtgaacacctcagctgcccgaacgtggatgccgcaggctgacccagcactgagctctaccaagaccaggggccagccgtgtgctccctccaggcctgtgcccagcgtggagaggcctcgtcccgtgggcgctggagtggagccttcctggtgtttgtggacatctctggagagggccagaggcaggtgggtgacacggggcatggctcaatcatgggtggtccagactggagaggtaccctcgggctgggagcggggaggctggccagggtagacttttggggcctccatggataccctcaccatctggaatcggagaggggcacggcacaaaggagggcggggccagggccaggactggagtcgggggcacctctgtgccaacaggggccttggatctggggtacagcatggttccccggccctgaaggggctggcgtgtgggacaggcttcccaggaatggataggcagggatggatgctgcctgattggggcgggaggctggaggcagggcaggtgcaggcacctgagggcagcactcacctccacaggggtccaggggcctccccagcctcagcacctggcctgggctcctgcctccagagagcctggccccaaggaagagtctagtaagcttagttcccatcgggcttccatgaaagcacaactggcccggcaggaaaccgaattaaaaagcaatatttgtatcagtggaagacatttgctgaaaggttaaatccacatccggcagtgtgggccatgagcctccggcgtggtgttcatcaggcatgtctctcctcctggcctgggcacctgagcactggggccgccctgggcagagctggggcggggtgctggggggcctggagctgcctcaccgagggatcctcagcagccgaccctgggggaggcaaatgagactctttctggggaccttgaggggagctcgggggagccatgcagagcttcaccaggcctggacactgggcatggaggctgggccacccaagggccatcaccagggactcaggtgggtgggcctcagccctgggtgacagaagctcacgggccgcagggcgaggccagaggctgagccttcaggctgaggtcttggaggcaaatccctccaacgcccttctgagcaggcacccagacctactgtgggcaggacccacaggaggtggaggcctttggggaacactgtggaggggcatagcatctccgagagaggacagggtctgcactgggtgctgagagacagcaggggccgagcggtaggcttccctgcccccagggatgttccagaggagcgcaagggaggggcattaatatcgtggcaagaaagggcaggcattgcagagtgagcagcgacggaactgggttttgtgggatgcataggagttcacccggataagaggtgggtgaggaatgacactgcaaaccggggatcacggagccccaaatccttctgggccaggaagtgggaagggttggggggtcttccctttgctttgactgagcactcagcctgcctgcagagggcagcgaggagccacggaggggtgtgggacagggatgccatggctgaagcagttttaggaaaggtcccaggggctattgttgaagagagaacggggagcggggagtcccacagctgacaggagcagagtgggccctgagagatgccagctctggctgccacagtgaccagccggggtaggccttcgagaagtcagggagcgtctagggcttctggctcctgctgggcccagggtgtcatcttgggctgccaacaccagaaagcccagcagatacaggaagccccaagccctgtcggaaacggttcttctccaggagggacagcggtggcagcgttcagccgcaggccatgcactctggggccacgtccttccctctgtacagtccagcattgtcaaggcaggctctggccatctctgctgaccccagagggatggggaggcctccccttccaccagaagggccagaagccaccctgggcaggggcatcactctccctgggtggggcagcggctgggagcaggaggtgccagtgggcgtgggctggatgcgggtgcctgcggggcggacatggaacttgggggaggctctaggctggggttgtcctcaagggagttctcaggtcaccccagggtcaccctcaacccggggcctggtggggtagaggagaaactgcaaaggtctctccaaggggaaggcatcagggccctcagcactgagggacgtgcgtgctctttaaagaaggggccacaggaccccgagggaagccaggagctagcagtgggccatagaggggctgagtggggtgggtggaagccgtccctggccctggtcgccctggcaaccctggtggggactgtgatgcaggaggtggcagccatttggaaacgcgtggcgtctccttagagatgtcttcttcagcctcccagggtcctccacactggacaggtgggccctcctgggacattctggaccccacggggcgagcttgggaagccgctgcaagggccacacctgcagggcccgggggctgtgggcagatggcactcctaggaaccacgtctatgagacacacggcctggaatcttctggagaagcaaacaaattgcctcctgacatctgaggctggaggctggattccccgtcttggggctttctgggtcggtctgccacgaggttctggtgttcattaaaagtgtgcccctgggctgccagaaagcccctccctgtgtgctctcttgagggctgtggggccaaggggaccctggctgtctcagccccccgcagagcacgagcccctggtccccgcaagcccgcgggctgaggatgattcagacagggctggggagtgaaggcaattagattccacggacgagccctttctcctgcgcctccctccttcctcacccacccccgcctccatcaggcacagcaggcaggggtgggggatgtaaggaggggaaggtgggggacccagagggggctttgacgtcagctcagcttataagaggctgctgggccagggctgtggagacggagcccggacctccacactgagccatgcccacccccgacgccaccacgccacaggccaagggcttccgcagggccgtgtctgagctggacgccaagcaggcagaggccatcatggtaagagggcaggtaggtgcccggcggccgcagtggaccggagcccagggctggtgccagctgcctctgctactccccagcctggctggcagccccaggctcagggtccatgcaaacccctgggacgcggcgtggatgtggaggcctgggcacagcggcatcccctgtgcctggtgtttgagtccctgttgggggagggtgaggtgatgcctgtccctgtgtgtgcccctcttaggccgacctctctcgggggtcgtgtgggtctctgtgtcttgtttcatcttgaatcttaacgatcggaatgtggaaacaaatccatccaaaaaatccaagatggccagaggtccccggctgctgcacccagcccccaccctactcccacctgcccctgcctccctctgccccagctgccctagtcagcaccccaaccagcctgcctgcttggggaggcagccccaaggcccttcccaggctctagcagcagctcatggtggggggtcctgggcaaatagggggcaaaattcaaagggtatctgggctctggggtgattcccattggcctgttcctcccttatttccctcattcattcattcattcattcattcattcaccatggagtctgtgttccctgtgacctgcactcggaagccctgtgtacaggggactgtgtgggccaggctggataatcgggagcttttcagcccacaggaggggtcttcggtgcctccttgggcactcagaaccttgggctccctggcacatttaaaatgggtttttatttatggaccttgattgaaatgtggtgtgagttgtagcagtgtcatttccaggtaccttctcagggacacagggcgccctcccccgtcctcccccgccctcccctaccctcccccaccaggctccccatcaggcatcccctccccagggcgccccggggcccagcctcacaggctctccgtggcctggaactgcagccccagctgcatcctacacccccaccccaagggtaagtaagaggggactctgggaggggcttctgctgctccccttcatgttccacaaccctggaagctcaggatgaagctgattcttctcttacaaggggcccagagccttcttgggagttcagctccaagggatgagccccaggtgtctgccaagtccccctctgtccaggcctgggacggctctgggatcgaggggtcagaggcgctgagcccagggagagacacctgcgcccagagctatgacaaagggtggagggatgacaaggcagccaggagcgggcgcctgcggggtggcacagaggggcagggcccgaggacaggtgtcctgatgggagtgtgagaaagggtcccctgtgcggcagccaggagggtaggggggttgttcactggggccctgtgggggcagctccttcctgagctgccgttccctccccggcagccgatgccactgtccatcaagacatcgccctcttcccatcactaatccagttagcgcctggcctggggatgagtgacacagcgtctctgtctgtctgctcgccacagagtggggagcaggcgagcaccttcccagcccccactcctcccccaccaccactgcttctgactgggctgcccccatcgggaagggcgtgcaatgcccgcaggcacctcggctagcatctgccccagcaggcacacagtaggcgctcaaaaacgtgctctcatcccctgcctctgtgtgccatcagcgctgcccgactgtgggaccagctgtgggtggaggtccccgggtctcagcaggtggaggaggcatgggtgccccttgtccccacagtccccgcggttcattgggcgcaggcagagcctcatcgaggacgcccgcaaggagcgggaggcggcggtggcagcagcggccgctgcagtcccctcggagcccggggaccccctggaggctgtggcctttgaggagaaggaggggaaggccgtgctaaacctgctcttctccccgagggccaccaagccctcggcgctgtcccgagctgtgaaggtgtttgaggtgagctggtggccttcgtgtccctggggcaagttcacctgtgggtggggctgtgtgggctgagttcctgacccctctatagcagaggtgcagctgcccaggcccccgaggccggcacaggatgcagcaggggagtctcaggcctcagctcagcccccatggcatctagccacacccccgtgtttttgagggatcctgagcccacccctagggctgaggctaccaagccccactgtgcctcttgccttgcccatcccctggatccccctcacccaccatttcccacgtggggggctcccagcagggcagcacaagaggcaggggcagggcagtgtgccctctcccacccacccagcacagtggctcaggtgaccactgattgcattagtcactccggccccactgtgccccgggaggcaggtgacccagctcccggaagaagctcccaaatgacattaaagccagactccccgccccccagctcccagagccagttttgtggcccgagggccactgcgacccaccgcccttgttgctaggcaacaggaggtgggggtggagcggacccttctggccagtgtcctggacgctcaggggccagtgagactcagggcccatcctacaaacctggatgaggccaccagggttgggggcaccttctgaccagtggctgaggagccggactgtgtggcatggccttgggacacacacaccgagccgcccagaaccaggttaagcctcaagcggtgacaactcctggttaggcacgtaacacaaaatccaacttgccagtggcaaaccctggcctggtggccgacagctgacctgagcctggaagaacgggatctgtgtgctgctagcacaaaagtcaagggcagggcctggccagccagccagatgtgcctcctccccgcccaccccaccctctctctccatctctgtctctttctccttctctctctcttcctgcttttgctccctaagacgtttgaagccaaaatccaccatctagagacccggcccgcccagaggccgcgagctgggggcccccacctggagtacttcgtgcgcctcgaggtgcgccgaggggacctggccgccctgctcagtggtgtgcgccaggtgtcagaggacgtgcgcagccccgcggggcccaagggtgaggcggttttctgtccttgagggccaccaaatgaccttgagaggctggggtgcaggggctcctgcagggggaccctacagtgaccacgtggtggtggcctggttccctctctgcgggctccactccgcaccccgttttgctacacatccgtgtccgggcctggggccactccaggatccccccgcagctctcacagccccggctgcctctgccccccggaagtcttgtaggggaggctgcttcaaggtgggtgacacagccccacggctccgagctcaccaagatctcttcctccatcacccataaagtcccctggttcccaagaaaagtgtcagagctggacaagtgtcatcacctggtcaccaagttcgaccctgacctggacttggaccacccggtgagtggtgcgcccctcactcaggcctcctgcccctgatcacatcccctacccttagcccaaccctggacaggagtctgtcggctccaggagcctccgtggcctgtgcccccaccccagcacagcctcctgacccgtgcatcccctctgccctcagggcttctcggaccaggtgtaccgccagcgcaggaagctgattgctgagatcgccttccagtacaggcagtgaggggcccctgcgctcgggacccagactccgtcctgcaggctgacgctggacctggggggtgggagggaaggacaaaggggaggacccatcttgtcaccagcatcagtgcctcctgccaggcagctctgctccagggctttccatgtccccaaatcccagtggggaaactgaggcccaggggggctagagcaacctgccgaggccacatagccggctcacggcacagtcagctggggtgcaccctcctgtccatcctccaacccaaaggcctcgctgcactaggcgggtgtggacctgtgcccagtgaagctccctccctccctcctgcccttctcactccccgaggggacctgctgaccactggccccctccccagcggcgacccgattccccgtgtggagtacaccgccgaggagattgccacctggtgagacctccgtgcagctaggggctggggaggagcccgggggatgcctcctggaatcctggcgtgtgagggccgcctccagggaccttggcacaacaggagagactaaggccgggaagaagagggacttgcagggctcagaatgttgggttgggaggaagaggctacccatcctgtcgggccatccccagtgtgctgagggaccgcccctcatggccccctatcccctgggattccctaaagccaccagcaaaagcccctcccgggggcctgggtcttcaggggtccccaagaggcctgcgttggtaggggctcaggcaggcagaggcacccacagttcaggaggggggtttcgggcactggggtggggcattagagggccctgagcctggctgcccgcaggaaggaggtctacaccacgctgaagggcctctacgccacgcacgcctgcggggagcacctggaggcctttgctttgctggagcgcttcagcggctaccgggaagacaatatcccccagctggaggacgtctcccgcttcctgaagggtgtgcccagacgggaggggcgcagagccggggggccggggatggtcagccaagcgccccaccccagcgcggctccagcccgtcccggctcggcagtgacccgcgtggccccttgcagagcgcacgggcttccagctgcggcctgtggccggcctgctgtccgcccgggacttcctggccagcctggccttccgcgtgttccagtgcacccagtatatccgccacgcgtcctcgcccatgcactcccctgagccgtgagtgcgcgccctggccgccagcccgagggtggggggtgcgacgggcggcccctcagcccccttctccctcctacgcgcagggactgctgccacgagctgctggggcacgtgcccatgctggccgaccgcaccttcgcgcagttctcgcaggtacgccgcggcctcggagggagccggggtcacccaggggctggcttggcgccgggggcgggcggggatcgatgtgcgggtgggtgaagtgtgctgcctgctcccgggccccgccaaggaggctcggcgccccgagggtcgcgcggcatagggcggggctggagcggagcctcccacggcctgtgctgccacctgccggctacctgggaacggcgcccacgggcttaggaatgtggtcaaggagggctgcctggaggaggaggcccggtggaggtgcggatcctgggcggccagggaaggtctctgccgccagggaagtgtcccagagacccctggaggggctgctgacacccccggtgcccccacctcgagcatgacccagggctgcctctccccatccttcatcctccctgctccacaggacattggcctggcgtccctgggggcctcggatgaggaaattgagaagctgtccacggtgggttgacccctccctgcagggcctggggtgtgggtttgggggtctgaatccaggcctcaccctcttgccgtccaggctgaggcctctccttccacccacgaattgtgaccctcaccctggcctgcctgcatcctggcctggcctccctgggggtggtatcctggtcacgggtgaccaggggctgcccggtgggcggcagctgtctctgggctgatgctgcccggcttccccgcagctgtactggttcacggtggagttcgggctgtgtaagcagaacggggaggtgaaggcctatggtgccgggctgctgtcctcctacggggagctcctggtgagagtctctccttgctgcagcccccagcagaggggcagggctgggggacggtgcagggaggggacaggctcccagtgggaggaaactgaggcctggacctccaggactcaggctctgtttgggagaaggcttgtctctgcccagtcctcaccccacattatcccaggcctccgaaggcccggcgggggagatgggggtgactctacccaaggaacccacccagcgtcaggccacggtgccccagttccctcggggacctgggtgcagtggagtcagtgatgccattggcctcctgccagcactgcctgtctgaggagcctgagattcgggccttcgaccctgaggctgcggccgtgcagccctaccaagaccagacgtaccagtcagtctacttcgtgtctgagagcttcagtgacgccaaggacaagctcaggtgggctaggctgctagggcaagccccccatggtgcccccaaactgggccagccaggccttccttctggccttgagcagggctggacctgtgagcccaggtcacagatgagaaaaccgacccctggttgcagcagcccccacacagcagggacaccatccgtgagaaggaccccagcgtctggggaggggcagacctacaggactgggggctgctgggtggccgggtcaaggccagtcttggaggtgctgacagagcctgagctttgtgaggacgtcctgtggaacctgtcccggccccctgccctgggatggggagaagtcagggggatagacagagtcaaggtgggggacagggcgggagtggggtccccagggctgggggcctttggtgcagtgaccagagtgtcaggagaggggagcaaagccctctagcctcatcctcataaaaggtctcatcattttccctccagcctcttatgcactggggaaactgaggccaggggctatgtgtccagcggacaggggtgctgaattccacccacaggcttagggatatggtcaaggaaagcttcctggaggaggcccagtggaggttcagggagggatggggtgcccggcagtctctagtggaaaaggcgcctagcctatctcccccatgaaccccctcacccagccctggaagaggcctcagtgtcccgcctgtgaccagttggctcagaaaagccctgggagctctgagccactgtgaaggtggaaacgcggcccctggcctcccctctcctggaggctgcagactctgcccgccagttgacgagggctctgccgctctcctccccaggagctatgcctcacgcatccagcgccccttctccgtgaagttcgacccgtacacgctggccatcgacgtgctggacagcccccaggccgtgcggcgctccctggagggtgtccaggatgagctggacacccttgcccatgcgctgagtgccattggctaggtgcacggcgtccctgagggcccttcccaacctcccctggtcctgcactgtcccggagctcaggccctggtgaggggctgggtcccgggtgccccccatgccctccctgctgccaggctcccactgcccctgcacctgcttctcagcgcaacagctgtgtgtgcccgtggtgaggttgtgctgcctgtggtgaggtcctgtcctggctcccagggtcctgggggctgctgcactgccctccgcccttccctgacactgtctgctgccccaatcaccgtcacaataaaagaaactgtggtctctacacctgcctggccccacatctgtgccacagagacagaccctgggatcctcagactcccacacccccaccccagcctcactcagaggtttcgccctggcctccttcctcctctgggagatggctggccgccctggccaggcagctggcccctccgggcctggtttccccgctcaccctgaggccccgcccagctctgagccccaagcagctccagaggctcgggcaccctggccgagctgccccatctccgtggggtgccctcccaaggtggggagccacgtgacagtgggagggcctctctcaggcctggcagggagcaggggtcacaaactgtgctggctgggggtggtctcagaggtgggcctgcaggcctaaccctccctgctgacagggctcccagcccttgagagaaacagggatggaggaacagctgccctgatgccctcacccacccggagcaggccctgcgaaccaaggggaacctcagtgtggcccccagcatgtgtgctgatggggagggtctggctgagctggtgcccaggcagatggtctgggcctgtctccccagcgaggcaggatgggggctggatttcagactctgtaagatgcccctggcttactcgaggggcctggacattgccctccagagagagcacccaacaccctccaggcttgaccggccagggtgtccccttcctaccttggagagagcagccccagggcatcctgcagggggtgctgggacaccagctggccttcaaggtctctgcctccctccagccaccccactacacgctgctgggatcctggatctcagctccccggccgacaacactggcaaactcctactcatccacgaaggccctcctgggcatggtggtccttcccagcctggcagtctgttcctcacacaccttgttagtgcccagcccctgaggttgcagctgggggtgtctctgaagggctgtgagcccccaggaagccctggggaagtgcctgccttgcctccccccggccctgccagcgcctggctctgccctcctacctgggctccccccatccagcctccctccctacacactcctctcaaggaggcacccatgtcctctccagctgccgggcctcagagcactgtggcgtcctggggcagccaccgcatgtcctgctgtggcatggctcagggtggaaagggcggaagggaggggtcctgcagatagctggtgcccactaccaaacccgctcggggcaggagagccaaaggctgggtgtgtgcagagcggccccgagaggttccgaggctgaggccagggtgggacatagggatgcgaggggccggggcacaggatactccaacctgcctgcccccatggtctcatcctcctgcttctgggacctcctgatcctgcccctggtgctaagaggcaggtaggggctgcaggcagcagggctcggagcccatgccccctcaccatgggtcaggctggacctccaggtgcctgttctggggagctgggagggccggaggggtgtaccccaggggctcagcccagatgacactatgggggtgatggtgtcatgggacctggccaggagaggggagatgggctcccagaagaggagtgggggctgagagggtgcctggggggccaggacggagctgggccagtgcacagcttcccacacctgcccacccccagagtcctgccgccacccccagatcacacggaagatgaggtccgagtggcctgctgaggacttgctgcttgtccccaggtccccaggtcatgccctccttctgccaccctggggagctgagggcctcagctggggctgctgtcctaaggcagggtgggaactaggcagccagcagggaggggacccctccctcactcccactctcccacccccaccaccttggcccatccatggcggcatcttgggccatccgggactggggacaggggtcctggggacaggggtgtggggacaggggtcctggg

SEQ ID NO: 9 - protein, 382 aa, Homo sapiens
MLDGLKMEENFQSAIDTSASFSSLLGRAVSPKSVCEGCQRVILDRFLLRLNDSFWHEQCVQCASCKEPLETTCFYRDKKLYCKYDYEKLFAVKCGGCFEAIAPNEFVMRAQKSVYHLSCFCCCVCERQLQKGDEFVLKEGQLLCKGDYEKERELLSLVSPAASDSGKSDDEESLCKSAHGAGKGTAEEGKDHKRPKRPRTILTTQQRRAFKASFEVSSKPCRKVRETLAAETGLSVRVVQVWFQNQRAKMKKLARRQQQQQQDQQNTQRLSSAQTNGGGSAGMEGIMNPYTALPTPQQLLAIEQSVYSSDPFRQGLTPPQMPGDHMHPYGAEPLFHDLDSDDTSLSNLGDCFLATSEAGPLQSRVGNPIDHLYSMQNSYFTS

SEQ ID NO: 10 - DNA, 5310 bp, Homo sapiens
  misc_difference 901..1000: note = a, c, t, g, unknown or other
  misc_difference 1301..1400: note = a, c, t, g, unknown or other
  misc_difference 1821..1920: note = a, c, t, g, unknown or other
  misc_difference 2881..2980: note = a, c, t, g, unknown or other
  misc_difference 3461..3560: note = a, c, t, g, unknown or other
  misc_difference 4401..4500: note = a, c, t, g, unknown or other
  misc_difference 4801..4900: note = a, c, t, g, unknown or other
  source 1..5310: mol_type = unassigned DNA
gtataggttggggcggagtcggattcgggatggaaaacctggggcaagggatgtaggtgggggtgaggggggcaggagaaggagaaacgcagttggggggcggaggcctaagtacataacgtgttgacttcaagtgaaatcagatcagccagagcagttcgctgtgactgatctctcctcccaccctacattctcttggctggaccctatcctcctggctgattctggtcgccctggacactccctcagttctttcccaggagtgcggtggctgctggcgccgagtcccagcgggcacggacgtcagacgcatcgtttcttctcctctacaggtcctcccggcccggcccgaacatgctggacggcctaaagatggaggagaacttccaaagcgcgatcgacacctcggcctccttctcctcgctgctgggtgagtgttcaggccgtgcgtcctgggcgcactctctttccgcttggcgctgagctctggagccccgctctctgggacctggtccgcgatagggaagctagcgcccctcttcatacactaaattgagccccatcactatctgtccgtcagtgcttgtgggtcgtccctacccaaataaatccaacaagccgccccaggcctcacgcactgggcaccgaattccccaaagccgcgaggggcgggcgagcttgttcgtaggcgtctgagtggcaagtgattaaaaatacccagggctggatttttaatctcggagctgatcgacgtctcataaatgccgccctcttctcgcggcctagaggcaatagcatccgagacccgaggcctggagcgcccaagttcgaggaggcttctctcccccaccaactccagccccaatttcagccatgggcaaggccgagagagacttttctnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngggtcccggccaggtttggcatggtctacctgcccgggctgctcacccgccaacgtctgttgtggctacaggcagagcggtgagccccaagtctgtctgcgagggctgtcagcgggtcatcttggacaggtttctgctgcggctcaacgacagcttctggcatgagcagtgcgtgcagtgcgcctcctgcaaagagcccctggagaccacctgcttctaccgggacaagaagctgtactgcaagtatgactacgagaagtaagtggccgcacccccgcagcgctccccgcgcactggcatnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnatcccagttcttgaagttccttttgctgttgacttcaggggagacccaggaccaagccagattttactcatggtgcatgtacttcctttctccctgctgccaggctgtttgctgttaaatgtgggggctgcttcgaggccatcgctcccaatgagtttgttatgcgggcccagaagagtgtataccacctgagctgcttctgctgctgtgtctgcgagcgacagcttcagaagggtgatgagtttgtcctgaaggaggggcagctgctctgcaaaggggactatgagaaggagcgggagctgctcagcctggtgagcccagcagcctcagactcaggtgagtgccaggtggtgggcagggctgcggtggggtgggtagagtggagttgggtggctgtctgcattgtttcttccctagatgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncatacagctccaggaactggctttcagggactcacaacattgtcttttgcttctttcaggtaaaagtgatgatgaagaaagtctctgcaagtcagcccatggggcagggaaaggaactgctgaggaaggcaaggaccataagcgccccaaacgtccgagaaccatcttgacaactcaacagaggcgagcattcaaggcctcatttgaagtatcctccaagccctgcaggaaggtataggagggagcagggaggaaaaggagctgggccccacttctctgtgtgcactcagacccctctgggatctcagtgggcattgggggtcacagtggtgaggaaggctgttcagacagagcctgcacaggcggctcaagcctgttggagactccagagatcactaagctgtggccagggtgtgatagactctcctgaagctttcatgcatgcacaccaactccaaatggcccctgtcacacctttcatttcatagagcacaatgggaacagtaataatgataggtgtccattgtggtgtagacccagatgctgtaaagcaaagagtataaaaacacagtggcttgcagtactcttttttgagtctggctttttccacttggtgtggtggtttggggattcattcattcctatttcagcattccactgtataggtgtgccatgattggtttgtccatgcacctgttgatgggtgtttggggttgtttctagtttgggactgtttcaaataggactgctatggacattcatgtaaaaaaaaatacagtggtttaatgagacaggagtttattctcttctgtcacagtccagaggtgagcaaggcaaggctggtgggtggctctgttatccatctcctgtgtccaagcgactgctccagttgtcaccatgtttccagtcaccaggtagagaaagaggaaatggagggcaagcgccctgctttttaaggatnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnatgcatatgcatggcttatagctaaagcacaacaatagactaaagtctaaaccacttgaaggcctaatttccagagcaagagaaatccagaaacacctcttgggaatgcacatgtaaattaataattattattttgtttctttacctggtgaaggactttctttctacctgaagggaagcaatgttctcgtgtttgtgtgtatgctcaacattaaaaactattcagctcctaaagcagatacagtcttttggcctcctcaagtattatataggagatgttctacctcctaccctgagatgccagtgtgtctacatttctcgttcaatttttccaaggtgagagagactctggctgcagagacagggctgagtgtccgtgtcgtccaggtgtggttccaaaaccagagagcgaaggtaacctgcttcttacttttatctgtccccatgttgctggtttcctgaaataatcacagtaggacannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagccctctcccggggaaggtgtcacttccaggcccccccttactttgtgaacatgctgcaggccacctgacttctaatcctatggtcctctccttatcagatgaagaagctggccaggcgacagcagcagcagcagcaagatcagcagaacacccagaggctgagctctggtaagctggtgcctcctcccaggcagttctggctggaatccaggctgttcctaccagaggcctcccactacccagctctttggatgacatatctggactcagtgaagcctagaccacacccactggagaaataaggccttcaagggaagactgagccacgaggaacttgtgagagggttgagggctcctgagctgcaggcttagaactgctgattggggatggcactgaccttatccacagcgtccaggcctggatcccaccacagcgtcagggactgcttgcagagtcacagatacgttcagtttctcatcttgcttagttctccttccaggctaattgatttaatagaagacacctcggtgacttggctctttccaaaataacataaagtagtaaaaataatgatagtaaaataacaatgccttcctttgttgaacactcttatagattggtgttctcatacatgctgacttgacttttacaacacccattcctggaggcgagtggagaagttgttattatccctatgtcacagatgagcaaacaaaggctctgcaagattgaatgtggccctagatcggtaagggcagggggctgggactagaactctaactgtgttccacaggccatgggccttctcatctctacccagatgtgcttttgaaaaagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncacgttgagaatgacctggcttcttctttgttccacagctcagacaaacggtggtgggagtgctgggatggaaggaatcatgaacccctacacggctctgcccaccccacagcagctcctggccatcgagcagagtgtctacagctcagatcccttccgacagggtctcaccccaccccagatgcctggagaccacatgcacccttatggtaagagggacttaagcccctcgggccctctcataacttgtgtgggtttctcattccctcctaaacacatctaggcagttcccagatgctcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaaatgagtcacttcttcaagaccctcatgccagtgtttcatctccatttcaggtgccgagccccttttccatgacctggatagcgacgacacctccctcagtaacctgggtgattgtttcctagcaacctcagaagctgggcctctgcagtccagagtgggaaaccccattgaccatctgtactccatgcagaattcttacttcacatcttgagtcttcccctagagttctgtgactaggctcccatatggaacaaccatattctttgaggggtcactggctttaggacagggaggccagggaagaggtgggttggggagggagttttgttggggatgctgttgtataatgatatggtgtagctcagcatttccaaagactgaatacattatggattgcatagtttaatg

SEQ ID NO: 11 - protein, 655 aa, Homo sapiens
MAEAPQVVEIDPDFEPLPRPRSCTWPLPRPEFSQSNSATSSPAPSGSAAANPDAAAGLPSASAAAVSADFMSNLSLLEESEDFPQAPGSVAAAVAAAAAAAATGGLCGDFQGPEAGCLHPAPPQPPPPGPLSQHPPVPPAAAGPLAGQPRKSSSSRRNAWGNLSYADLITKAIESSAEKRLTLSQIYEWMVKSVPYFKDKGDSNSSAGWKNSIRHNLSLHSKFIRVQNEGTGKSSWWMLNPEGGKSGKSPRRRAASMDNNSKFAKSRSRAAKKKASLQSGQEGAGDSPGSQFSKWPASPGSHSNDDFDNWSTFRPRTSSNASTISGRLSPIMTEQDDLGEGDVHSMVYPPSAAKMASTLPSLSEISNPENMENLLDNLNLLSSPTSLTVSTQSSPGTMMQQTPCYSFAPPNTSLNSPSPNYQKYTYGQSSMSPLPQMPIQTLQDNKSSYGGMSQYNCAPGLLKELLTSDSPPHNDIMTPVDPGVAQPNSRVLGQNVMMGPNSVMSTYGSQASHNKMMNPSSHTHPGHAQQTSAVNGRPLPHTVSTMPHTSGMNRLTQVKTPVQVPLPHPMQMSALGGYSSVSSCNGYGRMGLLHQEKLPSDLDGMFIERLDCDMESIIRNDLMDGDTLDFNFDNVLPNQSFPHSVKTTTHSWVSG

SEQ ID NO: 12 - DNA, 5738 bp, Homo sapiens
  source 1..5738: mol_type = unassigned DNA
gcagccgccacattcaacaggcagcagcgcagcgggcgcgccgctggggagagcaagcggcccgcggcgtccgtccgtccttccgtccgcggccctgtcagctggagcgcggcgcaggctctgccccggcccggcggctctggccggccgtccagtccgtgcggcggaccccgaggagcctcgatgtggatggccccgcgaagttaagttctgggctcgcgcttccactccgccgcgccttcctcccagtttccgtccgctcgccgcaccggcttcgttcccccaaatctcggaccgtcccttcgcgccccctccccgtccgcccccagtgctgcgttctccccctcttggctctcctgcggctgggggaggggcgggggtcaccatggccgaggcgcctcaggtggtggagatcgacccggacttcgagccgctgccccggccgcgctcgtgcacctggccgctgcccaggccggagtttagccagtccaactcggccacctccagcccggcgccgtcgggcagcgcggctgccaaccccgacgccgcggcgggcctgccctcggcctcggctgccgctgtcagcgccgacttcatgagcaacctgagcttgctggaggagagcgaggacttcccgcaggcgcccggctccgtggcggcggcggtggcggcggcggccgccgcggccgccaccggggggctgtgcggggacttccagggcccggaggcgggctgcctgcacccagcgccaccgcagcccccgccgcccgggccgctgtcgcagcacccgccggtgccccccgccgccgctgggccgctcgcggggcagccgcgcaagagcagctcgtcccgccgcaacgcgtggggcaacctgtcctacgccgacctcatcaccaaggccatcgagagctcggcggagaagcggctcacgctgtcgcagatctacgagtggatggtcaagagcgtgccctacttcaaggataagggtgacagcaacagctcggcgggctggaagaattcaattcgtcataatctgtccctacacagcaagttcattcgtgtgcagaatgaaggaactggaaaaagttcttggtggatgctcaatccagagggtggcaagagcgggaaatctcctaggagaagagctgcatccatggacaacaacagtaaatttgctaagagccgaagccgagctgccaagaagaaagcatctctccagtctggccaggagggtgctggggacagccctggatcacagttttccaaatggcctgcaagccctggctctcacagcaatgatgactttgataactggagtacatttcgccctcgaactagctcaaatgctagtactattagtgggagactctcacccattatgaccgaacaggatgatcttggagaaggggatgtgcattctatggtgtacccgccatctgccgcaaagatggcctctactttacccagtctgtctgagataagcaatcccgaaaacatggaaaatcttttggataatctcaaccttctctcatcaccaacatcattaactgtttcgacccagtcctcacctggcaccatgatgcagcagacgccgtgctactcgtttgcgccaccaaacaccagtttgaattcacccagcccaaactaccaaaaatatacatatggccaatccagcatgagccctttgccccagatgcctatacaaacacttcaggacaataagtcgagttatggaggtatgagtcagtataactgtgcgcctggactcttgaaggagttgctgacttctgactctcctccccataatgacattatgacaccagttgatcctggggtagcccagcccaacagccgggttctgggccagaacgtcatgatgggccctaattcggtcatgtcaacctatggcagccaggcatctcataacaaaatgatgaatcccagctcccatacccaccctggacatgctcagcagacatctgcagttaacgggcgtcccctgccccacacggtaagcaccatgccccacacctcgggtatgaaccgcctgacccaagtgaagacacctgtacaagtgcctctgccccaccccatgcagatgagtgccctggggggctactcctccgtgagcagctgcaatggctatggcagaatgggccttctccaccaggagaagctcccaagtgacttggatggcatgttcattgagcgcttagactgtgacatggaatccatcattcggaatgacctcatggatggagatacattggattttaactttgacaatgtgttgcccaaccaaagcttcccacacagtgtcaagacaacgacacatagctgggtgtcaggctgagggttagtgagcaggttacacttaaaagtacttcagattgtctgacagcaggaactgagagaagcagtccaaagatgtctttcaccaactcccttttagttttcttggttaaaaaaaaaaacaaaaaaaaaaaccctccttttttcctttcgtcagacttggcagcaaagacatttttcctgtacaggatgtttgcccaatgtgtgcaggttatgtgctgctgtagataaggactgtgccattggaaatttcattacaatgaagtgccaaactcactacaccatataattgcagaaaagattttcagatcctggtgtgctttcaagttttgtatataagcagtagatacagattgtatttgtgtgtgtttttggtttttctaaatatccaattggtccaaggaaagtttatactctttttgtaatactgtgatgggcctcatgtcttgataagttaaacttttgtttgtactacctgttttctgcggaactgacggatcacaaagaactgaatctccattctgcatctccattgaacagccttggacctgttcacgttgccacagaattcacatgagaaccaagtagcctgttatcaatctgctaaattaatggacttgttaaacttttggaaaaaaaaagattaaatgccagctttgtacaggtcttttctatttttttttgtttattttgttatttgcaaatttgtacaaacatttaaatggttctaatttccagataaatgatttttgatgttattgttgggacttaagaacatttttggaatagatattgaactgtaataatgttttcttaaaactagagtctactttgttacatagtcagcttgtaaattttgtggaaccacaggtatttggggcagcattcataattttcattttgtattctaactggattagtactaattttatacatgcttaactggtttgtacactttgggatgctacttagtgatgtttctgactaatcttaaatcattgtaattagtacttgcatattcaacgtttcaggccctggttgggcaggaaagtgatgtatagttatggacactttgcgtttcttatttaggataacttaatatgtttttatgtatgtattttaaagaaatttcatctgcttctactgaactatgcgtactgcatagcatcaagtcttctctagagacctctgtagtcctgggaggcctcataatgtttgtagatcagaaaagggagatctgcatctaaagcaatggtcctttgtcaaacgagggattttgatccacttcaccattttgagttgagctttagcaaaagtttcccctcataattctttgctcttgtttcagtccaggtggaggttggttttgtagttctgccttgaggaattatgtcaacactcatacttcatctcattctcccttctgccctgcagattagattacttagcacactgtggaagtttaagtggaaggagggaatttaaaaatgggacttgagtggtttgtagaatttgtgttcataagttcagatgggtagcaaatggaatagaacttacttaaaaattggggagatttatttgaaaaccagctgtaagttgtgcattgagattatgttaaaagccttggcttaagaatttgaaaatttctttagcctgtagcaacctaaactgtaattcctatcattatgttttattactttccaattacctgtaactgacagaccaaattaattggctttgtgtcctatttagtccatcagtattttcaagtcatgtggaaagcccaaagtcatcacaatgaagagaacaggtgcacagcactgttcctcttgtgttcttgagaaggatctaatttttctgtatatagcccacatcacacttgctttgtcttgtatgttaattgcatcttcattggcttggtatttcctaaatgtttaacaagaacacaagtgttcctgataagatttcctacagtaagccagctctattgtaagcttcccactgtgatgatcatttttttgaagattcattgaacagccaccactctatcatcctcattttggggcagtccaagacatagctggttttagaaacccaagttcctctaagcacagcctcccgggtatgtaactgaacttggtgccaaagtacttgtgtactaatttctattactacgtactgtcactttcctcccgtgccattactgcatcataatacaaggaacctcagagcccccatttgttcattaaagaggcaactacagccaaaatcactgttaaaatcttactacttcatggagtagctcttaggaaaatatatcttcctcctgagtctgggtaattatacctctcccaagcccccattgtgtgttgaaatcctgtcatgaatccttggtagctctctgagaacagtgaagtccagggaaaggcatctggtctgtctggaaagcaaacattatgtggcctctggtagtttttttcctgtaagaatactgactttctggagtaatgagtatatatcagttattgtacatgattgctttgtgaaatgtgcaaatgatatcacctatgcagccttgtttgatttattttctctggtttgtactgttattaaaagcatattgtattatagagctattcagatattttaaatataaagatgtattgtttccgtaatatagacgtatggaatatatttaggtaatagatgtattacttggaaagttctgctttgacaaactgacaaagtctaaatgagcacatgtatcccagtgagcagtaaatcaatggaacatcccaagaagaggataaggatgcttaaaatggaaatcattctccaacgatatacaaattggacttgttcaactgctggatatatgctaccaataaccccagccccaacttaaaattcttacattcaagctcctaagagttcttaatttataactaattttaaaagagaagtttcttttctggttttagtttgggaataatcattcattaaaaaaaatgtattgtggtttatgcgaacagaccaacctggcattacagttggcctctccttgaggtgggcacagcctggcagtgtggccaggggtggccatgtaagtcccatcaggacgtagtcatgcctcctgcatttcgctacccgagtttagtaacagtgcagattccacgttcttgttccgatactctgagaagtgcctgatgttgatgtacttacagacacaagaacaatctttgctataattgtataaagccataaatgtacataaattatgtttaaatggcttggtgtctttcttttctaattatgcagaataagctctttattaggaattttttgtgaagctattaaatacttgagttaagtcttgtcagccacaa

SEQ ID NO: 13 - protein, 457 aa, Homo sapiens
MLGAVKMEGHEPSDWSSYYAEPEGYSSVSNMNAGLGMNGMNTYMSMSAAAMGSGSGNMSAGSMNMSSYVGAGMSPSLAGMSPGAGAMAGMGGSAGAAGVAGMGPHLSPSLSPLGGQAAGAMGGLAPYANMNSMSPMYGQAGLSRARDPKTYRRSYTHAKPPYSYISLITMAIQQSPNKMLTLSEIYQWIMDLFPFYRQNQQRWQNSIRHSLSFNDCFLKVPRSPDKPGKGSFWTLHPDSGNMFENGCYLRRQKRFKCEKQLALKEAAGAAGSGKKAAAGAQASQAQLGEAAGPASETPAGTESPHSSASPCQEHKRGGLGELKGTPAAALSPPEPAPSPGQQQQAAAHLLGPPHHPGLPPEAHLKPEHHYAFNHPFSINNLMSSEQQHHHSHHHHQPHKMDLKAYEQVMHYPGYGSPMPGSLAMGPVTNKTGLDASPLAADTSYYQGVYSRPIMNSS

SEQ ID NO: 14 - DNA, 2428 bp, Homo sapiens
  source 1..2428: mol_type = unassigned DNA
cccgcccacttccaactaccgcctccggcctgcccagggagagagagggagtggagcccagggagagggagcgcgagagagggagggaggaggggacggtgctttggctgacttttttttaaaagagggtgggggtggggggtgattgctggtcgtttgttgtggctgttaaattttaaactgccatgcactcggcttccagtatgctgggagcggtgaagatggaagggcacgagccgtccgactggagcagctactatgcagagcccgagggctactcctccgtgagcaacatgaacgccggcctggggatgaacggcatgaacacgtacatgagcatgtcggcggccgccatgggcagcggctcgggcaacatgagcgcgggctccatgaacatgtcgtcgtacgtgggcgctggcatgagcccgtccctggcggggatgtcccccggcgcgggcgccatggcgggcatgggcggctcggccggggcggccggcgtggcgggcatggggccgcacttgagtcccagcctgagcccgctcggggggcaggcggccggggccatgggcggcctggccccctacgccaacatgaactccatgagccccatgtacgggcaggcgggcctgagccgcgcccgcgaccccaagacctacaggcgcagctacacgcacgcaaagccgccctactcgtacatctcgctcatcaccatggccatccagcagagccccaacaagatgctgacgctgagcgagatctaccagtggatcatggacctcttccccttctaccggcagaaccagcagcgctggcagaactccatccgccactcgctctccttcaacgactgtttcctgaaggtgccccgctcgcccgacaagcccggcaagggctccttctggaccctgcaccctgactcgggcaacatgttcgagaacggctgctacctgcgccgccagaagcgcttcaagtgcgagaagcagctggcgctgaaggaggccgcaggcgccgccggcagcggcaagaaggcggccgccggagcccaggcctcacaggctcaactcggggaggccgccgggccggcctccgagactccggcgggcaccgagtcgcctcactcgagcgcctccccgtgccaggagcacaagcgagggggcctgggagagctgaaggggacgccggctgcggcgctgagccccccagagccggcgccctctcccgggcagcagcagcaggccgcggcccacctgctgggcccgccccaccacccgggcctgccgcctgaggcccacctgaagccggaacaccactacgccttcaaccacccgttctccatcaacaacctcatgtcctcggagcagcagcaccaccacagccaccaccaccaccaaccccacaaaatggacctcaaggcctacgaacaggtgatgcactaccccggctacggttcccccatgcctggcagcttggccatgggcccggtcacgaacaaaacgggcctggacgcctcgcccctggccgcagatacctcctactaccagggggtgtactcccggcccattatgaactcctcttaagaagacgacggcttcaggcccggctaactctggcaccccggatcgaggacaagtgagagagcaagtgggggtcgagactttggggagacggtgttgcagagacgcaagggagaagaaatccataacacccccaccccaacacccccaagacagcagtcttcttcacccgctgcagccgttccgtcccaaacagagggccacacagataccccacgttctatataaggaggaaaacgggaaagaatataaagttaaaaaaaagcctccggtttccactactgtgtagactcctgcttcttcaagcacctgcagattctgatttttttgttgttgttgttctcctccattgctgttgttgcagggaagtcttacttaaaaaaaaaaaaaaattttgtgagtgactcggtgtaaaaccatgtagttttaacagaaccagagggttgtactattgtttaaaaacaggaaaaaaaataatgtaagggtctgttgtaaatgaccaagaaaaagaaaaaaaaagcattcccaatcttgacacggtgaaatccaggtctcgggtccgattaatttatggtttctgcgtgctttatttatggcttataaatgtgtattctggctgcaagggccagagttccacaaatctatattaaagtgttatacccggttttatcccttgaatcttttcttccagatttttcttttctttacttggcttacaaaatatacaggcttggaaattatttcaagaaggagggagggataccctgtctggttgcaggttgtattttattttggcccagggagtgttgctgttttcccaacattttattaataaaattttcagacataaaaaa

SEQ ID NO: 15 - protein, 505 aa, Homo sapiens
MDPGNENSATEAAAIIDLDPDFEPQSRPRSCTWPLPRPEIANQPSEPPEVEPDLGEKVHTEGRSEPILLPSRLPEPAGGPQPGILGAVTGPRKGGSRRNAWGNQSYAELISQAIESAPEKRLTLAQIYEWMVRTVPYFKDKGDSNSSAGWKNSIRHNLSLHSKFIKVHNEATGKSSWWMLNPEGGKSGKAPRRRAASMDSSSKLLRGRSKAPKKKPSVLPAPPEGATPTSPVGHFAKWSGSPCSRNREEADMWTTFRPRSSSNASSVSTRLSPLRPESEVLAEEIPASVSSYAGGVPPTLNEGLELLDGLNLTSSHSLLSRSGLSGFSLQHPGVTGPLHTYSSSLFSPAEGPLSAGEGCFSSSQALEALLTSDTPPPPADVLMTQVDPILSQAPTLLLLGGLPSSSKLATGVGLCPKPLEAPGPSSLVPTLSMIAPPPVMASAPIPKALGTPVLTPPTEAASQDRMPQDLDLDMYMENLECDMDNIISDLMDEGEGLDFNFEPDP

SEQ ID NO: 16 - DNA, 3365 bp, Homo sapiens
  source 1..3365: mol_type = unassigned DNA
aaaagggggagggaactgcggctaaggagacgttcggtgatgggagcgcaatatatgaggggatacagtgcctcaggtttaaaagagcaggaagctgagtgagaggttgcagaaaaagtgtcttcgctcggcagaggttacaggtggcatctcagaaagagctttgaggctacaggctgtagtcgggaaggggatcggagaactgtgtgaagggacagcttagggactagcgtcctgggactagggggaagttcgcgactttctgaagactggcaggaatgtgcctcctggccctcgatgcttcccccctgaggggaggcatcgtgagggactgtggcaggcttcactgaacgctgagccggggaggtccaactccacgtatggatccggggaatgagaattcagccacagaggctgccgcgatcatagacctagatcccgacttcgaaccccagagccgtccccgctcctgcacctggccccttccccgaccagagatcgctaaccagccgtccgagccgcccgaggtggagccagatctgggggaaaaggtacacacggaggggcgctcagagccgatcctgttgccctctcggctcccagagccggccgggggcccccagcccggaatcctgggggctgtaacaggtcctcggaagggaggctcccgccggaatgcctggggaaatcagtcatatgcagaactcatcagccaggccattgaaagcgccccggagaagcgactgacacttgcccagatctacgagtggatggtccgtactgtaccctacttcaaggacaagggtgacagcaacagctcagcaggatggaagaactcgatccgccacaacctgtccctgcacagcaagttcatcaaggttcacaacgaggccaccggcaaaagctcttggtggatgctgaaccctgagggaggcaagagcggcaaagccccccgccgccgggccgcctccatggatagcagcagcaagctgctccggggccgcagtaaagcccccaagaagaaaccatctgtgctgccagctccacccgaaggtgccactccaacgagccctgtcggccactttgccaagtggtcaggcagcccttgctctcgaaaccgtgaagaagccgatatgtggaccaccttccgtccacgaagcagttcaaatgccagcagtgtcagcacccggctgtcccccttgaggccagagtctgaggtgctggcggaggaaataccagcttcagtcagcagttatgcagggggtgtccctcccaccctcaatgaaggtctagagctgttagatgggctcaatctcacctcttcccattccctgctatctcggagtggtctctctggcttctctttgcagcatcctggggttaccggccccttacacacctacagcagctcccttttcagcccagcagaggggcccctgtcagcaggagaagggtgcttctccagctcccaggctctggaggccctgctcacctctgatacgccaccaccccctgctgacgtcctcatgacccaggtagatcccattctgtcccaggctccgactcttctgttgctgggggggcttccttcctccagtaagctggccacgggcgtcggcctgtgtcccaagcccctagaggctccaggccccagcagtctggttcccaccctttctatgatagcaccacctccagtcatggcaagtgcccccatccccaaggctctggggactcctgtgctcacaccccctactgaagctgcaagccaagacagaatgcctcaggatctagatcttgatatgtatatggagaacctggagtgtgacatggataacatcatcagtgacctcatggatgagggcgagggactggacttcaactttgagccagatccctgagtcatgcctggaagctttgtcccctgcttcagatgtggagccaggcgtgttcatatctactctttacccttgagccctccccaggaatttgggaccctgctttagagctagggtggggtctggtcacacacaggtgttgaagaaattataaagataaagctgccccatctggggacgatatggggagggagatgggaggggaaaggggagagggtttttctcactgtgccaattagggggtaaggccccctctcaggagccatcatcggctttccccattcctacccacttaggctttgtagcaagatgagcaatgctgttggaaatgtgaagtcaccagtggccttacccctgcctttgggagcaggatttttttgtagagagtcttatctgagctgagccaggctagctggagcctgggatttctatgcagtggccccttaggccagtgatgtgcggtgggtgggctgtttaggggatctggaagggccaaggtctgagcactggagtggctcgccaggccaaatcacccttagaaggctgcagataacagaaaggctttttataaacttttaaagaaatataaacacaaatatagagattttttaaccatggcagggtgctagtggtgggcagaatgcttttttttctttctgaaggctttgtgatagtgacatgatacaaacactacagacaataaatattaggagacacagggaagtggggagaggtggggagtaatagtaaacacagggaagagctcccctacggaccaggtatagagaaaggtctatgcagaaataggttagagtttccctaacaaaaaagctaacccaggtcccctcattccttcaacttgtgcctgggagtgtgtggtgttagggtgcagccacactcttctatgacccagcatgggttagtgctatggtgggagagtacattgaaggcctggaattagcttggggccagggaagggactgggaggggagagaagagaaggagggaaggatttaggatggtaaagttaggtacagagacctccctgttcaaggcccctgacagctgtccctgcccttcttccccttccctgactgcaggggttatgtggaagtgtgtgtggcagcaggcagcggggaggggaggaacagggaagggggagctggggagcttggctgagggtctgggaaatgagcagggatggggggggatgtggatcaggtttactagcacctgccagggaggccatctggggctccttctccaccccagcccccaaagcagcccttcccccagtgccctttgcatcgtcccctcccccacccctgctgtgggttcccatcatttcctgtgtcagcgcctggcctacccagattgtatcatgtgctagattggagtggggaagtgtgtcaaatcaataaatgaataaattcaataaatgcctataaccagcaaaaaaaaaaaaaaaaa

SEQ ID NO: 17 - protein, 421 aa, Homo sapiens
MNRGFSRKSHTFLPKIFFRKMSSSGAKDKPELQFPFLQDEDTVATLLECKTLFILRGLPGSGKSTLARVIVDKYRDGTKMVSADAYKITPGARGAFSEEYKRLDEDLAAYCRRRDIRILVLDDTNHERERLEQLFEMADQYQYQVVLVEPKTAWRLDCAQLKEKNQWQLSADDLKKLKPGLEKDFLPLYFGWFLTKKSSETLRKAGQVFLEELGNHKAFKKELRQFVPGDEPREKMDLVTYFGKRPPGVLHCTTKFCDYGKAPGAEEYAQQDVLKKSYSKAFTLTISALFVTPKTTGARVELSEQQLQLWPSDVDKLSPTDNLPRGSRAHITLGCAADVEAVQTGLDLLEILRQEKGGSRGEEVGELSRGKLYSLGNGRWMLTLAKNMEVRAIFTGYYGKGKPVPTQGSRKGGALQSCTII

SEQ ID NO: 18 - DNA, 2598 bp, Homo sapiens
  source 1..2598: mol_type = unassigned DNA
ctccgcgcaggcgggcggccccggagcgctggtgccggcagaggcggcgacggtggcgcccctcctcatcatgaggcttctcccgaaaaagccacacattcctgcccaagatcttcttccgcaagatgtcatcctcaggggccaaggacaagcctgagctgcagtttcccttccttcaggatgaggacacagtggccacgctgctagagtgcaagacgctcttcatcttgcgcggcctgccaggaagcggcaagtccacgctggcacgggtcatcgtggacaagtaccgtgatggcaccaagatggtgtcggctgacgcttacaagatcacccccggcgctcgaggagccttctccgaggagtacaagcggctcgatgaggacctggctgcctactgccgccgccgggacatcagaattcttgtgcttgatgacaccaaccacgaacgggaacggctggagcagctctttgaaatggccgaccagtaccagtaccaggtggtgctggtggagcccaagacggcgtggcggctggactgtgcccagctcaaggagaagaaccagtggcagctgtcggctgatgacctgaagaagctgaagcctgggctggagaaggacttcctgccgctctacttcggctggttcctgaccaagaagagctctgagaccctccgcaaagccggccaggtcttcctggaagagctggggaaccacaaggccttcaagaaggagctgcgacaattcgtccctggggatgagcccagggagaagatggacttggtcacctactttggaaagagacccccaggcgtgctgcattgcacaaccaagttttgtgactacgggaaggctcccggggcagaggagtacgctcaacaagatgtgttaaagaaatcttactccaaggccttcacgctgaccatctctgccctctttgtgacacccaagacgactggggcccgggtggagttaagcgagcagcaactgcagttgtggccgagtgatgtggacaagctgtcacccactgacaacctgccgcgggggagccgcgcccacatcaccctcggctgtgcagctgacgtagaggccgtgcagacgggccttgacctcttagagattctgcggcaggagaaggggggcagccgaggcgaggaggtgggcgagctaagccggggcaagctctattccttgggcaatgggcgctggatgctgaccctggccaagaacatggaggtcagggccatcttcacggggtactacgggaaaggcaaacctgtgcccacgcaaggtagccggaaggggggcgccttgcagtcctgcaccatcatatgagtgttctcaccaccacttatgcccctagaagggaaggggagagggaaacgtgccctctgtttgatccttgttttgtgacattttttttttttttttttttactcaaagttaacctacctgtaactttttaaaaacttgtaaaataactgaccctcccttcctgtccgccctcttcccctctaatgctcacgctcccaacacaaggtgggcagggaggcaccattcaggaacctggaccaaagctgacgaggctgggccaagccagggatggggccacagccagaaccccgagccctacttccaggttctggttagctcagccccagcccagcccagctgctctgcccagagctgggtgagtggggagacacctcagagccccgcaaaacccactgaccggaggcaaaaggcagtggggctgggggtagttttccatggtcacagagaactagtggtggctctgagaaggggaggacctctgggctttgattccatctccttgtcttttttctttgtttttagagacagggtcctgctatttcccaagctggagtgcagtggtgcgatcatggctcactgcagcctcgaactcctgggctcaagcaatcctcctgagtgatcccatttcttaatcagtgtagccccaagaaggctggggctatttaccagggtagaaaaaggagcttacctcccacctttggtcctaagtccctgccccctccccttcacaccataactaggtaacagtttgataactagggaagaaagcagaacagttaagcagccgccacatccccgctggctgggggcctcactccaggaaggggctggactggctgtcctttccagtggcctggctccgctgtgtggatggggagatcggggccagaggcagaaccctggtgaggaagctccagtcctgctctctacccagcccatcttgcctccatggtgcctctggaggcctctgggcctcctctaacaggggctggtgggcaccaagagccaatggagtagacccctggctggtaagggccaagtcccaccggttgcttctgggaaggggtttctaacactagtctgtgtgctgtggttcctggggtgccctccactgccctctgttcagtaacagggccttgctaatcgggttgtcactcaacaaaagtgctttggatttaagttactatcctggctttgcccaacctcagcaacctgtaagactgataatgaaataaatcatgttaatcctagcaaaaaaaaaaaaaaa

SEQ ID NO: 19 - protein, 304 aa, Homo sapiens
MGNHAGKRELNAEKASTNSETNRGESEKKRNLGELSRTTSEDNEVFGEADANQNNGTSSQDTAVTDSKRTADPKNAWQDAHPADPGSRPHLIRLFSRDAPGREDNTFKDRPSESDELQTIQEDSAATSESLDVMASQKRPSQRHGSKYLATASTMDHARHGFLPRHRDTGILDSIGRFFGGDRGAPKRGSGKDSHHPARTAHYGSLPQKSHGRTQDENPVVHFFKNIVTPRTPPPSQGKGRGLSLSRFSWGAEGQRPGFGYGGRASDYKSAHKGFKGVDAQGTLSKIFKLGGRDSRSGSPMARR

SEQ ID NO: 20 - DNA, 2139 bp, Homo sapiens
  source 1..2139: mol_type = unassigned DNA
gaaaacagtgcagccacctccgagagcctggatgtgatggcgtcacagaagagaccctcccagaggcacggatccaagtacctggccacagcaagtaccatggaccatgccaggcatggcttcctcccaaggcacagagacacgggcatccttgactccatcgggcgcttctttggcggtgacaggggtgcgccaaagcggggctctggcaaggactcacaccacccggcaagaactgctcactatggctccctgccccagaagtcacacggccggacccaagatgaaaaccccgtagtccacttcttcaagaacattgtgacgcctcgcacaccacccccgtcgcagggaaaggggagaggactgtccctgagcagatttagctggggggccgaaggccagagaccaggatttggctacggaggcagagcgtccgactataaatcggctcacaagggattcaagggagtcgatgcccagggcacgctttccaaaatttttaagctgggaggaagagatagtcgctctggatcacccatggctagacgctgaaaacccacctggttccggaatcctgtcctcagcttcttaatataactgccttaaaactttaatcccacttgcccctgttacctaattagagcagatgacccctcccctaatgcctgcggagttgtgcacgtagtagggtcaggccacggcagcctaccggcaatttccggccaacagttaaatgagaacatgaaaacagaaaacggttaaaactgtccctttctgtgtgaagatcacgttccttcccccgcaatgtgcccccagacgcacgtgggtcttcagggggccaggtgcacagacgtccctccacgttcacccctccacccttggactttcttttcgccgtggctcggcacccttgcgcttttgctggtcactgccatggaggcacacagctgcagagacagagaggacgtgggcggcagagaggactgttgacatccaagcttcctttgtttttttttcctgtccttctctcacctcctaaagtagacttcatttttcctaacaggattagacagtcaaggagtggcttactacatgtgggagctttttggtatgtgacatgcgggctgggcagctgttagagtccaacgtggggcagcacagagagggggccacctccccaggccgtggctgcccacacaccccaattagctgaattcgcgtgtggcagagggaggaaaaggaggcaaacgtgggctgggcaatggcctcacataggaaacagggtcttcctggagatttggtgatggagatgtcaagcaggtggcctctggacgtcaccgttgccctgcatggtggccccagagcagcctctatgaacaacctcgtttccaaaccacagcccacagccggagagtccaggaagacttgcgcactcagagcagaagggtaggagtcctctagacagcctcgcagccgcgccagtcgcccatagacactggctgtgaccgggcgtgctggcagcggcagtgcacagtggccagcactaaccctccctgagaagataaccggctcattcacttcctcccagaagacgcgtggtagcgagtaggcacaggcgtgcacctgctcccgaattactcaccgagacacacgggctgagcagacggcccctgtgatggagacaaagagctcttctgaccatatccttcttaacacccgctggcatctcctttcgcgcctccctccctaacctactgacccaccttttgattttagcgcacctgtgattgataggccttccaaagagtcccacgctggcatcaccctccccgaggacggagatgaggagtagtcagcgtgatgccaaaacgcgtcttcttaatccaattctaattctgaatgtttcgtgtgggcttaataccatgtctattaatatatagcctcgatgatgagagagttacaaagaacaaaactccagacacaaacctccaaatttttcagcagaagcactctgcgtcgctgagctgaggtcggctctgcgatccatacgtggccgcacccacacagcacgtgctgtgacgatggctgaacggaaagtgtacactgttcctgaatattgaaataaaacaataaactttt

SEQ ID NO: 21 - protein, 378 aa, Homo sapiens
MDSVRSGAFGHLFRPDNFIFGQSGAGNNWAKGHYTEGAELVDSVLDVVRKECENCDCLQGFQLTHSLGGGTGSGMGTLLISKVREEYPDRIMNTFSVVPSPKVSDTVVEPYNATLSIHQLVENTDETYCIDNEALYDICFRTLKLATPTYGDLNHLVSATMSGVTTSLRFPGQLNADLRKLAVNMVPFPRLHFFMPGFAPLTARGSQQYRALTVPELTQQMFDAKNMMAACDPRHGRYLTVATVFRGRMSMKEVDEQMLAIQSKNSSYFVEWIPNNVKVAVCDIPPRGLKMSSTFIGNSTAIQELFKRISEQFTAMFRRKAFLHWYTGEGMDEMEFTEAESNMNDLVSEYQQYQDATAEEEGEMYEDDEEESEAQGPK

SEQ ID NO: 22 - DNA, 1720 bp, Homo sapiens
  source 1..1720: mol_type = unassigned DNA
gcccggcccgcccgcgcccgtccgcagccgcccgccagacgcgcccagtatgagggagatcgtgcacatccaggccggccagtgcggcaaccagatcggggccaagttctgggaagtcatcagtgatgagcatggcatcgaccccagcggcaactacgtgggcgactcggacttgcagctggagcggatcagcgtctactacaacgaggcctcttctcacaagtacgtgcctcgagccattctggtggacctggaacccggaaccatggacagtgtccgctcaggggcctttggacatctcttcaggcctgacaatttcatctttggtcagagtggggccggcaacaactgggccaagggtcactacacggagggggcggagctggtggattcggtcctggatgtggtgcggaaggagtgtgaaaactgcgactgcctgcagggcttccagctgacccactcgctggggggcggcacgggctccggcatgggcacgttgctcatcagcaaggtgcgtgaggagtatcccgaccgcatcatgaacaccttcagcgtcgtgccctcacccaaggtgtcagacacggtggtggagccctacaacgccacgctgtccatccaccagctggtggagaacacggatgagacctactgcatcgacaacgaggcgctctacgacatctgcttccgcaccctcaagctggccacgcccacctacggggacctcaaccacctggtatcggccaccatgagcggagtcaccacctccttgcgcttcccgggccagctcaacgctgacctgcgcaagctggccgtcaacatggtgcccttcccgcgcctgcacttcttcatgcccggcttcgcccccctcacagcccggggcagccagcagtaccgggccctgaccgtgcccgagctcacccagcagatgttcgatgccaagaacatgatggccgcctgcgacccgcgccacggccgctacctgacggtggccaccgtgttccggggccgcatgtccatgaaggaggtggacgagcagatgctggccatccagagcaagaacagcagctacttcgtggagtggatccccaacaacgtgaaggtggccgtgtgtgacatcccgccccgcggcctcaagatgtcctccaccttcatcgggaacagcacggccatccaggagctgttcaagcgcatctccgagcagttcacggccatgttccggcgcaaggccttcctgcactggtacacgggcgagggcatggacgagatggagttcaccgaggccgagagcaacatgaacgacctggtgtccgagtaccagcagtaccaggacgccacggccgaggaagagggcgagatgtacgaagacgacgaggaggagtcggaggcccagggccccaagtgaagctgctcgcagctggagtgagaggcaggtggcggccggggccgaagccagcagtgtctaaacccccggagccatcttgctgccgacaccctgctttcccctcgccctagggctcccttgccgccctcctgcagtatttatggcctcgtcctccccacctaggccacgtgtgagctgctcctgtctctgtcttattgcagctccaggcctgacgttttacggttttgttttttactggtttgtgtttatattttcggggatacttaataaatctattgctgtcagatacccttaaaaaaaaaaaaaaaaaaaaaaaaa

SEQ ID NO: 23 - protein, 312 aa, Homo sapiens
MAQPYPPAQYPPPPQNGIPAEYAPPPPHPTQDYSGQTPVPTEHGMTLYTPAQTHPEQPGSEASTQPIAGTQTVPQTDEAAQTDSQPLHPSDPTEKQQPKRLHVSNIPFRFRDPDLRQMFGQFGKILDVEIIFNERGSKGFGFVTFETSSDADRAREKLNGTIVEGRKIEVNNATARVMTNKKTGNPYTNGWKLNPVVGAVYGPEFYAVTGFPYPTTGTAVAYRGAHLRGRGRAVYNTFRAAPPPPPIPTYGAVVYQDGFYGAEIYGGYAAYRYAQPAAAAAAYSDSYGRVYAAADPYHHTIGPAATYSIGTM

SEQ ID NO: 24 - DNA, 3007 bp, Homo sapiens
  source 1..3007: mol_type = unassigned DNA
gatacagcagcagctggtgctcctggccaggctgtgcgtgctctctctgcctctctctctcggactctctgctctctctctctgactctctcctctctctctgttggcctggtgaaatgttcttggctgtaggcacacagagccttggactcaaggctgttggagtcgaggacaccttgacttcggtcctggaggttgaaattctgcctctgagaagctaacagtcttcctgtggtcgccactcctccccagcagccccctccttgccaaggacggtccagaaggagccccactggggcctccccgctcagcaaagcagacctcacctcccactaccagcttgaagtcacagcagccagaggaaattctgccaccattttcccaggtctgcagcccctccagctgggaacctgctcctggagccatccctctgcaaacagagagcccagagtgcctcggggaaaattggctgaataaaagagcgatcaggacgccacggctccgcctgaagcgatggcccagccctacccccccgcccagtacccccctccgccacagaacggcatccctgccgagtacgccccgcccccaccgcaccccacgcaggactactccggccagaccccggtccccacagagcatggcatgaccctgtacacaccagcacagacccaccccgagcagccaggctccgaggccagcacacagcccatcgccgggacccagacagtgccgcagacagacgaggcggcacagacggacagccagccgctccacccctccgaccctacagagaagcagcagcccaagcggctacacgtctccaacatccccttccggttcagggaccccgacttgcggcaaatgttcgggcaattcggaaaaattttagacgtggagatcatttttaacgagcggggctccaagggttttgggtttgtaacttttgaaactagctcagatgctgaccgagcccgggagaagctgaatgggacgatcgtagagggacggaaaattgaggtcaataatgccacggcccgagtgatgaccaacaagaagacggggaacccctacaccaacggctggaagctaaatccagtggtcggcgcagtctacgggcctgaattctatgcagtgacggggttcccctaccccaccaccggcacagccgttgcctaccggggcgcacatcttcggggccggggccgggccgtgtataatacatttcgggctgcgccacccccaccccccatcccgacttacggagcggtcgtgtatcaggatggattttatggtgctgagatttatggaggctacgcagcctacagatacgctcagcccgctgcagcggcggcagcctacagcgacagttacggcagagtctacgcagctgccgacccgtaccatcacaccatcgggcccgcggcgacctacagcattggaaccatgtgaaaccttccaccgtttccttctcggaccatgaagggcaaaaacaaaaaaacaaaaaaaatcacaaaacaaaaaaaacaaaaaaagatgttaagatccaagcaacaaaaaaaaaaccaaccaaaccaagaggcatccaaccaagtccaagtcccgcgtcctggccacacgcccgcaccgagggagcacgccggcaggggcgccgaggagcggccccaggacaggacggccccaccgcgtcctggctggcagcacagtgggaacacgcccctccgtctcaggcagtgggggagttggaggggaaggggcctcccttgtgggacccgtggggggctctgttttccatccagtcttcctttcccagcccccaactcccaagacagacagtgtggagcccagcggcggcggagcaggcccgggcctgagcaggcaggcgctgctagcaagacttgatctttgtggccagctgtgccagggggccggcggggctgaggggtgcgggcagctttcatcccaggggctccactgggccccgtcaccctcctgtcgcgtcccctgcgtcccacctccctcctgcccggcagtcccgcccgtgcccccagcctggcgaggaagccgtccaacagtagccccggggccagctcccaacagaaagggctgacgtggctccaggactcaggggcgctccatgggaggacgaaggaagcccagccagccaggagccactcctcacacctccaagtgtggccaagtgggccctgaggccaaggacttacttgctcttcctggccatctctccctttctggaggaggcccggggcctgtgtacaccaaggctgacctcgtgctgcctgctgggacccagccctccctgccgctcccctgtgagcccagtccaccgtgggcgcccagggccagggacgggccagcgcccggctgcatcgcgaggttgggagtcacagtggctgtgggcctggacgggcacagccagagcaggggcccatgggaagggcaagggatggggaagcctgggccggccccttccctgctcccaaggcaggtgtccaggtggcgggagcagcaccaaggacagccaggcttacccggtgggaggagcaggagcagagcaggtggcagggaggaacccctggcgaggcagggagcactgaagtagggaagcagcaaaaaatacaggctcccaacgtggctccactgtctcatgaagtgtcaaaaatttaaaaatacacctcactttctattcagcatcagctattgaaatggaattctccttttctattcccgttgtacatagccccacgccctgcctccggctttgtcctctgtacagagccccctgtcccctctgctgttccggacccttttcttgcagcagctcaaccccccgactcactcagatccccaggactgcagccgagccccgggcttcctttcttaccattctgtatgcttccaaggtgtgaccattcaaactaacagtattattaagattattaataaagatttctttcttcaaaccaggaaaaaaaaaaaaaaaaaa

SEQ ID NO: 25 - protein, 564 aa, Homo sapiens
MSSHGNSLFLRESGQRLGRVGWLQRLQESLQQRALRTRLRLQTMTLEHVLRFLRRNAFILLTVSAVVIGVSLAFALRPYQLTYRQIKYFSFPGELLMRMLQMLVLPLIVSSLVTGMASLDNKATGRMGMRAAVYYMVTTIIAVFIGILMVTIIHPGKGSKEGLHREGRIETIPTADAFMDLIRNMFPPNLVEACFKQFKTQYSTRVVTRTMVRTENGSEPGASMPPPFSVENGTSFLENVTRALGTLQEMLSFEETVPVPGSANGINALGLVVFSVAFGLVIGGMKHKGRVLRDFFDSLNEAIMRLVGIIIWYAPVGILFLIAGKILEMEDMAVLGGQLGMYTLTVIVGLFLHAGIVLPLIYFLVTHRNPFPFIGGMLQALITAMGTSSSSATLPITFRCLEEGLGVDRRITRFVLPVGATVNMDGTALYEALAAIFIAQVNNYELNLGQITTISITATAASVGAAGIPQAGLVTMVIVLTSVGLPTEDITLIIAVDWFLDRLRTMTNVLGDSIGAAVIEHLSQRELELQEAELTLPSLGKPYKSLMAQEKGASRGRGGNESAM

SEQ ID NO: 26 - DNA, 2990 bp, Homo sapiens
  source 1..2990: mol_type = unassigned DNA
ggcatagcgcgtcccggctccgcgccggtgcctccacggtccggtccccgcgccggtgctgcacagtccctggcgggtccccgcggccccggccgggcgcttcgccgggctccggctcctgcatccgggcgcagcgcgcaggccgaggcgcgggcaggccgcccccgccgctccggacgccgggatgtaagaggctccgaaaagcagcccacgcatctcatcagatctaagtgtctagaggtcgggagaaccaagtgggaaagacccaccctcacccctcaccttgtagaaactgggaacactagaagggacattttctgagcaggaaacccaagagacagggttttacgctgtcacccaagttggagtgcagtggtacgatcatagctcattgcagcctcaaactcctgggttcaagcgatcctcctgctttagcctcttgagtagctaggactacaggcacaggccaccgtgcctggctaatttttaatttttaaaaaagagacagggtctggctatgttgcccaggctggccatgaactcctgggctcaagcggttctccagccttcacctcccaaagtgttgggattgcaggcatgagccactgcgtctggcccacagatgctaagtgctgtctgctcttctccaggggtcagcaaattttttcagcaaatggcccaagagtaaatattttgagctttgtggcccgtacaatctctgtcccaacaactcaactcaggcattgtagcttgaaagcagctgtagacaataggtaatccatgagtgtggctgtgtgccaataaaactttatttacaaaaacaagcagtaggctgaatttgactagcagaccatagtttgtcaataccgtattatgtcttgtaaggaagagaaaggaaccagacaaaactctagcctcgggagttttcctgactgttcagatcttagctgaatgatctcccttggtatctacaggcaacttcctgctgtggcttagggactggaaacataatatcccagagggattccctgtgtagtctgtggttcactctttgggattttttttttttttttcacagcaaggagaagcagcattgtggtttcaggagatgggtccatttggagcaggatcctaagtggggcttggcattgggaatttggattagctctagaggacgcaggatctggaaaatcagggcagatttcccatcccttggatatggtggggagttgaggagggcaaggaagatcccagaaaagccagtggcagcaaaacacaaaggccagggacctacgtactggtaaaactgagacctccaagaaacctgcagctcgacctggttgaattcagatagaccatgagcagccatggcaacagcctgttccttcgggagagcggccagcggctgggccgggtgggctggctgcagcggctgcaggaaagcctgcagcagagagcactgcgcacgcgcctgcgcctgcagaccatgaccctcgagcacgtgctgcgcttcctgcgccgaaacgccttcattctgctgacggtcagcgccgtggtcattggggtcagcctggcctttgccctgcgcccatatcagctcacctaccgccagatcaagtacttctcttttcctggagagcttctgatgaggatgctgcagatgctggtgttacctctcattgtctccagcctggtcacaggtatggcatccctggacaacaaggccacggggcggatggggatgcgggcagctgtgtactacatggtgaccaccatcatcgcggtcttcatcggcatcctcatggtcaccatcatccatcccgggaagggctccaaggaggggctgcaccgggagggccggatcgagaccatccccacagctgatgccttcatggacctgatcagaaatatgtttccaccaaaccttgtggaggcctgcttcaaacagttcaagacgcagtacagcacgagggtggtaaccaggaccatggtgaggacagagaacgggtctgagccgggtgcctccatgcctcctccattctcagtggagaacggaaccagcttcctggaaaatgtcactcgggccttgggtaccctgcaggagatgctgagctttgaggagactgtacccgtgcctggctccgccaatggcatcaacgccctgggcctcgtggtcttctctgtggcctttgggctggtcattggtggcatgaaacacaagggcagagtcctcagggacttcttcgacagcctcaatgaggctattatgaggctggtgggcatcattatctggtgagtcctggtctgtgcccacgggaaggtggagccagagctgggaagtcaggctgtggggaagctgccgaagggcttgctggggacctttggtcattcatttacgtattgggtgattcacttacccactcaccaactcattcattcatgtctttctgggatgatttcatcactagttcacttccttgttcatctgttcattcattcattcttctatgcattggttagttcatggaatatctcactctttcattcattcatgtccttctgcaatgattcattcactgctttgttcatctgttcattcactcattcttctatgcattgatgaaatcactcattcagtgatttattcatctatactcatgcttcaatgcattgatttactcatttcctcatgcatttattcattcatctatgcattggttaaatcactggccaactcactaactcattcattcattcacacttttctgcaatgatttgttcacttgttcactcccttgcttatctgttcattcactcattcttcaatacattgaccaagccattcactgacatttattcagctacatttattctttcatgcattggtctggatttatttggtcattcatttatttattttgcaaaattaatgtatttttaattgacaaataaaaactgtatatattttcatgtgcaaaaaaaaaaaaaaa

SEQ ID NO: 27 - protein, 1192 aa, Homo sapiens
MEDLDQSPLVSSSDSPPRPQPAFKYQFVREPEDEEEEEEEEEEDEDEDLEELEVLERKPAAGLSAAPVPTAPAAGAPLMDFGNDFVPPAPRGPLPAAPPVAPERQPSWDPSPVSSTVPAPSPLSAAAVSPSKLPEDDEPPARPPPPPPASVSPQAEPVWTPPAPAPAAPPSTPAAPKRRGSSGSVDETLFALPAASEPVIRSSAENMDLKEQPGNTISAGQEDFPSVLLETAASLPSLSPLSAASFKEHEYLGNLSTVLPTEGTLQENVSEASKEVSEKAKTLLIDRDLTEFSELEYSEMGSSFSVSPKAESAVIVANPREEIIVKNKDEEEKLVSNNILHNQQELPTALTKLVKEDEVVSSEKAKDSFNEKRVAVEAPMREEYADFKPFERVWEVKDSKEDSDMLAAGGKIESNLESKVDKKCFADSLEQTNHEKDSESSNDDTSFPSTPEGIKDRSGAYITCAPFNPAATESIATNIFPLLGDPTSENKTDEKKIEEKKAQIVTEKNTSTKTSNPFLVAAQDSETDYVTTDNLTKVTEEVVANMPEGLTPDLVQEACESELNEVTGTKIAYETKMDLVQTSEVMQESLYPAAQLCPSFEESEATPSPVLPDIVMEAPLNSAVPSAGASVIQPSSSPLEASSVNYESIKHEPENPPPYEEAMSVSLKKVSGIKEEIKEPENINAALQETEAPYISIACDLIKETKLSAEPAPDFSDYSEMAKVEQPVPDHSELVEDSSPDSEPVDLFSDDSIPDVPQKQDETVMLVKESLTETSFESMIEYENKEKLSALPPEGGKPYLESFKLSLDNTKDTLLPDEVSTLSKKEKIPLQMEELSTAVYSNDDLFISKEAQIRETETFSDSSPIEIIDEFPTLISSKTDSFSKLAREYTDLEVSHKSEIANAPDGAGSLPCTELPHDLSLKNIQPKVEEKISFSDDFSKNGSATSKVLLLPPDVSALATQAEIESIVKPKVLVKEAEKKLPSDTEKEDRSPSAIFSAELSKTSVVDLLYWRDIKKTGVVFGASLFLLLSLTVFSIVSVTAYIALALLSVTISFRIYKGVIQAIQKSDEGHPFRAYLESEVAISEELVQKYSNSALGHVNCTIKELRRLFLVDDLVDSLKFAVLMWVFTYVGALFNGLTLLILALISLFSVPVIYERHQAQIDHYLGLANKNVKDAMAKIQAKIPGLKRKAE

SEQ ID NO: 28 - DNA, 4871 bp, Homo sapiens
  source 1..4871: mol_type = unassigned DNA
agtccctgccctcccctggggagggtgagtcacgccaaactgggcggagagtccgctggcctcactcctagctcatctgggcggcggcggcaagtggggacagggcgggtggcgcatcaccggcgcggaggcaggaggagcagtctcattgttccgggagccgtcaccacagtaggtccctcggctcagtcggcccagcccctctcagtcctccccaacccccacaaccgcccgcggctctgagacgcggccccggcggcggcggcagcagctgcagcatcatctccaccctccagccatggaagacctggaccagtctcctctggtctcgtcctcggacagcccaccccggccgcagcccgcgttcaagtaccagttcgtgagggagcccgaggacgaggaggaagaagaggaggaggaagaggaggacgaggacgaagacctggaggagctggaggtgctggagaggaagcccgccgccgggctgtccgcggccccagtgcccaccgcccctgccgccggcgcgcccctgatggacttcggaaatgacttcgtgccgccggcgccccggggacccctgccggccgctccccccgtcgccccggagcggcagccgtcttgggacccgagcccggtgtcgtcgaccgtgcccgcgccatccccgctgtctgctgccgcagtctcgccctccaagctccctgaggacgacgagcctccggcccggcctccccctcctcccccggccagcgtgagcccccaggcagagcccgtgtggaccccgccagccccggctcccgccgcgcccccctccaccccggccgcgcccaagcgcaggggctcctcgggctcagtggatgagaccctttttgctcttcctgctgcatctgagcctgtgatacgctcctctgcagaaaatatggacttgaaggagcagccaggtaacactatttcggctggtcaagaggatttcccatctgtcctgcttgaaactgctgcttctcttccttctctgtctcctctctcagccgcttctttcaaagaacatgaataccttggtaatttgtcaacagtattacccactgaaggaacacttcaagaaaatgtcagtgaagcttctaaagaggtctcagagaaggcaaaaactctactcatagatagagatttaacagagttttcagaattagaatactcagaaatgggatcatcgttcagtgtctctccaaaagcagaatctgccgtaatagtagcaaatcctagggaagaaataatcgtgaaaaataaagatgaagaagagaagttagttagtaataacatccttcataatcaacaagagttacctacagctcttactaaattggttaaagaggatgaagttgtgtcttcagaaaaagcaaaagacagttttaatgaaaagagagttgcagtggaagctcctatgagggaggaatatgcagacttcaaaccatttgagcgagtatgggaagtgaaagatagtaaggaagatagtgatatgttggctgctggaggtaaaatcgagagcaacttggaaagtaaagtggataaaaaatgttttgcagatagccttgagcaaactaatcacgaaaaagatagtgagagtagtaatgatgatacttctttccccagtacgccagaaggtataaaggatcgttcaggagcatatatcacatgtgctccctttaacccagcagcaactgagagcattgcaacaaacatttttcctttgttaggagatcctacttcagaaaataagaccgatgaaaaaaaaatagaagaaaagaaggcccaaatagtaacagagaagaatactagcaccaaaacatcaaacccttttcttgtagcagcacaggattctgagacagattatgtcacaacagataatttaacaaaggtgactgaggaagtcgtggcaaacatgcctgaaggcctgactccagatttagtacaggaagcatgtgaaagtgaattgaatgaagttactggtacaaagattgcttatgaaacaaaaatggacttggttcaaacatcagaagttatgcaagagtcactctatcctgcagcacagctttgcccatcatttgaagagtcagaagctactccttcaccagttttgcctgacattgttatggaagcaccattgaattctgcagttcctagtgctggtgcttccgtgatacagcccagctcatcaccattagaagcttcttcagttaattatgaaagcataaaacatgagcctgaaaaccccccaccatatgaagaggccatgagtgtatcactaaaaaaagtatcaggaataaaggaagaaattaaagagcctgaaaatattaatgcagctcttcaagaaacagaagctccttatatatctattgcatgtgatttaattaaagaaacaaagctttctgctgaaccagctccggatttctctgattattcagaaatggcaaaagttgaacagccagtgcctgatcattctgagctagttgaagattcctcacctgattctgaaccagttgacttatttagtgatgattcaatacctgacgttccacaaaaacaagatgaaactgtgatgcttgtgaaagaaagtctcactgagacttcatttgagtcaatgatagaatatgaaaataaggaaaaactcagtgctttgccacctgagggaggaaagccatatttggaatcttttaagctcagtttagataacacaaaagataccctgttacctgatgaagtttcaacattgagcaaaaaggagaaaattcctttgcagatggaggagctcagtactgcagtttattcaaatgatgacttatttatttctaaggaagcacagataagagaaactgaaacgttttcagattcatctccaattgaaattatagatgagttccctacattgatcagttctaaaactgattcattttctaaattagccagggaatatactgacctagaagtatcccacaaaagtgaaattgctaatgccccggatggagctgggtcattgccttgcacagaattgccccatgacctttctttgaagaacatacaacccaaagttgaagagaaaatcagtttctcagatgacttttctaaaaatgggtctgctacatcaaaggtgctcttattgcctccagatgtttctgctttggccactcaagcagagatagagagcatagttaaacccaaagttcttgtgaaagaagctgagaaaaaacttccttccgatacagaaaaagaggacagatcaccatctgctatattttcagcagagctgagtaaaacttcagttgttgacctcctgtactggagagacattaagaagactggagtggtgtttggtgccagcctattcctgctgctttcattgacagtattcagcattgtgagcgtaacagcctacattgccttggccctgctctctgtgaccatcagctttaggatatacaagggtgtgatccaagctatccagaaatcagatgaaggccacccattcagggcatatctggaatctgaagttgctatatctgaggagttggttcagaagtacagtaattctgctcttggtcatgtgaactgcacgataaaggaactcaggcgcctcttcttagttgatgatttagttgattctctgaagtttgcagtgttgatgtgggtatttacctatgttggtgccttgtttaatggtctgacactactgattttggctctcatttcactcttcagtgttcctgttatttatgaacggcatcaggcacagatagatcattatctaggacttgcaaataagaatgttaaagatgctatggctaaaatccaagcaaaaatccctggattgaagcgcaaagctgaatgaaaacgcccaaaataattagtaggagttcatctttaaaggggatattcatttgattatacgggggagggtcagggaagaacgaaccttgacgttgcagtgcagtttcacagatcgttgttagatctttatttttagccatgcactgttgtgaggaaaaattacctgtcttgactgccatgtgttcatcatcttaagtattgtaagctgctatgtatggatttaaaccgtaatcatatctttttcctatctatctgaggcactggtggaataaaaaacctgtatattttactttgttgcagatagtcttgccgcatcttggcaagttgcagagatggtggagctagaaaaaaaaaaaaaaaagcccttttcagtttgtgcactgtgtatggtccgtgtagattgatgcagattttctgaaatgaaatgtttgtttagacgagatcataccggtaaagcaggaatgacaaagcttgcttttctggtatgttctaggtgtattgtgacttttactgttatattaattgccaatataagtaaatatagattatatatgtatagtgtttcacaaagcttagacctttaccttccagccaccccacagtgcttgatatttcagagtcagtcattggttatacatgtgtagttccaaagcacataagctagaagaagaaatatttctaggagcactaccatctgttttcaacatgaaatgccacacacatagaactccaacatcaatttcattgcacagactgactgtagttaattttgtcacagaatctatggactgaatctaatgcttccaaaaatgttgtttgtttgcaaatatcaaacattgttatgcaagaaattattaattacaaaatgaagatttataccattgtggtttaagctgtactgaactaaatctgtggaatgcattgtgaactgtaaaagcaaagtatcaataaagcttatagacttaaaaaaaaaaaaaaaaaaaaaaaaaaaaaaa

SEQ ID NO: 29 - protein, 271 aa, Homo sapiens
MYYAVSQARVNAVPGTMLRPQRPGDLQLGASLYELVGYRQPPSSSSSSTSSTSSTSSSSTTAPLLPKAAREKPEAPAEPPGPGPGSGAHPGGSARPDAKEEQQQQLRRKINSRERKRMQDLNLAMDALREVILPYSAAHCQGAPGRKLSKIATLLLARNYILLLGSSLQELRRALGEGAGPAAPRLLLAGLPLLAAAPGSVLLAPGAVGPPDALRPAKYLSLALDEPPCGQFALPGGGAGGPGLCTCAVCKFPHLVPASLGLAAVQAQFSK

SEQ ID NO: 30 - DNA, 2298 bp, Homo sapiens
  source 1..2298: mol_type = unassigned DNA
gttctagatcgtttccccgcgcgcaggtccgcggggaggggcggcctgccgaccggcccaccccagggcgttcctgaagggcgtcctcggccgcccccaccgcctcccagatgtactatgcggtttcccaggcgcgcgtgaacgcggtccccgggaccatgctgcggccacagcggcccggagacttgcagctcggggcctccctctacgagctggtgggctacaggcagccgccctcctcctcctcctcctccacctcctccacctcctccacttcctcctcctccacgacggcccccctcctccccaaggctgcgcgcgagaagccggaggcgccggccgagcctccaggccccgggcccgggtcaggcgcgcacccgggcggcagcgcccggccggacgccaaggaggagcagcagcagcagctgcggcgcaagatcaacagccgcgagcggaagcgcatgcaggacctgaacctggccatggacgccctgcgcgaggtcatcctgccctactcagcggcgcactgccagggcgcgcccggccgcaagctctccaagatagccacgctgctgctcgcccgcaactacatcctactgctgggcagctcgctgcaggagctgcgccgcgcgctgggcgagggcgccgggcccgccgcgccgcgcctgctgctggccgggctgcccctgctcgccgccgcgcccggctccgtgctgctggcgcccggcgccgtaggaccccccgacgcgctgcgccccgccaagtacctgtcgctggcgctggacgagccgccgtgcggccagttcgctctccccggcggcggcgcaggcggccccggcctctgcacctgcgccgtgtgcaagttcccgcacctggtcccggccagcctgggcctggccgccgtgcaggcgcaattctccaagtgagggcgggtctgggcctggggcgcgacctcggcccggcctcccttcgctcagcttctccgcgcccctgctccctgcgtctgggagagcgaggccgagcaaggaaagcatttcgaaccttccagtccagaggaagggactgtcgggcacccccttccccgcccccacccctgggacgttaaagtgaccagagcggatgttcgatggcgcctcggggcagtttggggttctgggtcggttccagcggctttaggcagaaagtgctcgctctcacccagcacatctctctccttgtccctggagttgcgcgcttcgcggggccgatgtagaacttagggcgccttgccgtggttggcgcgccccgggtgcagcgagaggccatccccgagcgctacctccccggagcggagcacgcgggctcccagtactaggggctgcgctcgagcagtggcgggggcggaggggtggttcttttccttctcctccgccagaggccacgggcgcccttgttcccgccggccaggtcctatcaaaggaggctgccggaactcaagaggcagaaaaagaccagttaggcggtgcagacggtctgggacgtggcagacggacggaccctcggcggacaggtggtcggcgtcggggtgcggtgggtaggggcgaggacaacgcagggtgcgctgggttgggacgtgggtccacttttgtagaccagctgtttggagagctgtatttaagactcgcgtatccagtgttttgtcgcagagagttttcactcttaaatcctgggggtttcttagaaagcaacttagaactcgagattcacctttcgtttccctttccccaaaagtagcgtaaccaacatttaagcttgcttaaaaacgaaaaccaaccgccttgcatccagtgttcccgatttactaaaataggtaaccaggcgtctcacagtcgccgtcctgtcaagagcgctaatgaacgttctcattaacacgcaggagtaccgggagccctgaaccgcccgctgctcggcggatcccagctgcggtggcgacggcgggaaggcgctttccgctgttcctcagcgggccgggcccttgaccagcgcggcccgcaggtcttccttctcgccgtcttgcagttgaagagctacatacgtagtcagtttcgatttgttacagacgttaacaaattcctttacccaaggttatgctatgacctttccgcagtttactttgattttctatgtttaaggttttggttgttggtagtagccgaatttaactggcactttattttacttctaaccttgtttcctgacggtgtacagaatcaacaaaataaaacatttaaagtctgattttttaaaaaaaaaaaaaaa

SEQ ID NO: 31 - protein, 323 aa, Homo sapiens
MDSDASLVSSRPSSPEPDDLFLPARSKGSSGSAFTGGTVSSSTPSDCPPELSAELRGAMGSAGAHPGDKLGGSGFKSSSSSTSSSTSSAAASSTKKDKKQMTEPELQQLRLKINSRERKRMHDLNIAMDGLREVMPYAHGPSVRKLSKIATLLLARNYILMLTNSLEEMKRLVSEIYGGHHAGFHPSACGGLAHSAPLPAATAHPAAAAHAAHHPAVHHPILPPAAAAAAAAAAAAAVSSASLPGSGLPSVGSIRPPHGLLKSPSAAAAAPLGGGGGGSGASGGFQHWGGMPCPCSMCQVPPPHHHVSAMGAGSLPRLTSDAK

SEQ ID NO: 32 - DNA, 3275 bp, Homo sapiens
  source 1..3275: mol_type = unassigned DNA
gggtgcttattatagatcgacgcgacaccagcgcccggtgccaggttctcccctgaggcttttcggagcgagctcctcaaatcgcatccagagtaagtgtccccgccccacagcagccgcagcctagatcccagggacagactctcctcaactcggctgtgacccagaatgctccgatacagggggtctggatccctactctgcgggccatttctccagagcgactttgctcttctgtcctccccacactcaccgctgcatctccctcaccaaaagcgagaagtcggagcgacaacagctctttctgcccaagccccagtcagctggtgagctccccgtggtctccagatgcagcacatggactctgggccccgcgccggctctgggtgcatgtgcgtgtgcgtgtgtttgctgcgtggtgtcgatggagataaggtggatccgtttgaggaaccaaatcattagttctctatttagatctccattctccccaaagaaaggccctcacttcccactcgtttattccagcccgggggctcagttttcccacacctaactgaaagcccgaagcctctagaatgccacccgcaccccgagggtcaccaacgctccctgaaataacctgttgcatgagagcagaggggagatagagagagcttaattataggtacccgcgtgcagctaaaaggagggccagagatagtagcgagggggacgaggagccacgggccacctgtgccgggaccccgcgctgtggtactgcggtgcaggcgggagcagcttttctgtctctcactgactcactctctctctctctccctctctctctctctcattctctctcttttctcctcctctcctggaagttttcgggtccgagggaaggaggaccctgcgaaagctgcgacgactatcttcccctggggccatggactcggacgccagcctggtgtccagccgcccgtcgtcgccagagcccgatgacctttttctgccggcccggagtaagggcagcagcggcagcgccttcactgggggcaccgtgtcctcgtccaccccgagtgactgcccgccggagctgagcgccgagctgcgcggcgctatgggctctgcgggcgcgcatcctgtggacaagctaggaggcagtggcttcaagtcatcctcgtccagcacctcgtcgtctacgtcgtcggcggctgcgtcgtccaccaagaaggacaagaagcaaatgacagagccggagctgcagcagctgcgtctcaagatcaacagccgcgagcgcaagcgcatgcacgacctcaacatcgccatggatggcctccgcgaggtcatgccgtacgcacacggcccttcggtgcgcaagctttccaagatcgccacgctgctgctggcgcgcaactacatcctcatgctcaccaactcgctggaggagatgaagcgactggtgagcgagatctacgggggccaccacgctggcttccacccgtcggcctgcggcggcctggcgcactccgcgcccctgcccgccgccaccgcgcacccggcagcagcagcgcacgccgcacatcaccccgcggtgcaccaccccatcctgccgcccgccgccgcagcggctgctgccgccgctgcagccgcggctgtgtccagcgcctctctgcccggatccgggctgccgtcggtcggctccatccgtccaccgcacggcctactcaagtctccgtctgctgccgcggccgccccgctggggggcgggggcggcggcagtggggcgagcgggggcttccagcactggggcggcatgccctgcccctgcagcatgtgccaggtgccgccgccgcaccaccacgtgtcggctatgggcgccggcagcctgccgcgcctcacctccgacgccaagtgagcctactggcgccggcgcgttctggcgacaggggagccaggggccgcggggaagcgaggactggcctgcgctgggctcgggagctctgtcgcgaggaggggcgcaggaccatggactgggggtggggcatggtggggatttcagcatctgcgaacccaagcaatgggggcgcccacagagcagtggggagtgaggggatgttctctccgggacctgatcgagcgctgtctggctttaacctgagctggtccagtagacatcgttttatgaaaaggtaccgctgtgtgcattcctcactagaactcatccgacccccgacccccacctccgggaaaagattctaaaaacttctttccctgagagcgtggcctgacttgcagactcggcttgggcagcacttcggggggggagggggtgttatgggagggggacacattggggccttgctcgtcttcctcctttcttggcgggtgggagactccgggtagccgcactgcagaagcaacagcccgaccgcgccctccagggtcgtccctggcccaaggccaggggccacaagttagttggaagccggcgttcggtatcagaagcgctgatggtcatatccaatctcaatatctgggtcaatccacaccctcttagaactgtggccgttcctccctgtctctcgttgatttgggagaatatggttttctaataaatctgtggatgttccttcttcaacagtatgagcaagtttatagacattcagagtagaaccacttgtggattggaataacccaaaactgccgatttcaggggcgggtgcattgtagttattattttaaaatagaaactaccccaccgactcatctttccttctctaagcacaaagtgatttggttattttggtacctgagaacgtaacagaattaaaaggcagttgctgtggaaacagtttgggttatttgggggttctgttggctttttaaaattttcttttttggatgtgtaaatttatcaatgatgaggtaagtgcgcaatgctaagctgtttgctcacgtgactgccagccccatcggagtctaagccggctttcctctattttggtttatttttgccacgtttaacacaaatggtaaactcctccacgtgcttcctgcgttccgtgcaagccgcctcggcgctgcctgcgttgcaaactgggctttgtagcgtctgccgtgtaacacccttcctctgatcgcaccgcccctcgcagagagtgtatcatctgttttatttttgtaaaaacaaagtgctaaataatatttattacttgtttggttgcaaaaacggaataaatgactgagtgttgagattttaaataaaatttaaagcaaaaaaaaaaaaaaaa

SEQ ID NO: 33 - protein, 432 aa, Homo sapiens
MERRRITSAARRSYVSSGEMMVGGLAPGRRLGPGTRLSLARMPPPLPTRVDFSLAGALNAGFKETRASERAEMMELNDRFASYIEKVRFLEQQNKALAAELNQLRAKEPTKLADVYQAELRELRLRLDQLTANSARLEVERDNLAQDLATVRQKLQDETNLRLEAENNLAAYRQEADEATLARLDLERKIESLEEEIRFLRKIHEEEVRELQEQLARQQVHVELDVAKPDLTAALKEIRTQYEAMASSNMHEAEEWYRSKFADLTDAAARNAELLRQAKHEANDYRRQLQSLTCDLESLRGTNESLERQMREQEERHVREAASYQEALARLEEEGQSLKDEMARHLQEYQDLLNVKLALDIEIATYRKLLEGEENRITIPVQTFSNLQIRETSLDTKSVSEGHLKRNIVVKTVEMRDGEVIKESKQEHKDVM

SEQ ID NO: 34 - DNA, 1488 bp, Homo sapiens
  source 1..1488: mol_type = unassigned DNA
gcaggatggagaggagacgcatcacctccgctgctcgccgctcctacgtctcctcaggggagatgatggtggggggcctggctcctggccgccgtctgggtcctggcacccgcctctccctggctcgaatgccccctccactcccgacccgagtggatttctccctggctggggcactcaatgctggcttcaaggagacccgggccagtgagcgggcagagatgatggagctcaatgaccgctttgccagctacatcgagaaggttcgcttcctggaacagcaaaacaaggcgctggctgctgagctgaaccagctgcgggccaaggagcccaccaagctggcagacgtctaccaggctgagctgcgagagctgcggctgcggctcgatcaactcaccgccaacagcgcccggctggaggttgagagggacaatctggcacaggacctggccactgtgaggcagaagctccaggatggaaccaacctgaggctggaagccgagaacaacctggctgcctatagacaggaagcagatgaagccaccctggcccgtctggatctggagaggaagattgagtcgctggaggaggagatccggttcttgaggaagatccacgaggaggaggttcgggaactccaggagcagctggcccgacagcaggtccatgtggagcttgacgtggccaagccagacctcaccgcagccctgaaagagatccgcacgcagtatgaggcaatggcgtccagcaacatgcatgaagccgaagagtggtaccgctccaagtttgcagacctgacagacgctgctgcccgcaacgcggagctgctccgccaggccaagcacgaagccaacgactaccggcgccagttgcagtccttgacctgcgacctggagtctctgcgcggcacgaacgagtccctggagaggcagatgcgcgagcaggaggagcggcacgtgcgggaggcggccagttatcaggaggcgctggcgcggctggaggaagaggggcagagcctcaaggacgagatggcccgccacttgcaggagtaccaggacctgctcaatgtcaagctggccctggacatcgagatcgccacctacaggaagctgctagagggcgaggagaaccggatcaccattcccgtgcagaccttctccaacctgcagattcgagaaaccagcctggacaccaagtctgtgtcagaaggccacctcaagaggaacatcgtggtgaagaccgtggagatgcgggatggagaggtcattaaggagtccaagcaggagcacaaggatgtgatgtgaggcaggacccacctggtggcctctgccccgtctcatgaggggcccgagcagaagcaggatagttgctccgcctctgctggcacatttccccagacctgagctccccaccaccccagctgctcccctccctcctctgtccctaggtcagcttgctgccctaggctccgtcagtatcaggcctgcc

SEQ ID NO: 35 - protein, 92 aa, Homo sapiens
MSELEKAMVALIDVFHQYSGREGDKHKLKKSELKELINNELSHFLEEIKEQEVVDKVMETLDNDGDGECDFQEFMAFVAMVTTACHEFFEHE

SEQ ID NO: 36 - DNA, 1135 bp, Homo sapiens
  source 1..1135: mol_type = unassigned DNA
gggcagagggaataagaggctgcctctgcccaccagtcctgccgcccaggacccgcagcagagacgacgcctgcagcaaggagaccaggaaggggtgagacaaggaagaggatgtctgagctggagaaggccatggtggccctcatcgacgttttccaccaatattctggaagggagggagacaagcacaagctgaagaaatccgaactgaaggagctcatcaacaatgagctttcccatttcttagaggaaatcaaagagcaggaggttgtggacaaagtcatggaaacactggacaatgatggagacggcgaatgtgacttccaggaattcatggcctttgttgccatggttactactgcctgccacgagttctttgaacatgagtgagattagaaagcagccaaacctttcctgtaacagagacggtcatgcaagaaagcagacagcaagggcttgcagcctagtaggagctgagctttccagccgtgttgtagctaattaggaagcttgatttgctttgtgattgaaaaattgaaaacctctttccaaaggctgttttaacggcctgcatcattctttctgctatattaggcctgtgtgtaagctgactggccccagggactcttgttaacagtaacttaggagtcaggtctcagtgataaagcgtgcaccgtgcagcccgccatggccgtgtagaccctaacccggagggaaccctgactacagaaattaccccggggcacccttaaaacttccactacctttaaaaaacaaagccttatccagcattatttgaaaacactgctgttctttaaatgcgttcctcatccatgcagataacagctggttggccggtgtggccctgcaagggcgtggtggcttcggcctgcttcccgggatgcgcctgatcaccaggtgaacgctcagcgctggcagcgctcctggaaaaagcaactccatcagaactcgcaatccgagccagctctgggggctccagcgtggcctccgtgacccatgcgattcaagtcgcggctgcaggatccttgcctccaacgtgcctccagcacatgcggcttccgagggcactaccgggggctctgagccaccgcgagggcctgcgttcaataaaaag

SEQ ID NO: 37 - protein, 466 aa, Homo sapiens
MAEEQDLSEVELSPVGSEEPRCLSPGSAPSLGPDGGGGGSGLRASPGPGELGKVKKEQQDGEADDDKFPVCIREAVSQVLSGYDWTLVPMPVRVNGASKSKPHVKRPMNAFMVWAQAARRKLADQYPHLHNAELSKTLGKLWRLLNESDKRPFIEEAERLRMQHKKDHPDYKYQPRRRKNGKAAQGEAECPGGEAEQGGTAAIQAHYKSAHLDHRHPGEGSPMSDGNPEHPSGQSHGPPTPPTTPKTELQSGKADPKRDGRSMGEGGKPHIDFGNVDIGEISHEVMSNMETFDVAELDQYLPPNGHPGHVSSYSAAGYGLGSALAVASGHSAWISKPPGVALPTVSPPGVDAKAQVKTETAGPQGPPHYTDQPSTSQIAYTSLSLPHYGSAFPSISRPQFDYSDHQPSGPYYGHSGQASGLYSAFSYMGPSQRPLYTAISDPSPSGPQSHSPTHWEQPVYTTLSRP

SEQ ID NO: 38 - DNA, 2882 bp, Homo sapiens
  source 1..2882: mol_type = unassigned DNA
gtccggccagggtggttggtggtaaggattcaggctccgtcctaacgaggccgtggcctgaggctcagggccccccgcccctccctcccagcccaccagcgtcacctcccagccccgagctggaccgcacaccttgggacacggttttccacttcctaaggacgagccccagactggaggagaggtccgaggaggtgggcgttggactctttgcgaggaccccggcggctggcccgggggaggcggccgaggcggcggcggcggcggccgggggcgacatggcggaggagcaggacctatcggaggtggagctgagccccgtgggctcggaggagccccgctgcctgtccccggggagcgcgccctcgctagggcccgacggcggcggcggcggatcgggcctgcgagccagcccggggccaggcgagctgggcaaggtcaagaaggagcagcaggacggcgaggcggacgatgacaagttccccgtgtgcatccgcgaggccgtcagccaggtgctcagcggctacgactggacgctggtgcccatgcccgtgcgcgtcaacggcgccagcaaaagcaagccgcacgtcaagcggcccatgaacgccttcatggtgtgggctcaggcagcgcgcaggaagctcgcggaccagtacccgcacctgcacaacgctgagctcagcaagacgctgggcaagctctggaggctgctgaacgaaagtgacaagcgccccttcatcgaggaggctgagcggctccgtatgcagcacaagaaagaccacccggactacaagtaccagcccaggcggcggaagaacgggaaggccgcccagggcgaggcggagtgccccggtggggaggccgagcaaggtgggaccgccgccatccaggcccactacaagagcgcccacttggaccaccggcacccaggagagggctcccccatgtcagatgggaaccccgagcacccctcaggccagagccatggcccacccacccctccaaccaccccgaagacagagctgcagtcgggcaaggcagacccgaagcgggacgggcgctccatgggggagggcgggaagcctcacatcgacttcggcaacgtggacattggtgagatcagccacgaggtaatgtccaacatggagacctttgatgtggctgagttggaccagtacctgccgcccaatgggcacccaggccatgtgagcagctactcagcagccggctatgggctgggcagtgccctggccgtggccagtggacactccgcctggatctccaagccaccaggcgtggctctgcccacggtctcaccacctggtgtggatgccaaagcccaggtgaagacagagaccgcggggccccaggggcccccacactacaccgaccagccatccacctcacagatcgcctacacctccctcagcctgccccactatggctcagccttcccctccatctcccgcccccagtttgactactctgaccatcagccctcaggaccctattatggccactcgggccaggcctctggcctctactcggccttctcctatatggggccctcgcagcggcccctctacacggccatctctgaccccagcccctcagggccccagtcccacagccccacacactgggagcagccagtatatacgacactgtcccggccctaaagggggccctgtcgccaccaccccccgcccagcccctgcccccagcctgtgtgccctgttccttgcccacctcaggcctggtggtggcagtggaggaggctgaggaggctgaagaggctgacaggtcggggggctttctgtctggctcactgccctgatgacccacccgccccatccaggctccagcagcaaagccccaggagaacaggctggacagaggagaaggaggttgactgttgcacccacactgaaagatgaggggctgcaccttcccccaggaatgaccctctatcccaggacctgagaagggcctgctcaccctcctcggggaggggaagcaccagggttggtggcatcggaggccttaccactcctatgactcctgttttctctctcacagatagtgagggtctgacatgcccatgccacctatgccacagtgcctaagggctaggccacccagagactgtgcccggagctggccgtgtctcccactcaggggctgagagtagctttgaggagcctcattggggagtggggggttcgagggacttagtggagttctcatcccttcaatgccccctccctttctgaaggcaggaaggagttggcacagaggccccctgatccaattctgtgccaataacctcattctttgtctgagaaacagcccccagtcctcctccactacaacctccatgaccttgagacgcatcccaggaggtgacgaggcaggggctccaggaaaggaatcagagacaattcacagagcctccctccctgggctccttgccagctccctcttcccttactaggctctatggcccctgctcagtcagccccactccctgggcttcccagagagtgacagctgctcaggccctaacccttggctccaggagacacagggcccagcacccaggttgctgtcggcaggctgaagacactagaatcctgacctgtacattctgcccttgcctcttaccccttgcctcccagtggtatttgaataaagtatgtagctatatctgcccctattttcctgttctgcagccccccaaatccacatgtaactcattactgtctcctgttatttatctcagtagtcccctctcctagccactctagcccctattaactctgcattaagcattccacataataaaattaaaggttccggttaaaaaaaaaaaaaaaaaaaaa

SEQ ID NO: 39 - protein, 705 aa, Homo sapiens [sequence omitted]

SEQ ID NO: 40 - DNA, 2470 bp, Homo sapiens
  source 1..2470: mol_type = unassigned DNA
ctcgagagagaaggagaggacattcctggcagaagttacaacacatgcaaaggtacagaggttgcccccttcctacccctctccttagaggtgggttagagatgtatcctttttacagatgaggaaaccaaatctcagaaagattaagtcactttcccaagtgtatggtggaggccccacttgaacccaggcactgtgtctccagaccccacactattactgccttgtttaaaccagccaactgatttaatgaataaaggatgaacaaatgaataagtggatgagtcacctgaaaattctgcaggcaaagagactccatatctacttacttcttgcctatcttctgccacctctcctagtccaccatcactgctcactatggtcaaggtcctacccaatctggcccctgctaccacaacccccttcagcttgttccagccacattggcactggatgtttcctcttcctggcacattcttaaaaaaatgtgttgatcataaagtgaacatgaccctttgggaattaactggagttcttgtattccctcatctgtaaaatagacattatattatccaccccactggattgttgtgagggtgggatgaaatgatgcatgtaaacacgcttagcttaagagttgggtacaatcagtgaacaaatgattatgaattagtgcttttattgtagtcagaatcataaagatttgacaggttcccatatcccacctctgcttggactacctcatttgctcatatgcaaagattatttggtacctactgtgtgtgcaccatgggatgggcctgcctctgtggaaagttcttgggtgcagggggagacagccatgggcactgatgacatcaggtagttatcgtgagttttggcggtgtccagagcaaagggatggtggcgtatataccaagtgtgttctggtgtgggggtggacacgcaccagggctagggctgcagagaatgtctgtgttgcagatctaggtttctccatgatcatcggtgggaatgtgttttgtctgcaagtgtatgctcatatgagtttccctgggtctctgtgtgtcagtgtgttacctgtgtgtgtgggggtatgggtgtatgcatgcatgtatgtaacatgcccatgtgtgttactctggacttgtatgtctgtatgtatacctagattggcgtgtgttctgtctgtacatgccctcgtatgtttcctcacttttgtgtgtgtttatatgtgtgtcatttcttgtgtgccctccaggccccccttgccaccttgggcaagggtgtgtacaccacccaagtgtccacctccgcttgtctgatgctgtctgtgacgcccccgctctctgcctagctgagcctgtgtggatgtgggagactaatctccccgcgggcactgcgtgtgacctcacccccctctgtgagggggttatttctctactttcgtgtctctgagtgtgcttccagtgcccccctccccccaaaaaatgccttctgagttgaatatcaacactacaaaccgagtatctgcagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtcgcgactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagctgtgctcctgggcaccgcgcagtccgcccccgcggctcctggccagaccacccctaggaccccctgccccaagtcgcagccatgaactacctgcggcgccgcctgtcggacagcaactttatggccaatctgccaaatgggtacatgacagacctgcagcgtccgcagccgcccccaccgccgcccggtgcccacagccccggagccacgcccggtcccgggaccgccactgccgagaggtcctccggggtcgccccagcggcctctccggccgcccctagccccgggtcctcggggggcggtggcttcttctcgtcgctgtccaacgcggtcaagcagaccacggcggcggcagctgccaccttcagcgagcaggtgggcggcggctctgggggcgcaggccgcgggggagccgcctccagggtgctgctggtcatcgacgagccgcacaccgactggtaag

SEQ ID NO: 41 - protein, 313 aa, Homo sapiens
MLLLADMDVVNQLVAGGQFRVVKEPLGFVKVLQWVFAIFAFATCGSYSGELQLSVDCANKTESDLSIEVEFEYPFRLHQVYFDAPTCRGGTTKVFLVGDYSSSAEFFVTVAVFAFLYSMGALATYIFLQNKYRENNKGPMLDFLATAVFAFMWLVSSSAWAKGLSDVKMATDPENIIKEMPVCRQTGNTCKELRDPVTSGLNTSVVFGFLNLVLWVGNLWFVFKETGWAAPFLRAPPGAPEKQPAPGDAYGDAGYGQGPGGYGPQDSYGPQGGYQPDYGQPAGSGGSGYGPQGDYGQQGYGPQGAPTSFSNQM

SEQ ID NO: 42 - DNA, 2449 bp, Homo sapiens
  source 1..2449: mol_type = unassigned DNA
gccccctgcattgctgatgctgctgctggcggacatggacgtggtgaatcagctggtggctgggggtcagttccgggtggtcaaggagcccctcggctttgtgaaggtgctgcaatgggtcttcgccatcttcgcctttgccacatgcggcagctacagtggggagctccagctgagcgtggattgtgccaacaagaccgagagtgacctcagcatcgaggtcgagttcgagtaccccttcaggctgcaccaagtgtactttgatgcacccacctgccgagggggcaccaccaaggtcttcttagttggggactactcctcgtcagccgaattctttgtcaccgtggccgtgtttgccttcctctactccatgggggctctggccacctacatcttcctgcagaacaagtaccgagagaataacaaagggcccatgctggactttctggccacggctgtgttcgccttcatgtggctagttagctcatcggcatgggccaaggggctgtcagatgtgaagatggccacagacccagagaacattatcaaggagatgcctgtctgccgccagacagggaacacatgcaaggagctgagagaccctgtgacctcgggactcaacacctcggtggtgttcggcttcctgaacctggtgctctgggtcggcaacctgtggttcgtgtttaaggagacaggctgggccgccccgttcctgcgcgcgcctcccggcgcccccgagaaacaaccggcacccggggacgcctacggcgatgcaggctacgggcagggccccggcgggtacgggccccaggattcctacgggcctcagggcggctaccagcctgactatggtcaaccagccggcagcggtggcagtggctacgggcctcagggcgactatgggcagcaaggctacggcccgcagggtgcacccacctccttctccaatcagatgtagtctggtcagtgaagcccaggaggacctggggggggcaagagctcaggagaaggcctgccccccttcccacccctataccctaggtctccacccctcaagccaggagaccctgtctttgctgtttatatatatatatattatatataaatatctatttatctgtctgagccctgccctcactccactcccctcatccactaggtgcccagtcttgagtgggccccctctcttaccccgtccctttccctgcatcccttggcccctctctgtttaccctccctgtcccctgaggttaaggggatctaaaaggaggacagggagggaacagacctcggctgtgtggggagggtgggcgtgacttcagactctctcctctctctccctccactcctcccaactctggccttggttcctccagcaatgcctgcctgaacaaaggccgttagggaaatccaactccagggttaaagaaaggcagagattgggggggcttggggtagagaggacagtttaggacccaaggtggtcttggagaggaggtgtggagtggaggggtcagcaggggggttgggttccagacagagtggatctggagtctgaaggagaggagtgcgctagagcattctggggtggggcttggaagggcgctgagggcagggttctagaaggggcgaggctttaagcgaggcagaatggtgggctccagagtaggtgggtcttggattggtaccagagcctatggaaagggtgtggcttggaacatttgggagactgagcttgattctaaaggggacagatcttgagcaaggcaagaagtgggattcaggaatgggccaagccagggttccagacagggtggggcttagaatggggcttccatggtggtttcagaaagggcagcccctccccatggtgcagtgaagaaaatgttttacaatggctgggtttgggcagtggagaggggacttggataggagcttccagatgggttttgttaggggtgggggagaatggctctggctacgacttgggacggaagtggcctgagaagagtcgagtgatatggcttgtagggtgaggcgtgggatccagagagaagcaccccaccacacacacccttccccactcccgtgatgaaacagctaggttaataggaggacagaaccaacgggtctgtgggactggcccacccctcttcccccttcccctgcgccctccctccctccacacctccacccgtcctggggtggttggaggcctggtctggagcccctatcctgcaccctctgctatgtctgtgatgtcagtagtgcctgtgatcgtgtgttgccattttgtctggctgtggcccctccttctcccctccagacccctaccctttcccaaacccttcggtattgttcaaagaacccccctccccaaggaagaacaaatatgattctcctctcccaaataaactccttaaccacctagtcaaaaaaaaaaaaaaaa

SEQ ID NO: 43 - protein, 1192 aa, Homo sapiens; identical to SEQ ID NO: 27

SEQ ID NO: 44 - DNA, 4871 bp, Homo sapiens; identical to SEQ ID NO: 28
  source 1..4871: mol_type = unassigned DNA

SEQ ID NO: 45 - protein, 432 aa, Homo sapiens; identical to SEQ ID NO: 33

SEQ ID NO: 46 - DNA, 3097 bp, Homo sapiens
  source 1..3097: mol_type = unassigned DNA
atcgccagtctagcccactccttcataaagccctcgcatcccaggagcgagcagagccagagcaggatggagaggagacgcatcacctccgctgctcgccgctcctacgtctcctcaggggagatgatggtggggggcctggctcctggccgccgtctgggtcctggcacccgcctctccctggctcgaatgccccctccactcccgacccgggtggatttctccctggctggggcactcaatgctggcttcaaggagacccgggccagtgagcgggcagagatgatggagctcaatgaccgctttgccagctacatcgagaaggttcgcttcctggaacagcaaaacaaggcgctggctgctgagctgaaccagctgcgggccaaggagcccaccaagctggcagacgtctaccaggctgagctgcgagagctgcggctgcggctcgatcaactcaccgccaacagcgcccggctggaggttgagagggacaatctggcacaggacctggccactgtgaggcagaagctccaggatgaaaccaacctgaggctggaagccgagaacaacctggctgcctatagacaggaagcagatgaagccaccctggcccgtctggatctggagaggaagattgagtcgctggaggaggagatccggttcttgaggaagatccacgaggaggaggttcgggaactccaggagcagctggcccgacagcaggtccatgtggagcttgacgtggccaagccagacctcaccgcagccctgaaagagatccgcacgcagtatgaggcaatggcgtccagcaacatgcatgaagccgaagagtggtaccgctccaagtttgcagacctgacagacgctgctgcccgcaacgcggagctgctccgccaggccaagcacgaagccaacgactaccggcgccagttgcagtccttgacctgcgacctggagtctctgcgcggcacgaacgagtccctggagaggcagatgcgcgagcaggaggagcggcacgtgcgggaggcggccagttatcaggaggcgctggcgcggctggaggaagaggggcagagcctcaaggacgagatggcccgccacttgcaggagtaccaggacctgctcaatgtcaagctggccctggacatcgagatcgccacctacaggaagctgctagagggcgaggagaaccggatcaccattcccgtgcagaccttctccaacctgcagattcgagaaaccagcctggacaccaagtctgtgtcagaaggccacctcaagaggaacatcgtggtgaagaccgtggagatgcgggatggagaggtcattaaggagtccaagcaggagcacaaggatgtgatgtgaggcaggacccacctggtggcctctgccccgtctcatgaggggcccgagcagaagcaggatagttgctccgcctctgctggcacatttccccagacctgagctccccaccaccccagctgctcccctccctcctctgtccctaggtcagcttgctgccctaggctccgtcagtatcaggcctgccagacggcacccacccagcacccagcaactccaactaacaagaaactcacccccaaggggcagtctggaggggcatggccagcagcttgcgttagaatgaggaggaaggagagaaggggaggagggcggggggcacctactacatcgccctccacatccctgattcctgttgttatggaaactgttgccagagatggaggttctctcggagtatctgggaactgtgcctttgagtttcctcaggctgctggaggaaaactgagactcagacaggaaagggaaggccccacagacaaggtagccctggccagaggcttgttttgtcttttggtttttatgaggtgggatatccctatgctgcctaggctgaccttgaactcctgggctcaagcagtctacccacctcagcctcctgtgtagctgggattatagattggagccaccatgcccagctcagagggttgttctcctagactgaccctgatcagtctaagatgggtggggacgtcctgccacctggggcagtcacctgcccagatcccagaaggacctcctgagcgatgactcaagtgtctcagtccacctgagctgccatccagggatgccatctgtgggcacgctgtgggcaggtgggagcttgattctcagcacttgggggatctgttgtgtacgtggagagggatgaggtgctgggagggatagaggggggctgcctggcccccagctgtgggtacagagaggtcaagcccaggaggactgccccgtgcagactggaggggacgctggtagagatggaggaggaggcaattgggatggcgctaggcatacaagtaggggttgtgggtgaccagttgcacttggcctctggattgtgggaattaaggaagtgactcatcctcttgaagatgctgaaacaggagagaaaggggatgtatccatgggggcagggcatgactttgtcccatttctaaaggcctcttccttgctgtgtcataccaggccgccccagcctctgagcccctgggactgctgcttcttaaccccagtaagccactgccacacgtctgaccctctccaccccatagtgaccggctgcttttccctaagccaagggcctcttgcggtcccttcttactcacacacaaaatgtacccagtattctaggtagtgccctattttacaattgtaaaactgaggcacgagcaaagtgaagacactggctcatattcctgcagcctggaggccgggtgctcagggctgacacgtccaccccagtgcacccactctgctttgactgagcagactggtgagcagactggtgggatctgtgcccagagatgggactgggagggcccacttcagggttctcctctcccctctaaggccgaagaagggtccttccctctccccaagacttggtgtcctttccctccactccttcctgccacctgctgctgctgctgctgctaatcttcagggcactgctgctgcctttagtcgctgaggaaaaataaagacaaatgctgcgcccttccccaaaaaaaaaaaa

SEQ ID NO: 47 - protein, 92 aa, Homo sapiens; identical to SEQ ID NO: 35

SEQ ID NO: 48 - DNA, 1135 bp, Homo sapiens; identical to SEQ ID NO: 36
  source 1..1135: mol_type = unassigned DNA

SEQ ID NO: 49 - protein, 422 aa, Homo sapiens [sequence omitted]

SEQ ID NO: 50 - DNA, 1399 bp, Homo sapiens
  source 1..1399: mol_type = unassigned DNA
agggggaagactttaactaggggcgcgcagatgtgtgaggccttttattgtgagagtggacagacatccgagatttcagagccccatattcgagccccgtggaatcccgcggcccccagccagagccagcatgcagaacagtcacagcggagtgaatcagctcggtggtgtctttgtcaacgggcggccactgccggactccacccggcagaagattgtagagctagctcacagcggggcccggccgtgcgacatttcccgaattctgcaggtgtccaacggatgtgtgagtaaaattctgggcaggtattacgagactggctccatcagacccagggcaatcggtggtagtaaaccgagagtagcgactccagaagttgtaagcaaaatagcccagtataagcgggagtgcccgtccatctttgcttgggaaatccgagacagattactgtccgagggggtctgtaccaacgataacataccaagcgtgtcatcaataaacagagttcttcgcaacctggctagcgaaaagcaacagatgggcgcagacggcatgtatgataaactaaggatgttgaacgggcagaccggaagctggggcacccgccctggttggtatccggggacttcggtgccagggcaacctacgcaagatggctgccagcaacaggaaggagggggagagaataccaactccatcagttccaacggagaagattcagatgaggctcaaatgcgacttcagctgaagcggaagctgcaaagaaatagaacatcctttacccaagagcaaattgaggccctggagaaagagtttgagagaacccattatccagatgtgtttgcccgagaaagactagcagccaaaatagatctacctgaagcaagaatacaggtatggttttctaatcgaagggccaaatggagaagagaagaaaaactgaggaatcagagaagacaggccagcaacacacctagtcatattcctatcagcagtagtttcagcaccagtgtctaccaaccaattccacaacccaccacaccggtttcctccttcacatctggctccatgttgggccgaacagacacagccctcacaaacacctacagcgctctgccgcctatgcccagcttcaccatggcaaataacctgcctatgcaacccccagtccccagccagacctcctcatactcctgcatgctgcccaccagcccttcggtgaatgggcggagttatgatacctacacccccccacatatgcagacacacatgaacagtcagccaatgggcacctcgggcaccacttcaacaggactcatttcccctggtgtgtcagttccagttcaagttcccggaagtgaacctgatatgtctcaatactggccaagattacagtaa

SEQ ID NO: 51 - protein, 1621 aa, Homo sapiens
MEGCMGEESFQMWELNRRLEAYLARVKALEEQNELLSAELGGLRAQSADTSWRAHADDELAALRALVDQRWREKHAAEVARDNLAEELEGVAGRCQQLRLARERTTEEVARNRRAVEAEKCARAWLSSQVAELERELEALRVAHEEERVGLNAQAACAPRCPAPPRGPPAPAPEVEELARRLGEAWRGAVRGYQERVAHMETSLGQARERLGRAVQGAREGRLELQQLQAERGGLLERRAALEQRLEGRWQERLRATEKFQLAVEALEQEKQGLQSQIAQVLEGRQQLAHLKMSLSLEVATYRTLLEAENSRLQTPGGGSKTSLSFQDPKLELQFPRTPEGRRLGSLLPVLSPTSLPSPLPATLETPVPAFLKNQEFLQARTPTLASTPIPPTPQAPSPAVDAEIRAQDAPLSLLQTQGGRKQAPEPLRAEARVAIPASVLPGPEEPGGQRQEASTGQSPEDHASLAPPLSPDHSSLEAKDGESGGSRVFSICRGEGEGQIWGLVEKETAIEGKVVSSLQQEIWEEEDLNRKEIQDSQVPLEKETLKSLGEEIQESLKTLENQSHETLERENQECPRSLEEDLETLKSLEKENKELLKDVEVVRPLEKEAVGQLKPTGKEDTQTLQSLQKENQELMKSLEGNLETFLFPGTENQELVSSLQENLESLTALEKENQEPLRSPEVGDEEALRPLTKENQEPLRSLEDENKEAFRSLEKENQEPLKTLEEEDQSIVRPLETENHKSLRSLEEQDQETLRTLEKETQQRRRSLGEQDQMTLRPPEKVDLEPLKSLDQEIARPLENENQEFLKSLKEESVEAVKSLETEILESLKSAGQENLETLKSPETQAPLWTPEEINQGAMNPLEKEIQEPLESVEVNQETFRLLEEENQESLRSLGAWNLENLRSPEEVDKESQRNLEEEENLGKGEYQESLRSLEEEGQELPQSADVQRWEDTVEKDQELAQESPPGMAGVENEDEAELNLREQDGFTGKEEVVEQGELNATEEVWIPGEGHPESPEPKEQRGLVEGASVKGGAEGLQDPEGQSQQVGAPGLQAPQGLPEAIEPLVEDDVAPGGDQASPEVMLGSEPAMGESAAGAEPGPGQGVGGLGDPGHLTREEVMEPPLEEESLEAKRVQGLEGPRKDLEEAGGLGTEFSELPGKSRDPWEPPREGREESEAEAPRGAEEAFPAETLGHTGSDAPSPWPLGSEEAEEDVPPVLVSPSPTYTPILEDAPGPQPQAEGSQEASWGVQGRAEALGKVESEQEELGSGEIPEGPQEEGEESREESEEDELGETLPDSTPLGFYLRSPTSPRWDPTGEQRPPPQGETGKEGWDPAVLASEGLEAPPSEKEEGEEGEEECGRDSDLSEEFEDLGTEAPFLPGVPGEVAEPLGQVPQLLLDPAAWDRDGESDGFADEEESGEEGEEDQEEGREPGAGRWGPGSSVGSLQALSSSQRGEFLESDSVSVSVPWDDSLRGAVAGAPKTALETESQDSAEPSGSEEESDPVSLEREDKVPGPLEIPSGMEDAGPGADIIGVNGQGPNLEGKSQHVNGGVMNGLEQSEEVGQGMPLVSEGDRGSPFQEEEGSALKTSWAGAPVHLGQGQFLKFTQREGDRESWSSGED

SEQ ID NO: 52 - DNA, 5591 bp, Homo sapiens
  source 1..5591: mol_type = unassigned DNA
gctactcccaccccgccccgccccgtcattgtccccgtcggtctcttttctcttccgtcctaaaagctctgcgagccgctcccttctcccggtgccccgcgtctgtccatcctcagtgggtcagacgagcaggatggagggctgcatgggggaggagtcgtttcagatgtgggagctcaatcggcgcctggaggcctacctggcccgggtcaaggcgctggaggagcagaatgagctgctcagcgcggagctcggggggctccgggcacaatccgcggacacctcctggcgggcgcatgccgacgacgagctggcggccctgcgggccctcgttgaccaacgctggcgggagaagcacgcggccgaggtggcgcgcgacaacctggctgaagagctggagggcgtggcaggccgatgccagcagctgcggctggcccgggagcggacgacggaggaggtagcccgcaaccggcgcgccgtcgaggcagagaaatgcgcccgggcctggctgagtagccaggtggcagagctggagcgcgagctagaggctctacgcgtggcgcacgaggaggagcgcgtcggcctgaacgcgcaggctgcctgtgccccccgctgccccgcgccgccccgcgggcctcccgcgccggccccggaggtagaggagctggcaaggcgactgggcgaggcgtggcgcggggcagtgcgcggctaccaggagcgcgtggcacacatggagacgtcgctgggccaggcccgcgagcggctgggccgggcggtgcagggtgcccgcgagggccgcctggagctgcagcagctccaggctgagcgcggaggcctcctggagcgcagggcagcgttggaacagaggttggagggccgctggcaggagcggctgcgggctactgaaaagttccagctggctgtggaggccctggagcaggagaaacagggcctacagagccagatcgctcaggtcctggaaggtcggcagcagctggcgcacctcaagatgtccctcagcctggaggtggccacgtacaggaccctcctggaggctgagaactcccggctgcaaacacctggcggtggctccaagacttccctcagctttcaggaccccaagctggagctgcaattccctaggaccccagagggccggcgtcttggatctttgctcccagtcctgagcccaacttccctcccctcacccttgcctgctacccttgagacacctgtgccagcctttcttaagaaccaagaattcctccaggcccgtacccctaccttggccagcacccccatcccccccacacctcaggcaccctctcctgctgtagatgcagagatcagagcccaggatgctcctctctctctgctccagacacagggtgggaggaaacaggctccagagcccctgcgggctgaagccagggtggccattcctgccagcgtcctgcctggaccagaggagcctgggggccagcggcaagaggccagtacaggccagtccccagaggaccatgcctccttggcaccacccctcagccctgaccactccagtttagaggctaaggatggagaatccggtgggtctagagtgttcagcatatgccgaggggaaggtgaagggcaaatctgggggttggtagagaaagaaacagccatagagggcaaagtggtaagcagcttgcagcaggaaatatgggaagaagaggatctaaacaggaaggaaatccaggactcccaggttcctttggaaaaagaaaccctgaagtctctgggagaggagattcaagagtcactgaagactctggaaaaccagagccatgagacactagaaagggagaatcaagaatgtccgaggtctttagaagaagacttagaaacactaaaaagtctagaaaaggaaaataaagagctattaaaggatgtggaggtagtgagacctctagaaaaagaggctgtaggccaacttaagcctacaggaaaagaggacacacagacattgcaatccctgcaaaaggagaatcaagaactaatgaaatctcttgaaggtaatctagagacatttttatttccaggaacggaaaatcaagaattagtaagttctctgcaagagaacttagagtcattgacagctctggaaaaggagaatcaagagccactgagatctccagaagtaggggatgaggaggcactgagacctctgacaaaggagaatcaggaacccctgaggtctcttgaagatgagaacaaagaggcctttagatctctagaaaaagagaaccaggagccactgaagactctagaagaagaggaccagagtattgtgagacctctagaaacagagaatcacaaatcactgaggtctttagaagaacaggaccaagagacattgagaactcttgaaaaagagactcaacagcgacggaggtctctaggggaacaggatcagatgacattaagacccccagaaaaagtggatctagaaccactgaagtctcttgaccaggagatagctagacctcttgaaaatgagaatcaagagttcttaaagtcactcaaagaagagagcgtagaggcagtaaaatctttagaaacagagatcctagaatcactgaagtctgcgggacaagagaacctggaaacactgaaatctccagaaactcaagcaccactgtggactccagaagaaataaatcagggggcaatgaatcctctagaaaaggaaattcaagaaccactggagtctgtggaagtgaaccaagagacattcagactcctggaagaggagaatcaggaatcattgagatctctgggagcatggaacctggagaatttgagatctccagaggaggtagacaaggaaagtcaaaggaatctggaagaggaagagaacctgggaaagggagagtaccaagagtcactgaggtctctggaggaggagggacaggagctgccgcagtctgcagatgtgcagaggtgggaagatacggtggagaaggaccaagaactggctcaggaaagccctcctgggatggctggagtggaaaatgaggatgaggcagagctgaatctgagggagcaggatggcttcactgggaaggaggaggtggtagagcagggagagctgaatgccacagaggaggtctggatcccaggcgaggggcacccagagagccctgagcccaaagagcagagaggcctggttgagggagccagtgtgaagggaggggctgagggcctccaggaccctgaagggcaatcacaacaggtgggggccccaggcctccaggctccccaggggctgccagaggcgatagagcccctggtggaagatgatgtggccccagggggtgaccaagcctccccagaggtcatgttggggtcagagcctgccatgggtgagtctgctgcgggagctgagccaggcccggggcagggggtgggagggctgggggacccaggccatctgaccagggaagaggtgatggaaccacccctggaagaggagagtttggaggcaaagagggttcagggcttggaagggcctagaaaggacctagaggaggcaggtggtctggggacagagttctccgagctgcctgggaagagcagagacccttgggagcctcccagggagggtagggaggagtcagaggctgaggcccccaggggagcagaggaggcgttccctgctgagaccctgggccacactggaagtgatgccccttcaccttggcctctggggtcagaggaagctgaggaggatgtaccaccagtgctggtctcccccagcccaacgtacaccccgatcctggaagatgcccctgggcctcagcctcaggctgaagggagtcaggaggctagctggggggtgcaggggagggctgaagccctggggaaagtagagagcgagcaggaggagttgggttctggggagatccccgagggcccccaggaggaaggggaggagagcagagaagagagcgaggaggatgagctcggggagacccttccagactccactcccctgggcttctacctcaggtcccccacctcccccaggtgggaccccactggagagcagaggccaccccctcaaggggagactggaaaggagggctgggatcctgctgtcctggcttccgagggccttgaggccccaccctcagaaaaggaggagggggaggagggagaagaggagtgtggccgtgactctgacctgtcagaagaatttgaggacctggggactgaggcaccttttcttcctggggtccctggggaggtggcagaacctctgggccaggtgccccagctgctactggatcctgcagcctgggatcgagatggggagtccgatgggtttgcagatgaggaagaaagtggggaggagggagaggaggatcaggaggaggggagggagccaggggctgggcggtgggggccagggtcttctgttggcagcctccaggccctgagtagctcccagagaggggaattcctggagtctgattctgtgagtgtcagtgtcccctgggatgacagcttgaggggtgcagtggctggtgcccccaagactgccctggaaacggagtcccaggacagtgctgagccttctggctcagaggaagagtctgaccctgtttccttggagagggaggacaaagtccctggccctctagagatccccagtgggatggaggatgcaggcccaggggcagacatcattggtgttaatggccagggtcccaacttggaggggaagtcacagcatgtgaatgggggagtgatgaacgggctggagcagtctgaggaagtggggcaaggaatgccgctagtctctgagggagaccgagggagcccctttcaggaggaggaggggagtgctctgaagacctcttgggcaggggctcctgttcacctgggccagggtcagttcctgaagttcactcagagggaaggagatagagagtcctggtcctcaggggaggactaggaaaagaccatctgcccggcactggggacttaggggtgcggggaggggaaggacgcctccaagcccgctccctgctcaggagcagcactcttaacttacgatctcttgacatatggtttctggctgagaggcctggcccgctaaggtgaaaaggggtgtggcaaaggagcctactccaagaatggaggctgtaggaatataacctcccaccctgcaaagggaatctcttgcctgctccatctcataggctaagtcagctgaatcccgatagtactaggtccccttccctccgcatcccgtcagctggaaaaggcctgtggcccagaggcttctccaaagggagggtgacatgctggcttttgtgcccaagctcaccagccctgcgccacctcactgcagtagtgcaccatctcactgcagtagcacgccctcctgggccgtctggcctgtggctaatggaggtgacggcactcccatgtgctgactccccccatccctgccacgctgtggccctgcctggctagtccctgcctgaataaagtaatgcctccgcttcaaaaaaaaaaaaaaaaaaaaaaaaaaaaaaaaaaaa

SEQ ID NO: 53 - protein, 348 aa, Homo sapiens
MAQPGSGCKATTRCLEGTAPPAMAQSDAEALAGALDKDEGQASPCTPSTPSVCSPPSAASSVPSAGKNICSSCGLEILDRYLLKVNNLIWHVRCLECSVCRTSLRQQNSCYIKNKEIFCKMDYFSRFGTKCARCGRQIYASDWVRRARGNAYHLACFACFSCKRQLSTGEEFGLVEEKVLCRIHYDTMIENLKRAAENGNGLTLEGAVPSEQDSQPKPAKRARTSFTAEQLQVMQAQFAQDNNPDAQTLQKLADMTGLSRRVIQVWFQNCRARHKKHTPQHPVPPSGAPPSRLPSALSDDIHYTPFSSPERARMVTLHGYIESHPFSVLTLPALPHLPVGAPQLPLSR

SEQ ID NO: 54 - DNA, 2835 bp, Homo sapiens
  source 1..2835: mol_type = unassigned DNA
cccgccaccgaccaggtgatggcccagccagggtccggctgcaaagcgaccacccgctgtcttgaagggaccgcgccgcccgccatggctcagtctgacgccgaggccctggcaggagctctggacaaggacgagggtcaggcctccccatgtacgcccagcacgccatctgtctgctcaccgccctctgccgcctcctccgtgccgtctgcaggcaagaacatctgctccagctgcggcctcgagatcctggaccgatatctgctcaaggtcaacaacctcatctggcacgtgcggtgcctcgagtgctccgtgtgtcgcacgtcgctgaggcagcagaacagctgctacatcaagaacaaggagatcttctgcaagatggactacttcagccgattcgggaccaagtgtgcccggtgcggccgacagatctacgccagcgactgggtgcggagagctcgcggcaacgcctaccacctggcctgcttcgcctgcttctcgtgcaagcgccagctgtccactggtgaggagttcggcctggtcgaggagaaggtgctctgccgcatccactacgacaccatgattgagaacctcaagagggccgccgagaacgggaacggcctcacgttggagggggcagtgccctcggaacaggacagtcaacccaagccggccaagcgcgcgcggacgtccttcaccgcggaacagctgcaggttatgcaggcgcagttcgcgcaggacaacaaccccgacgctcagacgctgcagaagctggcggacatgacgggcctcagccggagagtcatccaggtgtggtttcaaaactgccgggcgcgtcataaaaagcacacgccgcaacacccagtgccgccctcgggggcgcccccgtcccgccttccctccgccctgtccgacgacatccactacaccccgttcagcagccccgagcgggcgcgcatggtcaccctgcacggctacattgagagtcatcctttttcagtactaacgctgccggcacttccgcatctgcccgtgggcgccccacagctgcccctcagccgctgagatccagtgtccaagctgcggccaggagtccacccacctccgcatccacccccgtccgccatcctgcccaccaccaggtcggttcccgaggcctggcctttccctctcctgctgagaaccagaacccaccaggagcaccacagagtcctcctcttggaaggcagaactccctgaaatctggaatcagggtggaaacagcctgtttttcccatttaaacaggagtcctcttcaacttcagctgattacaataacaaaaggcggaattgaattgtgcgatgccaacggccttctcatttacaggtttttttcccccacattggcctttatttactacttccttggaaccatctctgaattctgaatagctgacaacccccaatgttatccactctgttgcttttgtctggaaaactctacagtgtttgtgggatgtccccaaaggtaagctatgttctaattttatcatttccatctgtctggttatgtcaagttaattcagaaagagaagagacagtgaccaaccctgagaggcctaatagggcagagatggaggcctgcccagactaggaggcagcggggatagacagggaatggggagaagaaagacccccattggtttggaaatcaaggagagggcggtgacatattggaccagaagaggcactagccattttaaggagaggaaagagaaaactctggggtcagggagagaccctacccccacctaattatccagcatatatgtaagaaacatagcagcgatggtattcgatctgtgccatgactcttctgaatgtttggacaggttagagttggggacccctgttggccacttgttgacctctcatagtggtgcttgggccaggtcttctcaatggaaggggaatcccttataggggagagggaacagagcccagtgaaatggcagtcagaatgttaaccctggatccatctctaagtagagagagggtgcccattgcctaggtgagtgtgccaagctcaggattccaactggtgcctctgagcttcccaatcaatacttcctggagccagccccacccacccctgagaacagaggtcagacacagctgcgtaacatccatcctgctacaactcttccaccccaaacaaaagggctcaggctacacacgaccatgatttatgttttcaggggatgcccatttgtcccaagcttatcctgtaattctagaattacctggtgtcctgatgcattttccactagaggttgctaatcagcatgttttagcccaagtccaccttcctgctgtggttaacctgttatgttgcttttggaaggagactctaagacagggaaagcaagttcatggtacatacgcagccattgtctctgtttttacccatggcagacattgctaatcaatggcagctctatttcactgagtctggataaggtttcagagttcaaatgcttgacgttggcacttaacatgaaagcctataggtcattcttgctctgggatctacaggcagggtaggcacaggtgcagcctaagaagggaacctgcttcctctcccttccaaagacagtgacagctgactgagggcaaagagcaggcaccactcagaacgtggtgagtacagctcagctcagcactcagtcagtggtaacttgtgcccagccctgtgctaggcgctgacattaacaggagcaaccagggcccaattcctggccttggagctcaaatctttcctttgatttttgctcctgatcatcaaggccccagtgg

SEQ ID NO: 55 - protein, 356 aa, Homo sapiens
MQILSRCQGLMSEECGRTTALAAGRTRKGAGEEGLVSPEGAGDEDSCSSSAPLSPSSSPRSMASGSGCPPGKCVCNSCGLEIVDKYLLKVNDLCWHVRCLSCSVCRTSLGRHTSCYIKDKDIFCKLDYFRRYGTRCSRCGRHIHSTDWVRRAKGNVYHLACFACFSCKRQLSTGEEFALVEEKVLCRVHYDCMLDNLKREVENGNGISVEGALLTEQDVNHPKPAKRARTSFTADQLQVMQAQFAQDNNPDAQTLQKLAERTGLSRRVIQVWFQNCRARHKKHVSPNHSSSTPVTAAPPSRLSPPMLEEMAYSAYVPQDGTMLTALHSYMDAHSPTTLGLQPLLPHSMTQLPISHT

SEQ ID NO: 56 - DNA, 2411 bp, Homo sapiens
  source 1..2411: mol_type = unassigned DNA
agcggcaagaggctagcggctggaccacttgtgctggagtggtaaagaactatcatgaatccatttactgaaagtgtccatttctgaactcaccctaaagaggacaaacaccgcaaagtagttaaaagtcaggcattcgcgtcggacgtctgggtttgaattctgccctggcttgactggaaacgcttcccctatttcttccgtagcggaccgggagagcttactggcgctctgcgaaccggctggaaagaaacaccgagtcactcgtacagactcttggtcgcagaacttggctttccgctattggtcctccagaaccgcttgaaacaactggccccagctggcgcatcagaccgcagtgaggaatgccgcggggcgggtggcgaaggcagggtctgcccgccagtggattcccgggtgtcccgcgtggagcaggcttgcccagctgggaagcccatcaaacctcagtcttggcccacagtgggagagagaccagtgggtcccagacggaggccatcgcccgcttttggcgacctccactggcgtgaataaaagcacccctctcttaccctcagaaactgtgggtagcaaggtataaaacggagtctgggaccggtaagtcccaaggtgagcccgtatacagctctgccatctctgaggggttatgcagattctgagcaggtgtcaggggctcatgtcagaggagtgcgggcggactacagccctggcggccgggaggactcgcaaaggcgccggggaagagggactggtgagccccgagggagcgggggacgaggactcgtgctcctcctcggccccgctgtccccgtcgtcctcgccccggtccatggcctcgggctccggctgccctcctggcaagtgtgtgtgcaacagttgcggcctggagatcgtggacaaataccttctcaaggtgaatgacctatgctggcatgtccggtgtctctcctgcagtgtttgcagaacctccctaggaaggcacaccagctgttatattaaagacaaagacattttctgcaaacttgattatttcagaaggtatggaactcgctgctctcgatgtgggagacacatccattctactgactgggtccggagagccaaggggaatgtctatcacttggcatgctttgcctgcttttcctgcaaaaggcaactttccacaggagaggagtttgctttggtggaagagaaagtcctctgcagagtacattatgactgcatgctggataatttaaaaagagaagtagaaaatgggaatgggattagtgtggaaggtgccctcctcacagagcaagatgttaaccatccaaaaccagcaaaaagagctcggaccagctttacagcagatcagcttcaggttatgcaagcacaatttgctcaggacaacaacccagatgcacagacactccagaaattggcagaaaggacaggcttgagcagacgtgtgatacaggtgtggtttcagaattgtagagcacgccacaagaaacacgtcagtcctaatcactcatcctccaccccagtcacagcagccccaccctccaggctgtctccacccatgttagaagaaatggcttattctgcctacgtgccccaagatggaacgatgttaactgcgctgcatagttatatggatgctcattcaccaacaactcttggactccagcccttgttaccccattcaatgacacaactgccaataagtcatacctaattcttttttcagggatagacttgattaaggatataaatttgtcatttattatgtataaaataccattgaaaagatattactgttaattttttatttaacacctaaagcatttccaacatcactttgctgcccaggtatgtatctatagttggcctgcaagacacttttattaattcttcattttttgtaaaacttatgtttacaagaagaaaacaaatcaaaacattttttgtattgtctggaaatagttcactctagtgtgtatctgttaatttatttgtcatcaaaagagcactttgcctaaaagaaaggactgacaagtgtgcaaaatgtttacaatcttttgtgaaattgtagtttatcattagtttgtatctgtaagttattgtaataaatattacctgtattttttgttatatacaactttatactttgaagcttgtatctgtgaatttgcaactgaaatttattttgccaatgttttctgaatgaactgaataaagcttctgttgtagcatgccatgcaaacacattattgtgtttgtggttgatgaattatggctgtaaataacactatagtttaataagcccaccattctgagtttattaaacattttccattcttgtgaaaatttcaaaaaaaaaaaaaaaaaaaaaagaaaaaaaaaaaaaaaaa

SEQ ID NO: 57 - protein, 682 aa, Homo sapiens
MQLEHCLSPSIMLSKKFLNVSSSYPHSGGSELVLHDHPIISTTDNLERSSPLKKITRGMTNQSDTDNFPDSKDSPGDVQRSKLSPVLDGVSELRHSFDGSAADRYLLSQSSQPQSAATAPSAMFPYPGQHGPAHPAFSIGSPSRYMAHHPVITNGAYNSLLSNSSPQGYPTAGYPYPQQYGHSYQGAPFYQFSSTQPGLVPGKAQVYLCNRPLWLKFHRHQTEMIITKQGRRMFPFLSFNISGLDPTAHYNIFVDVILADPNHWRFQGGKWVPCGKADTNVQGNRVYMHPDSPNTGAHWMRQEISFGKLKLTNNKGASNNNGQMVVLQSLHKYQPRLHVVEVNEDGTEDTSQPGRVQTFTFPETQFIAVTAYQNTDITQLKIDHNPFAKGFRDNYDTIYTGCDMDRLTPSPNDSPRSQIVPGARYAMAGSFLQDQFVSNYAKARFHPGAGAGPGPGTDRSVPHTNGLLSPQQAEDPGAPSPQRWFVTPANNRLDFAASAYDTATDFAGNAATLLSYAAAGVKALPLQAAGCTGRPLGYYADPSGWGARSPPQYCGTKSGSVLPCWPNSAAAAARMAGANPYLGEEAEGLAAERSPLPPGAAEDAKPKDLSDSSWIETPSSIKSIDSSDSGIYEQAKRRRISPADTPVSESSSPLKSEVLAQRDCEKNCAKDISGYYGFYSHS

SEQ ID NO: 58 - DNA, 4006 bp, Homo sapiens
  source 1..4006: mol_type = unassigned DNA
gtcgctaccaggagccaggtgattatcctaattaatgtctatctaattaaattactgtcagcagctaaccaatggcaggagccgtttcatcggctgcacaagcagcaagatcaaaagtgagccttttctgattgctgcatagtgtcaattggccaatctcttctcccagggaaaaaaaaaagtaaatcaaacctttgagaagcatttgctggttgaagtgctttctgtctagtgagggggtctgtggatttctagtttatgataaataggactttaaaaaccagggacgggagggcgagtgttcaggttctagagctatgcagctggagcactgcctttctccttctatcatgctctccaagaaatttctcaatgtgagcagcagctacccacattcaggcggatccgagcttgtcttgcacgatcatcccattatctcgaccactgacaacctggagagaagttcacctttgaaaaaaattaccagggggatgacgaatcagtcagatacagacaattttcctgactccaaggactcaccaggggacgtccagagaagtaaactctctcctgtcttggacggggtctctgagcttcgtcacagtttcgatggctctgctgcagatcgctacctcctctctcagtccagccagccacagtctgcggccactgctcccagtgccatgttcccgtaccccggccagcacggaccggcgcaccccgccttctccatcggcagccctagccgctacatggcccaccacccggtcatcaccaacggagcctacaacagcctcctgtccaactcctcgccgcagggataccccacggccggctacccctacccacagcagtacggccactcctaccaaggagctccgttctaccagttctcctccacccagccggggctggtgcccggcaaagcacaggtgtacctgtgcaacaggcccctttggctgaaatttcaccggcaccaaacggagatgatcatcaccaaacagggaaggcgcatgtttccttttttaagttttaacatttctggtctcgatcccacggctcattacaatatttttgtggatgtgattttggcggatcccaatcactggaggtttcaaggaggcaaatgggttccttgcggcaaagcggacaccaatgtgcaaggaaatcgggtctatatgcatccggattcccccaacactggggctcactggatgcgccaagaaatctcttttggaaaattaaaacttacgaacaacaaaggagcttcaaataacaatgggcagatggtggttttacagtccttgcacaagtaccagccccgcctgcatgtggtggaagtgaacgaggacggcacggaggacactagccagcccggccgcgtgcagacgttcactttccctgagactcagttcatcgccgtcaccgcctaccagaacacggatattacacaactgaaaatagatcacaacccttttgcaaaaggatttcgggataattatgacacgatctacaccggctgtgacatggaccgcctgaccccctcgcccaacgactcgccgcgctcgcagatcgtgcccggggcccgctacgccatggccggctctttcctgcaggaccagttcgtgagcaactacgccaaggcccgcttccacccgggcgcgggcgcgggccccgggccgggtacggaccgcagcgtgccgcacaccaacgggctgctgtcgccgcagcaggccgaggacccgggcgcgccctcgccgcaacgctggtttgtgacgccggccaacaaccggctggacttcgcggcctcggcctatgacacggccacggacttcgcgggcaacgcggccacgctgctctcttacgcggcggcgggcgtgaaggcgctgccgctgcaggctgcaggctgcactggccgcccgctcggctactacgccgacccgtcgggctggggcgcccgcagtcccccgcagtactgcggcaccaagtcgggctcggtgctgccctgctggcccaacagcgccgcggccgccgcgcgcatggccggcgccaatccctacctgggcgaggaggccgagggcctggccgccgagcgctcgccgctgccgcccggcgccgccgaggacgccaagcccaaggacctgtccgattccagctggatcgagacgccctcctcgatcaagtccatcgactccagcgactcggggatttacgagcaggccaagcggaggcggatctcgccggccgacacgcccgtgtccgagagttcgtccccgctcaagagcgaggtgctggcccagcgggactgcgagaagaactgcgccaaggacattagcggctactatggcttctactcgcacagctaggccgcccctgcccgcccggccccgccgcggcccggacccccagccagcccctcacagctcttccccagctccgcctccccacactcctccttgcgcacccactcattttatttgaccctcgatggccgtctgcagcgaataagtgcaggtctccgagcgtgattttaaccttttttgcacagcagtctctgcaattagctcaccgaccttcaactttgctgtaaaccttttggttttcctacttactcttcttctgtggagttatcctcctacaattcccctccccctcgtctttctcttacctcctacttctctttcttgtaatgaaactcttcacctttaggagacctgggcagtcctgtcaggcagcagcgattccgacccgccaagtctcggcctccacattaaccataggatgttgactctagaacctggacccacccagcgcgtcctttcttatccccgagtggatggatggatggatggatggtagggatgttaataattttagtggaacaaagcctgtgaaatgattgtacatagtgttaatttattgtaacgaatggctagtttttattctcgtcaaggcacaaaaccagttcatgcttaacctttttttcctttcctttctttgcttttctttctctcctctcatactttctcttctctctcttttaattttcttgtgagataatattctaagaggctctagaaacatgaaatactcagtagtgatgggtttcccacttctcctcaatccgttgcatgaaataattactatgtgccctaatgcacacaaatagctaaggagaatccacccaaacacctttaaaggataggtgtctgttcataggcaagtcgattaagtggcatgatgcctgcaaagcaaagtcaactggagttgtatgttccccccaccttctaaatagaatagctcgacatcagcaatattattttgccttatttgtttttccccaaagtgccaaatccattactggtctgtgcaggtgccaaatatgctgacaaactgtttctgaatatctttcagtaccccttcacctttatatgctgtaaatctttgtaatgaatactctattaatgatatagatgactgaattgttggtaactatagtgtagtctagtgaagatgaattgtgtgagttgtatattttactgcattttagttttgaaaatgacttccccaccacctagaaacagctgaaatttgacttccttgggagaacactagcattaatgcaagtaagactgattttcccctaagtcttgttatatttgataaggagcattaatccccctggaaatagattagtaggatttctaatgttgtgtagcaaacctatacttttttgtatttaaaaattaatgtgaaatatgcatcatacacaatattcaatctagattccagtccatggggggatttttcctaataggaattcagggtctaaacgtgtgtatattttggctcttctgtaaatctaatgttgtgatttttatatttgtttcgttttgtctgtgaactgaataatttatacaagaacacactccattgagaaacgttttgttttttgctcgtttgtatcgtctgtgtataacaagtaaaataaacctggtaaaaacgc

SEQ ID NO: 59 - protein, 542 aa, Homo sapiens
MTKSNGEEPKMGGRMERFQQGVRKRTLLAKKKVQNITKEDVKSYLFRNAFVLLTVTAVIVGTILGFTLRPYRMSYREVKYFSFPGELLMRMLQMLVLPLIISSLVTGMAALDSKASGKMGMRAVVYYMTTTIIAVVIGIIIVIIIHPGKGTKENMHREGKIVRVTAADAFLDLIRNMFPPNLVEACFKQFKTNYEKRSFKVPIQANETLVGAVINNVSEAMETLTRITEELVPVPGSVNGVNALGLVVFSMCFGFVIGNMKEQGQALREFFDSLNEAIMRLVAVIMWYAPVGILFLIAGKIVEMEDMGVIGGQLAMYTVTVIVGLLIHAVIVLPLLYFLVTRKNPWVFIGGLLQALITALGTSSSSATLPITFKCLEENNGVDKRVTRFVLPVGATINMDGTALYEALAAIFIAQVNNFELNFGQIITISITATAASIGAAGIPQAGLVTMVIVLTSVGLPTDDITLIIAVDWFLDRLRTTTNVLGDSLGAGIVEHLSRHELKNRDVEMGNSVIEENEMKKPYQLIAQDNETEKPIDSETKM

SEQ ID NO: 60 - DNA, 1873 bp, Homo sapiens
  source 1..1873: mol_type = unassigned DNA
gatagtaacttgcagtttcagagcacatgcacactgtcagggctagcctgcctgcttacgcgcgctgcggattgttgctccgttgtacctgctggggaattcacctcgttactgcttgatatcttccaccccttacaaaatcagaaaagttgtgttttctaataccaaagaggaggtttggctttctgtgggtgattcccagacactgaagtgcaaagaagagaccctcctagaaaagtaaaatatgactaaaagcaatggagaagagcccaagatggggggcaggatggagagattccagcagggagtccgtaaacgcacacttttggccaagaagaaagtgcagaacattacaaaggaggatgttaaaagttacctgtttcggaatgcttttgtgctgctcacagtcaccgctgtcattgtgggtacaatccttggatttaccctccgaccatacagaatgagctaccgggaagtcaagtacttctcctttcctggggaacttctgatgaggatgttacagatgctggtcttaccacttatcatctccagtcttgtcacaggaatggcggcgctagatagtaaggcatcagggaagatgggaatgcgagctgtagtctattatatgactaccaccatcattgctgtggtgattggcataatcattgtcatcatcatccatcctgggaagggcacaaaggaaaacatgcacagagaaggcaaaattgtacgagtgacagctgcagatgccttcctggacttgatcaggaacatgttccctccaaatctggtagaagcctgctttaaacagtttaaaaccaactatgagaagagaagctttaaagtgcccatccaggccaacgaaacgcttgtgggtgctgtgataaacaatgtgtctgaggccatggagactcttacccgaatcacagaggagctggtcccagttccaggatctgtgaatggagtcaatgccctgggtctagttgtcttctccatgtgcttcggttttgtgattggaaacatgaaggaacaggggcaggccctgagagagttctttgattctcttaacgaagccatcatgagactggtagcagtaataatgtggtatgcccccgtgggtattctcttcctgattgctgggaagattgtggagatggaagacatgggtgtgattggggggcagcttgccatgtacaccgtgactgtcattgttggcttactcattcacgcagtcatcgtcttgccactcctctacttcttggtaacacggaaaaacccttgggtttttattggagggttgctgcaagcactcatcaccgctctggggacctcttcaagttctgccaccctacccatcaccttcaagtgcctggaagagaacaatggcgtggacaagcgcgtcaccagattcgtgctccccgtaggagccaccattaacatggatgggactgccctctatgaggctttggctgccattttcattgctcaagttaacaactttgaactgaacttcggacaaattattacaatcagcatcacagccacagctgccagtattggggcagctggaattcctcaggcgggcctggtcactatggtcattgtgctgacatctgtcggcctgcccactgacgacatcacgctcatcatcgcggtggactggttcctggatcgcctccggaccaccaccaacgtactgggagactccctgggagctgggattgtggagcacttgtcacgacatgaactgaagaacagagatgttgaaatgggtaactcagtgattgaagagaatgaaatgaagaaaccatatcaactgattgcacaggacaatgaaactgagaaacccatcgacagtgaaaccaagatgtag

SEQ ID NO: 61 - protein, 497 aa, Homo sapiens
MPTPDATTPQAKGFRRAVSELDAKQAEAIMSPRFIGRRQSLIEDARKEREAAVAAAAAAVPSEPGDPLEAVAFEEKEGKAVLNLLFSPRATKPSALSRAVKVFETFEAKIHHLETRPAQRPRAGGPHLEYFVRLEVRRGDLAALLSGVRQVSEDVRSPAGPKVPWFPRKVSELDKCHHLVTKFDPDLDLDHPGFSDQVYRQRRKLIAEIAFQYRHGDPIPRVEYTAEEIATWKEVYTTLKGLYATHACGEHLEAFALLERFSGYREDNIPQLEDVSRFLKERTGFQLRPVAGLLSARDFLASLAFRVFQCTQYIRHASSPMHSPEPDCCHELLGHVPMLADRTFAQFSQDIGLASLGASDEEIEKLSTLYWFTVEFGLCKQNGEVKAYGAGLLSSYGELLHCLSEEPEIRAFDPEAAAVQPYQDQTYQSVYFVSESFSDAKDKLRSYASRIQRPFSVKFDPYTLAIDVLDSPQAVRRSLEGVQDELDTLAHALSAIG

SEQ ID NO: 62 - DNA, 1629 bp, Homo sapiens
  source 1..1629: mol_type = unassigned DNA
acccagagggggctttgacgtcagctcagcttataagaggctgctgggccagggctgtggagacggagcccggacctccacactgagccatgcccacccccgacgccaccacgccacaggccaagggcttccgcagggccgtgtctgagctggacgccaagcaggcagaggccatcatgtccccgcggttcattgggcgcaggcagagcctcatcgaggacgcccgcaaggagcgggaggcggcggtggcagcagcggccgctgcagtcccctcggagcccggggaccccctggaggctgtggcctttgaggagaaggaggggaaggccgtgctaaacctgctcttctccccgagggccaccaagccctcggcgctgtcccgagctgtgaaggtgtttgagacgtttgaagccaaaatccaccatctagagacccggcccgcccagaggccgcgagctgggggcccccacctggagtacttcgtgcgcctcgaggtgcgccgaggggacctggccgccctgctcagtggtgtgcgccaggtgtcagaggacgtgcgcagccccgcggggcccaaggtcccctggttcccaagaaaagtgtcagagctggacaagtgtcatcacctggtcaccaagttcgaccctgacctggacttggaccacccgggcttctcggaccaggtgtaccgccagcgcaggaagctgattgctgagatcgccttccagtacaggcacggcgacccgattccccgtgtggagtacaccgccgaggagattgccacctggaaggaggtctacaccacgctgaagggcctctacgccacgcacgcctgcggggagcacctggaggcctttgctttgctggagcgcttcagcggctaccgggaagacaatatcccccagctggaggacgtctcccgcttcctgaaggagcgcacgggcttccagctgcggcctgtggccggcctgctgtccgcccgggacttcctggccagcctggccttccgcgtgttccagtgcacccagtatatccgccacgcgtcctcgcccatgcactcccctgagccggactgctgccacgagctgctggggcacgtgcccatgctggccgaccgcaccttcgcgcagttctcgcaggacattggcctggcgtccctgggggcctcggatgaggaaattgagaagctgtccacgctgtactggttcacggtggagttcgggctgtgtaagcagaacggggaggtgaaggcctatggtgccgggctgctgtcctcctacggggagctcctgcactgcctgtctgaggagcctgagattcgggccttcgaccctgaggctgcggccgtgcagccctaccaagaccagacgtaccagtcagtctacttcgtgtctgagagcttcagtgacgccaaggacaagctcaggagctatgcctcacgcatccagcgccccttctccgtgaagttcgacccgtacacgctggccatcgacgtgctggacagcccccaggccgtgcggcgctccctggagggtgtccaggatgagctggacacccttgcccatgcgctgagtgccattggctaggtgcacggcgtccctgagggcccttcccaacctcccctggtcctgc

SEQ ID NO: 63 - protein, 1020 aa, Homo sapiens
MMSFGGADALLGAPFAPLHGGGSLHYALARKGGAGGTRSAAGSSSGFHSWTRTSVSSVSASPSRFRGAGAASSTDSLDTLSNGPEGCMVAVATSRSEKEQLQALNDRFAGYIDKVRQLEAHNRSLEGEAAALRQQQAGRSAMGELYEREVREMRGAVLRLGAARGQLRLEQEHLLEDIAHVRQRLDDEARQREEAEAAARALARFAQEAEAARVDLQKKAQALQEECGYLRRHHQEEVGELLGQIQGSGAAQAQMQAETRDALKCDVTSALREIRAQLEGHAVQSTLQSEEWFRVRLDRLSEAAKVNTDAMRSAQEEITEYRRQLQARTTELEALKSTKDSLERQRSELEDRHQADIASYQEAIQQLDAELRNTKWEMAAQLREYQDLLNVKMALDIEIAAYRKLLEGEECRIGFGPIPFSLPEGLPKIPSVSTHIKVKSEEKIKVVEKSEKETVIVEEQTEETQVTEEVTEEEEKEAKEEEGKEEEGGEEEEAEGGEEETKSPPAEEAASPEKEAKSPVKEEAKSPAEAKSPEKEEAKSPAEVKSPEKAKSPAKEEAKSPPEAKSPEKEEAKSPAEVKSPEKAKSPAKEEAKSPAEAKSPEKAKSPVKEEAKSPAEAKSPVKEEAKSPAEVKSPEKAKSPTKEEAKSPEKAKSPEKEEAKSPEKAKSPVKAEAKSPEKAKSPVKAEAKSPEKAKSPVKEEAKSPEKAKSPVKEEAKSPEKAKSPVKEEAKTPEKAKSPVKEEAKSPEKAKSPEKAKTLDVKSPEAKTPAKEEARSPADKFPEKAKSPVKEEVKSPEKAKSPLKEDAKAPEKEIPKKEEVKSPVKEEEKPQEVKVKEPPKKAEEEKAPATPKTEEKKDSKKEEAPKKEAPKPKVEEKKEPAVEKPKESKVEAKKEEAEDKKKVPTPEKEAPAKVEVKEDAKPKEKTEVAKKEPDDAKAKEPSKPAEKKEAAPEKKDTKEEKAKKPEEKPKTEAKAKEDDKTLSKEPSKPKAEKAEKSSSTDQKDSKPPEKATEDKAAKGK

SEQ ID NO: 64 - DNA, 3721 bp, Homo sapiens
  source 1..3721: mol_type = unassigned DNA
aaaagggccggcgccctggtgctgccgcagtgcctcccgccccgtcccggcctcgcgcacctgctcaggccatgatgagcttcggcggcgcggacgcgctgctgggcgccccgttcgcgccgctgcatggcggcggcagcctccactacgcgctagcccgaaagggtggcgcaggcgggacgcgctccgccgctggctcctccagcggcttccactcgtggacacggacgtccgtgagctccgtgtccgcctcgcccagccgcttccgtggcgcaggcgccgcctcaagcaccgactcgctggacacgctgagcaacgggccggagggctgcatggtggcggtggccacctcacgcagtgagaaggagcagctgcaggcgctgaacgaccgcttcgccgggtacatcgacaaggtgcggcagctggaggcgcacaaccgcagcctggagggcgaggctgcggcgctgcggcagcagcaggcgggccgctccgctatgggcgagctgtacgagcgcgaggtccgcgagatgcgcggcgcggtgctgcgcctgggcgcggcgcgcggtcagctacgcctggagcaggagcacctgctcgaggacatcgcgcacgtgcgccagcgcctagacgacgaggcccggcagcgagaggaggccgaggcggcggcccgcgcgctggcgcgcttcgcgcaggaggccgaggcggcgcgcgtggacctgcagaagaaggcgcaggcgctgcaggaggagtgcggctacctgcggcgccaccaccaggaagaggtgggcgagctgctcggccagatccagggctccggcgccgcgcaggcgcagatgcaggccgagacgcgcgacgccctgaagtgcgacgtgacgtcggcgctgcgcgagattcgcgcgcagcttgaaggccacgcggtgcagagcacgctgcagtccgaggagtggttccgagtgaggctggaccgactgtcggaggcagccaaggtgaacacagacgctatgcgctcagcgcaggaggagataactgagtaccggcgtcagctgcaggccaggaccacagagctggaggcactgaaaagcaccaaggactcactggagaggcagcgctctgagctggaggaccgtcatcaggccgacattgcctcctaccaggaagccattcagcagctggacgctgagctgaggaacaccaagtgggagatggccgcccagctgcgagaataccaggacctgctcaatgtcaagatggctctggatatagagatagccgcttacagaaaactcctggaaggtgaagagtgtcggattggctttggcccaattcctttctcgcttccagaaggactccccaaaattccctctgtgtccactcacataaaggtgaaaagcgaagagaagatcaaagtggtggagaagtctgagaaagaaactgtgattgtggaggaacagacagaggagacccaagtgactgaagaagtgactgaagaagaggagaaagaggccaaagaggaggagggcaaggaggaagaagggggtgaagaagaggaggcagaagggggagaagaagaaacaaagtctcccccagcagaagaggctgcatccccagagaaggaagccaagtcaccagtaaaggaagaggcaaagtcaccggctgaggccaagtccccagagaaggaggaagcaaaatccccagccgaagtcaagtcccctgagaaggccaagtctccagcaaaggaagaggcaaagtcaccgcctgaggccaagtccccagagaaggaggaagcaaaatctccagctgaggtcaagtcccccgagaaggccaagtccccagcaaaggaagaggcaaagtcaccggctgaggccaagtctccagagaaggccaagtccccagtgaaggaagaagcaaagtcaccggctgaggccaagtccccagtgaaggaagaagcaaaatctccagctgaggtcaagtccccggaaaaggccaagtctccaacgaaggaggaagcaaagtcccctgagaaggccaagtccccagagaaggaagaggccaagtcccctgagaaggccaagtccccagtgaaggcagaagcaaagtcccctgagaaggccaagtccccagtgaaggcagaagcaaagtcccctgagaaggccaagtccccagtgaaggaagaagcaaagtcccctgagaaggccaagtccccagtgaaggaagaagcaaagtcccctgagaaggccaagtccccagtgaaggaagaagcaaagacccccgagaaggccaagtccccagtgaaggaagaagctaagtccccagagaaggccaagtccccagagaaggccaagactcttgatgtgaagtctccagaagccaagactccagcgaaggaggaagcaaggtcccctgcagacaaattccctgaaaaggccaaaagccctgtcaaggaggaggtcaagtccccagagaaggcgaaatctcccctgaaggaggatgccaaggcccctgagaaggagatcccaaaaaaggaagaggtgaagtccccagtgaaggaggaggagaagccccaggaggtgaaagtcaaagagcccccaaagaaggcagaggaagagaaagcccctgccacaccaaaaacagaggagaagaaggacagcaagaaagaggaggcacccaagaaggaggctccaaagcccaaggtggaggagaagaaggaacctgctgtcgaaaagcccaaagaatccaaagttgaagccaagaaggaagaggctgaagataagaaaaaagtccccaccccagagaaggaggctcctgccaaggtggaggtgaaggaagacgctaaacccaaagaaaagacagaggtagccaagaaggaaccagatgatgccaaggccaaggaacccagcaaaccagcagagaagaaggaggcagcaccggagaaaaaagacaccaaggaggagaaggccaagaagcctgaggagaaacccaagacagaggccaaagccaaggaagatgacaagaccctctcaaaagagcctagcaagcctaaggcagaaaaggctgaaaaatcctccagcacagaccaaaaagacagcaagcctccagagaaggccacagaagacaaggccgccaaggggaagtaaggcagggagaaaggaacatccggaacagccaaagaaactcagaagagtcccggagctcaaggatcagagtaacacaattttcactttttctgtctttatgtaagaagaaactgcttagatgacggggcctccttcttcaaacaggaatttctgttagcaatatgttagcaagagagggcactcccaggcccctgcccccaggccctccccaggcgatggacaattatgatagcttatgtagctgaatgtgatacatgccgaatgccacacgtaaacacttgactataaaaactgcccccctcctttccaaataagtgcatttattgcctctatgtgcaactgacagatgaccgcaataatgaatgagcagttagaaatacattatgcttgagatgtcttaacctattcccaaatgccttctgttttccaaaggagtggtcaagcccttgcccagagctctctattctggaagagcggtccaggtggggccggggactggccactgaattatgccagggcgcactttccactggagttcactttcaattgcttctgtgcaataaaaccaagtgcttataaaatgaaaa

SEQ ID NO: 65 - protein, 559 aa, Homo sapiens
MADERKDEAKAPHWTSAPLTEASAHSHPPEIKDQGGAGEGLVRSANGFPYREDEEGAFGEHGSQGTYSNTKENGINGELTSADRETAEEVSARIVQVVTAEAVAVLKGEQEKEAQHKDQTAALPLAAEETANLPPSPPPSPASEQTVTVEEAAGGESALAPSVFKQAKDKVSNSTLSKIPALQGSTKSPRYSSACPSTTKRATFSDSLLIQPTSAGSTDRLPYSKSGNKDGVTKSPEKRSSLPRPSSILPPRRGVSGDRDENSFSLNSSISSSARRTTRSEPIRRAGKSGTSTPTTPGSTAITPGTPPSYSSRTPGTPGTPSYPRTPHTPGTPKSAILVPSEKKVAIIRTPPKSPATPKQLRLINQPLPDLKNVKSKIGSTDNIKYQPKGGQVRILNKKIDFSKVQSRCGSKDNIKHSAGGGNVQIVTKKIDLSHVTSKCGSLKNIRHRPGGGRVKIESVKLDFKEKAQAKVGSLDNAHHVPGGGNVKIDSQKLNFREHAKARVDHGAEIITQSPGRSSVASPRRLSNVSSSGSINLLESPQLATLAEDVTAALAKQGL

SEQ ID NO: 66 - DNA, 2286 bp, Homo sapiens
  source 1..2286: mol_type = unassigned DNA
ggcgctcgggctgcgcgggctctgggcagcagcagcagcagcagcagcatcctctcttcctttacttcccttccgcttctttctcttccttctccttctttttcccccccctccccttcttcccctaacccttctacccctctcctttttctccggagggcgctaagtccgtgagcggtggcagtcgcgaccgcgggtgcatccagtttctgcgcccagattttattgatctaatccaaagtatcttataacttctggctggaattaagattcttcagcttgtctctaaccgaggaagcattgattgggagctactcattcagaaaattaaaagaaagaagccagaaaatattatcaaccctttgagaacacgacacaacgaactttatattttaccacttccttgaatagttgcaggagaaataacaaggcattgaagaatggcagatgaacggaaagacgaagcaaaggcacctcactggacctcagcaccgctaacagaggcatctgcacactcacatccacctgagattaaggatcaaggcggagcaggggaaggacttgtccgaagcgccaatggattcccatacagggaggatgaagagggtgcctttggagagcatgggtcacagggcacctattcaaataccaaagagaatgggatcaacggagagctgacctcagctgacagagaaacagcagaggaggtgtctgcaaggatagttcaagtagtcactgctgaggctgtagcagtcctgaaaggtgaacaagagaaagaagctcaacataaagaccagactgcagctctgcctttagcagctgaagaaacagctaatctgcctccttctccacccccatcacctgcctcagaacagactgtcacagtggaggaagcagcaggtggggaatcagctctggctcccagtgtatttaaacaggcaaaggacaaagtctctaattctaccttgtcaaagattcctgctttacagggtagcacaaagtccccaagatacagctcagcctgccctagcacgactaaaagggctacattttctgacagtttattaatacagcccacctcagcaggctccacagaccgtttgccatactcaaaatcagggaacaaggacggagtaaccaagagcccagaaaagcgctcttctctcccaagaccttcctccattctccctcctcggcgaggtgtgtcaggagacagagatgagaattccttctctctcaacagttctatctcttcttcagcacggcggaccaccaggtcagagccaattcgcagagcagggaagagtggtacctcaacacccactacccctgggtctactgccatcactcctggcaccccaccaagttattcttcacgcacaccaggcactcctggaacccctagctatcccaggacccctcacacaccaggaacccccaagtctgccatcttggtgccgagtgagaagaaggtcgccatcatacgtactcctccaaaatctcctgcgactcccaagcagcttcggcttattaaccaaccactgccagacctgaagaatgtcaaatccaaaatcggatcaacagacaacatcaaataccagcctaaaggggggcaggttaggattttaaacaagaagatcgattttagcaaagttcagtccagatgtggttccaaggataacatcaaacattcggctgggggcggaaatgtacaaattgttaccaagaaaatagacctaagccatgtgacatccaaatgtggctctctgaagaacatccgccacaggccaggtggcggacgtgtgaaaattgagagtgtaaaactagatttcaaagaaaaggcccaagctaaagttggttctcttgataatgctcatcatgtacctggaggtggtaatgtcaagattgacagccaaaagttgaacttcagagagcatgctaaagcccgtgtggaccatggggctgagatcattacacagtccccaggcagatccagcgtggcatcaccccgacgactcagcaatgtctcctcgtctggaagcatcaacctgctcgaatctcctcagcttgccactttggctgaggatgtcactgctgcactcgctaagcagggcttgtgaatatttctcatttagcattgaaataataatatttaggcatgagctcttggcaggagtgggctctgagcagttgttatattcattctttataaaccataaaataaataatctcatccccaaaaaaaaaaaaaaaaaaaaaaaaaaaaaaaaaaaaaaaaaaaaaaaa

SEQ ID NO: 67 - protein, 360 aa, Homo sapiens
MELDFGHFDERDKTSRNMRGSRMNGLPSPTHSAHCSFYRTRTLQALSNEKKAKKVRFYRNGDRYFKGIVYAVSSDRFRSFDALLADLTRSLSDNINLPQGVRYIYTIDGSRKIGSMDELEEGESYVCSSDNFFKKVEYTKNVNPNWSVNVKTSANMKAPQSLASSNSAQARENKDFVRPKLVTIIRSGVKPRKAVRVLLNKKTAHSFEQVLTDITEAIKLETGVVKKLYTLDGKQVTCLHDFFGDDDVFIACGPEKFRYAQDDFSLDENECRVMKGNPSATAGPKASPTPQKTSAKSPGPMRRSKSPADSANGTSSSQLSTPKSKQSPISTPTSPGSLRKHKDLYLPLSLDDSDSLGDSM

SEQ ID NO: 68 - DNA, 9135 bp, Homo sapiens
  source 1..9135: mol_type = unassigned DNA
ctggcaggaatttcttgcttggagctcagacaacaaaggcatagagagattggttttctttctctcagcatctccacccaaccagcagaaaaccggtctctgaggttccaccaaaatatggaacttgattttggacactttgacgaaagagataagacatccaggaacatgcgaggctcccggatgaatgggttgcctagccccactcacagcgcccactgtagcttctaccgaaccagaaccttgcaggcactgagtaatgagaagaaagccaagaaggtacgtttctaccgcaatggggaccgctacttcaaggggattgtgtacgctgtgtcctctgaccgttttcgcagctttgacgccttgctggctgacctgacgcgatctctgtctgacaacatcaacctgcctcagggagtgcgttacatttacaccattgatggatccaggaagatcggaagcatggatgaactggaggaaggggaaagctatgtctgttcctcagacaacttctttaaaaaggtggagtacaccaagaatgtcaatcccaactggtctgtcaacgtaaaaacatctgccaatatgaaagccccccagtccttggctagcagcaacagtgcacaggccagggagaacaaggactttgtgcgccccaagctggttaccatcatccgcagtggggtgaagcctcggaaggctgtgcgtgtgcttctgaacaagaagacagcccactcttttgagcaagtcctcactgatatcacagaagccatcaaactggagaccggggttgtcaaaaaactctacactctggatggaaaacaggtaacttgtctccatgatttctttggtgatgatgatgtgtttattgcctgtggtcctgaaaaatttcgctatgctcaggatgatttttctctggatgaaaatgaatgccgagtcatgaagggaaacccatcagccacagctggcccaaaggcatccccaacacctcagaagacttcagccaagagccctggtcctatgcgccgaagcaagtctccagctgactcagcaaacggaacctccagcagccagctctctacccccaagtctaagcagtctcccatctctacgcccaccagtcctggcagcctccggaagcacaaggacctgtacctgcctctgtccttggatgactcggactcgcttggtgattccatgtaaaggaggggagagtgctcagagtccagagtacaaatccaagcctatcattgtagtagggtacttctgctcaagtgtccaacagggctattggtgctttcaagtttttattttgttgttgttgttattttgaaaaacacattgtaatatgttgggtttattttcctgtgatttctcctctgggccactgatccacagttaccaattatgagagatagattgataaccatcctttggggcagcattccagggatgcaaaatgtgctagtccatgacctttcaatggaaagcttaggtgcctgcgttatatttgccctgtctaattttgcccatacagtcttccttctgtagagggctgtttacatatacagcacttaaaatgtttgtgtgggaaaaaaaaaactcattggcagatccaagaatgacaaacacaagtgccccttttctctggatctcaagaatggtggaggaccctggaaggacagcaaggcagctccccagcctcactcttcactcctgattgaggcccgggtttgttgtccagcaccaattctggctgtcaatggggagaaataaaccaacaacttataattgtgacaccagatgcttaggatcctggtgctgggttagctaagagaatagacagaattggaaaatactgcagacatttccgaagagtttataaagcacagtgaattcctggtcaatctctccactgaggcaatttggaatcaataagcaattgataatagtttggagtaagggacttcatatacctgattcctctagaaggctgtctaacataccacatgattacatgaactgtatggtatccatctatctctgttctattgaatgccttgttaacagccaacactgaaaacactgtgagaatttgttttcaggtctgacacctttcagtctctttttatagcaagaaatcaatatcctttttataaaaattcatgtctgtatttcaggagcaaactcttcaggctccttttttataaactggtgatttttcttttgtctaaaaaacacatgaagaaaatttaccaaaaaaaaaaaaaaaagcagaagaataatgtagtttagaaattatgctgtcactgccaaacagtaacctccaggagaaaacaagatgaatagcagaggccaattcaatagaatcagttttttgatagctttttaacagttatgcttgcattaataatttcaatgtggaccagacattctaattatattttaaatgaaatgttacagcatattttaagcaactctttttatctataatcctaatatttcatactgaagacacagaaatctttcacttgtctttaacattagaaaggatttctctttactaaggactgatcatttgaaatagttttcagtcttttgagatacaggtttataacactgcttttttttttctgtaatcatagcccataatggcaaagacaactaaatttaagtgaaggtcatgcatgccaattctgtgtttgcttttagcagatatgaagatttccttatttctttgtaattgtgcagatattttgaaaggcacagcattcgaagccaagctgctgtttggctactgaatggcttgcagttgttcctccactctaaatggaatgagcttgctgtgtgtgtgtgtggtggtggtgggagggggtggtgcatgtgtgtgtgtgtgtgtgcatctgcagctgcttcaaaattaggaaatactacaggacacccctgtaatggattggtggcaactgggtggcactgctgatgtgcactgtgtaggggggaacccagtggtggtggggtagctcagatgcccctagacaagcttcagatgtctgtagctaccagaaacattttcggttcaggaaaagtgagatgatggtagtactggtttctggtgaaattgaagaaccccaaatgatgaggatctctttttgccccctctcctttttttgtagacccattcaaaaccattaataagcccattttactaagcccctatttctttctagaagctcagggttttcttagtgcctcccagaacattttgtagttaattgggaaaaagtgatacttggattagggggtgtgggcataaagaatggtgggaggcctgattttaaacttcaggccagaacccccaatgactccacccatagtctcactttaggtctcatttagtccatcacctttattttaagttgaggaagtggaggctggtaaagagcaggaccagaggaagaatccagatttccttatgcttgggcctcacactagctctctgagtatttccttgattgcggtatatgtactactagaaaataccaaatggatatattttctttaggataacctttgaaccaacaatcttcaataacaatagtacatcttccatcttacttttaatcgagtataaggaaatgtttctttatggccattttggagggagcaggggatgaggcttggcatagtccaaaatttaagtctccaataattaattgcattttaaattggcccactttcaaggcaattttttttgtgtgtctgtaactgagctcctccacccctgtcattcacttccaattttacccaatccaattttagcactcaagttccattgtgttaatttctgcacggtcaacaaacatcaagtcagcaagcatttgccaccactccctatacttctccctccttcttacacacacacacacacacacacacacaatccatctcttgcttgttcctacctcctgatttttcttccctacagaaatagaaatagggacaaagaaggggaaaatgtatatattggggctgggctgaacaactaacttcataagtagtattaactaggggtaaattgagagaaaagctccttttctcttcactgttttggaaaggatagccattagcatgactgctttgtgtccttatggactttagtattagcctagattgaattatagcgttttctagctggaaggaaccttaagatcacatcatctactcctctactccaaatttctcattcttcaggccaggaaaccgagacacagaggtaaagtaatttccccaaggtcacacagctggctggggcaggattgggtttacaacccacatctcctggctcttattccagggccttttcccactaagtagtattgccttccattaggctcctgagagttatttctcagggtcatgttgcatcttggagccacatgctgctgccctgatctcagtgggaaatccacccagcaacctaatacagccccttttccctgcattcacctggttcccatccacatgggttgcagatgtccttgaagagagtgaggcattgagggccaataggagcaatggggtccctggccttgtccatctgattcaggagatcactgctccatcgtgaggagccctctgaatagccccccactgaatgcttgccttgcccaaatggaatggaggaagattgattttctccatcagttcaccttgtgtcatctcataatggttggtctttccaggctgagggaaatgtttcttgtttccagagtagaaaaagaaagagtggaacaatagctttgttcatcctaactttctgagatggcttttcaacattttaaaaaaactagtgtggctaccattcactggcaatgatttcttttagaatatgggagtaagatgagctagagaaaataacctggtctcactgtggttgccctcatccacaatgtccccaaagccatcctgctctgatgaggacaatttccaggtataagcaaggggctttgtgacaaaaatgtaccctggctgatgttaaacattggctcctgtgtttgcaccaaaatagcaagctgtgtgctctatacactcttcccatcgtcttgtgtacactgctcctgtggccttccacagcagaaaccagggcaaaagggtccaaacacatggttttccttgctgcaaggctcttcctgggaactaagggggtatttattagttcagttctaagagacctccttctgggcttaccccactcctcaggtacttctctctccttcctccttctcctccacagtcacaagtaaccaaggaacctgaaagtggatgtgtagctatttgaagaaggcaaggaaccctgagattcttctttgaatcctctagtccaagtcttagaccagtgattggtgcttaccttgaacaaaattttgtctgtgttcctaatcccttcaatactctgggtacaatgctcccaatcaccctgcacatttgattctaaatggcttttattttttaaaaatccatatccctaggacaagagaacaggatgcctatatccccaaaatgagctccaggacactgatgggaatgatcccaaagatcaccccacctcagaaacgtctgtgccaagagacttccccagatagaaacactgggacagtggtttgaacgacttcttttatggttgtccagtttgctatggaaataaaaggcattgattttttaaaaagatgattggaacctgtctttggccacatagggccacttggatccatttccaggccttactcatatattgccttcactgaagggctttggctttaagtcccagactggtctcccaagtgaaccataagtgttttggagctcatctggggtgaggcatgagaatgttgccccatctatcccttcaggaaaaggtgccttccctccctttctcctaaagcctggtccccagaaattgtttttgtctccaaaagtctagtatggtctttatacacccagactcttagtgttgcgtcctgccttgtttccttgttaaggatctatgcagacctcccgctttggcttagctagcgtgacattggctatcatttgacaagactaacttttttttttttttttttttgactgagtctccctctgtcacctaggctggagtgcagtggcacaatcttggctcgctgcaaccttcacccttcacctcccaggtcgaagcgattctcctgcctcagtctcccgagtagctgggattacaggcgtgcgccaccaaatctggctatttttttattattattatttttagtagagatggggtttcaccatgttggccagactggtcttgaactcttggcctcaaattatctgcccacctcggcctcccaaagtgctgggattacaggcatgagccaccatgcccagctgacaagactaattttttatcccttggtttattggcttcaacatcttctggaatcagaggtgattttttcttaccttggatgcctgagactaggggagtatagaattccaattggtaattaaggcatctttctgctcctgatcagaagggcaggttagttgggagaggtcagatggcacaacagaagtcaccttgtaagtaaggcaaagacttgaaggcattagcgtttctcattactaggtcaataacctgagggaatcaatggctttttgccgctctacctcttgtgtatctctttgacttttctttctctgtctagtttcctctgttctcagtttatattctatgttatcagtctctctttccacagtacaaacatccatcctttctcctgtgcaattctgtctctccctcttattatctttatttgtactttttccttcctccctgtctaggcattgggcatgtgcctcttcttagcctgtgattttgccttgggactgatgataaattatttccagattcaatcagccctggtcctaccccagtccaatcagaagtatgttggtgggaatcaacctgatcctggccctttcttcttctccattttcattcgtaatccccctcagcagatctttacaagcagtttccttatagctcatgtatctttaggtctttgccttccaagcactgtacagaatactttgtggttcctttttagtctgacattttgtggagcagtgaagcgtgctcagagacataatcagctgaagagaaaaaatccacccatggatttatatcagctaaatactaataattgattttgtttgatgtgcccataatttttaaagctgcaatataatataatgagggaccacaggtaatttctcctgtcatttgttttggctggatgggggtgggggagtaattgcttaaagttttaccattacacattaaactctctataataatcttgtttggggcttgctaactgttgagctgttttaactaaactggtaggcaatcggagttgatttaaatgaaaagataatttaacaaatctatactataaaaagagacatttgcttaattgacatgtattttttccttctgagtcacctaaacatttactcttgacaccaactgttcatgatactgaatagacagtccatataagagaaattagtggacctaaagaagccagattgtaggtgttaatttattaaacagagtgcaaagcccttggaaatgtcactgcttggcaataccatatggaatgccaaaatttacaatgacttttctttataagttatccaaaagggatttgaacaagtaagaggttatgccaaaatgtctccaatgtatggtcctgtaatatattgcagcttgaagccaatgatcccttatgacttgtatacaactaatgcatgttttattgaattttgcatttcccacgtgtggtaagttctttaaaatgtttttgatcacctttttgtgccattaaacttgtacagaaaatgtttttatggccattttcaaagggagaaagtttaaaatggaaacagcccaccctttctgccctatagctgtagttagaattgagtacctgtagcaaaacagctgtaattggtggttgtagtgttagaggtgttagcttgctagtgactagctttggagagtaaatgcatggtattgtacatcacatttcttaactcgttttaacctctgaaaagaatatattcttctttgtagtccttcttcccacccccttgccctctccctctccctgctcccagttgtcttacagttgtaaatatctgatttgaggcccaataactcttgccaagtaaagtcagcaaacaacaaacaaaccaaaatgtggggaaaaggcatttctcaaccatctctcagcagttattgatcatttcttaaggaacagcattgtgatcaaagactcaactttacgtaaaaatcagtggtaaattggggttgtatttggccatttgattacatttcaggattgaatagttttcagaatcacatgtaatccaaagacagtaggtagtgatgtcccttatccctgcagctgttttaagatagagacctcagaagactctgcttgaccgatgaccaataattatttgaaaaaaaaagaaaaaatgagagaaataaaacagatatttaagaactttagccacctatttagaatagttatagccagaaaaaaaaacaagggcatgagttcaaatgcattactatcagtgtcctaggcaatacctaacctactctgaaattgtgattcaaaagcagtatttcaagaggcattctccttttttggtttgctgaccccacttggactggtaggtttggtgaggcccccataaaccagctggagcagacccttttcatctcctgtgcctgtaacacccctcttcccccaccccctccgcaattcaatgagggctttcttgggtcagaggacttcaaggttgtctagagaagtttgccatgtgtgtaaggtgctgtgaactgtgagtgctgaagattcgcagcattcaataccaggcagccaaagagctgctcttgcaattattttggctctcaagctctgttcttcatcgcattctcatttctgtgtacatttgcaagatgtgtgtaatgtcattttccaaaaataaaatttgatttcaataaaaaaaaaaaaaaaaaaaaaaaaaaaa

SEQ ID NO: 69 - protein, 456 aa, Homo sapiens
MRKSPGLSDCLWAWILLLSTLTGRSYGQPSLQDELKDNTTVFTRILDRLLDGYDNRLRPGLGERVTEVKTDIFVTSFGPVSDHDMEYTIDVFFRQSWKDERLKFKGPMTVLRLNNLMASKIWTPDTFFHNGKKSVAHNMTMPNKLLRITEDGTLLYTMRLTVRAECPMHLEDFPMDAHACPLKFGSYAYTRAEVVYEWTREPARSVVVAEDGSRLNQYDLLGQTVDSGIVQSSTGEYVVMTTHFHLKRKIGYFVIQTYLPCIMTVILSQVSFWLNRESVPARTVFGVTTVLTMTTLSISARNSLPKVAYATAMDWFIAVCYAFVFSALIEFATVNYFTKRGYAWDGKSVVPEKPKKVKDPLIKKNNTYAPTATSYTPNLARGDPGLATIAKSATIEPKEVKPETKPPEPKKTFNSVSKIDRLSRIAFPLLFGIFNLVYWATYLNREPQLKAPTPHQ

SEQ ID NO: 70 - DNA, 3440 bp, Homo sapiens
  source 1..3440: mol_type = unassigned DNA
agcggagcgggcgagcaagggagcgagcaggacaggagcctgatcccacagctgctgctccagcccgcgatgaggaaaagtccaggtctgtctgactgtctttgggcctggatcctccttctgagcacactgactggaagaagctatggacagccgtcattacaagatgaacttaaagacaataccactgtcttcaccaggattttggacagactcctagatggttatgacaatcgcctgagaccaggattgggagagcgtgtaaccgaagtgaagactgatatcttcgtcaccagtttcggacccgtttcagaccatgatatggaatatacaatagatgtatttttccgtcaaagctggaaggatgaaaggttaaaatttaaaggacctatgacagtcctccggttaaataacctaatggcaagtaaaatctggactccggacacatttttccacaatggaaagaagtcagtggcccacaacatgaccatgcccaacaaactcctgcggatcacagaggatggcaccttgctgtacaccatgaggctgacagtgagagctgaatgtccgatgcatttggaggacttccctatggatgcccatgcttgcccactaaaatttggaagttatgcttatacaagagcagaagttgtttatgaatggaccagagagccagcacgctcagtggttgtagcagaagatggatcacgtctaaaccagtatgaccttcttggacaaacagtagactctggaattgtccagtcaagtacaggagaatatgttgttatgaccactcatttccacttgaagagaaagattggctactttgttattcaaacatacctgccatgcataatgacagtgattctctcacaagtctccttctggctcaacagagagtctgtaccagcaagaactgtctttggagtaacaactgtgctcaccatgacaacattgagcatcagtgccagaaactccctccctaaggtggcttatgcaacagctatggattggtttattgccgtgtgctatgcctttgtgttctcagctctgattgagtttgccacagtaaactatttcactaagagaggttatgcatgggatggcaaaagtgtggttccagaaaagccaaagaaagtaaaggatcctcttattaagaaaaacaacacttacgctccaacagcaaccagctacacccctaatttggccaggggcgacccgggcttagccaccattgctaaaagtgcaaccatagaacctaaagaggtcaagcccgaaacaaaaccaccagaacccaagaaaacctttaacagtgtcagcaaaattgaccgactgtcaagaatagccttcccgctgctatttggaatctttaacttagtctactgggctacgtatttaaacagagagcctcagctaaaagcccccacaccacatcaatagatcttttactcacattctgttgttcagtcctctgcactgggaatttatttatgttctcaacgcagtaattcccatctgctttattgcctctgtcttaaagaatttgaaagtttccttattttcataattcatttaagaacaagagacccctgtctggcagtctggagcaaagcagactatgcagcttggagacaggattctgacagagcaagcgaaagagcaaagtcatgtcagaaggagacagaatgagagagaaaagagggggaagatggttcaaagatacaagaaaaagtagaaaaaaaaataacacttaactaaaacccctaggtcatttgtagatatatatttccaaatattctaaaaaagatactgtatatgtcaaaaatatttttatgtgaaggtgtttcaaagggtaaattataaatgtttcatgaagaaaaaattttaaaaatctacgtctttattacacaaactatggtgtgcttatgtttttgttttgctttttaaactgatgtatagctttaacattttgtttccaaagctgaagatccccattctttctctttgaaaaaaaaaaaggcctaatgcattattttgtcataaaatgctattttaaaattcatggaactttcatacgtaaaggtgcagttgctcattgtagagcacatttagtccaatgaagataaatgctttaaatagtttacttcactttcatctgagcttttaccactagactcaaggaagaataattttaacagacatgtatactccatagaaactaaattaaaatagtttaaaaatattccctttttcaccctattttcagatagcacatgagcccaacactcacttaattctcattatgaagatgtttttagaggggcaaaaatattttgcaagctctggaattgttgaatgtattcttttatataactacattaaaagctttagattgaaatttatgactagcaaacaaaaatagaatatataaacgatatatgtaaatatacagcatgagattgtacattttttacttttttaaaattgtgttcttaaaatattgtgtaagaatcactgcacttagctgttggaatgttgttaaatgctatggaaatacatttagaacctgcatttaagaacagaacagcaagtatgaaccacatggaacttaaaacatatgggtgtgaagtccacttatgtagacaaaacttataatttccaaactgttgtctagtatacagtgatcagttgctctctgttcaagtcattccacacatttccctattttaggctattataatatagaaagaaaatgggaagcattagttggagctagaaaatgaactgtatattattgctatatttgctaataccaactatttcaataagtgttgtaccatatgtagcattaaatataaaatacataaaagaatgtacagaaaatagcttttattgagtaatattacatttcatttatactgtagcaatatatttgtaggtatactatgtaagggctttaaataaaagaggtccattaatacttccttataaaaattctagtctgtttcattactgcccagatgttttagagataaatatttatgcagaaggtatttttgaagtctccttttgtctgatagagtttaacagatatttaaatttagtgctcagaatccacaagtcacggtctaaacacacttagaatactacagcataaatctgttagcattattgccaaataagacagttgggatccaaacccaagtcttgagcaatgtttttctcaaaaagctgctatccaatgatataggaaaatacattgtgttttcctaaacacacttttctttttaaatgtgcttcattgtttgatttggtcctgcctaaatttcacaagctaggccaatgaaggctgaatcaaagacatttcatccaccaatatcatgtgtagatattatgtatagaaaataaaataaattatggctccaaaaaaaaaaaaaaaa